SEQUENCE LISTING (416 sequences)

SEQ ID NO: 1 - protein, 365 aa, Caenorhabditis elegans
MDTEFAILDEEKYYDSVFKELNLKTRSELYEISSKFMPDSQFEAIKRRGISNRKRKIKETSENSNRMEQMALKIKNVGTELKIFKKKSILDNNLKSRKAAETALNVSIPSASASSEQIIEFQKSESLSNLMSNGMINNWVRCSGDKPGIIENSDGTKFYIPPKSTFHVGDVKDIEQYSRAHDLLFDLIIADPPWFSKSVKRKRTYQMDEEVLDCLDIPVILTHDALIAFWITNRIGIEEEMIERFDKWGMEVVATWKLLKITTQGDPVYDFDNQKHKVPFESLMLAKKKDSMRKFELPENFVFASVPMSVHSHKPPLLDLLRHFGIEFTEPLELFARSLLPSTHSVGYEPFLLQSEHVFTRNISL

SEQ ID NO: 2 - protein, 414 aa, Arabidopsis thaliana
MAKTDKLAQFLDSGIYESDEFNWFFLDTVRITNRSYTRFKVSPSAYYSRFFNSKQLNQHSSESNPKKRKRKQKNSSFHLPSVGEQASNLRHQEARLFLSKAHESFLKEIELLSLTKGLSDDNDDDDSSLLNKCCDDEVSFIELGGVWQAPFYEITLSFNLHCDNEGESCNEQRVFQVFNNLVVNEIGEEVEAEFSNRRYIMPRNSCFYMSDLHHIRNLVPAKSEEGYNLIVIDPPWENASAHQKSKYPTLPNQYFLSLPIKQLAHAEGALVALWVTNREKLLSFVEKELFPAWGIKYVATMYWLKVKPDGTLICDLDLVHHKPYEYLLLGYHFTELAGSEKRSDFKLLDKNQIIMSIPGDFSRKPPIGDILLKHTPGSQPARCLELFAREMAAGWTSWGNEPLHFQDSRYFLKV

SEQ ID NO: 3 - protein, 367 aa, Syncephalastrum racemosum
MIVASSDTCDIVDCEAAFGIDGTVRLRPGDFSLGTPYFTSRLGQKRPRPDDDTLDNTPSDTIHAIVQQLPVMAPDYWHDRPMEAVVMNAHVHFPSLVSLAEASLRFDPDNDEDEDNRQILRPDMALESLQVFYRHFEHPKDSPILIRVQDAYYWIPPRTAFMMGSLENIHLPTLGKFDCIVMDPPWPNKSVRRSAHYETQEDIYDLFAIPLPQLAQPNCLVAVWVTNKPKFIRFVQKLFAAWDVEPLTTWYWLKVTTHGEPVCPIDSPHRKPYEHLILGRKRPVKININDPPALPRVLVSVPSKHHSRKPPLNDILMRYLPSDARRLELFARCLTPGWTSWGNECLKFQHVDYFYDTNEAMEEGKQK

SEQ ID NO: 4 - protein, 269 aa, Hesseltinella vesiculosa
MANAARRFAQQDELPLDVSQDLQDLPLLDLFNRKVINDSDQCSSLHVASFGQYLVPRHTKFVMSDLDNIDLLRSENDVFDLIVMDPPWPNKSVHRSTDYETQDIYDLFHLPIKSLIKNQGLVAVWVTNKPKYRRFILDKLFKAWQMTCVGEWLWLKVTSSGEPVFPLDSPHRKPYEQLILGRYQPDDTSPTLPNPPQQHVLISVPSIRHSRKPPLGEVLADFLPKQPACLELFARCLTPGWTSWGNECLKFQHESYFISNDTPHSPSAS

SEQ ID NO: 5 - protein, 204 aa, Absidia repens
YDLVVMDPPWPNKSVHRSSHYETQDIYDLYQIPLTSLVHKNSLVAVWITNKPKYRRFVMDKLFKSWHVDCVAEWTWLKVTNDGEPVFPLNSTHRKPYEQLIIGRYNGGSGGGNDNNDSIQEESEVKPIPYQHSIVSVPSKRHSRKPPLQDLLQPYLPAKPRCLELFARCLTPGWSSWGNECLKFQNEYYYTRIENPLHIDRSDV

SEQ ID NO: 6 - protein, 268 aa, Lobosporangium transversale
MLHESTVSVLDRLILISHISLQTYLLAKDREGFDIIVMDPPWQNASVDRMSHYRTMDLYELFKIPIPDLLKANGSNVGGIVAVWITNKAKVKRVVVEKLFPAWGLDLVAHWFWLKVTTKGEPVLSLSNSHRRAYEGVLIGRQRQGSKLSNKTMHETSASNPVNRLLVSIPAQHSRKPSLNALIEEEFFTSKLESRADRDRNAYVDSEALVKKPLYRLELFARNLEEGVLSWGNEPLRYQYCGRGASNSQVVQDGYLIPCPIQSELVSQ

SEQ ID NO: 7 - protein, 450 aa, Danio rerio
MSVVCCNSWGWLLDSSSHIDKDFQRCVCYNEANGLEENTHFTCCFKRQYFNILMPHMQQSTAMSGFPLDSGKHDSAEHEKIELQTRKKRKRKHHDLNTGEIEANIYHDKVRSVVLEGSRALLEAGRQCGYFTEALTESQTISTPSESTSAHECQLAAFCDLAKQLPLSEESPVHTLSRDGQNPALDLFSSITENPFDCACEITFMRERYLLPPRCRFLLSDVTRMDPLVNSGDKFDLIVLDPPWENKSVKRSNRYSSLPSSQLKKLPVPALAAPGGLVVTWVTNRAKHRRFVREELYPHWAVEVLAEWLWVKVTRSGEFVFPLDSQHKKPYEVLVLGRCRSTSDHTDRCSAVNELPDQRLLVSVPSTLHSHKPSLAAVLKPYIRREPRCLELFARSLQSDWSCWGNEVLKFQHCSYFSRHTDQEPTSDTLQRTHSHLQSTGLLETPETAR

SEQ ID NO: 8 - protein, 472 aa, Homo sapiens
MSVVHQLSAGWLLDHLSFINKINYQLHQHHEPCCRKKEFTTSVHFESLQMDSVSSSGVCAAFIASDSSTKPENDDGGNYEMFTRKFVFRPELFDVTKPYITPAVHKECQQSNEKEDLMNGVKKEISISIIGKKRKRCVVFNQGELDAMEYHTKIRELILDGSLQLIQEGLKSGFLYPLFEKQDKGSKPITLPLDACSLSELCEMAKHLPSLNEMEHQTLQLVEEDTSVTEQDLFLRVVENNSSFTKVITLMGQKYLLPPKSSFLLSDISCMQPLLNYRKTFDVIVIDPPWQNKSVKRSNRYSYLSPLQIQQIPIPKLAAPNCLLVTWVTNRQKHLRFIKEELYPSWSVEVVAEWHWVKITNSGEFVFPLDSPHKKPYEGLILGRVQEKTALPLRNADVNVLPIPDHKLIVSVPCTLHSHKPPLAEVLKDYIKPDGEYLELFARNLQPGWTSWGNEVLKFQHVDYFIAVESGS

SEQ ID NO: 9 - protein, 470 aa, Sus scrofa
MSVVHQLSSGWLLDHLSFINKISYELHQHHEPCCSKNEPTSVHLDSLHKDSVFSFGASPAFIASSSKPENDDGGNREMSMQKYVFRSELFDVTKPYITSAIHKECQQSNEKEDLANDVKKEASISIKRKKRKRCVVFNQGELDAMEYHTKIRGLILDGSSQLIQEGLKSGFLHPLSEKCDKCSKPVTLPLDTCSLSELCEMAKHVPSLNEMELQTLQLMEDDISVTEQDLFSRIVENNSSFTKMITLMGQKYLLPPKSSFLLSDISCIYPLLNCRKTYDVIVIDPPWQNKSVKRSNRYSYLSPLQIKQIPIPKLAAPNCLVVTWVTNRQKHLRFVKEELYPSWSVEIVAEWHWVKITNSGEFVFPIDSPHKKPYEVLVLGRVRERAALLLSRNAEVKELSIPDHKLIVSVPCILHSHKPPLAEVLKDYIKPEGEYLELFARNLQPGWTSWGNEVLKFQHMDYFVALESRS

SEQ ID NO: 10 - protein, 453 aa, Mus musculus
MSVVHHLPPGWLLDHLSFINKVNYQLCQHQESFCSKNNPTSSVYMDSLQLDPGSPFGAPAMCFAPDFTTVSGNDDEGSCEVITEKYVFRSELFNVTKPYIVPAVHKERQQSNKNENLVTDYKQEVSVSVGKKRKRCIAFNQGELDAMEYHTKIRELILDGSSKLIQEGLRSGFLYPLVEKQDGSSGCITLPLDACNLSELCEMAKHLPSLNEMELQTLQLMGDDVSVIELDLSSQIIENNSSFSKMITLMGQKYLLPPQSSFLLSDISCMQPLLNCGKTFDAIVIDPPWENKSVKRSNRYSSLSPQQIKRMPIPKLAAADCLIVTWVTNRQKHLCFVKEELYPSWSVEVVAEWYWVKITNSGEFVFPLDSPHKKPYECLVLGRVKEKTPLALRNPDVRIPPVPDQKLIVSVPCVLHSHKPPLTGYLNSSFATLIPRVSNNMEYCRVVRTAFIA

SEQ ID NO: 11 - protein, 418 aa, Xenopus laevis
MSVVCETSAGWLVDELSLLRKWYQHSTSCQDAAHKKQLYDIKEDLFLILRPHIPVQSTPAPLPILCPETNPGTINQRKKRKRSCAFNQGELDAMEYHKKIIDFIMEGTQPLLQEGFKRLFLRPVLVNDDDHSQTEPRLCNNPCQLAELCNMAKCMPLLNPGEHAVQVLERGIYLPQETNVLSCITENKSECPEVIQFMGEKYIIPPKSTFLMSDVSCMEPLLHYKRYNIIVMDPPWENKSVKRSKRYSSLSPNEIQQLPVPVLAAPDCLVITWVTNKQKHLRFVKEDLYPHWSVKTLGEWHWVKITRSGEFVFPLDSTHKKPYEVLIIGRFKGAGNSTARKSEICLPPIPERKLIVSVPCKLHSHKPPLSEILKEYVKPDLECLELFARNLQPGWTSWGNEVLKFQHIDYFTPVDVED

SEQ ID NO: 12 - protein, 359 aa, Drosophila melanogaster
MLKLQKKTEDSKFAVFLDHKTLINEAYDEFKLKSELFQFHAKKTDKGIEEDKTRKRKRKAGVEDASSLEDLHLVNEYLELLSKPVEPEDSSPMKRHWEDGYNVPQLHGANESGRMQRFLRVDGSRGVYLIPNQSRFFNHNVDNLPALLHQLLPAYDLIVLDPPWRNKYIRRLKRAKPELGYSMLSNEQLSHIPLSKLTHPRSLVAIWCTNSTLHQLALEQQLLPSWNLRLLHKLRWYKLSTDHELIAPPQSDLTQKQPYEMLYVACRSDASENYGKDIQQTELIFSVPSIVHSHKPPLLSWLREHLLLDKDQLEPNCLELFARYLHPHFTSIGLEVLKLMDERLYEVRKVEHCNQEEVN

SEQ ID NO: 13 - protein, 331 aa, Chlamydomonas reinhardtii
MATLPGAAAAAPGANAEVGVPEPSLEPQDALQQRIALAEGLLALNEADAMQAWQQLPREALLEQVAKYRGAVRDMASALRSSTLPGGVPPHCVPIHANVTTFDWPSLYSHAQFDVIMMDPPWQLATANPTRGVALGYSQLNDDHISRLPVPQLQRQGGYLFVWVINAKYKWTLDLFDRWGYRLVDEVVWVKMTVNRRLAKSHGYYLQHAKEVCLVAKRGNPPVPPGCEGGVGSDIIFSERRGQSQKPEEIYHLIEQLVPNGRYLEIFARKNNLRNYWVSIGNEVTGTGLPDEDMQALRDLHHIPGAVYGKNAPHLVSKLFLYAPNSSREEG

SEQ ID NO: 14 - protein, 260 aa, Lobosporangium transversale
MLDQINIDIEQLEASLDIDEGKAHSNNASGTGCLIGTGTSSGNASNGAGVADEDLEEEVDDLEEFEAPEWCVPIKANVMTYDWDSLAAECQFDVILMDPPWQLATHAPTRGVAIAYQQLPDICIEELPVPKLSSNGFIFIWVINNKYAKAFDLMRRWGYSYVDDITWVKQTVNRRMAKGHGYYLQHAKETCLVGKKGEDPPGCRHSIGSDVIFSERRGQSQKPEELYELIEELVPNGRYLEIFGRKNNLRDYWVTVGNEL

SEQ ID NO: 15 - protein, 255 aa, Linderina pennispora
MDVDSSSPAVVLQALRQREQKIRSRILVLEQEISDLEKRCGVEGSGDAANKVTEADLEEFKAPEWSVPIRANVMNFDWEKLAQACQFDVILMDPPWQLASQAPTRGVAIAYQQLPDVCIESLPIDLLQTSGFIFIWVINNKYTKAFQLMKQWGYKYVDDIAWVKQTVNRRMAKGHGYYLQHAKETCLVGKKGPDPPNLRRSVASDVIFSERRGQSQKPEELYEIIEQLVPGGRYLEIFGRKNNLRDYWVTVGNEL

SEQ ID NO: 16 - protein, 752 aa, Basidiobolus meristosporus
MSAIIFTGNRVLFDSTSKVEPATIHVDPWTGRIVKITNKRSTKADFPGIEDKDFVDAGDDLIMPGVIDAHVHLNEPGRTDWEGFDTATRAAAAGGLTTVIDMPLNSIPPTTTLENLNTKKEAAKPQAWVDVGFYGGVIPGNADQLRPMIAAGVCGFKCFLIESGVDEFPCVNEEEVRKAFAEFDGTDNVFMFHAEMECDDHSHETAAPQSTDPSAYQTFLQSRPHALEVKAIEMIIRVCKDFPNVRAHIVHLSSAEALPMIRKAKAEGVKLTVETCYHYLTLNAEDIINGATHFKCCPPIREGSNRELLWEALLDGTIDYVVSDHSPCTPELKRFDSGDFTAAWGGISSLQFGLSLLWTEAKRRGCTLQDLTRWLSQNTARHAGILNRKGRLQIGSDADIVIWSPEETFVVDKKMIHFKNKVTPYENMTLHGAVKKTFVRGRNVYDKSTAQLFSAKPLGNLLARFQVYSNPITAMPSYAQPPSSDNGDFEEESEDYIESDEVDEDLRELLAKETSLRLRIDSLKEEILKLEREQRGETDGSKNEGEGGEEEIDLEEFEAPEWCVPIKANVMTFEWKRLAEAAQFDVILMDPPWQLATHAPTRGVAIGYQQLPDVCIEELPIPLLQKNGFIFIWVINNKYVKAFELMAKWGYRYVDDITWVKQTVNRRMAKGHGYYLQHAKETCLIGKKGEDPPNCRHSVCSDVIFSERRGQSQKPEELYEMIEQLVPNGKYLEIFGRKNNLRDYWVTIGNEL

SEQ ID NO: 17 - protein, 272 aa, Syncephalastrum racemosum
MSSREESPSSVSGFDLDTIDESTVTDTTLKNLLRREIELQLQIDALQTEILQIEESTAAGKNNKNDEELDPQDLEEFEAPEWCVPIKANVMTFDWEALASEVQFDVIVADPPWQLATHAPTRGVAIGYQQLPDVCIEEIPIQKLQKNGFIFIWVINNKYAKAFELMERWGYHYVDDITWVKQTVNRRMAKGHGYYLQHAKETCLVGKKGEDPPNCRHSVGSDVIFSERRGQSQKPEELYELIEELVPNGKYLEIFGRKNNLRDYWVTVGNEL

SEQ ID NO: 18 - protein, 342 aa, Absidia repens
MTSDTSAMTADVLNRKRKRSPAMNGDDLSNNSDEADNNTTTGTTTSVDSNENDYQEQDREPILRLPRLNDAKLLEEVVDDVDYEDQPERYDFDFKKLWLQERGLMERIDGLLKDIARLTDFKGHYRDMVIPSDDEDDLDDEDSKAQYDAPEWCVPIKANVMTFDWESLGKEVQFDVIMADPPWQLATHAPTRGVAISYQQLPDVCIEDLPLEKLQTNGFLFIWVINNKYAKAFEMMEKWGYKYVDDITWVKQTVNRRMAKGHGYYLQHAKETCLVGVKGTLPPYCRRSVGSDVIYSERRGQSQKPEQIYELIEEMMPGGKYLEIFGRKNNLRDYWITVGNEL

SEQ ID NO: 19 - protein, 334 aa, Hesseltinella vesiculosa
MASESNISRESSPASISSTNSESGIENVQSLTDEDLKQLILKEMNLKEHIEQLQRKISKLTANDLSTNQDSSDADDDLLNGDETMDDDSSSGSDSEVSGNEDIASVKSSPHAADKSESESESESDEGSSEDGNDEEDEFEAPKWCVPIKANVMTFDWEKLASETQFDVIVADPPWQLATHAPTRGVAIAYQQLPDVCIEDLPIEKLQTNGFIFIWVINNKYAKAFELMEKWGYTYVDDITWVKQTVNRRMAKGHGYYLQHAKETCLVGKKGVDPPSCRHSVGSDIIFSERRGQSQKPEELYELIEELVPNGKYLEIFGRKNNLRDYWVTVGNEL

SEQ ID NO: 20 - protein, 208 aa, Piromyces finnis
MMIVANEIDYEEFTAPEWCIPIKANVIDFEWDKLASECQFDAILMDPPWQLATHAPTRGVAIAYQQLPDQFIEELPIEKLQKNGFIFIWVINNKYVKAFELMKKWGYTFVDDITWVKQTVNRRMAKGHGYYLQHAKETCLVGKKGEDPVGCKHSISSDVIYSVRRGQSQKPEELYEMIEELIPNGKYLEIFGRKNNLRDYWVTIGNEL

SEQ ID NO: 21 - protein, 310 aa, Anaeromyces robustus
MDEKEVENSVLDSSNIEKSNATTSNMDVDETSNNETSTAIIKSEDGANSYDDFLKLDFTPEEEKDEVLKKLIERETELKLKIEKEIEGIKNLELKGFSALTQKDEDVQDIDYEEFTAPEWCIPIKANVIDFEWDKLASECQFDAILMDPPWQLATHAPTRGVAIAYQQLPDQFIEELPIEKLQKNGFIFIWVINNKYVKAFELMKKWGYTFVDDITWVKQTVNRRMAKGHGYYLQHAKETCLVGKKGDDPVGCRHKISSDVIYSVRRGQSQKPEELYEMIEELIPNGKYLEIFGRKNNLRDYWVTIGNEL

SEQ ID NO: 22 - protein, 428 aa, Tetrahymena thermophila
MKKEQQFLIFKKSLIIAQKRKEINIKQLKQQFKNFLFVQIFSIIKLKLQDIIIKFKMSKAVNKKGLRPRKSDSILDHIKNKLDQEFLEDNENGEQSDEDYDQKSLNKAKKPYKKRQTQNGSELVISQQKTKAKASANNKKSAKNSQKLDEEEKIVEEEDLSPQKNGAVSEDDQQQEASTQEDDYLDRLPKSKKGLQGLLQDIEKRILHYKQLFFKEQNEIANGKRSMVPDNSIPICSDVTKLNFQALIDAQMRHAGKMFDVIMMDPPWQLSSSQPSRGVAIAYDSLSDEKIQNMPIQSLQQDGFIFVWAINAKYRVTIKMIENWGYKLVDEITWVKKTVNGKIAKGHGFYLQHAKESCLIGVKGDVDNGRFKKNIASDVIFSERRGQSQKPEEIYQYINQLCPNGNYLEIFARRNNLHDNWVSIGNEL

SEQ ID NO: 23 - protein, 698 aa, Oxytricha trifallax
MNQSSQDITTQKSSNGFNPQTQPETLIQVIRKESTFIFKYRKNPYYVPPPISSQTSPNLEVETSNDLNQMSDYEGQIPNNYEINRNSTQFTNNDDQSDNDFYDNNSITTMQIDTSTAKILNNGPLEYNPDLPNKEQKLKDSQVMQNQPPTATSTNSQQRTLQELINIMPSIEDISQQCKQQQQLKIQAKANSTQSASTANAANGGKGRKRGRTVRFDQPLLGKVRQRNGDASDDEEPDEIEMLIRRLHTDILNDARNDPVEQAKKIRQARESQSDQTNSTTQLSVYERMILGSASQQSTDHQPGEFSNMFRTLEDEQIEINQNFLFDEYDSEDDSIADDKVEIASDDEQMLLQEHKKRGKKYLQDEIVKEEDFDEDDDSDEDIHMDDLENESLSFDRNNRKSHKPVCKRTREENILDADLGDEKDDEDTIFIDNLPSDEFSIRRQLQDVKSYIKQFEMLFFEEEDSDKEEQLKQITNVQKHEEALQNFKDRSHLKNFWCIPLSSDVREIDWDVLIARQQEHTNGQLFDVITCDPPWQLSSANPTRGVAIAYETLNDGEILKIPWGRLQKDGFLFIWVINAKYRFALDMMGAHGYRVVDEIQWVKQTCNGKIAKGHGYYLQHAKEVCLVGCKGDPAILAKKCRSNIESDVIFSERRGQSQKPEEIYELVEALVPNGYYMEIFGRRNNLHNGWVTVGNEL

SEQ ID NO: 24 - protein, 520 aa, Oxytricha trifallax
MHLPMQIITQNMFRQGNQHSCLNRTEILRTPRLTRSTKTELQEQTHFSKLPRRNYLKLQIDMREIQSLVDKKVKESAAAQQQLSQSGIEDSAIKRSLRPRKVENYKNMLEGDEITLKTIQDEQIEVKRKKREASSQNRLEDEDEDEDMLEVGQQIERASDDEDDDDFPISTRRSARKRTRRQDVDEDEEAIEVNQVESSDAEVEIPANDIDTESYTEGTNKRKQKLKAKKQVLDKKKNKTEGDIDKEDAVEEEETVFIDNLPNDEFEIRRMLKEVKKHIKSLEKQFFEEEDSEKEEELKQINNNSKHEEALQAFKETSHLKQFWCIPLSVNVTTLDFDLLAKSQMKQGGRLFDVITIDPPWQLSSANPTRGVAIAYDTLNDKEILNMPFEKVQTDGFLFIWVINAKYRFALEMMEKFGYKLVDEIAWVKQTVNGKIAKGHGYYLQHAKETCLVGVKGNVKGKARYNIESDVIFSQRRGQSQKPEEIYEIAEALVPNGYYLEIFGRRNNLHNGWVTIGNEL

SEQ ID NO: 25 - protein, 456 aa, Homo sapiens
MDSRLQEIRERQKLRRQLLAQQLGAESADSIGAVLNSKDEQREIAETRETCRASYDTSAPNAKRKYLDEGETDEDKMEEYKDELEMQQDEENLPYEEEIYKDSSTFLKGTQSLNPHNDYCQHFVDTGHRPQNFIRDVGLADRFEEYPKLRELIRLKDELIAKSNTPPMYLQADIEAFDIRELTPKFDVILLEPPLEEYYRETGITANEKCWTWDDIMKLEIDEIAAPRSFIFLWCGSGEGLDLGRVCLRKWGYRRCEDICWIKTNKNNPGKTKTLDPKAVFQRTKEHCLMGIKGTVKRSTDGDFIHANVDIDLIITEEPEIGNIEKPVEIFHIIEHFCLGRRRLHLFGRDSTIRPGWLTVGPTLTNSNYNAETYASYFSAPNSYLTGCTEEIERLRPKSPPPKSKSDRGGGAPRGGGRGGTSAGRGRERNRSNFRGERGGFRGGRGGAHRGGFPPR

SEQ ID NO: 26 - protein, 456 aa, Mus musculus
MDSRLQEIRERQKLRRQLLAQQLGAESADSIGAVLNSKDEQREIAETRETCRASYDTSAPNSKRKCLDEGETDEDKVEEYKDELEMQQEEENLPYEEEIYKDSSTFLKGTQSLNPHNDYCQHFVDTGHRPQNFIRDVGLADRFEEYPKLRELIRLKDELIAKSNTPPMYLQADIEAFDIRELTPKFDVILLEPPLEEYYRETGITANEKCWTWDDIMKLEIDEIAAPRSFIFLWCGSGEGLDLGRVCLRKWGYRRCEDICWIKTNKNNPGKTKTLDPKAVFQRTKEHCLMGIKGTVKRSTDGDFIHANVDIDLIITEEPEIGNIEKPVEIFHIIEHFCLGRRRLHLFGRDSTIRPGWLTVGPTLTNSNYNAETYASYFSAPNSYLTGCTEEIERLRPKSPPPKSKSDRGGGAPRGGGRGGTSAGRGRERNRSNFRGERGGFRGGRGGTHRGGFTPR

SEQ ID NO: 27 - protein, 456 aa, Sus scrofa
MDSRLQEIRERQKLRRQLLAQQLGAESADSIGAVLNSKDEQREIAETRETCRASYDTSTPNAKRKYQDEGETDEDKIEEYKDELEMQQEEENLPYEEEIYKDSSTFLKGTQSLNPHNDYCQHFVDTGHRPQNFIRDVGLADRFEEYPKLRELIRLKDELIAKSNTPPMYLQADIEAFDIRELTPKFDVILLEPPLEEYYRETGITANEKCWTWDDIMKLEIDEIAAPRSFIFLWCGSGEGLDLGRVCLRKWGYRRCEDICWIKTNKNNPGKTKTLDPKAVFQRTKEHCLMGIKGTVKRSTDGDFIHANVDIDLIITEEPEIGNIEKPVEIFHIIEHFCLGRRRLHLFGRDSTIRPGWLTVGPTLTNSNYNAETYASYFSAPNSYLTGCTEEIERLRPKSPPPKSKSDRGGGAPRGGGRGGTSAGRGRERNRSNFRGERGGFRGGRGGAHRGGFPPR

SEQ ID NO: 28 - protein, 481 aa, Xenopus laevis
MNSRLQEIRARQTLRRKLLAQQLGAESADSIGAVLNSKDEQREIAETRETSRASYDTSAAVSKRKLPEEGKADEEVVQECKDSVEPQKEEENLPYREEIYKDSSTFLKGTQSLNPHNDYCQHFVDTGHRPQNFIRDVGLADRFEEYPKLRELIRLKDELIAKSNTPPMYLQADLENFDLRELKSEFDVILLEPPLEEYFRETGIAANEKWWTWEDIMKLDIEGIAGSRAFVFLWCGSGEGLDFGRMCLRKWGFRRSEDICWIKTNKDNPGKTKTLDPKAIFQRTKEHCLMGIKGTVHRSTDGDFIHANVDIDLIITEEPEIGNIEKPVEIFHIIEHFCLGRRRLHLFGRDSTIRPDQSWEERLANSGGLREKEFLVGLLLGLLLPTATLIQRLMLLTLTLQIHLLLDAQRRSKDSVPKLHLLSQIVALGHREEEDEVEHLQVAERGAGKGTEAVLGETEGISEDVEDHIGVSLLPVDFKCF

SEQ ID NO: 29 - protein, 455 aa, Danio rerio
MNSRLQEIRERQKLRRQLLAQQLGAESPDSIGAVLNSKDEQKEIEETRETCRASFDISVPGAKRKCLNEGEDPEEDVEEQKEDVEPQHQEESGPYEEVYKDSSTFLKGTQSLNPHNDYCQHFVDTGHRPQNFIRDGGLADRFEEYPKQRELIRLKDELISATNTPPMYLQADPDTFDLRELKCKFDVILIEPPLEEYYRESGIIANERFWNWDDIMKLNIEEISSIRSFVFLWCGSGEGLDLGRMCLRKWGFRRCEDICWIKTNKNNPGKTKTLDPKAVFQRTKEHCLMGIKGTVRRSTDGDFIHANVDIDLIITEEPEMGNIEKPVEIFHIIEHFCLGRRRLHLFGRDSTIRPGWLTVGPTLTNSNFNIEVYSTHFSEPNSYLSGCTEEIERLRPKSPPPKSMAERGGGAPRGGRGGPAAGRGDRGRERNRPNFRGDRGGFRGRGGPHRGFPPR

SEQ ID NO: 30 - protein, 397 aa, Drosophila melanogaster
MSDVLKSSQERSRKRRLLLAQTLGLSSVDDLKKALGNAEDINSSRQLNSGGQREEEDGGASSSKKTPNEIIYRDSSTFLKGTQSSNPHNDYCQHFVDTGQRPQNFIRDVGLADRFEEYPKLRELIKLKDKLIQDTASAPMYLKADLKSLDVKTLGAKFDVILIEPPLEEYARAAPSVATVGGAPRVFWNWDDILNLDVGEIAAHRSFVFLWCGSSEGLDMGRNCLKKWGFRRCEDICWIRTNINKPGHSKQLEPKAVFQRTKEHCLMGIKGTVRRSTDGDFIHANVDIDLIISEEEEFGSFEKPIEIFHIIEHFCLGRRRLHLFGRDSSIRPGWLTVGPELTNSNFNSELYQTYFAEAPATGCTSRIELLRPKSPPPNSKVLRGRGRGFPRGRGRPR

SEQ ID NO: 31 - protein, 963 aa, Arabidopsis thaliana
MKKKQEESSLEKLSTWYQDGEQDGGDRSEKRRMSLKASDFESSSRSGGSKSKEDNKSVVDVEHQDRDSKRERDGRERTHGSSSDSSKRKRWDEAGGLVNDGDHKSSKLSDSRHDSGGERVSVSNEHGESRRDLKSDRSLKTSSRDEKSKSRGVKDDDRGSPLKKTSGKDGSEVVREVGRSNRSKTPDADYEKEKYSRKDERSRGRDDGWSDRDRDQEGLKDNWKRRHSSSGDKDQKDGDLLYDRGREREFPRQGRERSEGERSHGRLGGRKDGNRGEAVKALSSGGVSNENYDVIEIQTKPHDYVRGESGPNFARMTESGQQPPKKPSNNEEEWAHNQEGRQRSETFGFGSYGEDSRDEAGEASSDYSGAKARNQRGSTPGRTNFVQTPNRGYQTPQGTRGNRPLRGGKGRPAGGRENQQGAIPMPIMGSPFANLGMPPPSPIHSLTPGMSPIPGTSVTPVFMPPFAPTLIWPGARGVDGNMLPVPPVLSPLPPGPSGPRFPSIGTPPNPNMFFTPPGSDRGGPPNFPGSNISGQMGRGMPSDKTSGGWVPPRGGGPPGKAPSRGEQNDYSQNFVDTGMRPQNFIRELELTNVEDYPKLRELIQKKDEIVSNSASAPMYLKGDLHEVELSPELFGTKFDVILVDPPWEEYVHRAPGVSDSMEYWTFEDIINLKIEAIADTPSFLFLWVGDGVGLEQGRQCLKKWGFRRCEDICWVKTNKSNAAPTLRHDSRTVFQRSKEHCLMGIKGTVRRSTDGHIIHANIDTDVIIAEEPPYGSTQKPEDMYRIIEHFALGRRRLELFGEDHNIRAGWLTVGKGLSSSNFEPQAYVRNFADKEGKVWLGGGGRNPPPDAPHLVVTTPDIESLRPKSPMKNQQQQSYPSSLASANSSNRRTTGNSPQANPNVVVLHQEASGSNFSVPTTPHWVPPTAPAAAGPPPMDSFRVPEGGNNTRPPDDKSFDMYGFN

SEQ ID NO: 32 - protein, 545 aa, Chlamydomonas reinhardtii
MQDGQGPPGDGRGRGRGRSRGGRIMFAREGGRGPRPMHSDMGPPPPPMGMFPHDPSAMMGGPMPGMPPMDFTPEMLLTMMGAGLGGPMGLAGPMGMMMPDFGAAAAGAPGGMMVPPGAMMPPPPQPPSGGPGGMGGGGMGGMGGMMGHQQGMGGAGGPMGLPGGGMGMGMGGGGGGGGGGGYGGRGGHGEAGGGGGGGGRAGGAGGGGGAGGAAEHLSNDYSQNFVDTGLRPQNFLRDTHLTDRYEEYPKLKELIVRKDRQVSAHATPPLFLRTDLRSTRLSPELFGTKFDVILVDPPWEEYVRRAPGMVADPEVWSWQDIQALDIEAVADNPCFLFLWCGAEEGLEAGRVCMQKWGFRRVEDICWIKTNKEGGKGPGGGRRPYLTAANQHPESMLVHTKEHCLMGIKGSVRRATDGHIIHTNVDTDVIVSEEPELGSTRKPEEMYHIIERFCNGRRRLELFGEDHNIRNGWVTVGRSLTSSNFSAKAYADHFRNRDGSVWVQNTYGPKPPPGSVILVPTTDEIEDLRPKSPTGPHGGSSFHHSR

SEQ ID NO: 33 - protein, 392 aa, Tetrahymena thermophila
MQPQQNQNQQQQQQQQSQQQQQQNQQLPQLQQSMSSQQQQNQQQEKQIIIKRGTTSKRNDYCQNFVNTHERPQNFIMNIRPEERFIEYPKLQDLIKFKDDLIKKRNHPPVYLKADLKYYDLSKLGKFDVIMMDPPWKEYEERVQGLPIYSQYPEKFNSWDLNEIAALPIDEISDKPSFLFLWVGSDHLDQGRELFRKWGYKRCEDIVWVKTNKDKTKEYIELPHSNLLVRVKEHCLVGLRGDVKRASDSHFIHANIDTDVIVAEEPPLGSTQKPAEIYDIIERFCLGRKRLELFGEVHNVRQGWLTIGKLLDESNFNQDEYNSWFDGDKTYPQIQTYRGGRYVGTTPDIEQLRPKSPTKNNQMNSNQNMSGSQVSEFDLGIQQKQQKLNQQF

SEQ ID NO: 34 - protein, 335 aa, Saccharomyces cerevisiae
MAFQDPTYDQNKSRHINNSHLQGPNQETIEMKSKHVSFKPSRDFHTNDYSNNYIHGKSLPQQHVTNIENRVDGYPKLQKLFQAKAKQINQFATTPFGCKIGIDSIVPTLNHWIQNENLTFDVVMIGCLTENQFIYPILTQLPLDRLISKPGFLFIWANSQKINELTKLLNNEIWAKKFRRSEELVFVPIDKKSPFYPGLDQDDETLMEKMQWHCWMCITGTVRRSTDGHLIHCNVDTDLSIETKDTTNGAVPSHLYRIAENFSTATRRLHIIPARTGYETPVKVRPGWVIVSPDVMLDNFSPKRYKEEIANLGSNIPLKNEIELLRPRSPVQKAQ

SEQ ID NO: 35 - protein, 367 aa, Chlamydomonas reinhardtii
MRLGGGPGGSELDDLLGKRSVKEKVKVEKGSELLDILSKPTARESARVEQFRTAGGSAIREHCPHLTKDECRRVNGVPLACHRLHFLRVVQPHTDVALGNCSYLDTCRNMRTCKYVHYRPDPEPDVPGMGSEMARLRASVPKKPVGDGQTSRGALDPQWINCDVRSFDMTVLGKFGVIMADPPWEIHQDLPYGTMKDDEMVNLNVGCLQDNGVLFLWVTGRAMELARECMAKWGYKRVDELIWVKTNQLQRLIRTGRTGHWLNHSKEHCLVGIKGSPQLNRYVDTDVVVAEVRETSRKPDEMYSLLERLSPGTRKLEIFARVHNCKPGWVGLGNQLKNVNLIEPEVRQRFAARYGFEPDASKDCFVN

SEQ ID NO: 36 - protein, 685 aa, Arabidopsis thaliana
METESDDATITVVKDMRVRLENRIRTQHDAHLDLLSSLQSIVPDIVPSLDLSLKLISSFTNRPFVATPPLPEPKVEKKHHPIVKLGTQLQQLHGHDSKSMLVDSNQRDAEADGSSGSPMALVRAMVAECLLQRVPFSPTDSSTVLRKLENDQNARPAEKAALRDLGGECGPILAVETALKSMAEENGSVELEEFEVSGKPRIMVLAIDRTRLLKELPESFQGNNESNRVVETPNSIENATVSGGGFGVSGSGNFPRPEMWGGDPNMGFRPMMNAPRGMQMMGMHHPMGIMGRPPPFPLPLPLPVPSNQKLRSEEEDLKDVEALLSKKSFKEKQQSRTGEELLDLIHRPTAKEAATAAKFKSKGGSQVKYYCRYLTKEDCRLQSGSHIACNKRHFRRLIASHTDVSLGDCSFLDTCRHMKTCKYVHYELDMADAMMAGPDKALKPLRADYCSEAELGEAQWINCDIRSFRMDILGTFGVVMADPPWDIHMELPYGTMADDEMRTLNVPSLQTDGLIFLWVTGRAMELGRECLELWGYKRVEEIIWVKTNQLQRIIRTGRTGHWLNHSKEHCLVGIKGNPEVNRNIDTDVIVAEVRETSRKPDEMYAMLERIMPRARKLELFARMHNAHAGWLSLGNQLNGVRLINEGLRARFKASYPEIDVQPPSPPRASAMETDNEPMAIDSITA

SEQ ID NO: 37 - protein, 741 aa, Tetrahymena thermophila
MGSSVKDQEISNKKHKARNSSSGANNNSNSSNYQSSKRDIHQDRSYSKDDSQSRQYNSNNGGGGSSSKNSNRNSSQQGYNQNSSSNQGQNSEYGGSGSGKNSQANSQRNSSQQGLQQLNQQQQSQQQQQQMLQNQMNSMGMMNQFQNSFGLMGMQPSQPLQLLNPSMIIPSGKKQKYDFLEFPPSSQHEFRAILLDYFLSDLFDYPMHSAELFENFIEAFSDIKDSSSFIKKLELIPLLQELNDKKAIKLETCAVGTKLFDFIVDINKDKIKQLSREFSKDRPKFMPILDKKPQPSSSKTNSSSTTAPPKQAISKREIEDLLKKETGLQKEVITQSKEKSNLLNKISAAEESALAIFRKQGSRRIDYCDCGTRDKCIQIRNSTVPCNKAHFRKIIRPHTDENLGNCSYLDTCRHMDYCKFVHYELDVDINNMNNDNLLLDGIEKKLNPQWINCDLRQIDFNILGKFNCIMADPPWDIHMTLPYGTLKDREMKAMRVDLLQEEGVIFLWVTGRAMELGRECLTNWGYRRVEEIIWVKTNQLQRIIRTGRTGHWLNHSKEHCLVGIKGNPKINRKIDCDVIVSEVRETSRKPDEIYNLIERMCPGGKKIELFGRPHNTMPGWLTLGNQLPGIYLEDEEIIERYMDAYPDQDISRETMERNRIRMKNENDIDHIYNSHIQNIPPFKTKQLTKDLQLQQQSSSMQTTQQQSSSQMMPQMQQQQSSQSINSNTDLQMHGNGLYEQE

SEQ ID NO: 38 - protein, 453 aa, Basidiobolus meristosporus
MKLERALFKMADMWGYNTIGIKREYDNDKSAISVIYFDPRNLRNVQHIEKTLEDICDVDSIDPDIFLDKTTSAQVPSTYIPNEEARFSEDAEIEKLLSKPSFLEMEAFSSLIGVTELIERKTFREQEAEEMFKAQGNGGFREFCEYLIKEDCKKMNTSGQPCAMTASILLTNMKLHFRRIMRPQTDLELGDCSYLNTCHRMDTCKYVHYELDDFEHPSSANITKTTIPTSLIFRPPKKVLPAQWINCDVRKFDFSILGKFSVIMADPPWDIHMTLPYGTMTDDEMKAMAIHKLQDEGLIFLWVTARAMELGRECLATWGYDRVDEVVWIKTNQLQRLIRTGRTGHWLNHSKEHCLVGIKGDPSRFNIGLACDVLVAEVRETSRKPDQIYGMIDRLSPGTRKIEIFGRQHNTRPGWFTLGNQLKDVRIYEPEVLEAYNQRYPECPAQLSAIPES

SEQ ID NO: 39 - protein, 600 aa, Saccharomyces cerevisiae
MINDKLVHFLIQNYDDILRAPLSGQLKDVYSLYISGGYDDEMQKLRNDKDEVLQFEQFWNDLQDIIFATPQSIQFDQNLLVADRPEKIVYLDVFSLKILYNKFHAFYYTLKSSSSSCEEKVSSLTTKPEADSEKDQLLGRLLGVLNWDVNVSNQGLPREQLSNRLQNLLREKPSSFQLAKERAKYTTEVIEYIPICSDYSHASLLSTAVYIVNNKIVSLQWSKISACQENHPGLIECIQSKIHFIPNIKPQTDISLGDCSYLDTCHKLNMCRYIHYLQYIPSCLQERADRETAIENKRIRSNVSIPFYTLGNCSAHCIKKALPAQWIRCDVRKFDFRVLGKFSVVIADPAWNIHMNLPYGTCNDIELLGLPLHELQDEGIIFLWVTGRAIELGKESLNNWGYNVINEVSWIKTNQLGRTIVTGRTGHWLNHSKEHLLVGLKGNPKWINKHIDVDLIVSMTRETSRKPDELYGIAERLAGTHARKLEIFGRDHNTRPGWFTIGNQLTGNCIYEMDVERKYQEFMKSKTGTSHTGTKKIDKKQPSKLQQQHQQQYWNNMDMGSGKYYAEAKQNPMNQKHTPFESKQQQKQQFQTLNNLYFAQ

SEQ ID NO: 40 - protein, 608 aa, Drosophila melanogaster
MADAWDIKSLKTKRNTLREKLEKRKKERIEILSDIQEDLTNPKKELVEADLEVQKEVLQALSSCSLALPIVSTQVVEKIAGSSLEMVNFILGKLANQGAIVIRNVTIGTEAGCEIISVQPKELKEILEDTNDTCQQKEEEAKRKLEVDDVDQPQEKTIKLESTVARKESTSLDAPDDIMMLLSMPSTREKQSKQVGEEILELLTKPTAKERSVAEKFKSHGGAQVMEFCSHGTKVECLKAQQATAEMAAKKKQERRDEKELRPDVDAGENVTGKVPKTESAAEDGEIIAEVINNCEAESQESTDGSDTCSSETTDKCTKLHFKKIIQAHTDESLGDCSFLNTCFHMATCKYVHYEVDTLPHINTNKPTDVKTKLSLKRSVDSSCTLYPPQWIQCDLRFLDMTVLGKFAVVMADPPWDIHMELPYGTMSDDEMRALGVPALQDDGLIFLWVTGRAMELGRDCLKLWGYERVDELIWVKTNQLQRIIRTGRTGHWLNHGKEHCLVGMKGNPTNLNRGLDCDVIVAEVRATSHKPDEIYGIIERLSPGTRKIELFGRPHNIQPNWITLGNQLDGIRLVDPELITQFQKRYPDGNCMSPASANAASINGIQK

SEQ ID NO: 41 - protein, 573 aa, Xenopus laevis
MSDTWSSIQAHKKQLDNLRERLQRRRKDATSQLALDLQSSEGGIAPTFRSDSPVPSASSQPLKGPSGSAEVTPDPELEKKLLHHLSDLSLVLPADSVSIQLAITTPDFPVTRQGVESLLQKFAAQELIEVKGWGQEDDDRPTVVTFADYSKLSAMMGAVAERKGTTIPTGAKKRRLQEADPSASSLSSSLSASASREKKTSEPQKKARKHASHLDLEIESLLSQQSTKEQQSKKVSQEILELLSTSTAKEQSIVEKFRSRGRAQVQEFCDFGTKEECMKAAGADTPCRKLHFRRIINMHTDESLGDCSFLNTCFHMDTCKYVHYEIDAWVEPGGTAMGTEAIASLDTPLAKAVGDSSVGRLFPAQWIRCDIRYLDVSILGKFSVVMADPPWDIHMELPYGTLTDDEMRKLQIPVLQDDGFLFLWVTGRAMELGRECLKLWGYERVDEIIWVKTNQLQRIIRTGRTGHWLNHGKEHCLVGVKGSPQGFNRGLDCDVIVAEVRSTSHKPDEIYGMIERLSPGTRKIELFGRPHNIQPNWITLGNQLDGIHLLDPDVVAQFKQKYPDGVIGMPKNM

SEQ ID NO: 42 - protein, 580 aa, Homo sapiens
MSDTWSSIQAHKKQLDSLRERLQRRRKQDSGHLDLRNPEAALSPTFRSDSPVPTAPTSGGPKPSTASAVPELATDPELEKKLLHHLSDLALTLPTDAVSICLAISTPDAPATQDGVESLLQKFAAQELIEVKRGLLQDDAHPTLVTYADHSKLSAMMGAVAEKKGPGEVAGTVTGQKRRAEQDSTTVAAFASSLVSGLNSSASEPAKEPAKKSRKHAASDVDLEIESLLNQQSTKEQQSKKVSQEILELLNTTTAKEQSIVEKFRSRGRAQVQEFCDYGTKEECMKASDADRPCRKLHFRRIINKHTDESLGDCSFLNTCFHMDTCKYVHYEIDACMDSEAPGSKDHTPSQELALTQSVGGDSSADRLFPPQWICCDIRYLDVSILGKFAVVMADPPWDIHMELPYGTLTDDEMRRLNIPVLQDDGFLFLWVTGRAMELGRECLNLWGYERVDEIIWVKTNQLQRIIRTGRTGHWLNHGKEHCLVGVKGNPQGFNQGLDCDVIVAEVRSTSHKPDEIYGMIERLSPGTRKIELFGRPHNVQPNWITLGNQLDGIHLLDPDVVARFKQRYPDGIISKPKNL

SEQ ID NO: 43 - protein, 580 aa, Homo sapiens
MSDTWSSIQAHKKQLDSLRERLQRRRKQDSGHLDLRNPEAALSPTFRSDSPVPTAPTSSGPKPSTTSVAPELATDPELEKKLLHHLSDLALTLPTDAVSIRLAISTPDAPATQDGVESLLQKFAAQELIEVKRGLLQDDAHPTLVTYADHSKLSAMMGAVADKKGLGEVAGTIAGQKRRAEQDLTTVTTFASSLASGLASSASEPAKEPAKKSRKHAASDVDLEIESLLNQQSTKEQQSKKVSQEILELLNTTTAKEQSIVEKFRSRGRAQVQEFCDYGTKEECMKASDADRPCRKLHFRRIINKHTDESLGDCSFLNTCFHMDTCKYVHYEIDACVDSESPGSKEHMPSQELALTQSVGGDSSADRLFPPQWICCDIRYLDVSILGKFAVVMADPPWDIHMELPYGTLTDDEMRRLNIPVLQDDGFLFLWVTGRAMELGRECLNLWGYERVDEIIWVKTNQLQRIIRTGRTGHWLNHGKEHCLVGVKGNPQGFNQGLDCDVIVAEVRSTSHKPDEIYGMIERLSPGTRKIELFGRPHNVQPNWITLGNQLDGIHLLDPDVVARFKQRYPDGIISKPKNL

SEQ ID NO: 44 - protein, 580 aa, Sus scrofa
MSDTWSSIQAHKKQLDSLRERLRRRRKQDSGHLDLRNPEAALSPTFRSDSPVPTVPTSGGPKPSTASAVPELATDPELEKKLLHHLSDLALTLPTDAVSIRLAISTPDAPATQDGVESLLQKFAAQELIEVKRSLLQDDAHPTLVTYADHSKLSAMMGAVAEKKGPGEVAGTITGQKRRAEQDSTTVAAFASSLTSGLASSASEVAKEPTKKSRKHAASDVDLEIESLLNQQSTKEQQSKKVSQEILELLNTTTAKEQSIVEKFRSRGRAQVQEFCDYGTKEECMKASDADRPCRKLHFRRIINKHTDESLGDCSFLNTCFHMDTCKYVHYEIDACMDSEAPGSKDHTPSQELALTQSVGGDSNADRLFPPQWICCDIRYLDVSILGKFAVVMADPPWDIHMELPYGTLTDDEMRRLNIPVLQDDGFLFLWVTGRAMELGRECLNLWGYERVDEIIWVKTNQLQRIIRTGRTGHWLNHGKEHCLVGVKGNPQGFNQGLDCDVIVAEVRSTSHKPDEIYGMIERLSPGTRKIELFGRPHNVQPNWITLGNQLDGIHLLDPDVVARFKQRYPDGIISKPKNL

SEQ ID NO: 45 - protein, 212 aa, Afipia
MTLPAKDLLSFAGQRRFSTILADPPWQFTNKTGKVAPEHKRLSRYGTMKLDEIMMLPVADIAAPTSHLYLWCPNALLPEGLAVMKAWGFNYKSNIVWHKVRKDGGSDGRGVGFYFRNVTEVILFGVRGKNARTLAPGRRQVNLLATRKREHSRKPDEQYEIIESCSPGPFLELFARGTRKNWATWGNQADDDYKPTWKTYAHHSRAGLVAAE

SEQ ID NO: 46 - protein, 222 aa, Ethanoligenens harbinense
MSTAKETANNLLQFCGEKKYATVYADPPWRFQNRTGKVAPENKKLNRYPTMDLEDIKALPVGKIAAEKSHLYLWVPNALLPDGLEVMKAWGFEYKGNIIWEKVRKDGEPDGRGVGFYFRNVTEILLFGIRGGNNRTLAPARSQVNLIRTQKREHSRKPDEIITIIESCSPGPYLELFARGDRENWDMWGNQATAEYEPTWNTYKNHTTKETTSGVSGSQSET

SEQ ID NO: 47 - protein, 279 aa, Mycobacteroides abscessus
MAAPLREVNEPPPLPVTDGGFSTILADPPWRFTNRTGKVAPEHRRLDRYSTLSLDEICALGVSDVTADNAHLYLWVPNALLPDGLRVMEEWGFRYVSNIVWSKVRRDGLPDGRGVGFYFRNTTELLLFGVRGSMRTLQPARSQVNQIVTRKREHSRKPDEQYELIEACSPGPYLEMFGRYRRPNWAVWGDEANEDVEPRGQTHKGYGGGEITRLPALEPHSRIPQWLAKPIAAAIKSAYDDGMSIDAIAAETGYSISRVRHLLDQAGAKKRGRGRPAKA

SEQ ID NO: 48 - protein, 283 aa, Rothia
MLDPMNTNEEFAPLPTVEGGFQTVLADPPWRFTNRTGKVAPEHHRLGRYGTMSLDEIKALRVGDVTADNAHLYLWVPNALLPEGLEVMQAWGFRYVSNIIWAKRRKDGGPDGRGVGFYFRNVTEPILFGVKGSMRTLAPGRSTVNMIETRKREHSRKPDEQYDLIEACSPGPYLELFARYARPGWSVWGNEASNEIEPRGKAQKGYGGGEIDRLPILEPNERMSEWLSGRVGELLAEEYTKGASVQELANQSGYSIARVRTLLTHSGVPLRGRGRPKKGQVAS

SEQ ID NO: 49 - protein, 198 aa, Candidatus Entotheonella factor
MSNSPHSAADDLLACGFPPHSFSTVLADPPWRFTNRTGKMAPEHRRLSRYPTLTLEEIADLPLAQLVQPDSHLYLWVPNALLAEGLDVMRRWGFTYKTNLVWYKIRRDGGPDRRGVGFYFRNVTELVLFGVRGRMRTLAPGRRQENLLASQKQEHSRKPDTFYDLIERCSPGPYLELFARHPRPGWHQFGNEPLVSSS

SEQ ID NO: 50 - protein, 215 aa, Granulibacter bethesdensis
MTKQPDPIAEFRNQLNGGNFATVLADPPWRFQNRTGKMAPEHRRLSRYGTMELPEIMALPVSEVTAKTAHLYLWVPNALLPEGLAVMQAWGFNYKSNLVWHKIRKDGGSDGRGVGFYFRNVTELVLFGVKGKNARTEAPGRRQVNLLATQKREHSRKPDEFYDIVEACSPGPYLELFARGTRPGWCAWGNQAEEYDITWDTYSHHSQRQSLWVAE

SEQ ID NO: 51 - protein, 216 aa, Methylococcus capsulatus
MTENTLDPAADLLERLGDKRFRTILADPPWQFQNRTGKMAPEHKRLNRYGTMSLEAIAGLPVERLTADTAHLYLWVPNALLLEGLKVMEAWGFTYKTNLVWHKIRKDGGPDGRGVGFYFRNVTELVLFGVRGKNARTLAAGRRQVNFLATRKREHSRKPDEMYGIIEACSPGPYLELFARGARDRWSVWGNEADENYYPRWNTYANHSQAEICPFE

SEQ ID NO: 52 - protein, 230 aa, Xylella fastidiosa
MTKHKANTASDVGRDLLARHGGQRFHTILADPPWQFQNRTGKMAPEHKRLSRYGTMTLDDIMMLPVEQLVTDTAHLYLWVPNALLPEGIKVLEAWGFSYKSNIVWHKVRKDGGPDGRGVGFYFRNVTELVLFGVRGKNARTLAPGRRQVNFLATQKREHSRKPDEFYDIVESCSPGPFLELFARGPRDGWKVWGNQADKYYPTWPTYSNHSQAECELGRVEMIAQRLLSV

SEQ ID NO: 53 - protein, 220 aa, Rhizobium undicola
MLNRNTDAPSPSDDFTNFISGRKFATIMADPPWQFMNRTGKVAPEHKRLNRYGTMELDAIKALPVATACAPTAHLYLWVPNALLPEGLEVMKAWGFNYKANIVWHKLRKDGGSDGRGVGFYFRNVTELILFGTRGKNARTLPPGRSQVNYIGTRKREHSRKPDEQYPLIESCSPGPYLEMFGRGLRKGWTTWGNQADETYEPTWKTYGHNSSTDRLEAAE

SEQ ID NO: 54 - protein, 220 aa, Escherichia coli
MGWFMTKKYTLIYADPPWVYRDKAADGNRGAGFKYPVMSVLDICRLPVWDLADENCLLAMWWVPTQPLEALKVVEAWGFRLMTMKGFTWIKCGSRQPDKLVMGMGHMTRANSEDCLFAVKGKLPTRINAGIVQSFTAPRLEHSRKPDIVREKLVQLLGDVSRIELFARQTSHGFDVWGNQCEDPAVQLHPGYALDIGGLTNAFSNAPLSPTDIQGRERAA

SEQ ID NO: 55 - protein, 208 aa, Escherichia coli
MTKKYTLIYADPPWTFRDKATDGQRGASFKYPVMSLLDICRLPVWELAADNCLLAMWWVPTQPLEALKVVEAWGFRLVTMKGLTWNKCGKRQTDKLVMGMGSTTRANSEDCLFAVKGNLPERINAGIIQSFTAPRLDHSRKPDMAREKLVQLLGDVPRIELFARHTSHGFDVWGNQCGTPSIEMVPGIVKFLEKTNERKNDVDKGITS

SEQ ID NO: 56 - protein, 197 aa, Klebsiella aerogenes
MTGKYTLIYADPPWSYRDKAADGDRGAGFKYPVMNVMDICRLPVWELSADDCLLAMWWVPTQPVEALKVVEAWGFRLMTMKGFTWHKINKHKGNSAIGMGHMTRANSEDCLFAVRGKLPERMDASICQHVTAPRLENSRKPDVIREKLVQLLGDVPRIELFARQSSHGFDVWGNQCIAPAVELLPGCAVPVVKTEAA

SEQ ID NO: 57 - protein, 214 aa, Klebsiella pneumoniae subsp. pneumoniae KPNIH27
MNYDLIYCDPPWEYGNRISNGAACNHYSTMSIDDLKFLPVRKLAADNAVLAMWYTGTHNREAVELAESWGFRVRTMKGFTWVKLNQNAADRFNKALSTGELVDFNDLLEMLDRETRMNGGNHTRSNTEDVLIATRGTGLPRASASVKQVVHTCLGEHSAKPWEVRNRLEQLYGDVKRIELFAREEWKGWDRWGNQCNNSIEIITGLIKEVNHAA

SEQ ID NO: 58 - protein, 208 aa, Clostridioides difficile
MPAVLFLLELHRRRKGGYKIENNQKYNIIYADPPWRYQQKRLSGAAEHHYPTMSVKDICGLKVEEIAAKDCVLFLWATFPQLPEALRVIKAWGFQYKTVAFVWLKQNKSGKGWFFGLGFWTRGNAEICLLAIKGKPHRNSNRVHQFLISPIRGHSQKPEEAREKIVELMGDLPRVELFAREKTEGWDAWGNEVESDIEISSDTEKEWR

SEQ ID NO: 59 - protein, 194 aa, Xanthobacter autotrophicus
MNGLWQFGDLKMFGYDLIVADPPWDFELYSEAGEGKSAKAHYGTMKLDEIAALRVGDLARGDCLLLLWCCEWMPPAARQRVLDAWGFTYKTTIIWRKVTRAGKVRMGPGYRARTMHEPVIVATVGNPKHTPFSSVFDGVAREHSRKPEAFYRMVEAAAPKAARADLFSRQRRDGWDAFGNEVEKFDQPPAEAAE

SEQ ID NO: 60 - protein, 190 aa, Devosia riboflavina
MTAWPFGAMPMFSFDVVMADPPWSFDNWSEGGNAKNAKAQYDCMPTPDIKRLPVGHLAAGDCWLWLWATYPMLPDAIEVMDAWGFRYVTAGPWVKRGTSGKLAMGTGYVLRSCSEIFLIGKNGEPKTHARDVRNVLEAPRREHSRKPDEAYAMAEKLFGPGRRADLFSRETRPGWTSWGNESTKFDEVAA

SEQ ID NO: 61 - protein, 193 aa, Rhizobium phaseoli
MRLFPDLWPFGDLQPHSFDFIMADPPWKMQEWSDNGDKSKSTQSKYRLMPLDEIKAMPVLDLAAPNCLLWLWATNPMLPQALDVLHAWGFTFATAGSWMKTTRNGKQAFGTGYIFRTSNEPILIGKRGEPKTTRSVRSSFPGLAREHSRKPEEGYREAERLMPRARRLELFSRTNRVGWTTWGDEVGKFGDVA

SEQ ID NO: 62 - protein, 190 aa, Nitratireductor basaltis
MHLFDWPFGDLNPHSFDLIMADPPWAFELRSDKGEGKSAQSHYKCQTLDEIKALPVLDLAAPDCLLWLWATNPMLPQAFEVMAAWGFTFKTAGAWGKTTVNGKLAFGTGYIFRSAHEPILIGTRGEPRTTKSVRSLIMGQVREHSRKPEEAYAAAEKLIPNARRLELFSRTDRAGWEVWGDEAGKFGEAA

SEQ ID NO: 63 - protein, 360 aa, Tetrahymena thermophila
MSLKKGKFQHNQSKSLWNYTLSPGWREEEVKILKSALQLFGIGKWKKIMESGCLPGKSIGQIYMQTQRLLGQQSLGDFMGLQIDLEAVFNQNMKKQDVLRKNNCIINTGDNPTKEERKRRIEQNRKIYGLSAKQIAEIKLPKVKKHAPQYMTLEDIENEKFTNLEILTHLYNLKAEIVRRLAEQGETIAQPSIIKSLNNLNHNLEQNQNSNSSTETKVTLEQSGKKKYKVLAIEETELQNGPIATNSQKKSINGKRKNNRKINSDSEGNEEDISLEDIDSQESEINSEEIVEDDEEDEQIEEPSKIKKRKKNPEQESEEDDIEEDQEEDELVVNEEEIFEDDDDDEDNQDSSEDDDDDED

SEQ ID NO: 64 - protein, 328 aa, Oxytricha trifallax
MSSSISAAIIAGNQNKKIAESKSLWNYALSPGWTQQEVEILKIALMKFGVGRWKTIEQSQCLPTKTMSQMYLQTQRLVGQQSLAEFMGLHLDLEQIFIKNAERQGAGVFRKNGCIINTGDNMTKVQIAKLRKKNSKIFGLTQPFVQSLHLPKAKVKEWLKVLTLDQILSAKSNFSTAEKIHYLKILENALERKLKKILRLQELVSIYRPCNIGIVVQKRLGSSIGDEYFEYVDCVKIEEKSVGNLDFALPNRNTDSTSLNEDFSFLDSTQKPQKLKAGSGRENKRKKMRDGLKDERAQRQSLMEALDEQEFDETKFQDSDGEMPDLNM

SEQ ID NO: 65 - protein, 229 aa, Oxytricha trifallax
MSVHHKMADSKSLHNYTLSPGWTREEVDILKIALMKFGIGKWKKIQKSGCLPSKTISQMNLQTQRLLGQQSLAEFMGLHVYLDRVFRDNSLKTGPEIQRKNNFIINTGNNLTQPEKEKRLRLNKQKYGLDLAFIKTLRLPKPESATGGKREAILSMDQIFAQKSHFTVVEKLKHLEALKNALCSKLGKIERRRRNKELSKIYRPLGQLIVVQKNADDQYEFVDIIDENE

SEQ ID NO: 66 - protein, 206 aa, Linderina pennispora
MSSATPYAPRSMPTGQRNVVRSNDSASLWNCTLSPGWTQEEVQVLRKALMKFGVGNWMKIIESECLPGKTIAQMNLQTQRMLGQQSTAEFNGLHLDAFVIGELNSKKQGPGIKRKNNCIVNTGGKLTRDEVVKRQQKHREQYEVKAEVWRAIVLPKPDNPLILLEKKREELKKVRLELEEIMKQIEETEKLVDVPEHAPGTKRARE

SEQ ID NO: 67 - protein, 216 aa, Basidiobolus meristosporus
MTDVYKPRSMPVGARNVLRSNDSASLWNCTLSPGWTEPEVHILRKAVMKFGIGNWAKIIESQCLFGKTIAQMNLQLQRMLGQQSTAEFAGLHLDPFVIGEINSKKQGPGIKRKNNCIVNTGGKLTREEIKRRLLEHKRTYEISEEEWRSIELPKPEDPGAVLIAKKDELKMLEDELLRVVQKIQKAREERRSKSVDSSSVDGSVDDEARETKRRRK

SEQ ID NO: 68 - protein, 440 aa, Oxytricha trifallax
MSHATSHGNSTEKDKKNSGNMVAESKSLWNYALSPGWTPQEVDVLKIALMKFGIGKWTIIDKSGILPTKTIQQCYLQTQRILGQQSLAEFMGLHVDIDKIALDNRRKNGIRKMGFLVNQGGKLTPEEKAHYQEINRQKYGLSPEEVETIKLPPPCSVEIYDINKIINPKSKLTTIEKINHCIKLQDALLEKLENIKNKKIPTGAGFSSSRVYENMRGYDPQLLLNSHVTGQLDHSMQDLTIDERYSDLDEEEDPLAMASIIDSQATPQPQKIKSSVPNKASTTPSAKEMNQIKDIIDSVIAENSAQQSKNLAQEKPKLKFSLVKATESNLLQSAAQNSDDVVMEEDSKLQHIETFSTVTQTATDQSNSQSKSQNNIASDSLKDSLEQNDLSKSLTDSLEMQQYSAEKKLNQAPMSKNSDKPKKKRLNKRKLPSDDEFETL

SEQ ID NO: 69 - protein, 226 aa, Lobosporangium transversale
MSSGSTPRSMTAGARNILRSNDSASLWNYTVAPGWSMKEAEILRKALMKFGIGNWSKIIESNCLVGKTNAQMNLQTQRMLGQQSTAEFAGLHIDPRVIGQKNSLIQGDHIRRKNGCIVNTGAKLSREEIRRRVAENKEQYELPEEEWSSIELPLPDDPHLLLEAKKSEKVRLELELKNVQRQIAMLRKVGRKFETGSESPKTELDDDERDEFIEDQPLGKRARIEA

SEQ ID NO: 70 - protein, 426 aa, Oxytricha trifallax
MSSSISAAIMAGNQNKKIAESKSLWNYALSPGWTQQEVEILKIALMKFGVGRWSAINKSGVLPTKQIQQCYLQTQRLIGQQSLAEFMGLHLDIDRIAADNKQKRGIRKQGFLVNQGCKLTPEEKDELRKINQEKYGLSAEHVEAIKLPAPCHLVEIFQIDKIMHPRSTLSTMDKIKHLIKLEDALKSKLEMIREGKRQQKFEQLQQKLKTTEASGRGSVTRVQRQMSDLHLGSSHQNRNSDLDEENDESVMIIDESQQENLTPKGKAQAMLTHQKYNEVTQTMIKQGDDSRQQQHLPLDSTSASVSNPSSTSKSSTMKSNSMKQSETAIASMKPSSIGKKTKVDSSFVTKQSNQQSTAPIQKQAHQQNLDRNRSELGSTFAQQASVDTQNSNNQGTSTASGNFISQSDDEEALMPKLKRRRVEDSE

SEQ ID NO: 71 - protein, 417 aa, Oxytricha trifallax
MRVYLKFCNRKQIHYTHTMSSSISAAIMAGNQNKKIAESKSLWNYALSPGWTQQEVEILKIALMKFGVGRWSAINKSGVLPTKQIQQCYLQTQRLIGQQSLAEFMGLHLDIDRIAADNKQKRGIRKQGFLVNQGCKLTPEEKDELRKINQEKYGLTAEHVEAIKLPAPCHLVEIFQIDKIMHPRSTLSTMDKIKHLIKLEDALKSKLEMIREGKRQQKFEQLQQKLKTTEASGRGSVTRVQRQMSDLHLGSAHQNRNSDLDEENDQSVMIIDESQQQNLTPKGKAQTMLTNQTQTMKKQADDSRDEQHLPLISTSASVSNPSSTSKSSALKLNSMKQSDTAIASMKPSSSGKKTKVDSSFVSKQSNQQSTSYSETNVDTQNSNNQGTSTASGNFISQSDDEEALMPKLKRRRVEDSE

SEQ ID NO: 72 - protein, 439 aa, Oxytricha trifallax
MRVYLKFCNRKQIHYTHTMSSSISAAIMAGNQNKKIAESKSLWNYALSPGWTQQEVEILKIALMKFGVGRWSAINKSGVLPTKQIQQCYLQTQRLIGQQSLAEFMGLHLDIDRIAADNKQKRGIRKQGFLVNQGCKLTPEEKDELRKINQEKYGLTAEHVEAIKLPAPCHLVEIFQIDKIMHPRSTLSTMDKIKHLIKLEDALKSKLEMIREGKRQQKFEQLQQKLKTTEASGRGSVTRVQRQMSDLHLGSAHQNRNSDLDEENDQSVMIIDESQQQNLTPKGKAQTMLTNQTQTMKKQADDSREEQHLPLNSTSASVSNPSSTSKSSALKLNSMKQSDTAIASMKPSSSGKKTKVDSSFVSKQSNQQSTGPIQKQAHQQNLDRNRSELGSTFAQQTNVDTQNSNNQGTSTASGNFISQSDDEEALMPKLKRRRVKDSE

SEQ ID NO: 73 - protein, 305 aa, Piromyces finnis
MSIPKPRSMPVGFRNILRPNDSTSLWNCTLSPGWTQEESDILRDALIFYGIGNWKDIIEHGCLPDKTNAQMNLQLQRMLGQQSTAEFQNLHIDPYEIGKINSQKQGPNIRRKNGFIINTGGKLSREDIKRKIQENKENYELPEEVWSKIVLPNREVVTINEKRQKLNKLEEELDSVLKQIVNRRRELRGMTPLKETEMKSIVNRSNQNDTKTEEKEIKEEESTTVNEEKIENTETSSISIISTNENEQSENISSSSPIVKSEQKKKRVVSRRKNKRRVNSDDEDFLPPGKSRSKRTRRTPKKSSN

SEQ ID NO: 74 - protein, 311 aa, Anaeromyces robustus
MSIPKPRSMPTGFRNILRPNDSTSLWNCTLSPGWTQEESDILRDALIYYGIGNWKDIIEHGCLPDKTNAQMNLQLQRMLGQQSTAEFQNLHIDPYVIGKINSQKQGPNIRRKNGFIINTGGKLSREDIRRKIQENKENYELPKEEWSKIVLPNREVVIKNKVQEAINEKREKLNKLEDELDSVLKAIVNRRRELRGMIPLKDSEMKSLVNRSAKNEGENKTETTNNEESNNTNNSDDIKDENNETSTSSHIFTNNDNELSENNSSSSSSNSISNKKKRFLRREVRRGKRRYNYDDDDFMPSGNRSRKSRKI

SEQ ID NO: 75 - protein, 224 aa, Syncephalastrum racemosum
MSNNKENNVNKPRSMTAGARNVLRSNDSTSLWNCTLSPGWTQDESEVLRKALMKFGVGNWAKIIESGCLPGKTNAQMNLQLQRLLGQQSTAEFAGLHIDPKVIGEKNSKIQGPHIKRKNNCIVNTGDKLSRDKLRARVMSNKEEYELPEEVWKNIELPKVKDPLMLLEGKKEEMRKLKTELEKVQAKIQQLRQAQPARVQELQSQIEVARSPSPSAPDSPALSV

SEQ ID NO: 76 - protein, 458 aa, Chlamydomonas reinhardtii
MAFAAALAEKRGPRVGDAASLWNFTPAPGWSREEVQILRLCLMKHGVGQWMQILSTGLLPGKLIQQLNGQTQRLLGQQSLAAYTGLKVDVDRIRVDNETRTDATRKAGLIINDGPNLTKEMKEKMRQDAVAKYGLTPEQVAEVDEQLAEIAAAFNPASTSAAAGAGSGAAAAGQAAAAGSGAGGSGNLMAQPTEQLSAEQLGQLLLRLRNRLACLVDRARGRAGLPPRTAPRWATEAAAAACLAAMAAAEASAPQAPAAAAGGQEGAAGPVMVSVPFSREVLAEATACRVRSGTAAGARGNAPGAQGGVRKRTSKGGKAKGGDREWSPEGEENTAPQPRGGGKRKSGAVAGGEEADGVASGRAKRASRPKRGSSKHDPYVDDNDYGDEGIDPFDVGDDLDDMNPHGRYGNGGGRRADPSEAISALTAMGFTQSKARGALRECNFNVELAVEWLFANCL

SEQ ID NO: 77 - protein, 507 aa, Chlamydomonas reinhardtii
MAFAAALAEKRGPRVGDAASLWNFTPAPGWSREEVQILRLCLMKHGVGQWMQILSTGLLPGKLIQQLNGQTQRLLGQQSLAAYTGLKVDVDRIRVDNETRTDATRKAGLIINDGPNLTKEMKEKMRQDAVAKYGLTPEQVAEVDEQLAEIAAAFNPASTSAAAGAGSGAAAAGQAAAAGSGAGGSGQAATAADAGGAAGRGTGSAGGAAAAAPPRNALAISTGVLAATLLDASLGNLMAQPTEQLSAEQLGQLLLRLRNRLACLVDRARGRAGLPPRTAPRWATEAAAAACLAAMAAAEASAPQAPAAAAGGQEGAAGPVMVSVPFSREVLAEATACRVRSGTAAGARGNAPGAQGGVRKRTSKGGKAKGGDREWSPEGEENTAPQPRGGGKRKSGAVAGGEEADGVASGRAKRASRPKRGSSKHDPYVDDNDYGDEGIDPFDVGDDLDDMNPHGRYGNGGGRRADPSEAISALTAMGFTQSKARGALRECNFNVELAVEWLFANCL

SEQ ID NO: 78 - protein, 288 aa, Absidia repens
MSSPSSPSPIKPRSMLTGSRNVVRSNDSASLWNCTLSPGWNEEQSETLRHAVMKYGIGNWAKIIDSGYLPGKTNAQMNLQLQRLLGQQSTAEFAGLHIDPKVIGEQNSRIQGPEIRRKNNTIVNTGDKLSREALRERILRNKEKYELPESVWQAIELEHVTDEDALLEEKKKTLREMKSQLKVVQRQIKNLEFMHPLHAAKLKFELEKLAPSSSTSSSSSSPSPSSSSSPSSSSSKPSVSGTEEEMREAVDEERGSDEEIDELVEETDEEETSVSPKVGTRTKKVRTN

SEQ ID NO: 79 - protein, 245 aa, Hesseltinella vesiculosa
MIANSTATPKPRSMKAGARNVLRSNDSASLWNCTLSPGWTEQESEILRQLAIKFGIGNWAKIIESDCLPGKTNAQMNLQLQRLLGQQSTAEFAGLHIDPKVIGEKNSKIQGPHIKRKNTTIVNTGGKLSREELRERQAKNKEMYEMPKSAWDSIDLDELRDMNSLKLKKKEDKDALKKQKLTQLKTKLTKSQNNLKKVQAELKQIAMVDPERVAELKKELSRASSPLSNEVSVIEESPAKKQRTS

SEQ ID NO: 80 - protein, 174 aa, Piromyces finnis
MVVEKDLAQENKIKEELNKKHEWVKEMRKKFCVRKEFENTKNLILEDGTLNQEYFRLSKGTVLKTNEVRKWTSIERNLLIKGIEKYGIGHFREISESLLPKWSGNDLRIKTIHLIGRQNLKLYKDWKGGEEDIKREYNRNKEIGLKCNAWKNNCLIDDGNGKVKEMIEATEPKH

SEQ ID NO: 81 - protein, 175 aa, Anaeromyces robustus
MVVEKETNKENIKNIKEELDKKHAWVKEMRKKFCVRKEFENTKILILEDGTLNQDYFRLSKGTVLKTNEVRKWTSIERGLLIKGIEKYGIGHFREISENLLPKWSGNDLRIKTIHLIGRQNLKLYKDWKGNEEDIKREYNRNKEIGLKCNAWKNNCLVDDGHGKVKAMIEATENN

SEQ ID NO: 82 - protein, 199 aa, Syncephalastrum racemosum
MMTATDEDVDMKDVDIKLESNQETEQKILTPEEQKEKEKQDWIRQLRLKFCIRPEYEITKNMIFPDGTLNQDYFRPPKGAKVEEARKWTEVEKELLIQGIEKYGIGNFGEVSKALLPAWSTNDLRIKCIRLIGRQNLQLYRGWKGNADDIAREYNRNKELGLKYGTWKQGVLVYDDDGLVEKEILAQDAAAKGEDVDMN

SEQ ID NO: 83 - protein, 230 aa, Lobosporangium transversale
MEINQEQLPSSSSILHPTSTSSSSSPSPSPSPASPKPERVFDARQRRINEIRLKFCIRDEFPITKNMIHPDGTLNQDYFRPPRGSKPVEVARKWTDKERELLIKGIEKYGIGHFREISEEFLPLWSGNDLRIKTMRLVGRQNLQLYKDWKGNEQDLAREFELNKAIGLKYGAWKAGTLVADDDGLVAKAIEEQWPGSNSGTGKTTAVIGISSEENSEVSTPLNDEDVDME

SEQ ID NO: 84 - protein, 184 aa, Basidiobolus meristosporus
MEVDQNDSSVAKETAEQPETPEISKELLERQEWIKNMRLQFCVRPEFEVTKNIIHEDGMLNQEYFLPPKGAKLEAEPERKWTETERNLLIQGIQQYGIGHFREISEALLPQWSGNDLRVKSMRLMGRQNLQLYKDWKGSIEDIEREYERNKAIGLKYNTWKNSTLVYDDAGLVLKAIEASEPKP

SEQ ID NO: 85 - protein, 206 aa, Absidia repens
MAIDSLQDTEDDRTNDQNDESRESSPTPLSPEEQAQKERHDWINQIRLKFCIRPEFEVTKNIIHPDGRLNQEYFHPPKGYKPEDARKWTETEKQLLIKGIEEHGIGNFGLISKESLPKWSTNDLRVKCIRLIGRQNLQLYRGWKGNADDITREYERNKEIGLKYGTWKQGVLVYDDDGMVEKELLATAATPADSMSMEEDEDMATD

SEQ ID NO: 86 - protein, 171 aa, Linderina pennispora
MDTASPDDGAIAQPMLGVEDADFWRQKQEWVKQMRLQFSRRPEFPETHNMIDDEGMLNQEYFQPPKDAVAPKERKWGDDEKRRLLEGIEKHGIGHFREISEESLPEWSGNDLRMKAIRLMGRQNLQLYKGWKGDAAAIGLKHGTWKGGALVYDDDGVVLKAIQESNRANPP

SEQ ID NO: 87 - protein, 175 aa, Chlamydomonas reinhardtii
MAACSAACDSHVVPQPSPGSWGMPEDRDNYIVQMRRRYSPAGMLNADGSINQDFFKPRRVVLVADRAKWGDAEREGLYKGLEVHGVGKWREINRDYLKGQWDDQQVRIRAARLLGSQSLVRYMGWKGSKAKVDAEYAKNKAIGEATGCWKAGQLVEDDHGSVRKYFEAQQAGGEQ

SEQ ID NO: 88 - protein, 295 aa, Tetrahymena thermophila
MNQMGVIAIKRKQSYQLNVKINYINTAHQIKKPCQYIQKCILFRLLYKFCKQLIPLNFNLFLIFYFYHLLFHLIFNYLLKFAKKINKLIRNQRKNREKKEAFKHKKIQININHYNYLKQNIQQVGIIFQNKKSKLTLKLVQKKSLSEYYRKIKMKKNGKSQNQPLDFTQYAKNMRKDLSNQDICLEDGALNHSYFLTKKGQYWTPLNQKALQRGIELFGVGNWKEINYDEFSGKANIVELELRTCMILGINDITEYYGKKISEEEQEEIKKSNIAKGKKENKLKDNIYQKLQQMQ

SEQ ID NO: 89 - protein, 175 aa, Chlamydomonas reinhardtii; identical to SEQ ID NO: 87

SEQ ID NO: 90 - protein, 190 aa, Oxytricha trifallax
MSTAKQQQAQQHLLPKHSNMRVGSVSNELDYAKRNYIIKMRQSFIEVNKNIYFEDGSLNFKYFNVKKGHYWSKEINEELIKGVIKYGATNYKDIKNKMEIFKKEWSETEIRLRICRLLKCYNLKVYEGHKFNSREEILEQATLNKEEAIKQKKICGGILYNPPHEQDDGIMSSYFNLKNKNNTPVKASAQ

SEQ ID NO: 91 - protein, 206 aa, Absidia repens; identical to SEQ ID NO: 85

SEQ ID NO: 92 - protein, 199 aa, Syncephalastrum racemosum; identical to SEQ ID NO: 82

SEQ ID NO: 93 - protein, 230 aa, Lobosporangium transversale; identical to SEQ ID NO: 83

SEQ ID NO: 94 - protein, 184 aa, Basidiobolus meristosporus; identical to SEQ ID NO: 84

SEQ ID NO: 95 - protein, 171 aa, Linderina pennispora; identical to SEQ ID NO: 86

SEQ ID NO: 96 - protein, 175 aa, Anaeromyces robustus; identical to SEQ ID NO: 81

SEQ ID NO: 97 - protein, 174 aa, Piromyces finnis; identical to SEQ ID NO: 80

SEQ ID NO: 98 - protein, 122 aa, Hesseltinella vesiculosa
MLAGDAELVEKPHNALNAEDTEMEDVDHSSHPDTTVDLSPEQLRLQEKQAWINQMRLKFCVREEFEITKNMIHPDGTLNQDYFKPPKKSKKKKSKSKSKGTDETKDDTEAKGEDNKEDEDME

SEQ ID NO: 99 - protein, 507 aa, Chlamydomonas reinhardtii; identical to SEQ ID NO: 77

SEQ ID NO: 100 - protein, 458 aa, Chlamydomonas reinhardtii; identical to SEQ ID NO: 76

SEQ ID NO: 101 - protein, 1362 aa, Mus musculus
MPRRQAEAMDIDAEREKITQEIQELERILYPGSTSVHFEVSESSLSSDSEADSLPDEDLETAGAPILEEEGSSESSNDEEDPKDKALPEDPETCLQLNMVYQEVIREKLAEVSQLLAQNQEQQEEILFDLSGTKCPKVKDGRSLPSYMYIGHFLKPYFKDKVTGVGPPANEETREKATQGIKAFEQLLVTKWKHWEKALLRKSVVSDRLQRLLQPKLLKLEYLHEKQSRVSSELERQALEKQIKEAEKEIQDINQLPEEALLGNRLDSHDWEKISNINFEGARSAEEIRKFWQSSEHPSISKQEWSTEEVERLKAIAATHGHLEWHLVAEELGTSRSAFQCLQKFQQYNKTLKRKEWTEEEDHMLTQLVQEMRVGNHIPYRKIVYFMEGRDSMQLIYRWTKSLDPSLKRGFWAPEEDAKLLQAVAKYGAQDWFKIREEVPGRSDAQCRDRYIRRLHFSLKKGRWNAKEEQQLIQLIEKYGVGHWARIASELPHRSGSQCLSKWKILARKKQHLQRKRGQRPRHSSQWSSSGSSSSSSEDYGSSSGSDGSSGSENSDVELEASLEKSRALTPQQYRVPDIDLWVPTRLITSQSQREGTGCYPQHPAVSCCTQDASQNHHKEGSTTVSAAEKNQLQVPYETHSTVPRGDRFLHFSDTHSASLKDPACKPVLKVPLEKMPKLIRTRPPTQSHTLMKERPKQPLLPSSRSGSDPGNNTAGPHLRQLWHGTYQNKQRRKRQALHRRLLKHRLLLAVIPWVGDINLACTQAPRRPATVQTKADSIRMQLECARLASTPVFTLLIQLLQIDTAGCMEVVRERKSQPPALLQPGTRNTQPHLLQASSNAKNNTGCLPSMTGEQTAKRASHKGRPRLGSCRTEATPFQVPVAAPRGLRPKPKTVSELLREKRLRESHAKKATQALGLNSQLLVSSPVILQPPLLPVPHGSPVVGPATSSVELSVPVAPVMVSSSPSGSWPVGGISATDKQPPNLQTISLNPPHKGTQVAAPAAFRSLALAPGQVPTGGHLSTLGQTSTTSQKQSLPKVLPILRAAPSLTQLSVQPPVSGQPLATKSSLPVNWVLTTQKLLSVQVPAVVGLPQSVMTPETIGLQAKQLPSPAKTPAFLEQPPASTDTEPKGPQGQEIPPTPGPEKAALDLSLLSQESEAAIVTWLKGCQGAFVPPLGSRMPYHPPSLCSLRALSSLLLQKQDLEQKASSLAASQAAGAQPDPKAGALQASLELVQRQFRDNPAYLLLKTRFLAIFSLPAFLATLPPNSIPTTLSPDVAVVSESDSEDLGDLELKDRARQLDCMACRVQASPAAPDPVQSHLVSPGQRAPSPGEVSAPSPLDASDGLDDLNVLRTRRARHSRR

SEQ ID NO: 102 - protein, 1341 aa, Mus musculus
MPRRQAEAMDIDAEREKITQEIQELERILYPGSTSVHFEVSESSLSSDSEADSLPDEDLETAGAPILEEEGSSESSNDEEDPKDKALPEDPETCLQLNMVYQEVIREKLAEVSQLLAQNQEQQEEILFDLSGTKCPKVKDGRSLPSYMYIGHFLKPYFKDKVTGVGPPANEETREKATQGIKAFEQLLVTKWKHWEKALLRKSVVSDRLQRLLQPKLLKLEYLHEKQSRVSSELERQALEKQIKEAEKEIQDINQLPEEALLGNRLDSHDWEKISNINFEGARSAEEIRKFWQSSEHPSISKQEWSTEEVERLKAIAATHGHLEWHLVAEELGTSRSAFQCLQKFQQYNKTLKRKEWTEEEDHMLTQLVQEMRVGNHIPYRKIVYFMEGRDSMQLIYRWTKSLDPSLKRGFWAPEEDAKLLQAVAKYGAQDWFKIREEVPGRSDAQCRDRYIRRLHFSLKKGRWNAKEEQQLIQLIEKYGVGHWARIASELPHRSGSQCLSKWKILARKKQHLQRKRGQRPRHSSQWSSSGSSSSSSEDYGSSSGSDGSSGSENSDVELEASLEKSRALTPQQYRVPDIDLWVPTRLITSQSQREGTGCYPQHPAVSCCTQDASQNHHKEGSTTVSAAEKNQLQVPYETHSTVPRGDRFLHFSDTHSASLKDPACKSHTLMKERPKQPLLPSSRSGSDPGNNTAGPHLRQLWHGTYQNKQRRKRQALHRRLLKHRLLLAVIPWVGDINLACTQAPRRPATVQTKADSIRMQLECARLASTPVFTLLIQLLQIDTAGCMEVVRERKSQPPALLQPGTRNTQPHLLQASSNAKNNTGCLPSMTGEQTAKRASHKGRPRLGSCRTEATPFQVPVAAPRGLRPKPKTVSELLREKRLRESHAKKATQALGLNSQLLVSSPVILQPPLLPVPHGSPVVGPATSSVELSVPVAPVMVSSSPSGSWPVGGISATDKQPPNLQTISLNPPHKGTQVAAPAAFRSLALAPGQVPTGGHLSTLGQTSTTSQKQSLPKVLPILRAAPSLTQLSVQPPVSGQPLATKSSLPVNWVLTTQKLLSVQVPAVVGLPQSVMTPETIGLQAKQLPSPAKTPAFLEQPPASTDTEPKGPQGQEIPPTPGPEKAALDLSLLSQESEAAIVTWLKGCQGAFVPPLGSRMPYHPPSLCSLRALSSLLLQKQDLEQKASSLAASQAAGAQPDPKAGALQASLELVQRQFRDNPAYLLLKTRFLAIFSLPAFLATLPPNSIPTTLSPDVAVVSESDSEDLGDLELKDRARQLDCMACRVQASPAAPDPVQSHLVSPGQRAPSPGEVSAPSPLDASDGLDDLNVLRTRRARHSRR

SEQ ID NO: 103 - protein, 229 aa, Oxytricha trifallax
MSVHHKMADSKSLHNYTLSPGWTREEVDILKIALMKFGIGKWKKIQKSGCLPSKTISQMNLQTQRLLGQQSLAEFMGLHVYLDRVFRDNSLKTGPEIQRKNNFIINTGNNLTQPEKEKRLRLNKQKYGLDLAFIKTLRLPKPESATGGKREAILSMDQIFAQKSHFTVVEKLKHLEALKNALCSKLGKIERRRRNKELSKIYRPLCQLIVVQKNADDQYEFVDIIDENE

SEQ ID NO: 104 - protein, 206 aa, Linderina pennispora; identical to SEQ ID NO: 66

SEQ ID NO: 105 - protein, 1469 aa, Homo sapiens
MDVDAEREKITQEIKELERILDPGSSGSHVEISESSLESDSEADSLPSEDLDPADPPISEEERWGEASNDEDDPKDKTLPEDPETCLQLNMVYQEVIQEKLAEANLLLAQNREQQEELMRDLAGSKGTKVKDGKSLPPSTYMGHFMKPYFKDKVTGVGPPANEDTREKAAQGIKAFEELLVTKWKNWEKALLRKSVVSDRLQRLLQPKLLKLEYLHQKQSKVSSELERQALEKQGREAEKEIQDINQLPEEALLGNRLDSHDWEKISNINFEGSRSAEEIRKFWQNSEHPSINKQEWSREEEERLQAIAAAHGHLEWQKIAEELGTSRSAFQCLQKFQQHNKALKRKEWTEEEDRMLTQLVQEMRVGSHIPYRRIVYYMEGRDSMQLIYRWTKSLDPGLKKGYWAPEEDAKLLQAVAKYGEQDWFKIREEVPGRSDAQCRDRYLRRLHFSLKKGRWNLKEEEQLIELIEKYGVGHWAKIASELPHRSGSQCLSKWKIMMGKKQGLRRRRRRARHSVRWSSTSSSGSSSGSSGGSSSSSSSSSEEDEPEQAQAGEGDRALLSPQYMVPDMDLWVPARQSTSQPWRGGAGAWLGGPAASLSPPKGSSASQGGSKEASTTAAAPGEETSPVQVPARAHGPVPRSAQASHSADTRPAGAEKQALEGGRRLLTVPVETVLRVLRANTAARSCTQKEQLRQPPLPTSSPGVSSGDSVARSHVQWLRHRATQSGQRRWRHALHRRLLNRRLLLAVTPWVGDVVVPCTQASQRPAVVQTQADGLREQLQQARLASTPVFTLFTQLFHIDTAGCLEVVRERKALPPRLPQAGARDPPVHLLQASSSAQSTPGHLFPNVPAQEASKSASHKGSRRLASSRVERTLPQASLLASTGPRPKPKTVSELLQEKRLQEARAREATRGPVVLPSQLLVSSSVILQPPLPHTPHGRPAPGPTVLNVPLSGPGAPAAAKPGTSGSWQEAGTSAKDKRLSTMQALPLAPVFSEAEGTAPAASQAPALGPGQISVSCPESGLGQSQAPAASRKQGLPEAPPFLPAAPSPTPLPVQPLSLTHIGGPHVATSVPLPVTWVLTAQGLLPVPVPAVVSLPRPAGTPGPAGLLATLLPPLTETRAAQGPRAPALSSSWQPPANMNREPEPSCRTDTPAPPTHALSQSPAEADGSVAFVPGEAQVAREIPEPRTSSHADPPEAEPPWSGRLPAFGGVIPATEPRGTPGSPSGTQEPRGPLGLEKLPLRQPGPEKGALDLEKPPLPQPGPEKGALDLGLLSQEGEAATQQWLGGQRGVRVPLLGSRLPYQPPALCSLRALSGLLLHKKALEHKATSLVVGGEAERPAGALQASLGLVRGQLQDNPAYLLLRARFLAAFTLPALLATLAPQGVRTTLSVPSRVGSESEDEDLLSELELADRDGQPGCTTATCPIQGAPDSGKCSASSCLDTSNDPDDLDVLRTRHARHTRKRRRLV

SEQ ID NO: 106 - protein, 1441 aa, Homo sapiens
MDVDAEREKITQEIKELERILDPGSSGSHVEISESSLESDSEADSLPSEDLDPADPPISEEERWGEASNDEDDPKDKTLPEDPETCLQLNMVYQEVIQEKLAEANLLLAQNREQQEELMRDLAGSKGTKVKDGKSLPPSTYMGHFMKPYFKDKVTGVGPPANEDTREKAAQGIKAFEELLVTKWKNWEKALLRKSVVSDRLQRLLQPKLLKLEYLHQKQSKVSSELERQALEKQGREAEKEIQDINQLPEEALLGNRLDSHDWEKISNINFEGSRSAEEIRKFWQNSEHPSINKQEWSREEEERLQAIAAAHGHLEWQKIAEELGTSRSAFQCLQKFQQHNKALKRKEWTEEEDRMLTQLVQEMRVGSHIPYRRIVYYMEGRDSMQLIYRWTKSLDPGLKKGYWAPEEDAKLLQAVAKYGEQDWFKIREEVPGRSDAQCRDRYLRRLHFSLKKGRWNLKEEEQLIELIEKYGVGHWAKIASELPHRSGSQCLSKWKIMMGKKQGLRRRRRRARHSVRWSSTSSSGSSSGSSGGSSSSSSSSSEEDEPEQAQAGEGDRALLSPQYMVPDMDLWVPARQSTSQPWRGGAGAWLGGPAASLSPPKGSSASQGGSKEASTTAAAPGEETSPVQVPARAHGPVPRSAQASHSADTRPAGAEKQALEGGRRLLTVPVETVLRVLRANTAARSCTQWLRHRATQSGQRRWRHALHRRLLNRRLLLAVTPWVGDVVVPCTQASQRPAVVQTQADGLREQLQQARLASTPVFTLFTQLFHIDTAGCLEVVRERKALPPRLPQAGARDPPVHLLQASSSAQSTPGHLFPNVPAQEASKSASHKGSRRLASSRVERTLPQASLLASTGPRPKPKTVSELLQEKRLQEARAREATRGPVVLPSQLLVSSSVILQPPLPHTPHGRPAPGPTVLNVPLSGPGAPAAAKPGTSGSWQEAGTSAKDKRLSTMQALPLAPVFSEAEGTAPAASQAPALGPGQISVSCPESGLGQSQAPAASRKQGLPEAPPFLPAAPSPTPLPVQPLSLTHIGGPHVATSVPLPVTWVLTAQGLLPVPVPAVVSLPRPAGTPGPAGLLATLLPPLTETRAAQGPRAPALSSSWQPPANMNREPEPSCRTDTPAPPTHALSQSPAEADGSVAFVPGEAQVAREIPEPRTSSHADPPEAEPPWSGRLPAFGGVIPATEPRGTPGSPSGTQEPRGPLGLEKLPLRQPGPEKGALDLEKPPLPQPGPEKGALDLGLLSQEGEAATQQWLGGQRGVRVPLLGSRLPYQPPALCSLRALSGLLLHKKALEHKATSLVVGGEAERPAGALQASLGLVRGQLQDNPAYLLLRARFLAAFTLPALLATLAPQGVRTTLSVPSRVGSESEDEDLLSELELADRDGQPGCTTATCPIQGAPDSGKCSASSCLDTSNDPDDLDVLRTRHARHTRKRRRLV

SEQ ID NO: 107 - protein, 1378 aa, Sus scrofa
MDVDAEREKISKEIKELERILDPGSSGINDDVSESSLDSDSEAESLPDDDADATGPLLSEDERWGDASNDEDDAKERALPEDPETCLQLNMVYQEVVREKLAEVSLLLAQNREQQEEVSWALAGSGGRRVKDGRSPPARLYVGHFMKPYFKDKVTGAGPPANEDTREKAAQGVKAFEELLVTKWKSWEKALLRKAVVSDRLQRLLQPKLLKLEYLQQKQSRATSDAERQALEKQVREAEKEVQDISQLPEEALLGHRLDSHDWEKIANVNFEGGRSAEETRKFWQNHEHPSINKQEWSAQEVDRLKAIAAKHGHLRWQEIAEELGTRRSAFQCLQKYQQHNAALKRREWTQEEDRMLTQLVQAMGVGSHIPYRRIAYYMEGRDSTQLIYRWTKSLDPALKKGLWAPEEDAKLLQAVAKYGEQDWFKIREEVPGRSDAQCRDRYLRRLRLSLKKGRWSAQEEERLLELIGKHGVGHWAKIASELPHRTDSQCLSKWKIMARKQQSRGRRRRRPLRRVCWSSSSEDSEDSGDSGGSSSSSSSSEDVEPEGAPEARADGPAPPSAQHPVPDMDLWVPTRQSARVPWGVGPGAWPGHRSASPRPPEGSDVAPGEEAGRAQAPSETPSASLRGGGCPRSADARPSGSEGLADEGPRRPLTVPLETVLRVLRTNTAALCRALKEKLRRPRLLGSPLGPSPSDGSVARPRVQPRWRRRHALQRRLLERQLLMAVSPWVGDVTLPCAPWRPAVLHRRADGIGKQLQGARLASTPVFTLLIQLFRIDTAGCMEVVRERRAQPPALPSGGRVPSSARNSPGHLFQNGSARGAAKKSASHSGGGGPQSAPAPSGPRPKPKTVSELLREKRLREARARKAAQGPAVLPPQGLLSSPAILQPLPPQQLPVSGAVLSGPGGPAVASPGAPGPWASAKEGPPSLHALALAPASMAAGVTPAAPRAPALGPSQVPASCHLSSLGQSQAPATSRKQGLPEAPPFLPAAPSPIQLPVQPRSLTPALAAHTGASHVVASTPLPVTWVLTAQGLLPVPAVVGLPRPAGPPDPEGLSGTPPPSLTETRAGRGPKQPPAHVSVGPDPPAKTPPTAQSPAEGDGDVAHGPGGPSCPGEAQVAGEASVPRTLSPAKPLADHPEAEPCGSSQLPLPGGLSPGGAPTRHQGLERPPPPWPGPEKGAPDLRLLSQESEAAVRGWLTGQRGVCVPPLASRLPYQPPTLCSLRALSGLLLHKKALEHRAASLVPSGAAGAQQAPLGQVRERLQSSPAYLLLKARFLAAFALPALLATLPPHGVPTTLSAAAGVDSESDDDSLDELELADNGGPLGGWPSGRQAGPAAPTPTQGAPGEGSAAPGLDSDDLDILRTRHAWHARKRRRLV

SEQ ID NO: 108 - protein, 226 aa, Lobosporangium transversale; identical to SEQ ID NO: 69

SEQ ID NO: 109 - protein, 417 aa, Oxytricha trifallax; identical to SEQ ID NO: 71

SEQ ID NO: 110 - protein, 440 aa, Oxytricha trifallax; identical to SEQ ID NO: 68

SEQ ID NO: 111 - protein, 328 aa, Oxytricha trifallax; identical to SEQ ID NO: 64

SEQ ID NO: 112 - protein, 426 aa, Oxytricha trifallax; identical to SEQ ID NO: 70

SEQ ID NO: 113 - protein, 439 aa, Oxytricha trifallax; identical to SEQ ID NO: 72

SEQ ID NO: 114 - protein, 216 aa, Basidiobolus meristosporus; identical to SEQ ID NO: 67

SEQ ID NO: 115 - protein, 311 aa, Anaeromyces robustus; identical to SEQ ID NO: 74

SEQ ID NO: 116 - protein, 305 aa, Piromyces finnis; identical to SEQ ID NO: 73

SEQ ID NO: 117 - protein, 360 aa, Tetrahymena thermophila; identical to SEQ ID NO: 63

SEQ ID NO: 118 - protein, 1423 aa, Sus scrofa
MDVDAEREKISKEIKELERILDPGSSGINDDVSESSLDSDSEAESLPDDDADATGPLLSEDERWGDASNDEDDAKERALPEDPETCLQLNMVYQEVVREKLAEVSLLLAQNREQQEEVSWALAGSGGRRVKDGRSPPARLYVGHFMKPYFKDKVTGAGPPANEDTREKAAQGVKAFEELLVTKWKSWEKALLRKAVVSDRLQRLLQPKLLKLEYLQQKQSRATSDAERQALEKQVREAEKEVQDISQLPEEALLGHRLDSHDWEKIANVNFEGGRSAEETRKFWQNHEHPSINKQEWSAQEVDRLKAIAAKHGHLRWQEIAEELGTRRSAFQCLQKYQQHNAALKRREWTQEEDRMLTQLVQAMGVGSHIPYRRIAYYMEGRDSTQLIYRWTKSLDPALKKGLWAPEEDAKLLQAVAKYGEQDWFKIREEVPGVTFEARAFPASRQRTSLPCAPLWPPALWVSRLGNRRGGRQPRGFSRTPRSVCRRYLRRLRLSLKKGRWSAQEEERLLELIGKHGVGHWAKIASELPHRTDSQCLSKWKIMARKQQSRGRRRRRPLRRVCWSSSSEDSEDSGDSGGSSSSSSSSEDVEPEGAPEARADGPAPPSAQHPVPDMDLWVPTRQSARVPWGVGPGAWPGHRSASPRPPEGSDVAPGEEAGRAQAPSETPSASLRGGGCPRSADARPSGSEGLADEGPRRPLTVPLETVLRVLRTNTAALCRALKEKLRRPRLLGSPLGPSPSDGSVARPRVQPRWRRRHALQRRLLERQLLMAVSPWVGDVTLPCAPWRPAVLHRRADGIGKQLQGARLASTPVFTLLIQLFRIDTAGCMEVVRERRAQPPALPSGGRVPSSARNSPGHLFQNGSARGAAKKSASHSGGGGPQSAPAPSGPRPKPKTVSELLREKRLREARARKAAQGPAVLPPQGLLSSPAILQPLPPQQLPVSGAVLSGPGGPAVASPGAPGPWASAKEGPPSLHALALAPASMAAGVTPAAPRAPALGPSQVPASCHLSSLGQSQAPATSRKQGLPEAPPFLPAAPSPIQLPVQPRSLTPALAAHTGASHVVASTPLPVTWVLTAQGLLPVPAVVGLPRPAGPPDPEGLSGTPPPSLTETRAGRGPKQPPAHVSVGPDPPAKTPPTAQSPAEGDGDVAHGPGGPSCPGEAQVAGEASVPRTLSPAKPLADHPEAEPCGSSQLPLPGGLSPGGAPTRHQGLERPPPPWPGPEKGAPDLRLLSQESEAAVRGWLTGQRGVCVPPLASRLPYQPPTLCSLRALSGLLLHKKALEHRAASLVPSGAAGAQQAPLGQVRERLQSSPAYLLLKARFLAAFALPALLATLPPHGVPTTLSAAAGVDSESDDDSLDELELADNGGPLGGWPSGRQAGPAAPTPTQGAPGEGSAAPGLDSDDLDILRTRHAWHARKRRRLV

SEQ ID NO: 119 - protein, 1598 aa, Danio rerio
MKCLSVNMTHLSRDSWLYTHDVQVTYNSFIKVSPCPKMASDDLRAQRDKIQREILALESTLGADSSIADQLSSDNSSDYESDDSGPTVKRVERDDLETERLRIQREIEELENALGADAALENVLQDSDHDTDSSEDSADDLELPQNVETCLQMNLVYQEVLKEKLAELEQLLIENQQQQKEIEVQLSGPGNSIFSVPGVPPQKQFLGYFLKPYFKDKLTGLGPPANEETKERMKHGSIPVDNLKIKRWEGWQKTLLTNAVARDTMKRMLQPKLSKMEYLSNKLCRAEGEEKEQLKAQIELIEKQIAEIRTLKDDQLLGDLQDDHDWDKISNIDFEGLRQADDLKRFWQNFLHPSINKSVWKQDEIYKLQAVAEEFKMCHWDKIAEALGTNRTAFMCFQTYQRYISKTFRRTHWTEEEDDLLRELVEKMRIGNFIPYIQMSHFMVGRDGSQLAYRWTSVLDPSLKKGPWSKEEDQLLRNAVAKYGTREWGRIRTEVPGRTDSACRDRYLDCLRETVKKGTWSYAEMELLKEKVAKYGVGKWAKIASEIPNRVDAQCLHKWKLMTRSKKPLKRPLSSITTSYPRNKRQKLLKTVKEEMFFNSSSDDESQINYMNSDESDDLAEDENLEIPQKEYVQTEMKEWIPRNAMVWTITPGSFRTLWVRLPTNEEELRESTKESGLGSDSSENSACPNDEPIMERNTILDRFGDVERTYVGMNTVVLHRRTDDEKAMFKVCMSDVKQFIQMKATEFAVKKKKKIKNKKRTLRDVFSLNTDLQKAVIPWIGNVIISTPANEAIFCEGDIVGIKAASIRLQKTSVFTFFIKAFHVDVNGCRTVIEIHKKLDIKMPLAINGNPKPTPISTSPKTVAVLLQQSKAASEHKKPAEPSQQPSLPPSQKPSLPPAQQPTQPPSLPPSVPPSQQPTLPPPSQPSQPPPQPPSLPPSQPPAQQPPQQPSLPPPQPPSLPPPQPPSLPTSQQQSLPPSQQHSLPPFQNPSLPPSQQPSLPPSKQPPQPLPVRQITTPTLIYPNNLVITNPNMEGEVQHLVFKGLLLPQQPSKAVSHIPLPVMQPKTPAQPIVVSKSPSVQDSNSVKSSKRICKPTKKAQALMEQSKVKSRKKEPQKQNQGNKNVVFPTVTLQTSPVIKILSPARLVQVTGLSPNFSSNQTINMPDKSLTIKSPQPCSSGNLHQSAPVVVHSSTNPTFVHSSVSNVSRDNLNVSSTINISPRVSRDALNPTSFLNSTTFPLPQNLSVQQSVQIVPQIPINVVHKATCTKAAKTSSDSSSDESVVKQHQLSPSTGRSIPPAVFNIQPNPSTPPTLSSGPVIFNPNNKVVAPKLCGLNVSSSQLPTVSTQKTKYRPIRPLGPLPVVAPPSRKVTSMSRIRAQSEGEPLISLRDLPAAGVNFDSHLIFPEKSSEVDDWMDGKGGIPLPHLDTSLPYLPPSAATIKTMTDLLRAKQPLLLAAKKVLPAQYQDECNEEVEVEAIRKVVAERFASNPAYLLCKARFLSCFTLPALLATINPCEERQLLSEDDEEDDHLATINPSEEHQSSTEDDEEDLQTNERSQPPTARTELNMNENEASAKQFSGIGPKRQRNQRIKRLIK

SEQ ID NO: 120 - DNA, 38 bp, Artificial Sequence
  primer
actagtcttaaatatgagaaagatgatttgaataagat

SEQ ID NO: 121 - DNA, 42 bp, Artificial Sequence
  primer
atcctagcaatattatctacttataattctattgactattag

SEQ ID NO: 122 - DNA, 45 bp, Artificial Sequence
  primer
ctaattaactaatagtcaatagaattataagtagataatattgct

SEQ ID NO: 123 - DNA, 40 bp, Artificial Sequence
  primer
cattaaatcattaacagagtaatgtcgtcatatatttgtc

SEQ ID NO: 124 - DNA, 37 bp, Artificial Sequence
  primer
tttagtgagcatagacaaatatatgacgacattactc

SEQ ID NO: 125 - DNA, 29 bp, Artificial Sequence
  primer
gcggagatgtctttttgaccttttgatag

SEQ ID NO: 126 - DNA, 36 bp, Artificial Sequence
  primer
atgttaacatgcttattattactatcaaaaggtcaa

SEQ ID NO: 127 - DNA, 33 bp, Artificial Sequence
  primer
ggctgctactgatatttatgttctttatgttta

SEQ ID NO: 128 - DNA, 34 bp, Artificial Sequence
  primer
caaagaacacgaagctcataaacataaagaacat

SEQ ID NO: 129 - DNA, 25 bp, Artificial Sequence
  primer
tggagcaaatgctgctaataacgag

SEQ ID NO: 130 - DNA, 29 bp, Artificial Sequence
  primer
acctccagcagctccgtttctattatttg

SEQ ID NO: 131 - DNA, 24 bp, Artificial Sequence
  primer
ggcctgggtattttccctgcttta

SEQ ID NO: 132 - DNA, 34 bp, Artificial Sequence
  primer
cttcccaggtaaaatttaaggtaaataaagcagg

SEQ ID NO: 133 - DNA, 25 bp, Artificial Sequence
  primer
tcaaggtggaggactcttcggtaac

SEQ ID NO: 134 - DNA, 33 bp, Artificial Sequence
  primer
attacgaacccactacctgaattattgttaccg

SEQ ID NO: 135 - DNA, 21 bp, Artificial Sequence
  primer
aaacgtcctgcaggacaacgc

SEQ ID NO: 136 - DNA, 29 bp, Artificial Sequence
  primer
ttgattgaagttttaatttggtactgggc

SEQ ID NO: 137 - DNA, 29 bp, Artificial Sequence
  primer
ttggatgctgatctgttttgtttagaaag

SEQ ID NO: 138 - DNA, 32 bp, Artificial Sequence
  primer
ttgggatttcttaactggatttctttctaaac

SEQ ID NO: 139 - DNA, 40 bp, Artificial Sequence
  primer
ctgcttaaattaagtacttctatgtttgaaattaatgttc

SEQ ID NO: 140 - DNA, 36 bp, Artificial Sequence
  primer
caattaaaacacgttgaacattaatttcaaacatag

SEQ ID NO: 141 - DNA, 29 bp, Artificial Sequence
  primer
tgaggatccaaggtaaatttcatacaatc

SEQ ID NO: 142 - DNA, 37 bp, Artificial Sequence
  primer
gactgcatgtatatgctaatgattgtatgaaatttac

SEQ ID NO: 143 - DNA, 28 bp, Artificial Sequence
  primer
agtggcatttccaaggaaacattaatac

SEQ ID NO: 144 - DNA, 25 bp, Artificial Sequence
  primer
cagtgtttccctttgtgtaaatggg

SEQ ID NO: 145 - DNA, 29 bp, Artificial Sequence
  primer
tcagtggataaactagcctaaggaaacac

SEQ ID NO: 146 - DNA, 29 bp, Artificial Sequence
  primer
ttttacagactggacacagtagtgtttcc

SEQ ID NO: 147 - DNA, 25 bp, Artificial Sequence
  primer
ccagtggtatcaacatgcggtcatc

SEQ ID NO: 148 - DNA, 31 bp, Artificial Sequence
  primer
gatatatacactcccagcagtaaagatgacc

SEQ ID NO: 149 - DNA, 27 bp, Artificial Sequence
  primer
gaataggctcactctaaattcgagtgc

SEQ ID NO: 150 - DNA, 28 bp, Artificial Sequence
  primer
attcgctaggtctaagcaaatattgcac

SEQ ID NO: 151 - DNA, 39 bp, Artificial Sequence
  primer
taaatagccaaaacaaccaataaaattaacaataacctc

SEQ ID NO: 152 - DNA, 34 bp, Artificial Sequence
  primer
ctttttgagggcgaggttattgttaattttattg

SEQ ID NO: 153 - DNA, 37 bp, Artificial Sequence
  primer
gatccattaattacagaaataaataataggcagcata

SEQ ID NO: 154 - DNA, 36 bp, Artificial Sequence
  primer
atattgcctgaattattatgctgcctattatttatt

SEQ ID NO: 155 - DNA, 24 bp, Artificial Sequence
  primer
aaatgtgcaccgtcatcaaatacc

SEQ ID NO: 156 - DNA, 31 bp, Artificial Sequence
  primer
ggatcactataatcatctggatgactattgg

SEQ ID NO: 157 - DNA, 32 bp, Artificial Sequence
  primer
aagtgtaatgtagtttcaatggtagtgatgtg

SEQ ID NO: 158 - DNA, 25 bp, Artificial Sequence
  primer
tgacttcttccagtggattcacatc

SEQ ID NO: 159 - DNA, 34 bp, Artificial Sequence
  primer
gccaattaattcatttgttcgtagagatatgtaa

SEQ ID NO: 160 - DNA, 43 bp, Artificial Sequence
  primer
cactttataataaataagaattattacatatctctacgaacaa

SEQ ID NO: 161 - DNA, 23 bp, Artificial Sequence
  primer
ctcaccagtaatttgcagacacc

SEQ ID NO: 162 - DNA, 23 bp, Artificial Sequence
  primer
ggctgactggggttgagttaatc

SEQ ID NO: 163 - DNA, 43 bp, Artificial Sequence
  primer
aatataaacaaaatggaatatacaaaacttgaataagaaatag

SEQ ID NO: 164 - DNA, 33 bp, Artificial Sequence
  primer
gagactgaggatctatttcttattcaagttttg

SEQ ID NO: 165 - DNA, 53 bp, Artificial Sequence
  primer
attaatacattattaacttaaatataaatatttaaagaattatgaacaataat

SEQ ID NO: 166 - DNA, 54 bp, Artificial Sequence
  primer
cattttgtttatattattgttcataattctttaaatatttatatttaagttaat

SEQ ID NO: 167 - DNA, 44 bp, Artificial Sequence
  primer
acaagataacattgctaattttcaataaattaaattaatacatt

SEQ ID NO: 168 - DNA, 56 bp, Artificial Sequence
  primer
ccccaaaaccccaaaaccccactagtcttaaatatgagaaagatgatttgaataag

SEQ ID NO: 169 - DNA, 55 bp, Artificial Sequence
  primer
ccccaaaaccccaaaaccccacaagataacattgctaattttcaataaattaaat

SEQ ID NO: 170 - DNA, 52 bp, Artificial Sequence
  primer
ccccaaaaccccaaaaccccgatttatgaaagtgctgtattattaaggaatg

SEQ ID NO: 171 - DNA, 53 bp, Artificial Sequence
  primer
ccccaaaaccccaaaaccccattattcctacttttagctatattagaaattcg

SEQ ID NO: 172 - DNA, 55 bp, Artificial Sequence
  primer
ccccaaaaccccaaaaccccatgatgatacatagattcattaaaataaaaaaaag

SEQ ID NO: 173 - DNA, 54 bp, Artificial Sequence
  primer
ccccaaaaccccaaaaccccttagatgaattaaataaagaattcaaataaatac

SEQ ID NO: 174 - DNA, 49 bp, Artificial Sequence
  primer
ccccaaaaccccaaaaccccatgaatctgaaatcgggcagttgaatacg

SEQ ID NO: 175 - DNA, 52 bp, Artificial Sequence
  primer
ccccaaaaccccaaaaccccatttatcataattatagagaagatagtgatgc

SEQ ID NO: 176 - DNA, 47 bp, Artificial Sequence
  primer
ccccaaaaccccaaaaccccatgagagtttgtgaaaaattaagtttg

SEQ ID NO: 177 - DNA, 53 bp, Artificial Sequence
  primer
ccccaaaaccccaaaacccctatattaaatatcaagaaaaagtaaaaagacag

SEQ ID NO: 178 - DNA, 52 bp, Artificial Sequence
  primer
ccccaaaaccccaaaaccccaagtctcattttggttagtgatgtttggattg

SEQ ID NO: 179 - DNA, 53 bp, Artificial Sequence
  primer
ccccaaaaccccaaaaccccgtatgatcgatgaatacaaaatcaagttggaag

SEQ ID NO: 180 - DNA, 55 bp, Artificial Sequence
  primer
ccccaaaaccccaaaaccccacttaaaaggattgcatgattgtaagggaaatgtg

SEQ ID NO: 181 - DNA, 53 bp, Artificial Sequence
  primer
ccccaaaaccccaaaaccccaataatcgcacttacattatatctggagaaatg

SEQ ID NO: 182 - DNA, 53 bp, Artificial Sequence
  primer
ccccaaaaccccaaaaccccttctactaaatttcattgatttttttcaatttc

SEQ ID NO: 183 - DNA, 52 bp, Artificial Sequence
  primer
ccccaaaaccccaaaaccccatttgatagaatagaagagaaattatggaatg

SEQ ID NO: 184 - DNA, 55 bp, Artificial Sequence
  primer
ccccaaaaccccaaaaccccaagtataaataagggagttgatatataatatactt

SEQ ID NO: 185 - DNA, 56 bp, Artificial Sequence
  primer
ccccaaaaccccaaaaccccatgagaattcctattcaaaaatgaaaaagtagattg

SEQ ID NO: 186 - DNA, 56 bp, Artificial Sequence
  primer
ccccaaaaccccaaaaccccataaggtagtatatttttattaaggattggaaatta

SEQ ID NO: 187 - DNA, 56 bp, Artificial Sequence
  primer
ccccaaaaccccaaaaccccataagactaaatttattgaaattatcttgttaatag

SEQ ID NO: 188 - DNA, 56 bp, Artificial Sequence
  primer
ccccaaaaccccaaaaccccttgagccaatactgaaaaggatgatagtgaatagtg

SEQ ID NO: 189 - DNA, 60 bp, Artificial Sequence
  primer
ccccaaaaccccaaaacccctcattttttaaattggatagtaagaaaaattataataaag

SEQ ID NO: 190 - DNA, 55 bp, Artificial Sequence
  primer
ccccaaaaccccaaaaccccaaggaataaaattcaattccaaaatgtaaggtgag

SEQ ID NO: 191 - DNA, 52 bp, Artificial Sequence
  primer
ccccaaaaccccaaaaccccgttaaaagaaccaagtgatatattataagcca

SEQ ID NO: 192 - DNA, 60 bp, Artificial Sequence
  Primer
ccccaaaaccccaaaacccctttatcaattataaataaaaagttttaagtctatttttaa

SEQ ID NO: 193 - DNA, 58 bp, Artificial Sequence
  primer
ccccaaaaccccaaaaccccataagacaaatgcaactttataaagtaaataaattatc

SEQ ID NO: 194 - DNA, 54 bp, Artificial Sequence
  primer
ccccaaaaccccaaaaccccaatgcaacatttacttttaacattagagattatc

SEQ ID NO: 195 - DNA, 54 bp, Artificial Sequence
  primer
ccccaaaaccccaaaaccccataagagcaaaagttaatataaaaattcaaggtg

SEQ ID NO: 196 - DNA, 52 bp, Artificial Sequence
  primer
ccccaaaaccccaaaaccccgatttgcacagttaatttgaatttggtatttg

SEQ ID NO: 197 - DNA, 59 bp, Artificial Sequence
  primer
ccccaaaaccccaaaacccctcatttttagtattttaaatatcatttagttttaagtaa

SEQ ID NO: 198 - DNA, 57 bp, Artificial Sequence
  primer
ccccaaaaccccaaaaccccttgattgattcctgaatacaaatgaaataatataaag

SEQ ID NO: 199 - DNA, 53 bp, Artificial Sequence
  primer
ccccaaaaccccaaaaccccaagaccaaaataaagaggaataatgagaagtac

SEQ ID NO: 200 - DNA, 55 bp, Artificial Sequence
  primer
ccccaaaaccccaaaaccccatgtagaattaatatgagaacatcattttttaagc

SEQ ID NO: 201 - DNA, 56 bp, Artificial Sequence
  primer
ccccaaaaccccaaaaccccataatgtaagaaatctgatacaatagagagataaac

SEQ ID NO: 202 - DNA, 54 bp, Artificial Sequence
  primer
ccccaaaaccccaaaaccccgaatggaaaatttgtatgaagttcagagagaaag

SEQ ID NO: 203 - DNA, 55 bp, Artificial Sequence
  primer
ccccaaaaccccaaaaccccataagattatcagttataaaaattgataggggatg

SEQ ID NO: 204 - DNA, 56 bp, Artificial Sequence
  primer
ccccaaaaccccaaaaccccatcatacgatatcttaagtgttgatctgaattaaat

SEQ ID NO: 205 - DNA, 54 bp, Artificial Sequence
  primer
ccccaaaaccccaaaaccccgttaggtttaagagtagaaataaaaggagataag

SEQ ID NO: 206 - DNA, 51 bp, Artificial Sequence
  primer
ccccaaaaccccaaaacccctctcactatcttttgtaaaaagttggtagat

SEQ ID NO: 207 - DNA, 58 bp, Artificial Sequence
  primer
ccccaaaaccccaaaaccccgttggtttagaataaagaattgtattaaccaaatttat

SEQ ID NO: 208 - DNA, 57 bp, Artificial Sequence
  primer
ccccaaaaccccaaaaccccgtgaattaaaatataaacgaataagatataaagattg

SEQ ID NO: 209 - DNA, 54 bp, Artificial Sequence
  primer
ccccaaaaccccaaaaccccttaattactgaattgtttattataagattataag

SEQ ID NO: 210 - DNA, 51 bp, Artificial Sequence
  primer
ccccaaaaccccaaaaccccgtaatgaataaattgtaaaggtaaattgcaa

SEQ ID NO: 211 - DNA, 58 bp, Artificial Sequence
  primer
ccccaaaaccccaaaaccccaatggcaaacatttaaaataaatattaatataaattac

SEQ ID NO: 212 - DNA, 49 bp, Artificial Sequence
  primer
ccccaaaaccccaaaacccctaaaaggaaaacaaatagaagaaactgaa

SEQ ID NO: 213 - DNA, 49 bp, Artificial Sequence
  primer
ccccaaaaccccaaaaccccatttggatattatgattagcagtttagtg

SEQ ID NO: 214 - DNA, 52 bp, Artificial Sequence
  primer
ccccaaaaccccaaaacccctttaaataaaaatcgcatgaattaaatgcaag

SEQ ID NO: 215 - DNA, 52 bp, Artificial Sequence
  primer
ccccaaaaccccaaaacccctaggtaaatgcaaattggagaatttccaatag

SEQ ID NO: 216 - DNA, 53 bp, Artificial Sequence
  primer
ccccaaaaccccaaaaccccatattaagaattgtgtaatttttgagtaaattg

SEQ ID NO: 217 - DNA, 54 bp, Artificial Sequence
  primer
ccccaaaaccccaaaaccccatttagtagaatcttcaataaataagcgttattg

SEQ ID NO: 218 - DNA, 56 bp, Artificial Sequence
  primer
ccccaaaaccccaaaacccctagcattaaatttgtaaaaagaatgaaatttaatat

SEQ ID NO: 219 - DNA, 53 bp, Artificial Sequence
  primer
ccccaaaaccccaaaaccccaatatacatgattttagataaacaacaaataat

SEQ ID NO: 220 - DNA, 53 bp, Artificial Sequence
  primer
ccccaaaaccccaaaaccccatcaagaatggattagaatttttaatgctttgc

SEQ ID NO: 221 - DNA, 51 bp, Artificial Sequence
  primer
ccccaaaaccccaaaaccccgaggaactagggattactcattttacttcag

SEQ ID NO: 222 - DNA, 55 bp, Artificial Sequence
  primer
ccccaaaaccccaaaaccccatgcatgtaattttctgtcaaaattgagtaaatag

SEQ ID NO: 223 - DNA, 50 bp, Artificial Sequence
  primer
ccccaaaaccccaaaaccccgtaagctaaataagtagactaaataggtag

SEQ ID NO: 224 - DNA, 52 bp, Artificial Sequence
  primer
ccccaaaaccccaaaaccccaaccgcaaatagaatatataaaggataattta

SEQ ID NO: 225 - DNA, 60 bp, Artificial Sequence
  primer
ccccaaaaccccaaaaccccgaagtactaaaaataaaaagtaaagtattaaaataaaatc

SEQ ID NO: 226 - DNA, 53 bp, Artificial Sequence
  primer
ccccaaaaccccaaaaccccgtagacagattttccagtttatagctgtgtttg

SEQ ID NO: 227 - DNA, 59 bp, Artificial Sequence
  primer
ccccaaaaccccaaaacccctttatgaattttcttaaatctgtaaataaataaaataat

SEQ ID NO: 228 - DNA, 53 bp, Artificial Sequence
  primer
ccccaaaaccccaaaaccccgtatgttaattttatgctttaaatgatagttta

SEQ ID NO: 229 - DNA, 51 bp, Artificial Sequence
  primer
ccccaaaaccccaaaacccctggattccattttgaagaataatttattaac

SEQ ID NO: 230 - DNA, 53 bp, Artificial Sequence
  primer
ccccaaaaccccaaaaccccttgtttcgattatattcaaaataggaaatttag

SEQ ID NO: 231 - DNA, 55 bp, Artificial Sequence
  primer
ccccaaaaccccaaaaccccatgaatttcaataactttttatgaaaatgaattta

SEQ ID NO: 232 - DNA, 55 bp, Artificial Sequence
  primer
ccccaaaaccccaaaacccctaggaagaaaatcttgtgtgcaatttgagattaac

SEQ ID NO: 233 - DNA, 55 bp, Artificial Sequence
  primer
ccccaaaaccccaaaaccccttgataaaaacatagattaaatactagtgtataaa

SEQ ID NO: 234 - DNA, 58 bp, Artificial Sequence
  primer
ccccaaaaccccaaaaccccatatggaatatttaatttgatttaaatgaaacgaaata

SEQ ID NO: 235 - DNA, 56 bp, Artificial Sequence
  primer
ccccaaaaccccaaaaccccttgtaacagtaaatagaatattttaattaccaaaac

SEQ ID NO: 236 - DNA, 53 bp, Artificial Sequence
  primer
ccccaaaaccccaaaacccctcattttagaattatctgtacttaattattttg

SEQ ID NO: 237 - DNA, 54 bp, Artificial Sequence
  primer
ccccaaaaccccaaaaccccatgagcatgttattttacttcattagtcaatttg

SEQ ID NO: 238 - DNA, 50 bp, Artificial Sequence
  primer
ccccaaaaccccaaaaccccatgaaatgaattctaagattgaattgcatg

SEQ ID NO: 239 - DNA, 50 bp, Artificial Sequence
  primer
ccccaaaaccccaaaaccccagaagagatcaataaattgagaaggaattg

SEQ ID NO: 240 - DNA, 55 bp, Artificial Sequence
  primer
ccccaaaaccccaaaaccccgtgttacaatttgcgtttgaaatagttggttgata

SEQ ID NO: 241 - DNA, 55 bp, Artificial Sequence
  primer
ccccaaaaccccaaaaccccatatggtaaaaattgaagaaagaaattcaagagaa

SEQ ID NO: 242 - DNA, 53 bp, Artificial Sequence
  primer
ccccaaaaccccaaaaccccgtattgatgataaaattgtatacaagttgatag

SEQ ID NO: 243 - DNA, 53 bp, Artificial Sequence
  primer
ccccaaaaccccaaaacccctagatgcttaattattaagaagattctggaatg

SEQ ID NO: 244 - DNA, 54 bp, Artificial Sequence
  primer
ccccaaaaccccaaaaccccataaaccaatgtaattaatttattgggtgtgttg

SEQ ID NO: 245 - DNA, 58 bp, Artificial Sequence
  primer
ccccaaaaccccaaaaccccttagattaaatttagagagttatagaaatgtagtaaat

SEQ ID NO: 246 - DNA, 54 bp, Artificial Sequence
  primer
ccccaaaaccccaaaaccccatctcaatttataaaatcagaataagagattgtc

SEQ ID NO: 247 - DNA, 52 bp, Artificial Sequence
  primer
ccccaaaaccccaaaaccccagaataaaacaactgaagtaaatatgagttac

SEQ ID NO: 248 - DNA, 55 bp, Artificial Sequence
  primer
ccccaaaaccccaaaacccctttcaaatataaaataaacagaagaatggcaaacg

SEQ ID NO: 249 - DNA, 55 bp, Artificial Sequence
  primer
ccccaaaaccccaaaaccccaaattcaatattaaatgaaataattttcaaaagtg

SEQ ID NO: 250 - DNA, 51 bp, Artificial Sequence
  primer
ccccaaaaccccaaaaccccatgagatcaaatttttttattaaaattcttc

SEQ ID NO: 251 - DNA, 52 bp, Artificial Sequence
  primer
ccccaaaaccccaaaaccccttggattcatatttttgtttaaggcttagata

SEQ ID NO: 252 - DNA, 53 bp, Artificial Sequence
  primer
ccccaaaaccccaaaaccccattagaaaagaggatttcaataaaagcaaatat

SEQ ID NO: 253 - DNA, 55 bp, Artificial Sequence
  primer
ccccaaaaccccaaaaccccatcgatttattattgttgaatttaaaagtattgaa

SEQ ID NO: 254 - DNA, 58 bp, Artificial Sequence
  primer
ccccaaaaccccaaaaccccgagaggtttgataagtagaattagtaaaatctataaag

SEQ ID NO: 255 - DNA, 57 bp, Artificial Sequence
  primer
ccccaaaaccccaaaaccccattagtactattttcatagatctatgtataaattgaa

SEQ ID NO: 256 - DNA, 58 bp, Artificial Sequence
  primer
ccccaaaaccccaaaaccccaatggaaagataaacagattttaatttggaaataaaat

SEQ ID NO: 257 - DNA, 58 bp, Artificial Sequence
  primer
ccccaaaaccccaaaacccctttaagcagtatttctaaaatgttgatgaaataaaaat

SEQ ID NO: 258 - DNA, 53 bp, Artificial Sequence
  primer
ccccaaaaccccaaaaccccataagataaaatttaacgaaaaaaagttaagtc

SEQ ID NO: 259 - DNA, 51 bp, Artificial Sequence
  primer
ccccaaaaccccaaaaccccataagatgaaatatagagataattgagccta

SEQ ID NO: 260 - DNA, 51 bp, Artificial Sequence
  primer
ccccaaaaccccaaaaccccaattacatattaatgtacttatgatagaatg

SEQ ID NO: 261 - DNA, 49 bp, Artificial Sequence
  primer
ccccaaaaccccaaaacccctaatgatcaaataacctgagttaaagaag

SEQ ID NO: 262 - DNA, 52 bp, Artificial Sequence
  primer
ccccaaaaccccaaaaccccaaattatgaaaatagacactaattggatgttc

SEQ ID NO: 263 - DNA, 53 bp, Artificial Sequence
  primer
ccccaaaaccccaaaacccctgattcgtcatatgaaattgaaaaggagtaaat

SEQ ID NO: 264 - DNA, 51 bp, Artificial Sequence
  primer
ccccaaaaccccaaaaccccagcgccatgaatctgatgcatttattttaag

SEQ ID NO: 265 - DNA, 53 bp, Artificial Sequence
  primer
ccccaaaaccccaaaaccccgtagatcatttatgtaaaagattttgagagatg

SEQ ID NO: 266 - DNA, 54 bp, Artificial Sequence
  primer
ccccaaaaccccaaaaccccatacaattattataaatgaaaaagcgcactaatc

SEQ ID NO: 267 - DNA, 58 bp, Artificial Sequence
  primer
ccccaaaaccccaaaaccccatagttactatgaaaggactggtacatagaaataatag

SEQ ID NO: 268 - DNA, 57 bp, Artificial Sequence
  primer
ccccaaaaccccaaaaccccttaagtcaatatctaaatcaaatattagtagtataat

SEQ ID NO: 269 - DNA, 58 bp, Artificial Sequence
  primer
ccccaaaaccccaaaaccccgtcatatggttttataaaataaaattgagatttttttg

SEQ ID NO: 270 - DNA, 54 bp, Artificial Sequence
  primer
ccccaaaaccccaaaaccccataaggataaattctatcatataagtggaagtgc

SEQ ID NO: 271 - DNA, 57 bp, Artificial Sequence
  primer
ccccaaaaccccaaaaccccattcttgaatattgattatgcatattgtgtaaaatag

SEQ ID NO: 272 - DNA, 52 bp, Artificial Sequence
  primer
ccccaaaaccccaaaaccccaagcgttgaattttttataatatatgataaac

SEQ ID NO: 273 - DNA, 55 bp, Artificial Sequence
  primer
ccccaaaaccccaaaaccccttaatgccaataaacagatgaaagtagagttatag

SEQ ID NO: 274 - DNA, 55 bp, Artificial Sequence
  primer
ccccaaaaccccaaaaccccatagagagtgttttattgaaggacagagaatattg

SEQ ID NO: 275 - DNA, 55 bp, Artificial Sequence
  primer
ccccaaaaccccaaaaccccgagcgtaagaaatattcttagataaatggaaactg

SEQ ID NO: 276 - DNA, 47 bp, Artificial Sequence
  primer
ccccaaaaccccaaaaccccatggcaatatctttgcgtgtttctggc

SEQ ID NO: 277 - DNA, 57 bp, Artificial Sequence
  primer
ccccaaaaccccaaaaccccataagaataaattaaagaagatttgagaaagatatgc

SEQ ID NO: 278 - DNA, 50 bp, Artificial Sequence
  primer
ccccaaaaccccaaaaccccaaatgctaaaaataatgaaaaatctgaggg

SEQ ID NO: 279 - DNA, 49 bp, Artificial Sequence
  primer
ccccaaaaccccaaaacccctaatgacaggtttagtaataatttagctg

SEQ ID NO: 280 - DNA, 53 bp, Artificial Sequence
  primer
ccccaaaaccccaaaaccccacgacttaacattgctgttaaatattcagaaat

SEQ ID NO: 281 - DNA, 51 bp, Artificial Sequence
  primer
ccccaaaaccccaaaacccctaaaattggaaaggggcaaatttgcttatga

SEQ ID NO: 282 - DNA, 59 bp, Artificial Sequence
  primer
ccccaaaaccccaaaaccccatgagtaatatatacaaattttaaatgtattttgattta

SEQ ID NO: 283 - DNA, 54 bp, Artificial Sequence
  primer
ccccaaaaccccaaaaccccattgagtgagtatttttatatttattgcgagtta

SEQ ID NO: 284 - DNA, 53 bp, Artificial Sequence
  primer
ccccaaaaccccaaaaccccacaataggcatatttaataattaattgttaaag

SEQ ID NO: 285 - DNA, 51 bp, Artificial Sequence
  primer
ccccaaaaccccaaaaccccactcattatataaggctgaaaaaatcagagg

SEQ ID NO: 286 - DNA, 50 bp, Artificial Sequence
  primer
ccccaaaaccccaaaacccctaaatgtaagagtaaactatcatatgaaag

SEQ ID NO: 287 - DNA, 52 bp, Artificial Sequence
  primer
ccccaaaaccccaaaaccccataatgcgaaatattcatcagagtaaataatg

SEQ ID NO: 288 - DNA, 57 bp, Artificial Sequence
  primer
ccccaaaaccccaaaaccccatacgtcatgattataagattattatagaatgcttac

SEQ ID NO: 289 - DNA, 56 bp, Artificial Sequence
  primer
ccccaaaaccccaaaacccctcttgtaaaataataagtttaagaaattgaatttag

SEQ ID NO: 290 - DNA, 59 bp, Artificial Sequence
  primer
ccccaaaaccccaaaaccccataatatcaaattaatgaatatttatcaattttattaat

SEQ ID NO: 291 - DNA, 53 bp, Artificial Sequence
  primer
ccccaaaaccccaaaaccccccctaatgtccataatttatgtatcaaataagg

SEQ ID NO: 292 - DNA, 53 bp, Artificial Sequence
  primer
ccccaaaaccccaaaaccccatgatggtggaggagtgaagataaattagaatg

SEQ ID NO: 293 - DNA, 54 bp, Artificial Sequence
  primer
ccccaaaaccccaaaaccccaaagtgcaataaaaagagtgaaaataaatttttg

SEQ ID NO: 294 - DNA, 58 bp, Artificial Sequence
  primer
ccccaaaaccccaaaaccccatataccaatgttaaaaatgaatattgatatagaatag

SEQ ID NO: 295 - DNA, 56 bp, Artificial Sequence
  primer
ccccaaaaccccaaaaccccataatacaaagtaaaattgttttttatagttcataa

SEQ ID NO: 296 - DNA, 52 bp, Artificial Sequence
  primer
ccccaaaaccccaaaaccccacatagtgaatgaattaatgaataagtttgag

SEQ ID NO: 297 - DNA, 55 bp, Artificial Sequence
  primer
ccccaaaaccccaaaaccccgtgataataaattcctgagtatatagtttaagaag

SEQ ID NO: 298 - DNA, 49 bp, Artificial Sequence
  primer
ccccaaaaccccaaaaccccgtgattgcatttttttgcgaaatatttgc

SEQ ID NO: 299 - DNA, 49 bp, Artificial Sequence
  primer
ccccaaaaccccaaaacccctgagttctcatgtaataaaagaatccatg

SEQ ID NO: 300 - DNA, 53 bp, Artificial Sequence
  primer
ccccaaaaccccaaaaccccatgatgctacaaaaacgctatataatctataac

SEQ ID NO: 301 - DNA, 51 bp, Artificial Sequence
  primer
ccccaaaaccccaaaaccccttgaactttcaatagatgtttgattaaattc

SEQ ID NO: 302 - DNA, 53 bp, Artificial Sequence
  primer
ccccaaaaccccaaaaccccaaagatatgtggctggattttaaaatatggttg

SEQ ID NO: 303 - DNA, 57 bp, Artificial Sequence
  primer
ccccaaaaccccaaaaccccaagactaatgaatttgagaattataaaataatgaatc

SEQ ID NO: 304 - DNA, 59 bp, Artificial Sequence
  primer
ccccaaaaccccaaaaccccatcaactttaattcattgtaggaattaaagatgtaatac

SEQ ID NO: 305 - DNA, 55 bp, Artificial Sequence
  primer
ccccaaaaccccaaaaccccgtgagaacaaataataataaaaataaaggaattaa

SEQ ID NO: 306 - DNA, 55 bp, Artificial Sequence
  primer
ccccaaaaccccaaaaccccaattctttatctgaattagataagaattcataagc

SEQ ID NO: 307 - DNA, 52 bp, Artificial Sequence
  primer
ccccaaaaccccaaaaccccgtgagtatgcaatagattgttaattaaatttg

SEQ ID NO: 308 - DNA, 54 bp, Artificial Sequence
  primer
ccccaaaaccccaaaaccccaagttgctaaaaatagttgatagcaacaagttat

SEQ ID NO: 309 - DNA, 59 bp, Artificial Sequence
  primer
ccccaaaaccccaaaacccctggatgtgtttttttccaaattaatgaacaaaaattaaa

SEQ ID NO: 310 - DNA, 53 bp, Artificial Sequence
  primer
ccccaaaaccccaaaaccccaacattctaaatttcttctttataagattattg

SEQ ID NO: 311 - DNA, 52 bp, Artificial Sequence
  primer
ccccaaaaccccaaaaccccatctaaactaatctgaaaccaaagatagtatg

SEQ ID NO: 312 - DNA, 53 bp, Artificial Sequence
  primer
ccccaaaaccccaaaaccccgttatccatatatacgtaagcattttgcgattg

SEQ ID NO: 313 - DNA, 58 bp, Artificial Sequence
  primer
ccccaaaaccccaaaaccccgaaacctatgcattatttttaaagaaatattaaattaa

SEQ ID NO: 314 - DNA, 57 bp, Artificial Sequence
  primer
ccccaaaaccccaaaacccctcgtacattaatagttgaaattgcttttattaaattg

SEQ ID NO: 315 - DNA, 53 bp, Artificial Sequence
  primer
ccccaaaaccccaaaaccccgtagtctaaaataaattttattttgggttttaa

SEQ ID NO: 316 - DNA, 52 bp, Artificial Sequence
  primer
ccccaaaaccccaaaaccccgttaaatgataatcatagcaaaattgcggtat

SEQ ID NO: 317 - DNA, 59 bp, Artificial Sequence
  primer
ccccaaaaccccaaaaccccaaggataaatattgaaagtaaatgttctaattaatttgc

SEQ ID NO: 318 - DNA, 52 bp, Artificial Sequence
  primer
ccccaaaaccccaaaaccccagaaatgaaaagaatgatttttgaggggattc

SEQ ID NO: 319 - DNA, 53 bp, Artificial Sequence
  primer
ccccaaaaccccaaaacccctaaaggcaaaagtcgatttaaatgctcagtttc

SEQ ID NO: 320 - DNA, 52 bp, Artificial Sequence
  primer
ccccaaaaccccaaaaccccttaaggctaaaatacttgttttactagagaac

SEQ ID NO: 321 - DNA, 50 bp, Artificial Sequence
  primer
ccccaaaaccccaaaaccccataaatcaaattaaattgcataacatgaac

SEQ ID NO: 322 - DNA, 52 bp, Artificial Sequence
  primer
ccccaaaaccccaaaaccccagaggatgtaaattacaataaatcgtaaaaac

SEQ ID NO: 323 - DNA, 50 bp, Artificial Sequence
  primer
ccccaaaaccccaaaaccccttctaaaaaatataaagataaattgacgtc

SEQ ID NO: 324 - DNA, 58 bp, Artificial Sequence
  primer
ccccaaaaccccaaaaccccatccagttgaaatctaaaacaattttgtatatttaaag

SEQ ID NO: 325 - DNA, 53 bp, Artificial Sequence
  primer
ccccaaaaccccaaaaccccttaagagattgcattataaataagataggattc

SEQ ID NO: 326 - DNA, 55 bp, Artificial Sequence
  primer
ccccaaaaccccaaaaccccattgattgataaacttggaagttaagaaagatttg

SEQ ID NO: 327 - DNA, 51 bp, Artificial Sequence
  primer
ccccaaaaccccaaaaccccatgaataacagatggaatgcttcaagatatg

SEQ ID NO: 328 - DNA, 54 bp, Artificial Sequence
  primer
ccccaaaaccccaaaaccccaaatgttagtatttgaattaaagagaggtaaaac

SEQ ID NO: 329 - DNA, 54 bp, Artificial Sequence
  primer
ccccaaaaccccaaaaccccttatgaaaatgaaatggttttgattggctaataa

SEQ ID NO: 330 - DNA, 54 bp, Artificial Sequence
  primer
ccccaaaaccccaaaaccccatgagtaaaatttagcttaagtaatgtaagaatc

SEQ ID NO: 331 - DNA, 59 bp, Artificial Sequence
  primer
ccccaaaaccccaaaaccccatatatcaaaatatcaacatttttttgtgtgattgttac

SEQ ID NO: 332 - DNA, 51 bp, Artificial Sequence
  primer
ccccaaaaccccaaaaccccttgatgaaatttgaaaatgaatagagagtac

SEQ ID NO: 333 - DNA, 52 bp, Artificial Sequence
  primer
ccccaaaaccccaaaaccccgtaatgctacatttgcaaaaaagtacaaacag

SEQ ID NO: 334 - DNA, 50 bp, Artificial Sequence
  primer
ccccaaaaccccaaaaccccgtaaggccagaatcaatgaataaaaaggtc

SEQ ID NO: 335 - DNA, 59 bp, Artificial Sequence
  primer
ccccaaaaccccaaaaccccgaaaagggagatttacaaaaatttgtagatgttatattg

SEQ ID NO: 336 - DNA, 52 bp, Artificial Sequence
  primer
ccccaaaaccccaaaaccccattgatcattaataaagaagaattgctaatat

SEQ ID NO: 337 - DNA, 51 bp, Artificial Sequence
  primer
ccccaaaaccccaaaaccccaatgcgatgaaatgttttttattatgaaaag

SEQ ID NO: 338 - DNA, 51 bp, Artificial Sequence
  primer
ccccaaaaccccaaaaccccaaggaagttcaatgctatttagcaaattagg

SEQ ID NO: 339 - DNA, 54 bp, Artificial Sequence
  primer
ccccaaaaccccaaaaccccttgattcaaaatatgcacaagattaaaaattcac

SEQ ID NO: 340 - DNA, 53 bp, Artificial Sequence
  primer
ccccaaaaccccaaaaccccataagaaagataagttgcaattaaataataagg

SEQ ID NO: 341 - DNA, 50 bp, Artificial Sequence
  primer
ccccaaaaccccaaaaccccatgaagacaagtctgatgaaaatagaatgg

SEQ ID NO: 342 - DNA, 60 bp, Artificial Sequence
  primer
ccccaaaaccccaaaaccccatagtcttaaaattttatactatcatgaaataatattaag

SEQ ID NO: 343 - DNA, 55 bp, Artificial Sequence
  primer
ccccaaaaccccaaaaccccgtaagtctaaagtttaacagtttttagtaaatatc

SEQ ID NO: 344 - DNA, 55 bp, Artificial Sequence
  primer
ccccaaaaccccaaaaccccttatgctagttgagtgattgaaaatatatttgtgc

SEQ ID NO: 345 - DNA, 48 bp, Artificial Sequence
  primer
ccccaaaaccccaaaaccccttgacgtagaataatgggcttatagaag

SEQ ID NO: 346 - DNA, 53 bp, Artificial Sequence
  primer
ccccaaaaccccaaaaccccttaatcaactcactttacccactaatcaaacac

SEQ ID NO: 347 - DNA, 55 bp, Artificial Sequence
  primer
ccccaaaaccccaaaaccccatatttaagatatacagaaatatagagaatacaac

SEQ ID NO: 348 - DNA, 54 bp, Artificial Sequence
  primer
ccccaaaaccccaaaaccccattggatcaattttgaagagaattcatggaaaat

SEQ ID NO: 349 - DNA, 52 bp, Artificial Sequence
  primer
ccccaaaaccccaaaaccccatcagaaaaaatatttgaaaattcgataaagc

SEQ ID NO: 350 - DNA, 58 bp, Artificial Sequence
  primer
ccccaaaaccccaaaaccccatttcactttatttatatatagatttgaaattaaagtt

SEQ ID NO: 351 - DNA, 53 bp, Artificial Sequence
  primer
ccccaaaaccccaaaaccccagttgacatgttatttccaaattttcatggata

SEQ ID NO: 352 - DNA, 53 bp, Artificial Sequence
  primer
ccccaaaaccccaaaaccccatgataacaggaatattttataaaatagttaag

SEQ ID NO: 353 - DNA, 51 bp, Artificial Sequence
  primer
ccccaaaaccccaaaacccctcactctatgcaataaatttgttgatatatt

SEQ ID NO: 354 - DNA, 54 bp, Artificial Sequence
  primer
ccccaaaaccccaaaaccccttaaaaaaagaatagttggaataaaaatgaattt

SEQ ID NO: 355 - DNA, 56 bp, Artificial Sequence
  primer
ccccaaaaccccaaaaccccaatagataaagatgccttttttaataagtatttaac

SEQ ID NO: 356 - DNA, 55 bp, Artificial Sequence
  primer
ccccaaaaccccaaaaccccgagaggataaatttatatgaaaataaaaataaagc

SEQ ID NO: 357 - DNA, 56 bp, Artificial Sequence
  primer
ccccaaaaccccaaaaccccataaataagaaattttaagaataacgggcaaattag

SEQ ID NO: 358 - DNA, 57 bp, Artificial Sequence
  primer
ccccaaaaccccaaaaccccttgaattttaaataaacttctttgtatgatttaaatg

SEQ ID NO: 359 - DNA, 53 bp, Artificial Sequence
  primer
ccccaaaaccccaaaaccccatagattacttttcaaagaatttcttgacattc

SEQ ID NO: 360 - DNA, 56 bp, Artificial Sequence
  primer
ccccaaaaccccaaaaccccaaagcaaagaaatctgatgttttattagaaaaagtg

SEQ ID NO: 361 - DNA, 53 bp, Artificial Sequence
  primer
ccccaaaaccccaaaaccccatgagatgataatattgcctttttgcatataat

SEQ ID NO: 362 - DNA, 52 bp, Artificial Sequence
  primer
ccccaaaaccccaaaaccccatccttatacaaattcagaaaacttagcaaat

SEQ ID NO: 363 - DNA, 54 bp, Artificial Sequence
  primer
ccccaaaaccccaaaaccccgtggagaattttctaaagaattttcggaaatttg

SEQ ID NO: 364 - DNA, 56 bp, Artificial Sequence; identical to SEQ ID NO: 168
  primer

SEQ ID NO: 365 - DNA, 55 bp, Artificial Sequence; identical to SEQ ID NO: 169
  primer

SEQ ID NO: 366 - DNA, 53 bp, Artificial Sequence
  primer
gtcagtggtctcagtatgaaatttaccttggatcctcagtgtttccctttgtg

SEQ ID NO: 367 - DNA, 48 bp, Artificial Sequence
  primer
aacgctcggtctcgcagaaataaataataggcagcataataattcagg

SEQ ID NO: 368 - DNA, 39 bp, Artificial Sequence
  primer
gtcagtggtctctccagtggattcacatcactaccattg

SEQ ID NO: 369 - DNA, 55 bp, Artificial Sequence; identical to SEQ ID NO: 169
  primer

SEQ ID NO: 370 - DNA, 56 bp, Artificial Sequence; identical to SEQ ID NO: 168
  primer

SEQ ID NO: 371 - DNA, 54 bp, Artificial Sequence
  primer
acgctcggtctcgatacaatcattagcatatacatgcagtctgcttaaattaag

SEQ ID NO: 372 - DNA, 73 bp, Artificial Sequence
  primer
tctgtaattaatggatcactataatcatctggatgactattggtatttgatgacggtgcacatttgacttctt

SEQ ID NO: 373 - DNA, 73 bp, Artificial Sequence
  primer
attaattacctagtgatattagtagacctactgataaccataaactactgccacgtgtaaactgaagaaggtc

SEQ ID NO: 374 - DNA, 42 bp, Artificial Sequence
  primer
agcctaggtctcgttctttttgagggcgaggttattgttaat

SEQ ID NO: 375 - DNA, 32 bp, Artificial Sequence
  primer
ccccaaaaccccaaaaccccacaagataacat

SEQ ID NO: 376 - DNA, 44 bp, Artificial Sequence
  primer
tagtcaggtctctagaataggctcactctaaattcgagtgcaat

SEQ ID NO: 377 - DNA, 42 bp, Artificial Sequence
  primer
tctactggtctcagtatgaaatttaccttggatcctcagtgt

SEQ ID NO: 378 - DNA, 40 bp, Artificial Sequence
  primer
atcgtaggtctcaatacaatcattagcatatacatgcagt

SEQ ID NO: 379 - DNA, 33 bp, Artificial Sequence
  primer
ccccaaaaccccaaaaccccactagtcttaaat

SEQ ID NO: 380 - DNA, 54 bp, Artificial Sequence; identical to SEQ ID NO: 246
  primer

SEQ ID NO: 381 - DNA, 52 bp, Artificial Sequence; identical to SEQ ID NO: 247
  primer

SEQ ID NO: 382 - DNA, 30 bp, Artificial Sequence
  primer
aagaagaactagccagctctcactcagttc

SEQ ID NO: 383 - DNA, 30 bp, Artificial Sequence
  primer
tgtctatctcatcaggctcatcagcatagg

SEQ ID NO: 384 - DNA, 30 bp, Artificial Sequence; identical to SEQ ID NO: 382
  primer

SEQ ID NO: 385 - DNA, 30 bp, Artificial Sequence
  primer
cctctctgcccactaaattattctgacagc

SEQ ID NO: 386 - DNA, 60 bp, Artificial Sequence
  primer
ctacttgatataatacgactcactatagggaattcctaaggggagtgaagccaacaacag

SEQ ID NO: 387 - DNA, 30 bp, Artificial Sequence; identical to SEQ ID NO: 383
  primer

SEQ ID NO: 388 - DNA, 34 bp, Artificial Sequence
  primer
gtgctatgcattttaaatttattcgcattgaaga

SEQ ID NO: 389 - DNA, 33 bp, Artificial Sequence
  primer
attcagaattttagtgtgtggagtatgatagta

SEQ ID NO: 390 - DNA, 34 bp, Artificial Sequence
  primer
ggtctatattattttagtattctttctataaatg

SEQ ID NO: 391 - DNA, 35 bp, Artificial Sequence
  primer
gttacaagaatataagaaaagaaagggtgaatagg

SEQ ID NO: 392 - DNA, 46 bp, Artificial Sequence
  primer
taatacgactcactatagggggtctatattattttagtattctttc

SEQ ID NO: 393 - DNA, 35 bp, Artificial Sequence; identical to SEQ ID NO: 391
  primer

SEQ ID NO: 394 - DNA, 46 bp, Artificial Sequence; identical to SEQ ID NO: 392
  primer

SEQ ID NO: 395 - DNA, 41 bp, Artificial Sequence
  primer
taatacgactcactataggggttacaagaatataagaaaag

SEQ ID NO: 396 - DNA, 27 bp, Artificial Sequence
  primer
aacttctgtcattacattaagctttaa

SEQ ID NO: 397 - DNA, 27 bp, Artificial Sequence
  primer
ttaaagcttaatgtaatgacagaagtt

SEQ ID NO: 398 - DNA, 27 bp, Artificial Sequence
  primer
aacttctgtcattaacttaagctttaa

SEQ ID NO: 399 - DNA, 27 bp, Artificial Sequence
  primer
ttaaagcttaagttaatgacagaagtt

SEQ ID NO: 400 - DNA, 27 bp, Artificial Sequence
  primer
aacttctgtacttacattaagctttaa

SEQ ID NO: 401 - DNA, 27 bp, Artificial Sequence
  primer
ttaaagcttaatgtaagtacagaagtt

SEQ ID NO: 402 - DNA, 27 bp, Artificial Sequence
  primer
aacttctgtacttaacttaagctttaa

SEQ ID NO: 403 - DNA, 27 bp, Artificial Sequence
  primer
ttaaagcttaagttaagtacagaagtt

SEQ ID NO: 404 - DNA, 47 bp, Artificial Sequence
  primer
aacttctgtcattacattaagctttaaaaaattcaattccttttatt

SEQ ID NO: 405 - DNA, 47 bp, Artificial Sequence
  primer
aataaaaggaattgaattttttaaagcttaatgtaatgacagaagtt

SEQ ID NO: 406 - DNA, 35 bp, Artificial Sequence
  primer
tgtcattacattaagctttaaaaaattcaattcct

SEQ ID NO: 407 - DNA, 35 bp, Artificial Sequence
  primer
aggaattgaattttttaaagcttaatgtaatgaca

SEQ ID NO: 408 - DNA, 27 bp, Artificial Sequence
  primer
aacttctgtcattacattaagctttaa

SEQ ID NO: 409 - DNA, 27 bp, Artificial Sequence
  primer
ttaaagcttaatgtaatgacagaagtt

SEQ ID NO: 410 - DNA, 27 bp, Artificial Sequence
  primer
tattagaattatgttcttcatgaaatt

SEQ ID NO: 411 - DNA, 27 bp, Artificial Sequence
  primer
aatttcatgaagaacataattctaata

SEQ ID NO: 412 - protein, 372 aa, Tetrahymena
MSKAVNKKGLRPRKSDSILDHIKNKLDQEFLEDNENGEQSDEDYDQKSLNKAKKPYKKRQTQNGSELVISQQKTKAKASANNKKSAKNSQKLDEEEKIVEEEDLSPQKNGAVSEDDQQQEASTQEDDYLDRLPKSKKGLQGLLQDIEKRILHYKQLFFKEQNEIANGKRSMVPDNSIPICSDVTKLNFQALIDAQMRHAGKMFDVIMMDPPWQLSSSQPSRGVAIAYDSLSDEKIQNMPIQSLQQDGFIFVWAINAKYRVTIKMIENWGYKLVDEITWVKKTVNGKIAKGHGFYLQHAKESCLIGVKGDVDNGRFKKNIASDVIFSERRGQSQKPEEIYQYINQLCPNGNYLEIFARRNNLHDNWVSIGNEL

SEQ ID NO: 413 - protein, 449 aa, Tetrahymena
MAPKKQEQEPIRLSTRTASKKVDYLQLSNGKLEDFFDDLEEDNKPARNRSRSKKRGRKPLKKADSRSKTPSRVSNARGRSKSLGPRKTYPRKKNLSPDNQLSLLLKWRNDKIPLKSASETDNKCKVVNVKNIFKSDLSKYGANLQALFINALWKVKSRKEKEGLNINDLSNLKIPLSLMKNGILFIWSEKEILGQIVEIMEQKGFTYIENFSIMFLGLNKCLQSINHKDEDSQNSTASTNNTNNEAITSDLTLKDTSKFSDQIQDNHSEDSDQARKQQTPDDITQKKNKLLKKSSVPSIQKLFEEDPVQTPSVNKPIEKSIEQVTQEKKFVMNNLDILKSTDINNLFLRNNYPYFKKTRHTLLMFRRIGDKNQKLELRHQRTSDVVFEVTDEQDPSKVDTMMKEYVYQMIETLLPKAQFIPGVDKHLKMMELFASTDNYRPGWISVIEK

SEQ ID NO: 414 - protein, 360 aa, Tetrahymena; identical to SEQ ID NO: 63

SEQ ID NO: 415 - protein, 142 aa, Tetrahymena
MKKNGKSQNQPLDFTQYAKNMRKDLSNQDICLEDGALNHSYFLTKKGQYWTPLNQKALQRGIELFGVGNWKEINYDEFSGKANIVELELRTCMILGINDITEYYGKKISEEEQEEIKKSNIAKGKKENKLKDNIYQKLQQMQ

SEQ ID NO: 416 - protein, 584 aa, Xenopus laevis
MSDTWSHIQAHKKQLDSLRERLQRRRKDPTQLGTEVGSVESGSARSDSPGPAIQSPPQVEVEHPPDPELEKRLLGYLSELSLSLPTDSLTITNQLNTSESPVSHSCIQSLLLKFSAQELIEVRQPSITSSSSSTLVTSVDHTKLWAMIGSAGQSQRTAVKRKADDITHQKRALGSSPSIQAPPSPPRKSSVSLATASISQLTASSGGGGGGADKKGRSNKVQASHLDMEIESLLSQQSTKEQQSKKVSQEILELLNTSSAKEQSIVEKFRSRGRAQVQEFCDYGTKEECVQSGDTPQPCTKLHFRRIINKHTDESLGDCSFLNTCFHMDTCKYVHYEIDSPPEAEGDALGPQAGAAELGLHSTVGDSNVGKLFPSQWICCDIRYLDVSILGKFAVVMADPPWDIHMELPYGTLTDDEMRKLNIPILQDDGFLFLWVTGRAMELGRECLSLWGYDRVDEIIWVKTNQLQRIIRTGRTGHWLNHGKEHCLVGVKGNPQGFNRGLDCDVIVAEVRSTSHKPDEIYGMIERLSPGTRKIELFGRPHNVQPNWITLGNQLDGIHLLDPEVVARFKKRYPDGVISKPKNM

(19) United States
(12) Patent Application Publication
(10) Pub. No.: US 2021/0163900 A1
(43) Pub. Date: Jun. 3, 2021

(54) NUCLEIC ACID MODIFICATION WITH TOOLS FROM OXYTRICHA

(71) Applicant: THE TRUSTEES OF COLUMBIA UNIVERSITY IN THE CITY OF NEW YORK, New York, NY (US)

(72) Inventors: Laura Landweber, Princeton, NJ (US); Yee Ming Leslie Beh, Singapore (SG)

(21) Appl. No.: 17/153,761

(22) Filed: Jan. 20, 2021

Related U.S. Application Data
(63) Continuation of application No. PCT/US2019/042625, filed on Jul. 19, 2019.
(60) Provisional application No. 62/701,536, filed on Jul. 20, 2018.
(60) Provisional application No. 62/848,414, filed on May 15, 2019.

(51) Int. Cl. C12N 9/10; A61K 45/06; A61K 38/45; C12P 19/34; C12Q 1/6869

(52) U.S. Cl. CPC C12N 9/1007; A61K 45/06; C12Q 1/6869; C12Y 201/01072; C12P 19/34; A61K 38/45

(57) ABSTRACT

The present disclosure provides, inter alia, methods for treating a disease characterized by an abnormal level of m6dA in a subject, such as cancer, methods of modifying a nucleic acid from a cell, methods for identifying protein binding sites on DNA, methods of mediating DNA N6-adenine methylation, methods of modulating nucleosome organization and/or transcription in a cell, using MTA1c or any components thereof. The present disclosure also provides methods of generating a synthetic chromosome and synthetic chromosomes made by such methods. Pharmaceutical compositions comprising MTA1c or any components thereof and kits containing such compositions or for carrying out such processes are further provided. Eukaryotic cells, vectors and transgenic organisms comprising MTA1c or any components thereof are also provided. Synthetic chromosomes and methods of making same are also provided.

### CROSS REFERENCE TO RELATED APPLICATIONS

The present application is a continuation of PCT international application no. PCT/US2019/042625, filed on Jul. 19, 2019, which claims benefit of claims benefit of U.S. Provisional patent Application Ser. No. 62/701,536, filed on Jul. 20, 2018, and U.S. Provisional patent Application Ser. No. 62/848,414, filed on May 15, 2019. The entire contents of the aforementioned applications are incorporated by reference as if recited in full herein.

### GOVERNMENT FUNDING

This invention was made with government support under GM059708 and GM122555 awarded by the National Institutes of Health. The government has certain rights in the invention.

### FIELD OF DISCLOSURE

The present disclosure provides, inter alia, various methods, kits and compositions for modifying nucleic acid using MTA1c or any components thereof. Such embodiments may be used to treat disease and as research tools.

### INCORPORATION BY REFERENCE OF SEQUENCE LISTING

This application contains references to amino acids and/or nucleic acid sequences that have been filed concurrently herewith as sequence listing text file “CU19015-PCT-seq.txt”, file size of 478 KB, created on Aug. 28, 2019. The aforementioned sequence listing is hereby incorporated by reference in its entirety.

### BACKGROUND OF THE DISCLOSURE

Covalent modifications on DNA have long been recognized as a hallmark of epigenetic regulation. DNA N6-methyladenine (6 mA) has recently come under scrutiny in eukaryotic systems, with proposed roles in retrotransposon or gene regulation, transgenerational epigenetic inheritance, and chromatin organization (Luo et al., 2015). 6 mA exists at low levels in Arabidopsis thaliana (0.006%-0.138% 6 mA/dA), rice (0.2%), C. elegans (0.01%-0.4%), Drosophila (0.001%-0.07%), Xenopus laevis (0.00009%), mouse embryonic stem cells (ESCs) (0.0006-0.007%), human cells (Greer et al., 2015; Koziol et al., 2016; Liang et al., 2018; Wu et al., 2016; Xiao et al., 2018; Zhang et al., 2015; Zhou et al., 2018), and the mouse brain (Yao et al., 2017), although it accumulates in abundance (0.1%-0.2%) during vertebrate embryogenesis (Liu et al., 2016). Disruption of DMAD, a 6 mA demethylase, in the Drosophila brain leads to the accumulation of 6 mA and Polycomb-mediated silencing (Yao et al., 2018). The existence of 6 mA in mammals remains a subject of debate. Quantitative liquid chromatography-tandem mass spectrometry (LC-MS/MS) analysis of HeLa and mouse ESCs failed to detect 6 mA above background levels (Schiffers et al., 2017). A recent study, however, reported that loss of 6 mA in human cells promotes tumor formation (Xiao et al., 2018), suggesting that 6 mA is a biologically relevant epigenetic mark.

In contrast to metazoa, 6 mA is abundant in various unicellular eukaryotes, including ciliates (0.18%-2.5%) (Ammermann et al., 1981; Cummings et al., 1974; Gorovsky et al., 1973; Rae and Spear, 1978), and the green algae Chlamydomonas (0.3%-0.5%) (Fu et al., 2015; Hattman et al., 1978). High levels of 6 mA (up to 2.8%) were also recently reported in basal fungi (Mondo et al., 2017). Ciliates have long served as powerful models for the study of chromatin modifications (Brownell et al., 1996; Liu et al., 2007; Strahl et al., 1999; Taverna et al., 2002; Wei et al., 1998). They possess two structurally and functionally distinct nuclei within each cell (Bracht et al., 2013; Yerlici and Landweber, 2014). In the ciliate Oxytricha trifallax, the germline micronucleus is transcriptionally silent and contains ˜100 megabase-sized chromosomes (Chen et al., 2014). In contrast, the somatic macronucleus is transcriptionally active, being the sole locus of Pol II-dependent RNA production in non-developing cells (Khurana et al., 2014). The Oxytricha macronuclear genome is extraordinarily fragmented, consisting of ˜16,000 unique chromosomes with a mean length of ˜3.2 kb, most encoding a single gene. Macronuclear chromatin yields a characteristic ˜200 bp ladder upon digestion with micrococcal nuclease, indicative of regularly spaced nucleosomes (Gottschling and Cech, 1984; Lawn et al., 1978; Wada and Spear, 1980). Yet it remains unknown how and where nucleosomes are organized within these miniature chromosomes and if this in turn regulates (or is regulated by) 6 mA deposition.

### SUMMARY OF THE DISCLOSURE

The ciliate Oxytricha is a natural source of tools for RNA-guided genome reorganization and other nucleic acid modification. Long template RNAs instruct new linkages between pieces of DNA (Nowacki et al. 2008), and small RNAs instruct which DNA segments to keep (Fang et al. 2012) or eliminate. Foreseeable uses of these or other machinery derived from the Oxytricha genome include in vitro and/or in vivo modification of nucleic acids.

Intriguingly, in green algae, basal yeast, and ciliates, 6 mA is enriched in ApT dinucleotide motifs within nucleosome linker regions near promoters (Fu et al., 2015; Hattman et al., 1978; Karrer and VanNuland, 1999; Mondo et al., 2017; Pratt and Hattman, 1981; Wang et al., 2017). In the present disclosure, four ciliate proteins-named MTA1, MTA9, p1, and p2—have been identified as being necessary for 6 mA methylation in a complex form termed MTA1c. MTA1 and MTA9 contain divergent MT-A70 domains, while p1 and p2 are homeobox-like proteins that likely function in DNA binding. The present disclosure delineates key biochemical properties of this methyltransferase and dissects the function of 6 mA in vitro and in vivo.

The present disclosure provides a novel ciliate enzyme “MTA1” effective for N6-methyladenine (m6dA) methylation of DNA (see, e.g., Appendix 4). MTA1 has been identified in a ciliate, Tetrahymena thermophila, and its functional role validated in m6dA methylation in Oxytricha. (See, Genbank ID: XP 001032074.3 [Tetrahymena MTA1] and EJY79437.1 [Oxytricha MTA1]). MTA1 is evolutionarily distinct from all known m6dA methyltransferases. Evolutionary analysis reveals that it is present in ciliates (including Oxytricha and Tetrahymena), algae, and basal fungi, but not multicellular eukaryotes. MTA1 exhibits a unique substrate specificity in vivo, being essential for the deposition of dimethylated AT (5′-A*T-3′/3′-TA*-5′), as well as a wide range of other motifs in vivo (FIGS. 1A-1B). The inventors have been actively characterizing the biochemical properties and enzymology of Tetrahymena and Oxytricha MTA1, including its binding partners, in vitro substrate specificity (DNA vs. RNA and sequence motifs therein), methylation kinetics, and structural basis of these activities.

The present disclosure provides that MTA1c or any components thereof presents immediate commercial applications in: 1) generation of DNA substrates containing m6dA at locations distinct from known m6dA methyltransferases, circumventing the need for slow, expensive synthesis of methylated DNA; and 2) rational design of N6-adenine methylating enzymes with novel substrate specificities.

Accordingly, one embodiment of the present disclosure is a method of modifying a nucleic acid from a cell, the cell derived from a multicellular eukaryote. This method comprises the steps of: (a) obtaining the nucleic acid from the cell; and (b) contacting the nucleic acid with MTA1c or any components thereof under conditions effective to methylate the nucleic acid.

The modified base, m6dA, has been discovered in a wide range of eukaryotes, including humans. m6dA levels are significantly reduced in gastric and liver cancer tissues, and disruption of m6dA promotes tumor formation (Xiao et al. 2018). As disclosed herein, MTA1 is a novel m6dA “writer”, paving the way for cost-effective methods to understand mechanisms of m6dA function in biomedically relevant models.

Accordingly, another embodiment of the present disclosure is a method of treating or ameliorating the effects of a disease characterized by an abnormal level of m6dA in a subject. This method comprises administering to the subject an amount of MTA1c or any components thereof effective to modulate m6dA levels in the subject. In some embodiments, the modulation comprises restoring m6dA levels to normal or near-normal ranges in the subject.

Another embodiment of the present disclosure is a pharmaceutical composition comprising MTA1c or any components thereof that is effective to modulate m6dA levels in a subject in need thereof and a pharmaceutically acceptable carrier, diluent, adjuvant or vehicle.

Yet another embodiment of the present disclosure is a kit for treating or ameliorating the effects of a disease characterized by an abnormal level of m6dA in a subject, such as, e.g., cancer, comprising an effective amount of MTA1c or any components thereof, packaged together with instructions for its use.

Another embodiment of the present disclosure is a cell line obtained from a multicellular eukaryote comprising a nucleic acid encoding MTA1c or any components thereof and/or an MTA1c protein complex or any components thereof. As used herein, a “cell line” refers to all types of cell lines such as, e.g., immortalized cell lines and primary cell lines. In certain embodiments, the nucleic acid encoding MTA1c or any components thereof is operably linked to a recombinant expression vector.

Another embodiment of the present disclosure is a recombinant expression vector comprising a polynucleotide encoding MTA1c or any components thereof.

Still another embodiment of the present disclosure is a transgenic organism whose genome comprises a transgene comprising a nucleotide sequence encoding MTA1c or any components thereof. Non-limiting examples of possible organism include an archaea, a bacterium, a eukaryotic single-cell organism, algae, a plant, an animal, an invertebrate, a fly, a worm, a cnidarian, a vertebrate, a fish, a frog, a bird, a mammal, an ungulate, a rodent, a rat, a mouse, and a non-human primate.

The present disclosure also provides a method of identifying protein binding sites on DNA. This method comprises the steps of: (a) providing DNA; (b) contacting the DNA with MTA1c or any components thereof under conditions effective to methylate the DNA; (c) contacting the DNA with one or more proteins; (d) contacting the DNA with an enzyme effective to hydrolize the DNA in positions where no protein binding occurs; (e) removing the DNA bound protein; and (f) isolating and sequencing the DNA fragments. In certain embodiments, the one or more proteins in step (c) comprise histone octamers.

Another embodiment of the present disclosure is a method of mediating DNA N6-adenine methylation. This method comprises the steps of: (a) providing DNA; and (b) contacting the DNA with MTA1c or any components thereof under conditions effective to methylate the DNA.

Another embodiment of the present disclosure is a method of modulating nucleosome organization and/or transcription in a cell, comprising providing to the cell an agent that is effective to modulate the expression of MTA1c or any components thereof.

The present disclosure also provides a method of generating a synthetic chromosome. This method comprises the steps of: (a) generating chromosome segments containing terminal restriction sites, wherein the chromosome segments comprise one or more m6dA bases; (b) digesting the chromosome segments with a restriction enzyme; and (c) purifying and ligating the digested chromosome segments to form a synthetic chromosome. In some embodiments, the method further comprises enriching the synthetic chromosome. A synthetic chromosome made by the method above is also provided.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The following drawings form part of the present specification and are included to further demonstrate certain aspects of the present disclosure. The disclosure may be better understood by reference to one or more of these drawings in combination with the detailed description of specific embodiments presented herein.

FIGS. 1A-1E show epigenomic profiles of Oxytricha chromosomes.

FIG. 1A shows meta-chromosome plots of chromatin organization at Oxytricha macronuclear chromosome ends. Heterodimeric telomere end-binding protein complexes (orange ovals) protect each end in vivo. Horizontal red bar: promoter. The 5′ chromosome end is proximal to TSSs. Nucleosome occupancy, normalized Mnaseseq coverage; 6 mA, total 6 mA number; Transcription start sites, total number of called TSSs.

FIG. 1B shows histograms of the total number of 6 mA marks within each linker in Oxytricha chromosomes. Distinct linkers are depicted as horizontal blue lines.

FIG. 1C shows that poly(A)-enriched RNA-seq levels positively correlate with 6 mA. Genes are sorted according to the total number of 6 mA marks 0-800 bp downstream of the TSS. FPKM, fragments per kilobase of transcript per million mapped RNA-seq reads. Notch in the boxplot denotes median, ends of boxplot denote first and third quartiles, upper whisker denotes third quantile+1.5× interquartile range (IQR), and lower whisker denotes data quartile 1-1.5×IQR.

FIG. 1D shows that composite analysis of 65,107 methylation sites reveals that 6 mA (marked with 1 occurs within a 5′-ApT-3′ dinucleotide motif.

FIG. 1E provides the distribution of various 6 mA dinucleotide motifs across the genome. Asterisk, 6 mA.

FIGS. 2A-2G show purification and characterization of the ciliate 6 mA methyltransferase.

FIG. 2A provides phylogenetic analysis of MT-A70 proteins. Bold MTA1 and MTA9 genes are experimentally characterized in this study. Paralogs of MTA1 and MTA9 are labeled as “-B.” Posterior probabilities >0.65 are shown. Gray triangle represents outgroup of bacterial sequences. The complete phylogenetic tree is shown in FIG. 9G. Gene names are in Table 5. Tth, Tetrahymena thermophila; Otri, Oxytricha trifallax.

FIG. 2B shows the phylogenetic distribution of the occurrence of ApT 6 mA motifs and MT-A70 protein families. Filled square denotes its presence in a taxon. The basal yeast clade is comprised of L. transversale, A. repens, H. vesiculosa, S. racemosum, L. pennispora, B. meristosporus, P. finnis, and A. robustus.

FIG. 2C is an experimental scheme depicting the partial purification of DNA methyltransferase activity from Tetrahymena nuclear extracts.

FIG. 2D show gene expression and protein abundance of candidate genes in partially purified Tetrahymena nuclear extracts. UniProt IDs are listed in Table 5. RNA-seq data are from (Xiong et al. 2012). FPKM, fragments per kilobase of transcript per million mapped RNA-seq reads. Low, Mid, and High DNA methylase activity correspond to fractions eluting from the Nuvia cPrime and Superdex 200 columns in FIG. 2C. Total spectrum counts, total number of LC-MS/MS fragmentation spectra that match peptides from a target protein.

FIG. 2E shows DNA methyltransferase assay using [3H]SAM. Vertical axis represents scintillation counts. Error bars represent SEM (n=3).

FIG. 2F shows dot blot assay using cold SAM.

FIG. 2G shows DNA methyltransferase assay performed on different nucleic acid substrates in the presence of MTA1, MTA9, p1, and p2. Sense ssDNA are 5′→3′; antisense are 3′→5′. ApT dinucleotides are labeled in bold red. Horizontal blue lines in hemimethylated dsDNA substrates denote possible locations where 6 mA may be installed by EcoGII (prior to this assay). Relative activity denotes scintillation counts normalized against the unmethylated 27 bp dsDNA substrate with two ApT motifs (top-most dsDNA substrate). An enlarged bar plot of relative activity on 27 bp unmethylated dsDNA substrates is included in FIG. 10K. Error bars represent SEM (n=3).

FIGS. 3A-3E show genome-wide loss of 6 mA in mta1 mutants.

FIG. 3A shows schematic depicting the disruption of Oxytricha MTA1 open reading frame. Flanking dark blue bars: 5′ and 3′ UTR; yellow, open reading frame; red, retention of 62 bp ectopic DNA segment; gray bar, intron; Internal light blue bar, annotated MT-A70 domain; ATG, start codon; TGA, stop codon. Agarose gel analysis shows PCR confirmation of ectopic DNA retention.

FIG. 3B shows dot blot analysis of RNase-treated genomic DNA.

FIG. 3C shows histogram of 6 mA counts near 5′ and 3′ Oxytricha chromosome ends. Inset depicts histogram of fold change in total 6 mA in each chromosome, between mutant and wild-type cell lines.

FIG. 3D shows that chromosomes are sorted into 10 groups according to total 6 mA in wild-type cells (blue boxplots). For each group, the total 6 mA per chromosome in mutants and the difference in total 6 mA per chromosome are plotted below. Boxplot features are as described in FIG. 1C.

FIG. 3E shows motif distribution in wild-type and mta1 mutants. Loss of ApT dimethylated motif is underlined.

FIGS. 4A-4E show effects of 6ma on nucleosome organization in vitro and in vivo.

FIG. 4A shows the experimental workflow for the generation of mini-genome DNA.

FIG. 4B shows agarose gel analysis of Oxytricha gDNA (Native) and mini-genome DNA before chromatin assembly.

FIG. 4C shows that methylated regions exhibit lower nucleosome occupancy in vitro but not in vivo. Overlapping 51 bp windows were analyzed across 98 chromosomes. For each window, the change in nucleosome occupancy in the absence versus presence of 6 mA was calculated. Boxplot features are as described in FIG. 1C. p values were calculated using a two-sample unequal variance t test. N.S., non-significant, with p>0.05.

FIG. 4D shows the reduction in nucleosome occupancy at methylated loci in vitro (black arrowheads). For in vitro MNase-seq, +6 mA refers to chromatin assembled on Oxytricha gDNA, while −6 mA denotes chromatin assembled on mini-genome DNA. The vertical axis for SMRT-seq data denotes confidence score [−10 log(p value)] of detection of 6 mA, while that for in vitro MNase-seq data denotes nucleosome occupancy.

FIG. 4E shows no change in nucleosome occupancy in linker regions despite loss of 6 mA in mta1 mutants. Vertical axes are the same as FIG. 4D.

FIGS. 5A-5C show modular synthesis of full-length Oxytricha chromosomes.

FIG. 5A shows features of the chromosome selected for synthesis. Gray boxes represent exons. All data tracks represent normalized coverage except for SMRT-seq, which represents the confidence score [−10 log(p value)] of detection of each methylated base.

FIG. 5B shows the schematic of chromosome construction. Different colors denote DNA building blocks ligated to form the full-length chromosome. Precise 6 mA sites (bold red) represent cognate 6 mA positions revealed by SMRT-seq in native genomic DNA. These are introduced via oligonucleotide synthesis. For chromosome 5, 6 mA sites (non-bold red) represent possible locations ectopically installed by a bacterial 6 mA methyltransferase, EcoGII. Intervening sequence within chromosomes 5 and 6 is represented as “ . . . ”.

FIG. 5C shows native polyacrylamide gel analysis and anti-6 mA dot blot analysis of building blocks and purified synthetic chromosomes.

FIGS. 6A-6E show quantitative modulation of nucleosome occupancy by 6 mA.

FIG. 6A shows the experimental workflow. Chromatin is assembled using either salt dialysis or the NAP1 histone chaperone. Italicized blue steps are selectively included.

FIG. 6B shows the tiling qPCR analysis of synthetic chromosome with cognate 6 mA sites. Horizontal gray box represents annotated gene, and vertical black lines depict native 6 mA positions. Horizontal blue bars span −100 bp regions amplified by qPCR. Red horizontal lines represent the region containing 6 mA. Hemi methyl chromosomes contain 6 mA on the antisense and sense strands, respectively, while the Full methyl chromosome has 6 mA on both strands. Black arrowheads: decrease in nucleosome occupancy specifically at the 6 mA cluster.

FIG. 6C shows the tiling qPCR analysis of ectopically methylated synthetic chromosome. Vertical black lines illustrate possible 6 mA sites installed enzymatically. Red arrowheads: decrease in nucleosome occupancy in the ectopically methylated region. Black arrowheads: position of cognate 6 mA sites (not in this construct).

FIG. 6D shows the tiling qPCR analysis of chromatin from FIG. 6B that is subsequently incubated with ACF and/or ATP. ACF equalizes nucleosome occupancy between the 6 mA cluster and flanking regions in the presence of ATP (black line). Nucleosome occupancy at the methylated region is not restored to the same level as the unmethylated control (black arrowheads).

FIG. 6E shows that MNase-seq analysis of chromatin is assembled on native gDNA (“+” 6 mA) and mini-genome DNA (“−” 6 mA) using NAP1±ACF and ATP. p values were calculated using a two-sample unequal variance t test.

FIGS. 7A-7F show effects of 6 mA on gene expression and cell viability in vivo.

FIG. 7A shows the following: Horizontal axis: the mean RNA-seq counts across all biological replicates from wild-type and mta1 mutant data for each gene. Vertical axis: log 2(fold change) in gene expression (mutant/wild type).

FIG. 7B shows that upregulated genes tend to be sparsely methylated compared to randomly subsampled genes (gray lines).

FIG. 7C shows RNA-seq analysis of MTA1 expression during the sexual cycle of Oxytricha. RNA-seq time course data are from Swart et al. (2013). The total duration of the sexual cycle is ˜60 h.

FIG. 7D shows survival analysis of Oxytricha cells during the sexual cycle. The total cell number at each time point is normalized to 27 h data to obtain the percentage survival. Error bars represent SEM (n=4).

FIG. 7E is a model illustrating the impact of 6 mA methylation by MTA1c on nucleosome organization and gene expression.

FIG. 7F shows the comparison of DNA and RNA N6-adenine methyltransferases. Blue denotes catalytic subunit; yellow denotes subunit with predicted DNA or RNA binding domain.

FIGS. 8A-8B show MS analysis of 6 mA in ciliate DNA.

FIG. 8A shows that Oxytricha and Tetrahymena genomic DNA were digested into nucleosides using degradase enzyme mix, followed by analysis using reverse-phase HPLC and mass spectrometry. Isotopically labeled dA and 6 mA standards (¹⁵N5-dA and D3-6 mA) were mixed with each sample to allow quantitative measurement of endogenous dA and 6 mA concentrations. MS/MS analysis of labeled dA and 6 mA standards confirmed the mass of the nucleobase. Fluted peaks with expected masses of dA and 6 mA, and with highly similar retention times (RT) to internal standards are detected in Oxytricha and Tetrahymena nucleosides.

FIG. 8B shows the quantitation of dA and 6 mA levels in Oxytricha and Tetrahymena gDNA using internal isotopically labeled nucleoside standards. The detected level of 6 mA in Tetrahymena gDNA agrees with earlier reports (Gorovsky et al., 1973; Pratt and Hallman, 1981). The calculated abundance of 6 mA relative to (dA+6 mA) in Oxytricha is ˜0.71%, which is similar to the estimate from SMRT-seq base calls (0.78-1.04%). Note that the calculation from SMRT-seq data is expected to be an overestimate because 6 mA is scored at being present or absent at each site in the genome for this purpose. In actual fact, 6 mA sites may be partially methylated (FIG. 11A). Neither 6 mA nor dA was detected from LC-MS analysis of Oxytricha culture media, arguing against spurious signal arising from contamination or overall technical handling. The PacBio and LC-MS measurements of % 6 mA in Oxytricha are both similar to thin layer chromatography analysis of nucleotides (0.6-0.7%) from a distinct but closely related species, Oxytricha fallax (Rae and Spear, 1978).

FIGS. 9A-9K show analysis of 6 mA and methyltransferase components in Tetrahymena.

FIG. 9A shows Tetrahymena MNase-seq data from (Beh et al., 2015), while SMRT-seq data were generated in the present disclosure. Meta-chromosome plots overlaying in vivo MNase-seq (nucleosome occupancy) and SMRT-seq (6 mA), relative to annotated transcription start sites. 6 mA lies mainly within nucleosome linker regions, between the +1, +2, +3, and +4 nucleosomes.

FIG. 9B shows histograms of the total number of 6 mA marks within each linker in Tetrahymena genes. Calculations are performed as described in FIG. 1B. Distinct linkers are highlighted with horizontal bold blue lines.

FIG. 9C shows the relationship between transcriptional activity and total number of 6 mA marks in Tetrahymena genes. Analysis is performed as in FIG. 1C. RNA-seq data was obtained from (Xiong et al., 2012).

FIG. 9D shows that composite analysis of 441,618 methylation sites reveals that 6 mA occurs within a 5′-ApT-3′ dinucleotide motif in Tetrahymena, consistent with previous experiments (Bromberg et al., 1982; Wang et al., 2017) and similar to Oxytricha.

FIG. 9E shows distribution of various 6 mA dinucleotide motifs across the genome.

FIG. 9F shows organization of transcription (mRNA-seq), nucleosome organization (MNase-seq), and 6 mA (SMRT-seq) in a Tetrahymena gene.

FIG. 9G shows that all sequences used for phylogeny construction are listed in Table 1. Abbreviations: Cel: Caenorhabditis elegans; Ath: Arabidopsis thaliana; Sra: Syncephalastrum racemosum; Hve: Hesseltinella vesiculosa; Are: Absidia repens; Dre: Danio redo; Has: Homo sapiens; Ssc: Sus scrota; Mmu: Mus musculus; Xla: Xenopus laevis; Dme: Drosophila melanogaster; Cre: Chlamydomonas reinhardtii; Ltr: Lobosporangium transversale; Lpe: Linderina pennispora; Bme: Basidiobolus meristosporus; Pfi: Piromyces finnis; Aro: Anaeromyces robustus; Tth: Tetrahymena thermophila; Otri: Oxytricha trifallax. This Bayesian phylogenetic tree of MT-A70 proteins is the same as in FIG. 2A, except that all sequences are now included and labeled. TAMT-1 proteins are named according to (Luo et al., 2018).

FIG. 9H shows Bayesian phylogenetic tree of p1 proteins.

FIG. 9I shows Bayesian phylogenetic tree of p2 proteins. Dashed box depicts outgroup consisting of vertebrate SNAPC4 genes. These genes bear weak similarity to the homeobox-like domain of p2 proteins, but do not group phylogenetically with them and are therefore unlikely to be functionally homologs.

FIG. 9J shows phylogenetic distribution of ApT 6 mA motif and various proteins, as depicted in FIG. 2B, but now also including TAMT-1, p1, and p2 proteins. Filled boxes denote the presence of a particular protein in a taxon. Open dashed boxes indicate the presence of SNAPC4 genes in vertebrates.

FIG. 9K shows the gene expression profiles of Tetrahymena MTA1, MTA9, p1 and p2. Microarray counts represent poly(A)′ expression levels, and are obtained from TetraFGD (Miao et al., 2009; Xiong et al., 2011). MTA1, MTA9, p1 and p2 were found in our study to co-elute with 6 mA methylase activity. On the other hand, TAMT-1 is a putative DNA methyltransferase described by (Luo et al., 2018). The horizontal axis categories beginning with “S” and “C” represent the number of hours since the onset of starvation and conjugation (mating), respectively. “Low,” “Med,” and “High” denote relative cell densities during log-phase growth. Blue and orange traces represent data from two biological replicates. Green and red shaded regions show the peaks in poly(A)* RNA expression in vegetative growth and conjugation, respectively, for MTA1, MTA9, p1 and p2. Note that their expression pattern differs from TAMT-1.

FIGS. 10A-10N show further characterization of 6 mA methyltransferase activity and MTA1c.

FIG. 10A shows that fractionation of nuclear extracts on a Q Sepharose column results in two distinct peaks of DNA methyltransferase activity, denoted as “Low Salt sample” and “High Salt sample” by black horizontal bars. FT denotes column flow-through. The DNA methyltransferase assay is performed as in FIG. 2E. The salt concentration at which individual fractions elute from the column is plotted against DNA methyltransferase activity of each fraction (counts per minute). Inset shows DNA methyltransferase activity of the input nuclear extract, flowthrough from the Q Sepharose column, and blank control (nuclear extract buffer). Orange and blue plots denote replicates derived from independent preparations of nuclear extract.

FIG. 10B is DNA methyltransferase assay showing that the activity from nuclear extracts is heat-sensitive and requires addition of DNA and SAM. Error bars represent s.e.m. (n=3).

FIG. 10C is dot blot showing that nuclear extracts mediate 6 mA methylation. Note that the low salt sample has substantial DNase activity, resulting in a lower amount of DNA available for dot blot analysis. DNA substrate, nuclear extract, and SAM cofactor were mixed as in panels A and B. The DNA was subsequently purified and used for dot blot analysis.

FIG. 10D shows domain organization of Tetrahymena MTA1, MTA9, p1, and p2. Protein domains are predicted using hmmscan on the EMBL-EBI webserver (Finn et al., 2015). “aa” denotes amino acids. Start and end coordinates of each domain are stated below each polypeptide.

FIG. 10E shows the sequence alignment of human (Hsa) METTL3 with Tetrahymena (Tth) and Oxytricha (Otri) MTA1/MTA9, within the MT-A70 domain. Horizontal black bars underscore the DPPW catalytic motif, and the N549/0550 residues in human METTL3 that interact with the ribose moiety of the SAM cofactor. Note that the DPPW catalytic motif is conserved in MTA1 but not MTA9.

FIG. 10F shows dot blot analysis of hemimethylated dsDNA substrates. Sense or antisense oligonucleotides were first individually methylated using the EcoGII bacterial 6 mA methyltransferase. Each methylated ssDNA was subsequently purified and annealed with an unmethylated complementary strand to form hemimethylated constructs.

FIG. 10G shows SDS-PAGE analysis of recombinant proteins. Full length proteins were expressed and purified from E. coli. Bands of expected size are indicated with a black arrowhead.

FIG. 10H is methyttransferase assay using radiolabeled SAM on DNA and RNA substrates, coupled with gel analysis of nucleic acid integrity. ssRNA and dsRNA were produced by in vitro transcription from the 350 bp dsDNA template using 17 RNA polymerase, and subsequently purified before use in this assay. Methyltransferase activity on equimolar amounts of each substrate was measured after incubation at 37° C. for 6 hr, and depicted as either scintillation counts (Counts per minute), or normalized to the 350 bp dsDNA sample (Relative activity). Only dsDNA, and not dsRNA or ssRNA, was methylated. Activity measurements are represented as scintillation counts (counts per minute). In addition, aliquots from each reaction containing DNA or RNA substrate and recombinant MTA1c (ie. MTA1, MTA7, p1 and p2 proteins) were withdrawn at 0, 1, 2, 3, or 6 hr during the 37° C. incubation, purified using phenol:chloroform extraction and ethanol precipitation, and subsequently analyzed on a non-denaturing agarose gel. Both dsDNA and dsRNA substrates remained intact after 6 hr. The ssRNA migrates more diffusely on a nondenaturing agarose gel, with some decrease in size over time, suggesting partial degradation and/or RNA folding; however, there is no detectable methylation of ssRNA despite a significant presence on the agarose gel after 6 hr at 37° C. It is unlikely that this species is too short to be methylated, since MTA1c can methylate significantly shorter substrates such as 27 bp dsDNA (FIGS. 2G, 10I, 10J, and 10K). Error bars represent s.e.m. (n=3).

FIG. 10I is DNA methyltransferase assay using radiolabeled SAM, on ssDNA oligonucleotides or annealed dsDNA substrates. All four recombinant MTA1c protein components—MTA1, MTA9, p1, and p2—were included in each sample. Activity measurements are represented as scintillation counts (counts per minute). dsDNA substrates were prepared by annealing ssDNA oligonucleotides, as in FIG. 2G. Sense ssDNA nucleotide sequences are depicted in the 5′ 3′ direction, while antisense ssDNA is depicted as 3′ 5′. Error bars represent s.e.m. (n=3).

FIG. 10J is control [³H]SAM assay using hemimethylated dsDNA. Reactions depicted in red represent hemimethylated dsDNA incubated with [3H]SAM in the absence of recombinant MTA1c (MTA1, MTA9, p1, and p2 proteins). These reactions showed no methyltransferase activity, verifying that there is no contaminating EcoGII methyltransferase in hemimethylated dsDNA preparations. Activity measurements are shown as scintillation counts, or as “Relative Activity” (normalized against the sample containing unmethylated DNA substrate, [3H]SAM, and MTA1c protein). Hemimethylated dsDNA substrates in this panel are the same as those used in FIG. 2G. The unmethylated dsDNA substrate used in this panel is the same as the top-most dsDNA substrate in FIG. 2G, with two uninterrupted ApT dinucleotides. Error bars represent s.e.m. (n=3).

FIG. 10K is DNA methyltransferase assay using radiolabeled SAM, on dsDNA substrates with disrupted ApT dinucleotides. All four recombinant MTA1c protein components—MTA1, MTA9, p1, and p2—were included in each sample. Activity measurements are normalized against the parent dsDNA construct with two uninterrupted ApT dinucleotides (top-most construct in this panel). ApT dinucleotide positions are labeled in bold red. Note that the parent dsDNA construct is identical to that in FIG. 10L. Error bars represent s.e.m. (n=3).

FIG. 10L is DNA methyitransferase assay using radiolabeled SAM, on dsDNA substrates with shifted ApT dinucleotides. All four recombinant MTA1c protein components—MTA1, MTA9, p1, and p2—were included in each sample. Activity measurements are normalized against the parent dsDNA construct with two uninterrupted ApT dinucleotides (top-most construct in this panel). The parent construct is identical to that in FIG. 10K. ApT dinucleotides are labeled in bold red. The adjacent nucleotides are labeled in bold black to highlight the 4-mer sequence that contains each ApT dinucleotide. Error bars represent s.e.m. (n=3).

FIG. 10M shows motif frequencies of all 4-mer sequences containing methylated ApT dinucleotides in the Tetrahymena and Oxytricha genomes. A′ denotes 6 mA. The 4-mers TA′TA and CKTT are colored in red and blue, respectively, to highlight their large difference in genomic frequencies.

FIG. 10N shows motif frequencies of 4-mer sequences—regardless of methylation state—in Tetrahymena and Oxytricha. These were calculated from genomic sequence between the 5′ chromosome end and the +4 nucleosome peak (Oxytricha), or between the TSS and the +4 nucleosome peak (Tetrahymena). Analysis was restricted to these regions in order to serve as “background” frequencies for comparison to A′T methylated 4-mers, which are also mainly found downstream of TSSs. The 4-mers TATA and GATT are colored in red and blue, respectively, to facilitate comparison with methylated TA′TA and CA*TT in panel M.

FIGS. 11A-11D show supplemental SMRT-seq data analyses.

FIG. 11A shows the following: Top two panels depict PacBio coverage (horizontal axis) plotted against fractional methylation at each called 6 mA site (vertical axis). Bottom left panel is a histogram of fractional methylation of all 6 mA sites. Bottom right panel is a histogram of IPD ratios of all 6 mA sites. Mutant datasets show significantly lower fractional methylation and IPD ratios at 6 mA sites than wild-type data.

FIG. 11B shows that wild-type SMRT-seq data are randomly subsampled 15 times, such that the resulting coverage is lower than ‘Mal mutant data. The difference in PacBio coverage between mutant and subsampled wild-type data is calculated for each chromosome, and is collectively represented as an olive boxplot (top panel). This set of calculations is repeated 15 times for each subsampled dataset, resulting in a series of 15 boxplots. The difference in PacBio coverage between mutant and fully sampled wild-type data is represented as a violet boxplot. Separately, the difference in total 6 mA marks per chromosome is calculated for respective datasets, and boxplots are shown in the bottom panel. Mutant datasets consistently yield lower numbers of called 6 mA marks than subsampled wild-type, despite the former having higher coverage than the latter.

FIG. 11C shows the scatterplot of total number of 6 mA marks per chromosome in wild-type versus mutant data. PacBio cutoffs for calling 6 mA marks are varied as shown. A greater number of 6 mA marks per chromosome are consistently detected in wild-type than mutant data.

FIG. 11D shows the boxplot of PacBio chromosome coverage in individual wild-type and mutant biological replicates (left panel). Only chromosomes with 100-150× PacBio coverage are shown. The total number of 6 mA marks in each of these chromosomes are plotted in the right panel. Wild-type replicates show consistently higher numbers of 6 mA marks per chromosome than mutant replicates.

FIGS. 12A-12H show analysis of nucleosome organization and confirmation of ectopic DNA insertion in mta1 mutants. Description of analysis in panels A-G: Nucleosomes are grouped according to their “starting” 6 mA level, defined as the total number of 6 mA marks±200 bp from the nucleosome dyad in wild-type cells (WT). The dyad is assigned to be the peak position of MNase-seq reads. Similarly, linkers are grouped according to their “starting” methylation level, defined as the total number of 6 mA marks between two flanking nucleosome dyads (or between the 5′ chromosome end and the terminal nucleosome) in wild-type cells. Loci with high starting 6 mA have methylation greater than or equal to the 90th percentile of starting 6 mA levels, and show greater changes in methylation between mutant and wild-type cells (FIG. 3D). Those with low starting 6 mA are in the lowest 10th percentile. if 6 mA impacts nucleosome organization in vivo, then loci with high starting 6 mA should show a greater change in nucleosome organization. Possible effects are illustrated in panels A-C. Vertical green lines depict 6 mA marks, while blue and red peaks denote nucleosome occupancy. The plots shown in panels A-C illustrate the idealized result if 6 mA disfavors nucleosomes in vivo. Actual effects are shown in panels D-G. “Wild type” is abbreviated as WT. Analyses are restricted to the 5′ chromosome end.

FIG. 12A shows that 6 mA loss may result in an increase in nucleosome fuzziness (highlighted with bold red double-sided arrow). The effect should be greater for nucleosomes with high starting 6 mA due to greater change in 6 mA between mutant and wild-type cells (“Change in nucleosome fuzziness” Box). Nucleosomes should, in turn, exhibit lower occupancy near the peak position, and higher occupancy in flanking regions (“Change in Nucleosome occupancy” Box; highlighted with red arrowheads and plotted ±73 bp from the dyad). Nucleosome fuzziness is calculated as the standard deviation of MNase-seq read locations ±73 bp from the dyad.

FIG. 12B shows that 6 mA loss from nucleosome linker regions may result in a decrease in linker length (highlighted with bold red bracket). If so, the magnitude of decrease in linker length should be greater for linkers with high starting 6 mA (“Change in linker length” Box).

FIG. 12C shows that 6 mA loss may result in an increase in occupancy directly over the methylated linker region (highlighted with bold red bracket). If so, the magnitude of increase in linker occupancy should be greater for regions with high starting 6 mA (“Change in linker occupancy” Box). Linker occupancy denotes the average MNase-seq coverage ±25 bp from the midpoint between flanking nucleosome dyads or chromosome end. As an example, for the +1/+2 nucleosome linker, occupancy is calculated ±25 bp from the midpoint of the +1 and +2 nucleosome dyad positions. Since nucleosome linker length in Oxytricha is ˜200 bp (FIG. 12F, bottom panels), the genomic window used to calculate linker occupancy has minimal overlap with that for calculating nucleosome fuzziness and occupancy in panel A.

FIG. 12D shows the impact of 6 mA loss on nucleosome fuzziness. For each nucleosome, the change in fuzziness between mutant and wild-type cells is calculated. Boxplots represent the distribution of changes in fuzziness scores. “MNase-seq” denotes sequencing of nucleosomal DNA obtained from Oxytricha chromatin in vivo, while “Control gDNA-seq” represents sequencing of MNase-digested, naked genomic DNA in vitro. Boxplot features are as described in FIG. 1C. Distributions are compared using a Wilcoxon rank-sum test. N.S denotes “non-significant,” with p>0.01.

FIG. 12E shows the impact of 6 mA loss on nucleosome occupancy. For each nucleosome, the difference in nucleosome occupancy between mutant and wild-type cells is calculated at individual basepairs±73 bp around the nucleosome dyad. Data are averaged and depicted as line plots. The change in occupancy at the dyad is compared between nucleosomes with high and low starting 6 mA using a Wilcoxon rank-sum test.

FIG. 12F shows the impact of 6 mA loss on linker length. Three types of linkers are analyzed: between the 5′ chromosome end and +1 nucleosome dyad, between the +1 and +2 nucleosome dyads, and between the +2 and +3 nucleosome dyads. For each linker, the difference in its length between mutant and wild-type cells is calculated. The resulting distribution of linker length differences is plotted as a histogram (top-most row of this panel). Distributions of linker length differences are compared using two-sample unequal variance t test. N.S. indicates “not significant,” with p>0.01. Separately, the respective distributions of linker lengths in mutant and wild-type cells are plotted in the bottom two rows of this panel. The median linker length from each group is included as an inset.

FIG. 12G shows the impact of 6 mA loss on linker occupancy. Linkers are binned as in panel F. For each linker, the difference in occupancy between mutant and wild-type cells is calculated. The resulting distribution of changes in linker occupancy is represented as a boxplot. Distributions are compared using two-sample unequal variance t test. N.S. indicates “not significant,” with p>0.01. Boxplot features are as described in FIG. 1C.

FIG. 12H shows poly(A)⁺ RNaseq analysis of wild-type and mta1 mutants. “ATG” denotes start codon of MTA1 gene. A 62 bp ectopic DNA insertion results in a frameshift mutation in the MTA1 coding region. Three wild-type (WTI, WT2, wr3) and mutant (mta1′, mta12, mta13) biological replicates are analyzed. Short horizontal bars represent RNaseq reads, which are, −.75 nt in length and mapped to the reference sequence. For a read to be successfully mapped, it must have no more than 2 mismatches relative to the reference sequence. Unmapped reads are discarded. Blue and red bars denote RNaseq reads that map to native and ectopic regions, respectively. RNaseq reads overlapping the ectopic region are detected in mutant but not wild-type replicates. These reads span junctions between the ectopic and flanking coding regions, confirming the site of ectopic insertion.

FIGS. 13A-13I show gel analysis of histone octamers and assembled chromatin. Description for panels A-D: Xenopus unmodified core histones were recombinantly expressed. Oxytricha histones were acid-extracted from vegetative nuclei. Oxytricha and Xenopus histones were subsequently refolded into octamers and purified through size exclusion chromatography. Description for panels E-I: Xenopus or Oxytricha histone octamers were assembled on DNA and subsequently digested with MNase to obtain ˜150 bp mononucleosome-sized fragments (labeled with red arrowheads). The resulting products were visualized by agarose gel electrophoresis. Mononucleosomal DNA was gel-excised and analyzed using Illumina sequencing or tiling qPCR analysis in FIGS. 4A-4E, 6A—6E, and 14A—14F.

FIG. 13A shows reverse-phase HPLC purification of acid-extracted Oxytricha histones. Fractions 1-5 were individually collected and analyzed by Coomassie staining and western blotting.

FIG. 13B shows SDS-PAGE analysis of purified Oxytricha histone fractions.

FIG. 13C shows Western blot analysis of Oxytricha histone fractions 1-5. The fraction that is most enriched in each type of histone is colored in red. Arrowheads indicate likely histone bands.

FIG. 13D shows SDS-PAGE analysis of purified Oxytricha and Xenopus histone octamers.

FIG. 13E shows that chromatin was assembled on PCR-amplified Oxytricha mini-genome DNA, digested with MNase, and analyzed by agarose gel electrophoresis.

FIG. 13F shows that chromatin was assembled on native Oxytricha genomic DNA, digested with MNase, and analyzed by agarose gel electrophoresis.

FIG. 13G shows that chromatin was assembled with synthetic chromosome DNA, digested with MNase, and visualized by agarose gel electrophoresis. All assemblies with synthetic chromosomes were performed in the presence of an approximately 100-fold mass excess of buffer DNA relative to synthetic chromosome (see Example 1). This applies to panels G, H, and I. Representative assemblies with the unmethylated chromosome are shown. Methylated chromosome assemblies were separately performed in place of the unmethylated variant.

FIG. 13H shows that chromatin was assembled on unmethylated synthetic chromosomes by salt dialysis and subsequently incubated with ACF and/or ATP. The resulting mixture was digested with MNase and visualized by agarose gel electrophoresis. Regularly spaced nucleosomes (labeled with red dots) are observed only when chromatin was incubated with both ACF and ATP.

FIG. 13I shows chromatin assembled on unmethylated synthetic chromosomes using the NAP1 histone chaperone in the presence of ACF and/or ATP. The resulting mixture was digested with MNase and visualized by agarose gel electrophoresis. Nucleosomes are regularly spaced (labeled with red dots) in the presence of both ACF and ATP, although less apparent than in panel H.

FIGS. 14A-14F show control MNase-Seq and tiling qPCR analysis.

FIG. 14A is the same analysis as FIG. 4C, showing that 6 mA quantitatively disfavors nucleosome occupancy in vitro but not in vivo. Here, the extent of MNase digestion was 40% of that in FIG. 4C. P-values were calculated using a two-sample unequal variance t test. N.S denotes “non-significant,” with p>0.05.

FIG. 14B is the same analysis as FIG. 6E, showing that the ACF complex restores nucleosome occupancy over methylated DNA in an ATP-dependent manner in vitro. Here, the extent of MNase digestion was 25% of that in FIG. 6E. P-values were calculated using a two-sample unequal variance t test. N.S denotes “non-significant,” with p>0.05.

FIG. 14C is the same analysis as FIG. 12D, showing that nucleosomes with high starting 6 mA show larger changes in fuzziness. Here, the extent of MNase digestion was 40% of that in FIG. 12D. Distributions are compared using a Wilcoxon rank-sum test. N.S denotes “non-significant,” with p>0.01.

FIG. 14D is the same analysis as FIG. 12E, showing that nucleosomes with high starting 6 mA exhibit characteristic changes in nucleosome occupancy at and around the nucleosome dyad. Here, the extent of MNase digestion was 40% of that in FIG. 12E. The change in dyad occupancy is compared between nucleosomes with high and low starting 6 mA using a Wilcoxon rank-sum test. N.S denotes “non-significant,” with p>0.01.

FIG. 14E shows tiling qPCR analysis of nucleosome occupancy in spike-in and homogeneous synthetic chromosome preparations. The blunt, unmethylated synthetic chromosome (construct #1 in FIG. 5B) was used for chromatin assembly with (“Spike-in”) or without (“Homogeneous”) a 100-fold excess of buffer DNA. In the latter case, an equivalent mass of synthetic chromosome was added in place of buffer DNA to maintain the same DNA concentration for chromatin assembly. The tiling qPCR assay was performed as in FIG. 6B. Shaded red bars depict the regions where 6 mA modulates nucleosome occupancy in separate methylated chromosomes analyzed in FIGS. 6B and 6C. Note that methylated chromosomes were not used to generate qPCR data for this figure. Black arrowheads indicate no decrease in nucleosome occupancy in these regions when buffer DNA is used. Thus, the decrease in nucleosome occupancy in methylated chromosomes reported in FIGS. 6A-6E cannot be attributed to spike-in versus homogeneous addition of DNA for chromatin assembly. Error bars in all panels represent s.e.m. (n=3-4).

FIG. 14F shows that chromatin was assembled on synthetic chromosomes using the NAP1 histone chaperone in the presence of ACF and/or ATP, instead of set dialysis. qPCR analysis was performed as in FIG. 6B. Methylated chromosomes used in this experiment contain 6 mA in native sites. The addition of ACF and ATP results in a partial restoration of nucleosome occupancy over the methylated region. These results are similar to FIG. 6D, where chromatin was assembled by sat dialysis instead of NAP1.

FIG. 15 shows that ciliate methyltransferase MTA1c mediates DNA N6-adenine methylation (6 mA) in vivo and 6 mA directly disfavors nucleosome occupancy in vitro.

### DETAILED DESCRIPTION OF THE DISCLOSURE

DNA N6-adenine methylation (6 mA) has recently been described in diverse eukaryotes, spanning unicellular organisms to metazoa. In the present disclosure, it's reported a DNA 6 mA methyltransferase complex in ciliates, termed MTA1c. It consists of two MT-A70 proteins and two homeobox-like DNA-binding proteins and specifically methylates dsDNA. Disruption of the catalytic subunit, MTA1, in the ciliate Oxytricha leads to genome-wide loss of 6 mA and abolishment of the consensus ApT dimethylated motif. Mutants fail to complete the sexual cycle, which normally coincides with peak MTA1 expression. The present disclosure investigates the impact of 6 mA on nucleosome occupancy in vitro by reconstructing complete, full-length Oxytricha chromosomes harboring 6 mA in native or ectopic positions. It's shown that 6 mA directly disfavors nucleosomes in vitro in a local, quantitative manner, independent of DNA sequence. Furthermore, the chromatin remodeler ACF can overcome this effect. The present disclosure identifies a diverged DNA N6-adenine methyltransferase and defines the role of 6 mA in chromatin organization.

One embodiment of the present disclosure is a method of modifying a nucleic acid from a cell, the cell derived from a multicellular eukaryote. This method comprises the steps of: (a) obtaining the nucleic acid from the cell; and (b) contacting the nucleic acid with MTA1c or any components thereof under conditions effective to methylate the nucleic acid.

In some embodiments, the nucleic acid is RNA or DNA. In some embodiments, the eukaryotic cell is mammalian. In some embodiments, the multicellular eukaryote is a human. In some embodiments, the modification is a DNA N6-adenine methylation including one of more of the following motifs: dimethylated AT (5′-A*T-3′/3′-TA*-5′), dim ethylated TA (5′-TA*-3′/3′-A*T-5′), dim ethylated AA (5′-A*A*-3′/3′-TT-5′), methylated AT (5′-A*T-3′/3′-TA-5′), methylated AA (5′-A*A-3′/3′-TT-5′), methylated AC (5′-A*C-3′/3′-TG-5′), methylated AG (5′-A*G-3′/3′-TC-5′), methylated TA (5′-TA*-3′/3′-AT-5′), methylated AA (5′-AA*-3′/3′-TT-5′), methylated CA (5′-CA*-3′/3′-GT-5′), and methylated GA (5′-GA*-3′/3′-CT-5′). In certain embodiments, the MTA1 or an ortholog thereof comprises a mutation effective to abrogate dimethylation of the nucleic acid. Preferably, the mutation comprises loss of a C-terminal methyltransferase domain. In some embodiments, the MTA1c or any components thereof is obtained from ciliates, algae, or basal fungi. Preferably, the MTA1c or any components thereof is obtained from Oxytricha or Tetrahymena.

As used herein, an “ortholog,” or orthologous gene, is a gene with a sequence that has a portion with similarity to a portion of the sequence of a known gene, but found in a different species than the known gene. An ortholog and the known gene originated by vertical descent from a single gene of a common ancestor. As used herein an ortholog encodes a protein that has a portion of at least about 50%, such as at least about 55%, at least about 60%, at least about 65%, at least about 70%, at least about 75%, at least about 80% or at least about 80% of the total length of the sequence of the encoded protein that is similar to a portion of a length of at least about 50%, such as at least about 55%, at least about 60%, at least about 65%, at least about 70%, at least about 75%, at least about 80% or at least about 80% of a known protein. The respective portion of the ortholog and the respective portion of the known protein to which it is similar may be a continuous sequence or be fragmented a number, for example, into 1 to about 3, including 2, individual regions within the sequence of the respective protein. For example, the 1 to about 3 regions are arranged in the same order in the amino acid sequence of the ortholog and the amino acid sequence of the known protein. Such a portion of an ortholog has an amino acid sequence that has at least about 40%, at least about 45%, such as at least about 55%, at least about 60%, at least about 65%, at least about 70%, at least about 75% or at least about 80% sequence identity to the amino acid sequence of the known protein encoded by a MTA1 gene.

As used herein, an asterisk “*” indicates the presence of a methylated base. For example, “A*” represents a methylated adenine.

The modified base, m6dA, has been discovered in a wide range of eukaryotes, including humans. m6dA levels are significantly reduced in gastric and liver cancer tissues, and disruption of m6dA promotes tumor formation (Xiao et al. 2018). As disclosed herein, MTA1 is a novel m6dA “writer”, paving the way for cost-effective methods to understand mechanisms of m6dA function in biomedically relevant models.

Accordingly, another embodiment of the present disclosure is a method of treating or ameliorating the effects of a disease characterized by an abnormal level of m6dA in a subject. This method comprises administering to the subject an amount of MTA1c or any components thereof effective to modulate m6dA levels in the subject. In some embodiments, the modulation comprises restoring m6dA levels to normal or near-normal ranges in the subject.

In some embodiments, the subject is a mammal that can be selected from the group consisting of humans, veterinary animals, and agricultural animals. Preferably, the subject is a human.

In some embodiments, the disease is a cancer, e.g., gastric cancer or liver cancer. In certain embodiments, the method further comprises administering to the subject one or more of anti-gastric cancer and anti-liver cancer drugs. Non-limiting examples of anti-liver cancer drugs include Nexavar™ (Sorafenib Tosylate) and Stivarga™ (Regorafenib). Non-limiting examples of anti-gastric cancer drugs include Cyramza™ (Ramucirumab), Doxorubicin Hydrochloride, 5-FU (Fluorouracil Injection), Fluorouracil Injection, Herceptin™ (Trastuzumab), Mitomycin C, Taxotere™ (Docetaxel), Trastuzumab, Afinitor™ (Everolimus), Somatuline Depot™ (Lanreotide Acetate), FU-LV, TPF, and XELIRI.

In some embodiments, the method furthering comprises co-administering to the subject an epigenetic agent that is selected from the group consisting of methylation inhibiting drugs, Bromodomain inhibitors, histone acetylase (HAT) inhibitors, protein methyltransferase inhibitors, histone methylation inhibitors, histone deacetlyase (HDAC) inhibitors, histone acetylases, histone deacetlyases, and combinations thereof.

Another embodiment of the present disclosure is a pharmaceutical composition comprising MTA1c or any components thereof that is effective to modulate m6dA levels in a subject in need thereof and a pharmaceutically acceptable carrier, diluent, adjuvant or vehicle.

Yet another embodiment of the present disclosure is a kit for treating or ameliorating the effects of a disease characterized by an abnormal level of m6dA in a subject, such as, e.g., cancer, comprising an effective amount of MTA1c or any components thereof, packaged together with instructions for its use.

Another embodiment of the present disclosure is a cell line obtained from a multicellular eukaryote comprising a nucleic acid encoding MTA1c or any components thereof and/or an MTA1c protein complex or any components thereof. As used herein, a “cell line” refers to all types of cell lines such as, e.g., immortalized cell lines and primary cell lines. In certain embodiments, the nucleic acid encoding MTA1c or any components thereof is operably linked to a recombinant expression vector.

Another embodiment of the present disclosure is a recombinant expression vector comprising a polynucleotide encoding MTA1c or any components thereof.

Still another embodiment of the present disclosure is a transgenic organism whose genome comprises a transgene comprising a nucleotide sequence encoding MTA1c or any components thereof. Non-limiting examples of possible organism include an archaea, a bacterium, a eukaryotic single-cell organism, algae, a plant, an animal, an invertebrate, a fly, a worm, a cnidarian, a vertebrate, a fish, a frog, a bird, a mammal, an ungulate, a rodent, a rat, a mouse, and a non-human primate.

The present disclosure also provides a method of identifying protein binding sites on DNA. This method comprises the steps of: (a) providing DNA; (b) contacting the DNA with MTA1c or any components thereof under conditions effective to methylate the DNA; (c) contacting the DNA with one or more proteins; (d) contacting the DNA with an enzyme effective to hydrolize the DNA in positions where no protein binding occurs; (e) removing the DNA bound protein; and (f) isolating and sequencing the DNA fragments. In certain embodiments, the one or more proteins in step (c) comprise histone octamers.

Another embodiment of the present disclosure is a method of mediating DNA N6-adenine methylation. This method comprises the steps of: (a) providing DNA; and (b) contacting the DNA with MTA1c or any components thereof under conditions effective to methylate the DNA.

Another embodiment of the present disclosure is a method of modulating nucleosome organization and/or transcription in a cell, comprising providing to the cell an agent that is effective to modulate the expression of MTA1c or any components thereof.

The present disclosure also provides a method of generating a synthetic chromosome. This method comprises the steps of: (a) generating chromosome segments containing terminal restriction sites, wherein the chromosome segments comprise one or more m6dA bases; (b) digesting the chromosome segments with a restriction enzyme; and (c) purifying and ligating the digested chromosome segments to form a synthetic chromosome. In some embodiments, the method further comprises enriching the synthetic chromosome. A synthetic chromosome made by the method above is also provided.

The following examples are provided to further illustrate certain aspects of the present disclosure. These examples are illustrative only and are not intended to limit the scope of the disclosure in any way.

### EXAMPLES

### Example 1

### Materials and Methods

KEY RESOURCES TABLE

| | | |
|---|---|---|
| REACIENT or RESOURCE | SOURCE | IDENTIFIER |

Antibodies

| | | |
|---|---|---|
| Anti-H2A | Active Motif | Cat #: 39111 |
| Anti-H2B | Abcam | Cat #: 1790 |
| Anti-H3 | Abcam | Cat #: 1791 |
| Anti-H4 | Active Motif | Cat #: 39269 |
| Anti-N6-methyladenosine | Cedarlane | Cat #: 202003(SY) |
| antibody | Labs/Synaptic | |
| | Systems | |
| Goat Anti-Rabbit IgG | Bio-Rad | 1706515 |
| (H + L)-HRP Conjugate | | |

Bacterial and Virus Strains

| | | |
|---|---|---|
| One Shot TOP10 chemically | Thermo Fisher | Cat #: C404006 |
| competent E. coli | | |
| BL21(DE3) pLysS | Thermo Fisher | Cat #: 70-236-4 |
| SHuffle T7 Express | NEB | Cat #: C3029J |
| Competent E. coli | | |
| Lemo21 (DE3) Competent | NEB | Cat #: C2S28J |
| E. coli | | |

Chemicals, Peptides, and Recombinant Proteins

| | | |
|---|---|---|
| Micrococcal nuclease | NEB | Cat #: M0247S |
| Q5 Site-Directed | NEB | Cat #: E0554S |
| Mutagenesis Kit | | |
| ProBlock Gold bacterial | GoldBio | Cat #: GB-330-5 |
| protease inhibitor | | |
| cocktail | | |
| Proteinase K | Roche | Cat #: 3113879001 |
| Phenol:Chloroform:IAA, | Thermo Fisher | Cat #: AM9732 |
| 25:24:1 | | |
| TRIzol reagent | Thermo Fisher | Cat #: 15596026 |
| DNA Polymerase I, Large | NEB | Cat #: M0210S |
| (Klenow) Fragment | | |
| Klenow Fragment | NEB | Cat #: M0212S |
| (3′ → 5′ exo-) | | |
| Bsal | NEB | Cat #: R3535S |
| EcoGII | NEB | Cat #: M0603S |
| T4 DNA ligase | NEB | Cat #: M0202M |
| Phusion DNA polymerase | NEB | Cat #: M0530L |
| S-adenosyl-L-methionine | NEB | Cat #: B9003S |
| Mouse NAP1 | This study | N/A |
| Drosophila ACF complex | Active Motif | Cat #: 31509 |
| Xenopus histones | This study | N/A |
| Polyvinyl alcohol | Sigma Aldrich | Cat #: P8136 |
| Polyethylene glycol 8000 | Sigma Aldrich | Cat #: P2139 |
| Adenosine | Sigma Aldrich | Cat #: A6559-25UMO |
| 5′-triphosphate (ATP) | | |
| Creatine phosphate | Sigma Aldrich | Cat #: 10621714001 |
| Creatine kinase | Sigma Aldrich | Cat #: 10127566001 |
| Power SYBR Green PCR master | Thermo Fisher | Cat #: 4367659 |
| mix | | |
| Gum Arabic | Sigma Aldrich | Cat #: G9752-1KG |
| 3H-labeled | PerkinElmer | Cat #: NET155V250UC |
| S-adenosyl-L-methionine | | |
| ([3H]SAM) | | |
| Ultima Gold | PerkinElmer | Cat #: 6013326 |
| DNA degradase plus enzyme | Zymo Research | Cat #: E2020 |
| ¹⁵N₅-dA nucleoside | Cambridge | Cat #: NLM-3895-25 |
| | Isotope | |
| | Laboratories | |
| D₃-6mA | Synthesized | N/A |
| | in this study | |

Critical Commercial Assays

| | | |
|---|---|---|
| QIAquick gel extraction kit | QIAGEN | Cat #: 28706 |
| NEBNext Poly(A) mRNA | NEB | Cat #: E7490S |
| Magnetic Isolation | | |
| Module | | |
| ScriptSeq v2 RNA-Seq | Illumina | Cat #: SSV21124 |
| Library Prep Kit | | |
| Nucleospin Tissue Kit | Takara Bio | Cat #: 740952.250 |
| | USA | |
| MinElute Reaction Cleanup | QIAGEN | Cat #: 28206 |
| Kit | | |
| NEBNext Ultra II DNA | NEB | Cat #: E7645S |
| Library Prep Kit | | |
| Hi-Scribe T7 High Yield | NEB | Cat #: E2040S |
| RNA Synthesis Kit | | |
| Dynabeads Protein A | Thermo Fisher | Cat #: 10001D |
| TOPO TA cloning kit | Thermo Fisher | Cat #: K457501 |

Deposited Data

| | | |
|---|---|---|
| Oxytricha trifallax | This study | SRA: SRX2335608 and |
| SMRT-seq | | SRX2335607 |
| Tetrahymena thermophila | This study | GEO: GSE94421 |
| SMRT-seq | | |
| Oxytricha trifallax, all | This study | GEO: GSE94421 |
| Illumina data (RNA- | | |
| seq, 6mA-IP-seq, MNase-seq, | | |
| gDNA-seq) | | |

Experimental Models: Organisms/Strains

| | | |
|---|---|---|
| Oxytricha trifallax cells, | Lab collection | N/A |
| strain JRB310 | | |
| Oxytricha trifallax cells, | Lab collection | N/A |
| strain JRB510 | | |
| Oxytricha trifallax cells, | Lab collection | N/A |
| mtal mutant | | |
| Tetrahymena thermophila | Tetrahymena | Cat #: SD00703 |
| cells, strain SB210 | stock center | |

Oligonucleotides

| | | |
|---|---|---|
| All are listed in Table S4 | IDT | N/A |

Recombinant DNA

| | | |
|---|---|---|
| pET-His-NAP1 (expression | This study | N/A |
| vector for recombinant | | |
| NAP1) | | |
| pET-XenH2A (expression | This study | N/A |
| vector for recombinant | | |
| Xenopus histone H2A) | | |
| pET-XenH2B (expression | This study | N/A |
| vector for recombinant | | |
| Xenopus histone H2B) | | |
| pET-XenH3 (expression | This study | N/A |
| vector for recombinant | | |
| Xenopus histone H3) | | |
| pET-XenH4 (expression | This study | N/A |
| vector for recombinant | | |
| Xenopus histone H4) | | |
| pET-HisSUMO-MTA1 | This study | N/A |
| (expression vector for | | |
| recombinant Tetrahymena | | |
| MTA1) | | |
| pET-HisSUMO-MTA7 | This study | N/A |
| (expression vector for | | |
| recombinant Tetrahymena | | |
| MTA7) | | |
| pET-HisSUMO-p1 | This study | N/A |
| (expression vector for | | |
| recombinant Tetrahymena p1) | | |
| pET-HisSUMO-p2 | This study | N/A |
| (expression vector for | | |
| recombinant Tetrahymena p2) | | |
| pCR-TOPO- | This study | N/A |
| syntheticChromosome (cloned | | |
| synthetic chromosomes to | | |
| verify accuracy of ligation | | |
| of component DNA building | | |
| blocks) | | |

Software and Algorithms

| | | |
|---|---|---|
| Galaxy | Galaxy | https://usegalaxy.org/ |
| | Community Hub | |
| Bowtie2 | Langmead and | http://bowtie-bio.sourceforge.net/bowtie2/index.shtml |
| | Salzberg, 2012 | |
| TopHat2 | TopHat2 | https://ccb.jhu.edu/software/tophat/index.shtml |
| | (Mortazavi et | |
| | al., 2008) | |
| Python 2.7.10 | Python Software | https://www.python.org/download/releases/2.7/ |
| | Foundation | |
| CAGEr | Haberle et | https://bioconductor.org/packages/release/bioc/html/CAGEr.html |
| | al.. 2015 | |
| SMRT Analysis 2.3.0 | Pacific | https://www.pacb.com/documentation/smrt-analysis-software-installation-v2-3-0/ |
| | Biosciences | |
| PSI-BLAST | NCBI/NIH | https://blast.ncbi.nlm.nih.gov/ |
| | | Blast.cgi?CMD=Web&PAGE-Proteins&PROGRAM-blastp&RUN_PSIBLAST=on |
| CD-HIT | Huang et al., | http://weizhong-lab.ucsd.edu/cdhit-web-server/cgi-bin/index.cgi |
| | 2010 | |
| MAFFT | Katoh et al., | https://mafft.cbrc.jp/alignment/software/ |
| | 2017; Kuraku | |
| | et al., 2013 | |
| MrBayes/CIPRES Science | Miller et al., | https://www.phylo.org/ |
| Gateway | 2010 | |
| R (v3.2.5) | The R Foundation | https://www.r-project.org/ |
| hmmscan | Finn et al., | https://www.ebi.ac.uk/Tools/hmmer/search/hmmscan |
| | 2015 | |

Other

| | | |
|---|---|---|
| Agencourt Ampure XP beads | Beckman Coulter | Cat #: A63880 |
| Acid-extracted Oxytricha | This study | N/A |
| histones | | |
| Slide-A-Lyzer 3.5K MWCO | Thermo Fisher | Cat #: PI66110 |
| cassette | | |
| Amersham Hybond-XL membrane | GE Healthcare | Cat #: RPN303S |
| Amersham Hybond-N+ | GE Healthcare | Cat #: RPN119B |
| membrane | | |
| Volvic water | Amazon | https://www.amazon.com/Volvic-500m1-6-Pack/dp/B013PCK8M4/ |
| | | ref=sr_1_1_a_it?_ie=UTF8&qid=1538873999&sr=8- |
| | | 1&keyword_s=volvic&dpID=418qEyu6yrUpreST=_SY300 |
| | | QL70 &dpSrc=srch |

Oxytricha trifallax

Vegetative Oxytricha trifallax strain J RB310 was cultured at a density of 1.5×10⁷ cells/L to 2.5×10⁷ cells/L in Pringsheim media (0.11 mM Na₂HPO₄, 0.08 mM MgSO₄, 0.85 mM Ca(NO₃)₂, 0.35 mM KCl, pH 7.0) and fed daily with Chlamydomonas reinhardtii. Cells were filtered through cheesecloth to remove debris and collected on a 10 pm Nitex mesh for subsequent experiments.

Tetrahymena thermophila

Stock cultures of vegetative Tetrahymena thermophila strain SB210 were maintained in Neff medium (0.25% w/v proteose peptone, 0.25% w/v yeast extract, 0.5% glucose, 33.3 pM FeCl₃). These cultures were inoculated into SSP medium (2% w/v proteose peptone, 0.1% w/v yeast extract, 0.2% w/v glucose, 33 pM FeCl₃) and grown to log-phase (˜3.5×10⁵ cells/mL) through constant shaking at 125 rpm/30° C.

### In Vivo MNase-Seq

3×10⁵ vegetative Oxytricha cells were fixed in 1% w/v formaldehyde for 10 min at room temperature with gentle shaking, and then quenched with 125 mM glycine. Cells were lysed by dounce homogenization in lysis buffer (20 mM Tris pH 6.8, 3% w/v sucrose, 0.2% v/v Triton X-100, 0.01% w/v spermidine trihydrochloride) and centrifuged in a 10%-40% discontinuous sucrose gradient (Lauth et al., 1976) to purify macronuclei. The resulting macronuclear preparation was pelleted by centrifugation at 4000×g, washed in 50 ml TMS buffer (10 mM Tris pH 7.5, 10 mM MgCl₂, 3 mM CaCl₂), 0.25M sucrose), resuspended in a final volume of 300 μL, and equilibriated at 37° C. for 5 min. Chromatin was then digested with MNase (New England Biolabs) at a final concentration of 15.7 Kunitz Units/μL at 37° C. for 1 min 15 s, 3 min, 5 min, 7 min 30 sec, 10 min 30 s, and 15 min respectively. Reactions were stopped by adding ½ volume of PK buffer (300 mM NaCl, 30 mM Tris pH 8, 75 mM EDTA pH 8, 1.5% w/v SDS, 0.5 mg/mL Proteinase K). Each sample was incubated at 65° C. overnight to reverse crosslinks and deproteinate samples. Subsequently, nucleosomal DNA was purified through phenol:chloroform:isoamyl alcohol extraction and ethanol precipitation. Each sample was loaded on a 2% agarose-TAE gel to check the extent of MNase digestion. The sample exhibiting −80% mononucleosomal species was selected for MNase-seq analysis, in accordance with previous guidelines (Zhang and Pugh, 2011). Mononucleosome-sized DNA was gel-purified using a QIAquick gel extraction kit (QIAGEN). Illumina libraries were prepared using an NEBNext Ultra II DNA Library Prep Kit (New England Biolabs) and subjected to paired-end sequencing on an Illumina HiSeq 2500 according to manufacturer's instructions. All vecietative Tetrahymena MNase-sea data were obtained from (Beh et al., 2015).

### Poly(A)⁺ RNA-Seq and TSS Sequencing

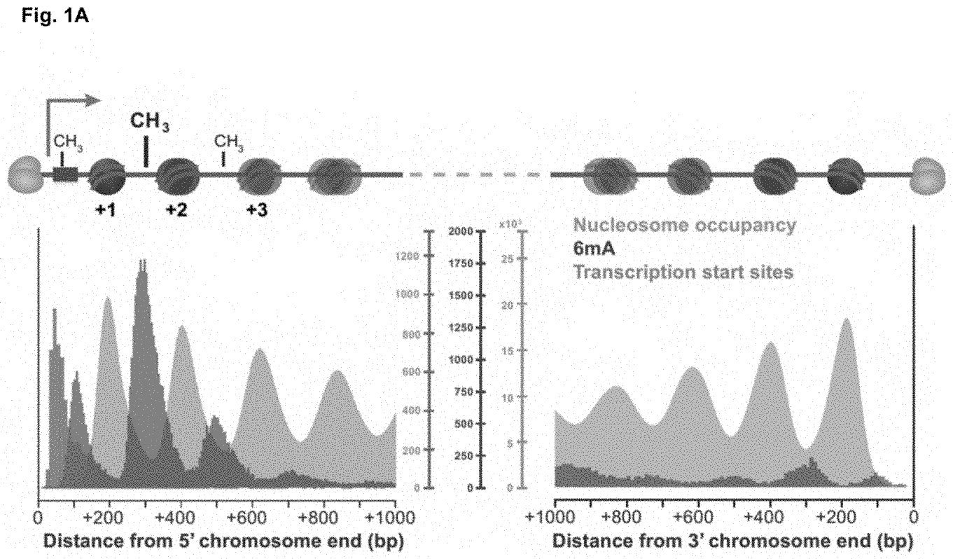

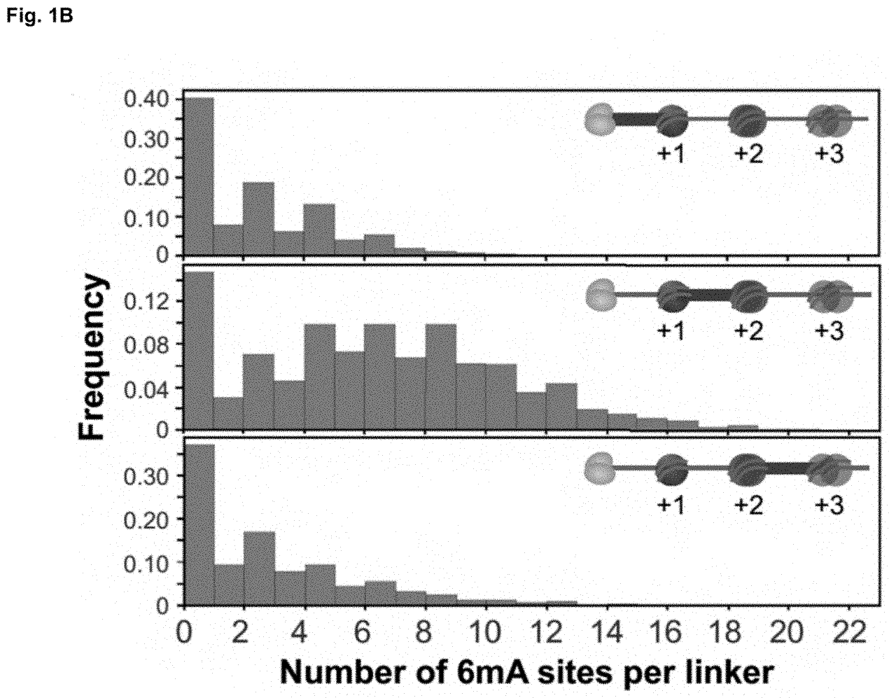

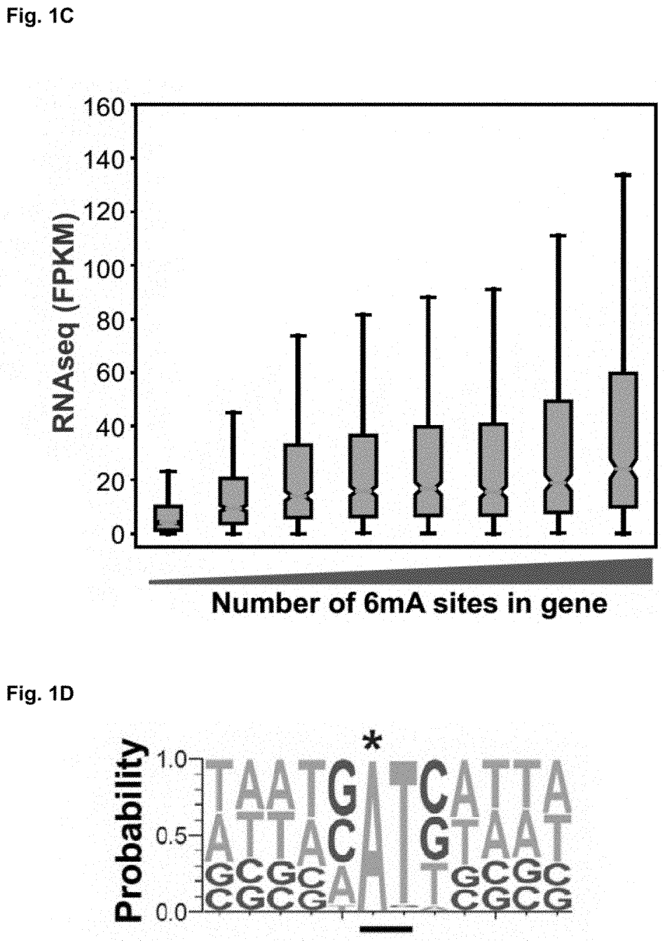

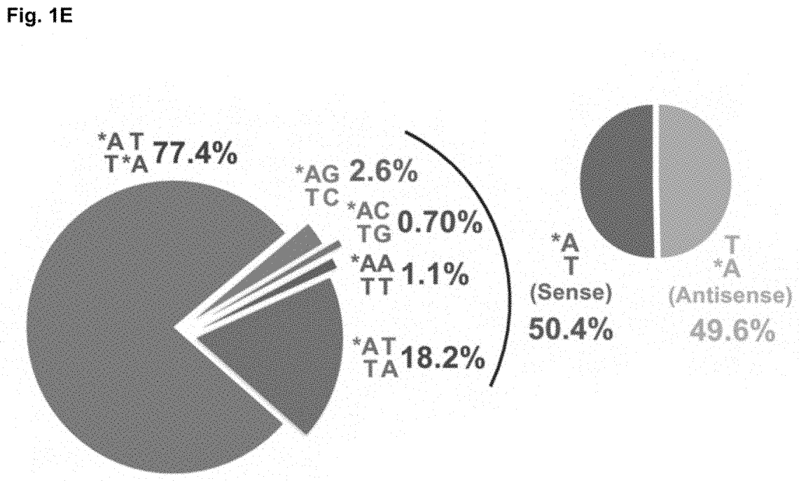

Oxytricha cells were lysed in TRIzol reagent (Thermo Fisher Scientific) for total RNA isolation according to manufacturer's instructions. Poly(A)⁺ RNA was then purified using the NEBNext Poly(A) mRNA Magnetic Isolation Module (New England Biolabs). Oxytricha poly(A)⁺ RNA was prepared for RNA-seq using the ScriptSeq v2 RNA-Seq Library Preparation Kit (Illumina). Tetrahymena poly(A)⁺ RNA-seq data was obtained from (Xiong et al., 2012). The 5′ ends of capped RNAs were enriched from vegetative Oxytricha total RNA using the RAMPAGE protocol (Batut et al., 2013), and used for library preparation, Illumina sequencing and subsequent transcription start site determination (ie. “TSS-seq”). These data were used to plot the distribution of Oxytricha TSS positions in FIG. 1A. TSS positions used for analysis outside of FIG. 1A were obtained from (Swart et al., 2013) and (Beh et al., 2015). For RNaseq analysis of genes grouped according to “starting” methylation level level: total 6 mA was counted between 100 bp upstream to 250 bp downstream of the TSS. Genes with high starting methylation have total 6 mA in the 90th percentile and higher. Genes with low starting methylation have total 6 mA at or below the 10th percentile.

### Immunoprecipitation and Illumina Sequencing of Methylated DNA (6 mA IP-Seq)

Genomic DNA was isolated from vegetative Oxytricha cells using the Nucleospin Tissue Kit (Takara Bio USA, Inc.). DNA was sheared into 150 bp fragments using a Covaris LE220 ultra-sonicator (Covaris). Samples were gel-purified on a 2% agarose-TAE gel, blunted with DNA polymerase I (New England Biolabs), and purified using MinElute spin columns (QIAGEN). The fragmented DNA was dA-tailed using Klenow Fragment (3′->5′ exo-) (New England Biolabs) and ligated to Illumina adaptors following manufacturer's instructions. Subsequently, 2.2 μg of adaptor-ligated DNA containing 6 mA was immunoprecipitated using an anti-N6-methyladenosine antibody (Cedarlane Labs) conjugated to Dynabeads Protein A (Invitrogen). The anti-6 mA antibody is commonly used for RNA applications, but has also been demonstrated to recognize 6 mA in DNA (Fioravanti et al., 2013; Xiao and Moore, 2011). The immunoprecipitated and input libraries were treated with proteinase K, extracted with phenol:chloroform, and ethanol precipitated. Finally, they were PCR-amplified using Phusion Hot Start polymerase (New England Biolabs) and used for Illumina sequencing.

### Sample Preparation for SMRT-Seq

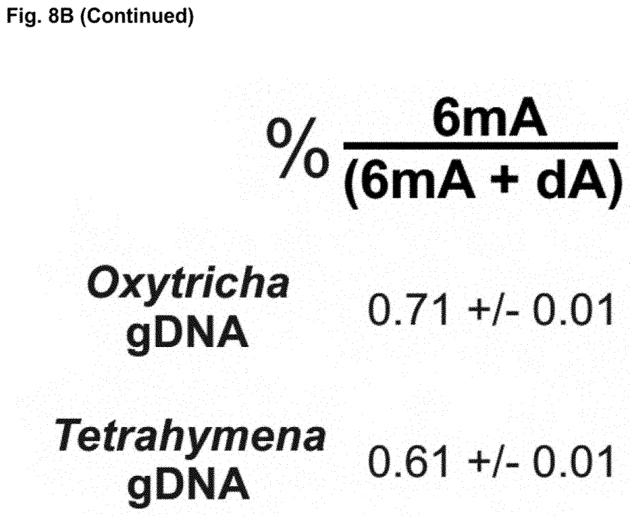

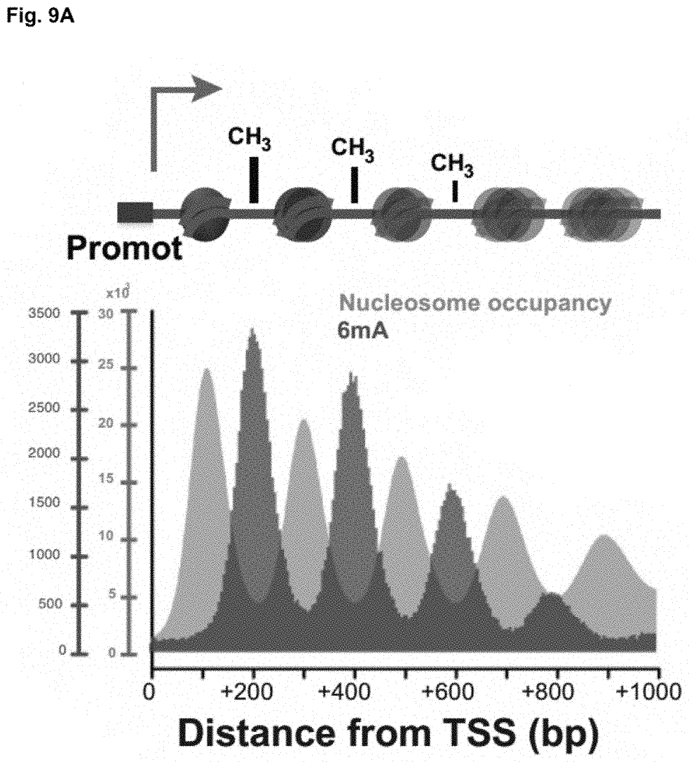

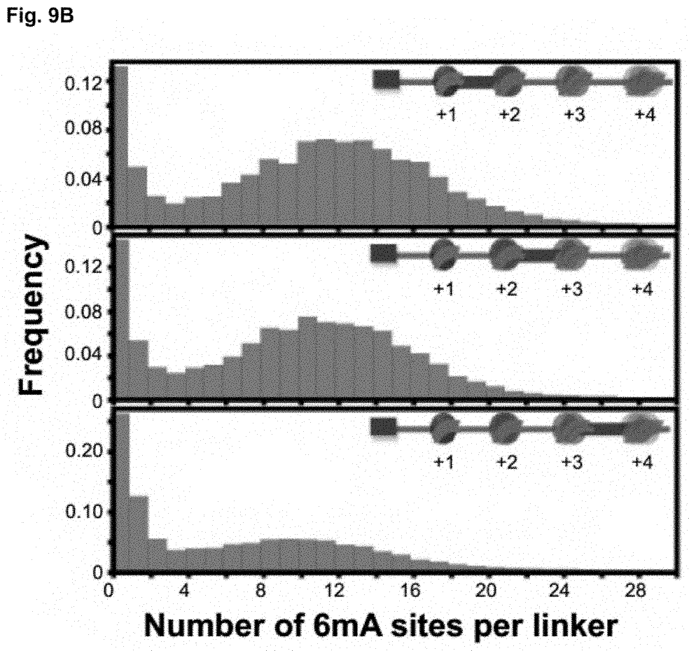

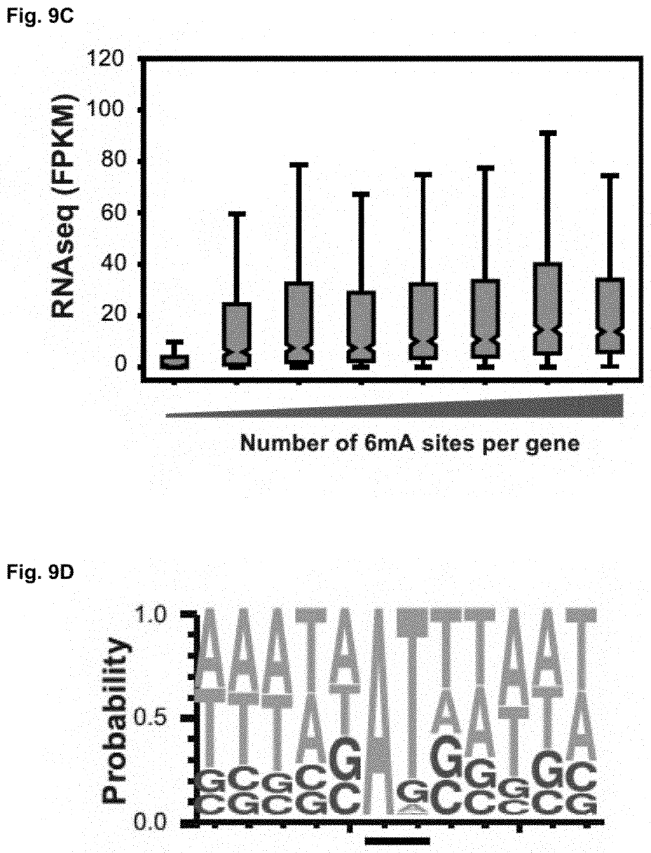

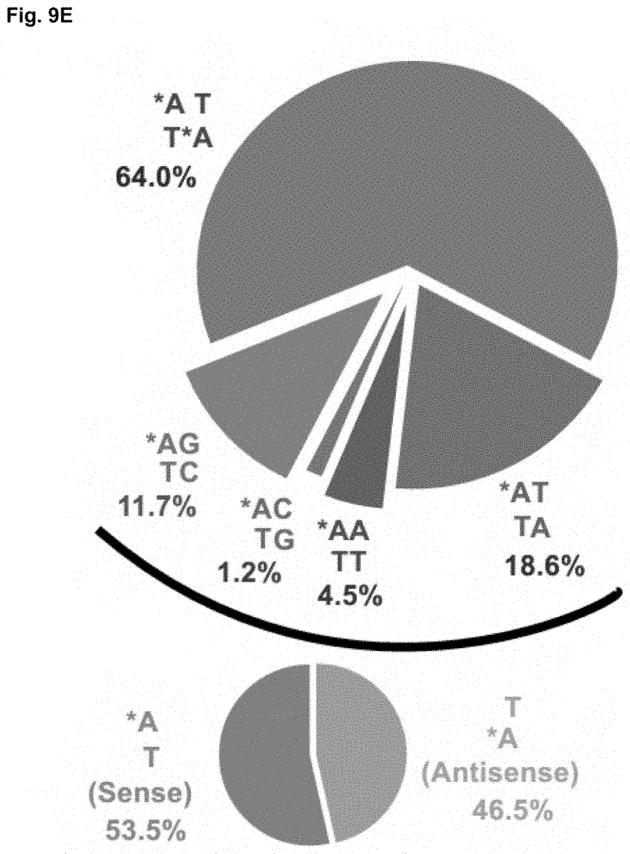

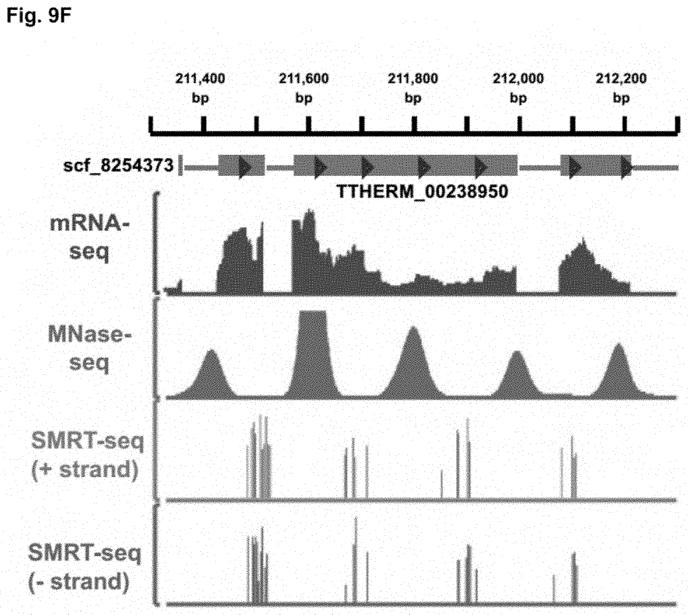

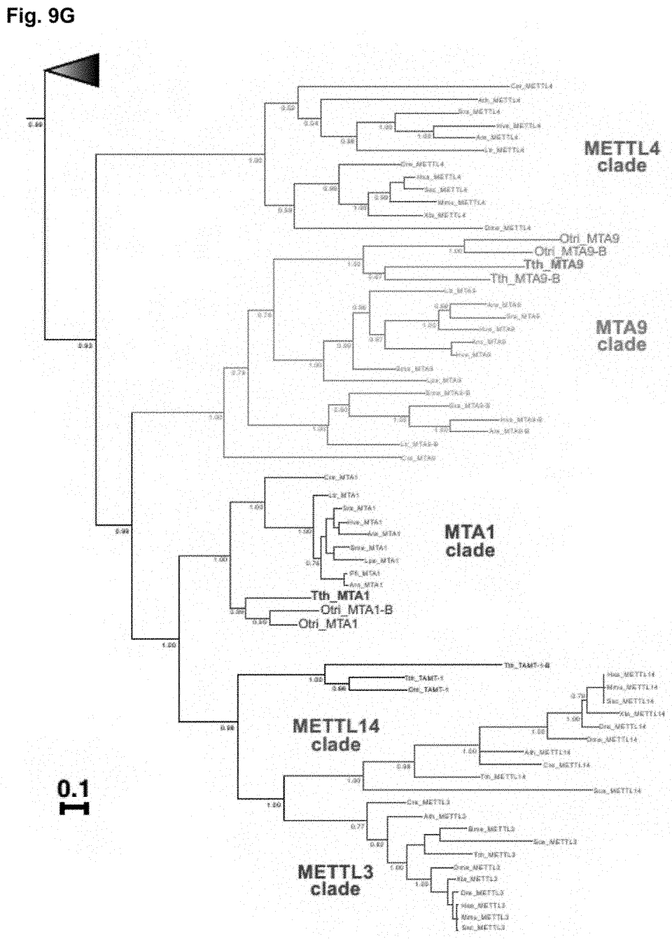

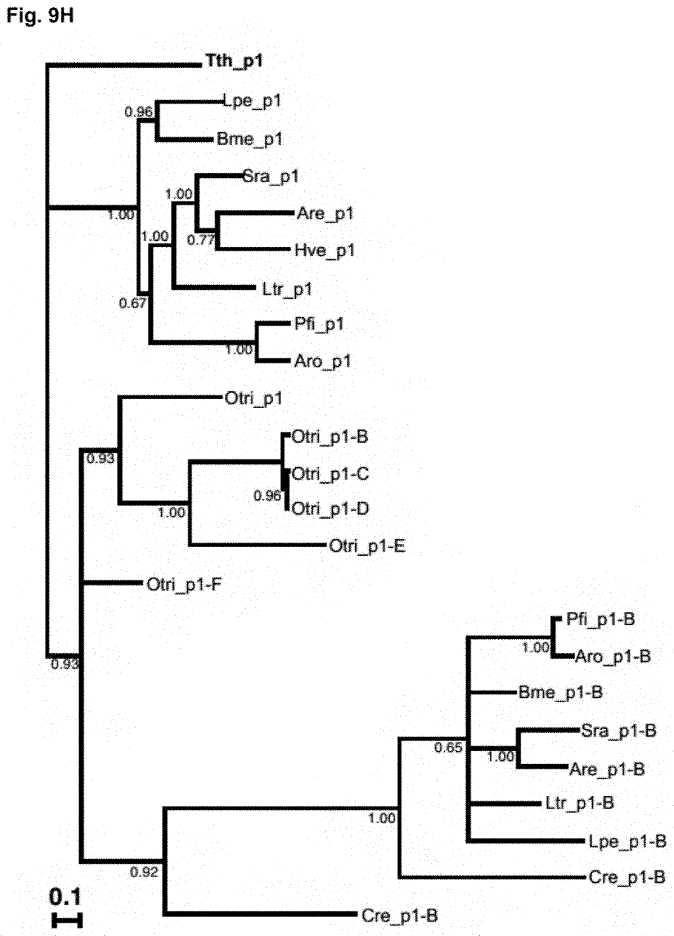

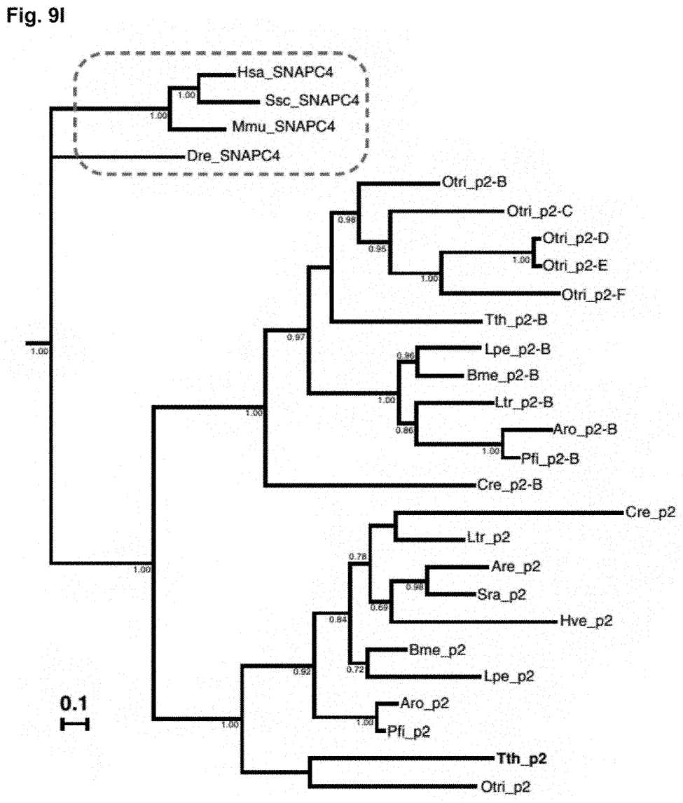

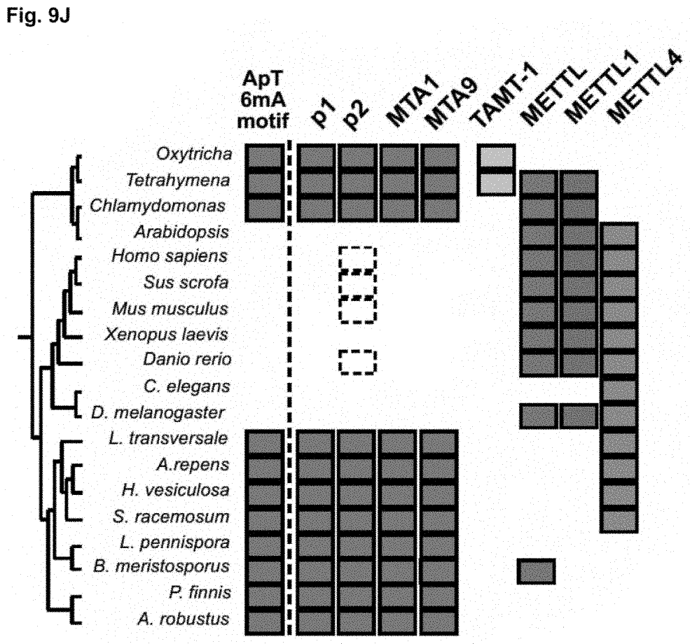

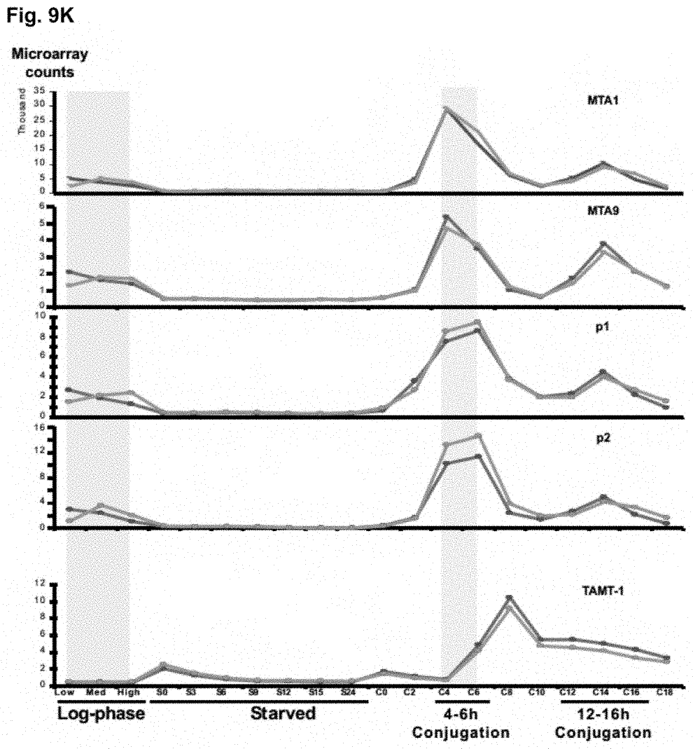

Vegetative Oxytricha macronuclei were isolated as described in the subheading “in vivo MNase-seq” of this study. Vegetative Tetrahymena macronuclei were isolated by differential centrifugation (Beh et al., 2015). Oxytricha and Tetrahymena cells were not fixed prior to nuclear isolation. Genomic DNA was isolated from Oxytricha and Tetrahymena macronuclei using the Nucleospin Tissue Kit (Macherey-Nagel). Alternatively, whole Oxytricha cells instead of macronuclei were used. SMRT-seq according to manufacturer's instructions, using P5-C3 and P6-C4 chemistry, as in (Chen et al., 2014). Oxytricha and Tetrahymena macronuclear DNA were used for SMRT-seq in FIGS. 1A-1E and 9A-9F, while Oxytricha whole cell DNA was used for all other Figures. Since almost all DNA in Oxytricha cells is derived from the macronucleus (Prescott, 1994), similar results are expected between the use of purified macronuclei or whole cells.

### Illumina Data Processing

Reads from all biological replicates were merged before downstream processing. All Illumina sequencing data were quality trimmed (minimum quality score=20) and length-filtered (minimum read length=40nt) using Galaxy (Blankenberg et al., 2010; Giardine et al., 2005; Goecks et al., 2010). MNase-seq and 6 mA IP-seq reads were mapped to complete chromosomes in the Oxytricha trifallax JRB310 (August 2013 build) or Tetrahymena thermophila SB210 macronuclear reference genomes (June 2014 build) using Bowtie2 (Langmead and Salzberg, 2012) with default settings, while poly(A). RNA-seq and TSS-seq reads were mapped using TopHat2 (Mortazavi et al., 2008) with August 2013 Oxytricha gene models or June 2014 Tetrahymena gene models, with default settings.

MNase-seq datasets were generated by paired-end sequencing. Within each MNase-seq dataset, the read pair length of highest frequency was identified. All read pairs with length±25 bp from this maximum were used for downstream analysis. On the other hand, 6 mA IP-seq datasets were generated by single-read sequencing. 6 mA IP-seq single-end reads were extended to the mean fragment size, computed using cross-correlation analysis (Kharchenko et al., 2008). The per-basepair coverage of Oxytricha MNase-seq read pair centers and extended 6 mA IP-seq reads were respectively computed across the genome. Subsequently, the per-basepair coverage values were normalized by the average coverage within each chromosome to account for differences in DNA copy number (and hence, read depth) between Oxytricha chromosomes (Swart et al., 2013). The per-basepair coverage values were then smoothed using a Gaussian filter of standard deviation=15. This smoothed data is denoted as “normalized coverage” or “nucleosome occupancy.” Tetrahymena MNase-seq data were processed similarly to Oxytricha, except that DNA copy number normalization was omitted as Tetrahymena chromosomes have uniform copy number (Eisen et al., 2006).

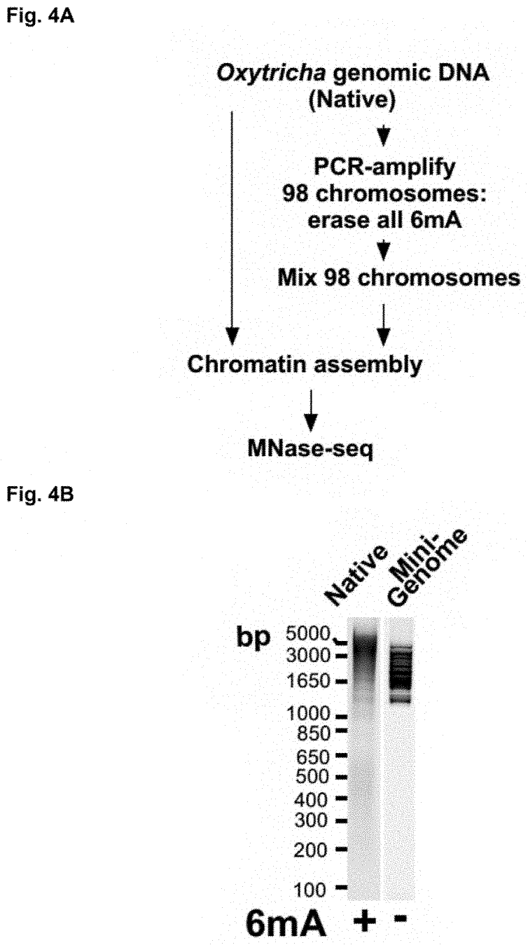

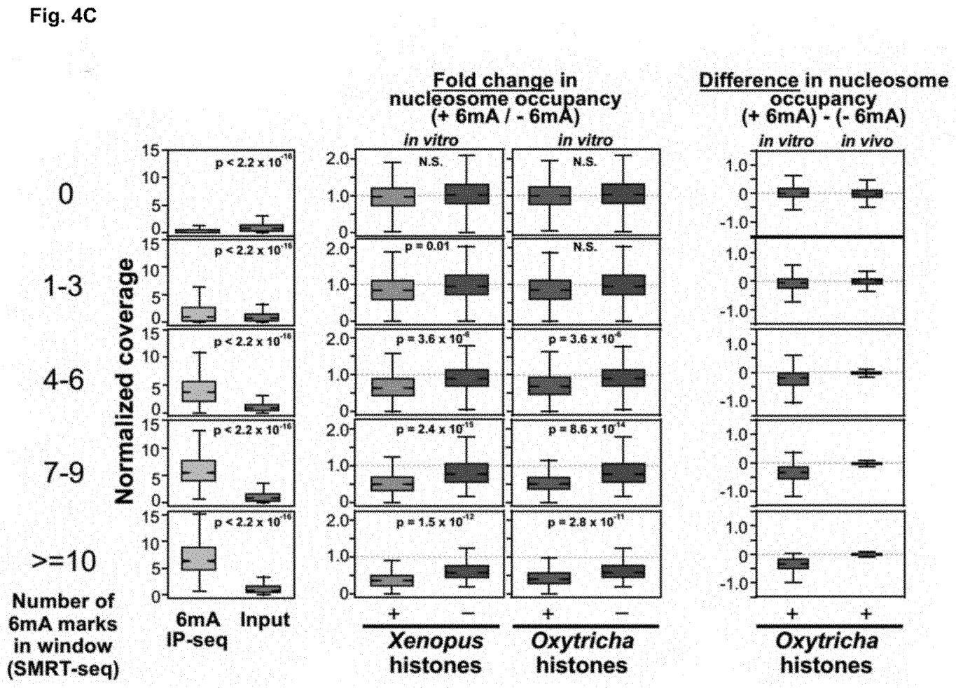

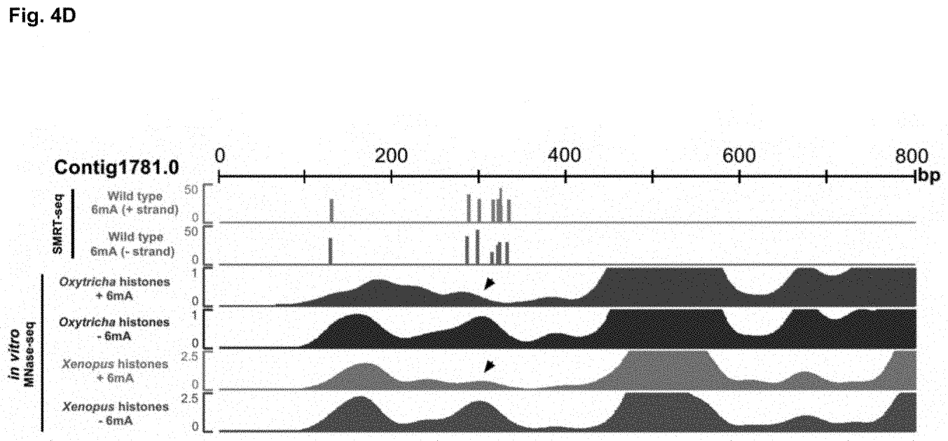

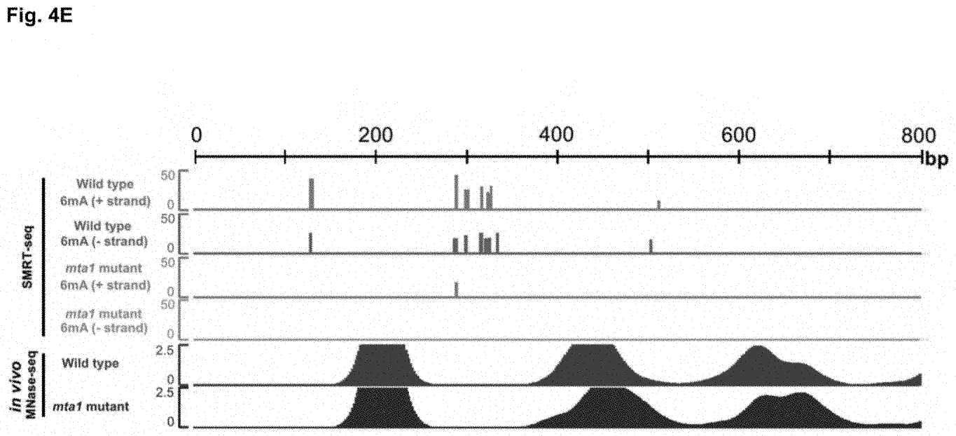

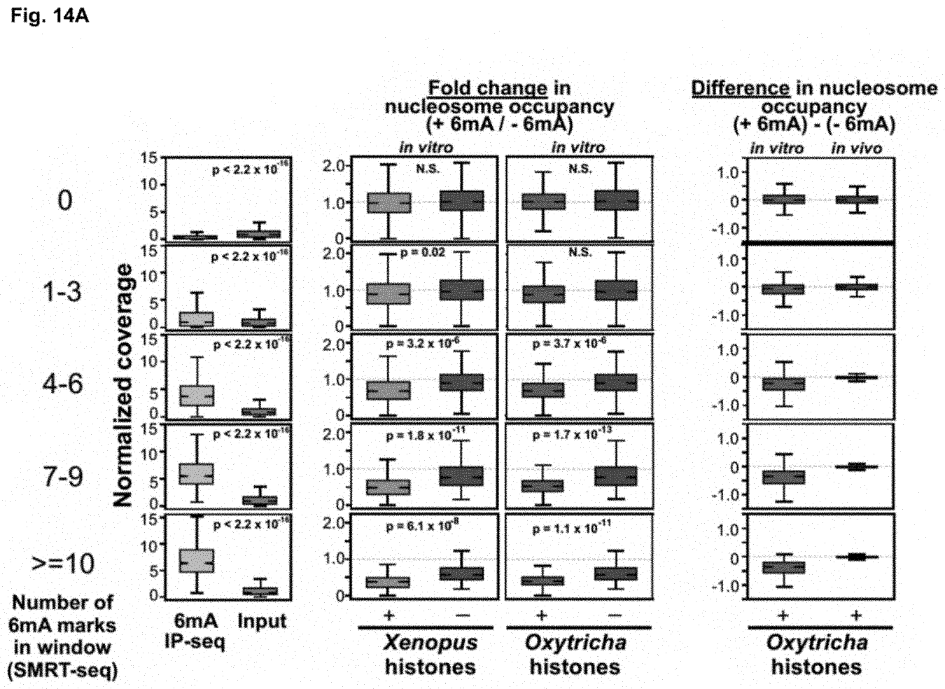

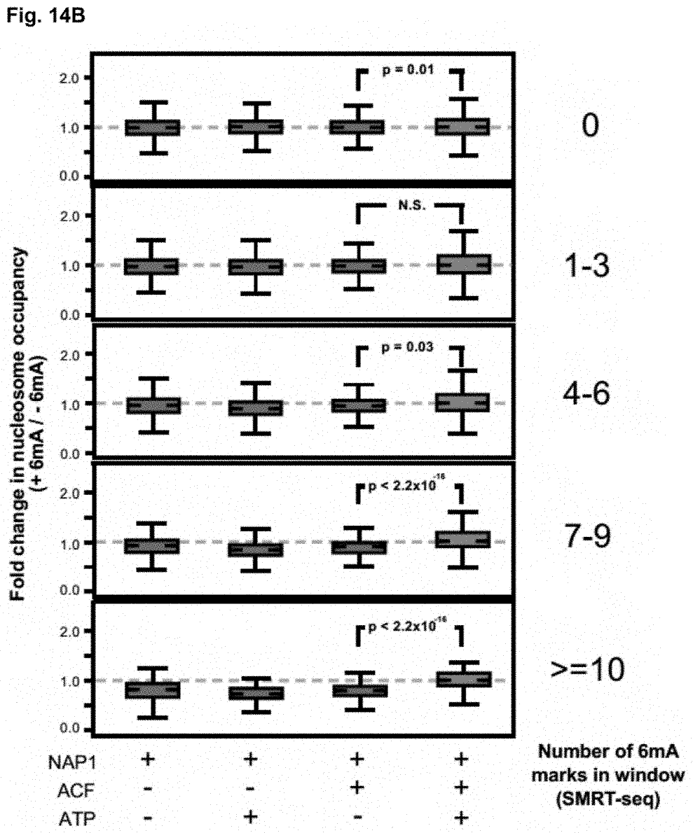

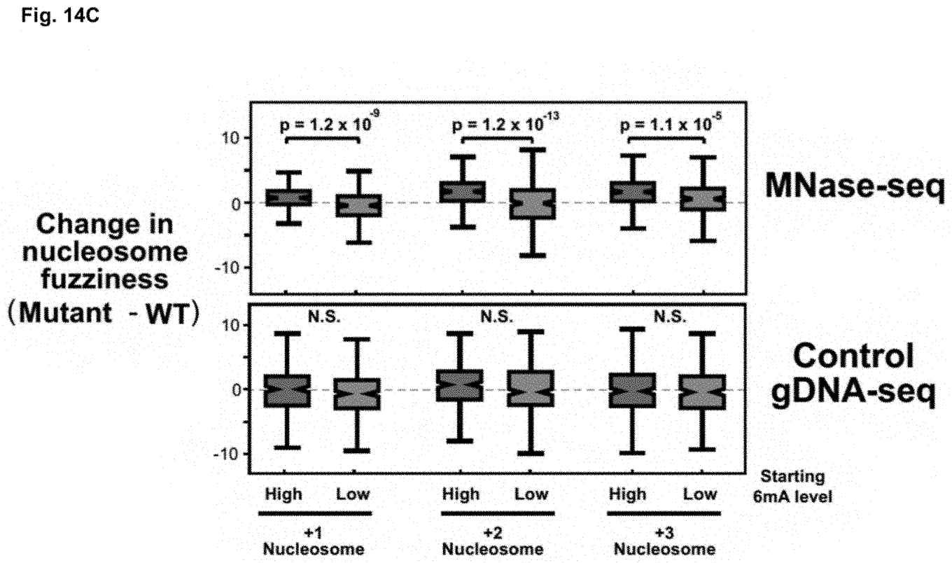

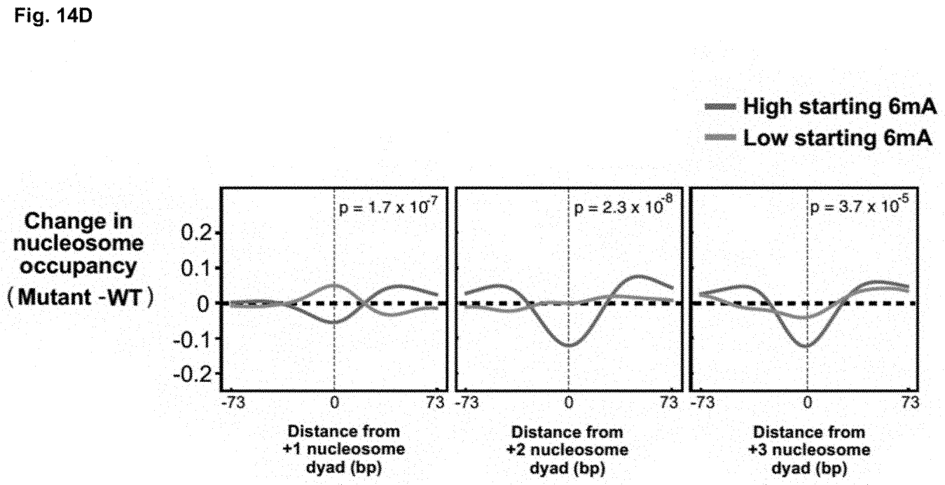

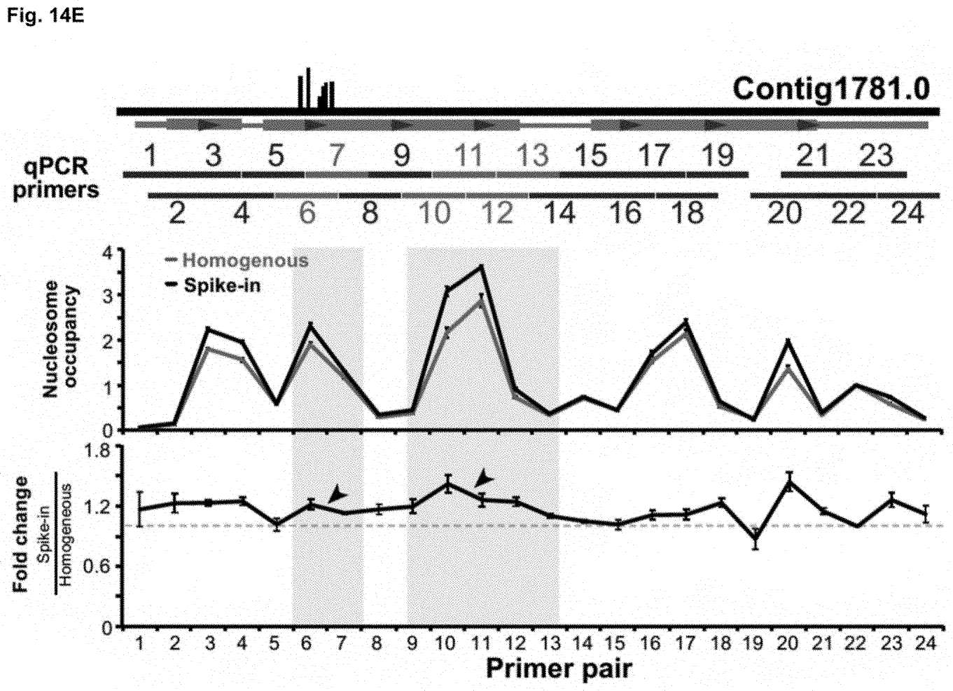

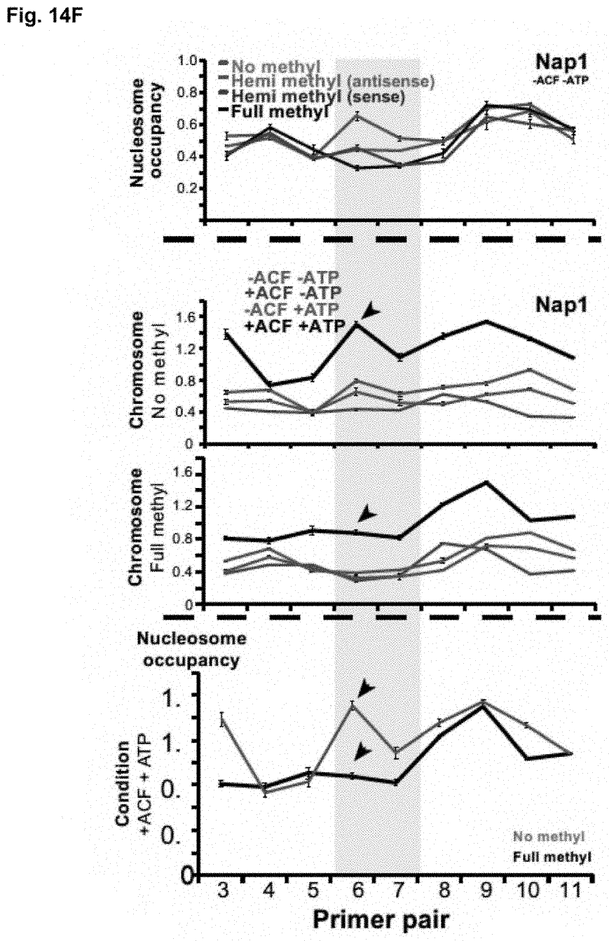

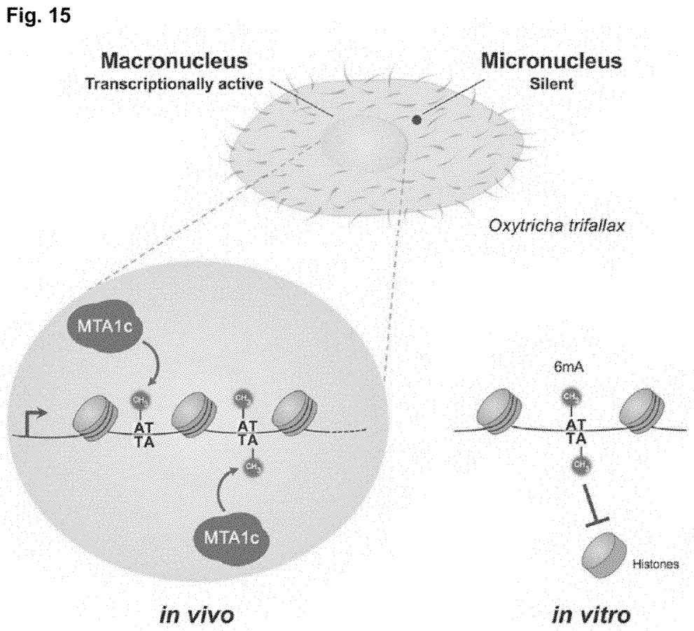

For the MNase-seq analysis in FIGS. 4C, 6E, 14A, and 14B, nucleosome occupancy and 6 mA IP-seq coverage were calculated within overlapping 51 bp windows across the 98 assayed chromosomes. Windows were binned according to the number of 6 mA residues within. The in vitro MNase-seq coverage from chromatinized native gDNA (“+” 6 mA) was divided by the corresponding coverage from chromatinized mini-genome DNA (“−” 6 mA) to obtain the fold change in nucleosome occupancy in each window. Alternatively, a subtraction was performed on these datasets to obtain the difference in nucleosome occupancy in vitro. Identical DNA sequences were compared for each calculation. These data are labeled as (“+” histones) in FIGS. 4C and 14A. Naked native gDNA and mini-genome DNA were also MNase-digested, sequenced and analyzed in the same manner to control for Mnase sequence preferences (“−” histones). Nucleosome occupancy in vivo corresponds to normalized MNase-seq coverage from wild type and mta1 mutant cells.

Nucleosome positions were iteratively called as local maxima in normalized MNase-seq coverage, as previously described (Beh et al., 2015). “Consensus”+1, +2, +3 nucleosome positions downstream of the TSS were inferred from aggregate MNase-seq profiles across the genome (FIG. 1A for Oxytricha and FIG. 9A for Tetrahymena). Each gene was classified as having a +1, +2, +3 and/or +4 nucleosome if there is a called nucleosome dyad within 75 bp of the consensus nucleosome position.

RNA-seq and TSS-seq read coverage were calculated without normalization by DNA copy number since there is no correlation between Oxytricha DNA and transcript levels (Swart et al., 2013).

Oxytricha TSSs were called from TSS-seq data using CAGEr (Haberle et al., 2015); with clusterCTSS parameters (threshold=1.6, thresholdlsTpm=TRUE, nrPassThreshold=1, method=“paraclu,” removeSingletons=TRUE, keepSingletonsAbove=5). Only TSSs with tags per million counts>0.1 were used for downstream analysis. Tetrahymena TSSs were obtained from (Beh et al., 2015).

### SMRT-Seq Data Processing

We processed SMRT-seq data with SMRTPipe v1.87.139483 in the SMRT Analysis 2.3.0 environment using, in order, the P Fetch, P Filter (with minLength=50, minSubreadLength=50, readScore=0.75, and artifact=−1000), P FilterReports, P Mapping (with gff2Bed=True, pulsemetrics=DeletionQV, IPD, InsertionQV, PulseWidth, QualityValue, MergeQV, SubstitutionQV, DeletionTag, and load PulseOpts=byread), P_MappingReports, P_GenomicConsensus (with algorithm=quiver, outputConsensus=True, and enableMapQVFilter=True), P_ConsensusReports, and P Mod ificationDetection (with identifyModifcations=True, enableMapQVFilter=False, and mapQvThreshold=10) modules. All other parameters were set to the default. The Oxytricha August 2013 reference genome build was used for mapping Oxytricha SMRT-seq reads, with Contig10040.0.1, Contig1527.0.1, Contig4330.0.1, and Contig54.0.1 removed, as they are perfect duplicates of other Contigs in the assembly. Tetrahymena SMRT-seq reads were mapped to the June 2014 reference genome build. Only chromosomes with high SMRT-seq coverage (>=80× for Oxytricha; >=100× for Tetrahymena) were used for all 6 mA-related analyses.

### Chromosome Synthesis

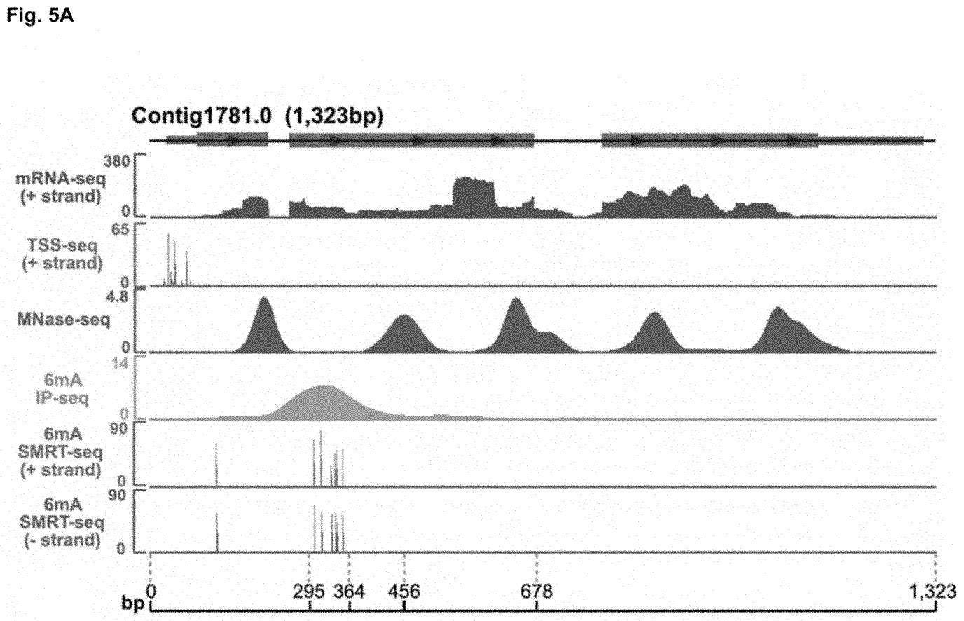

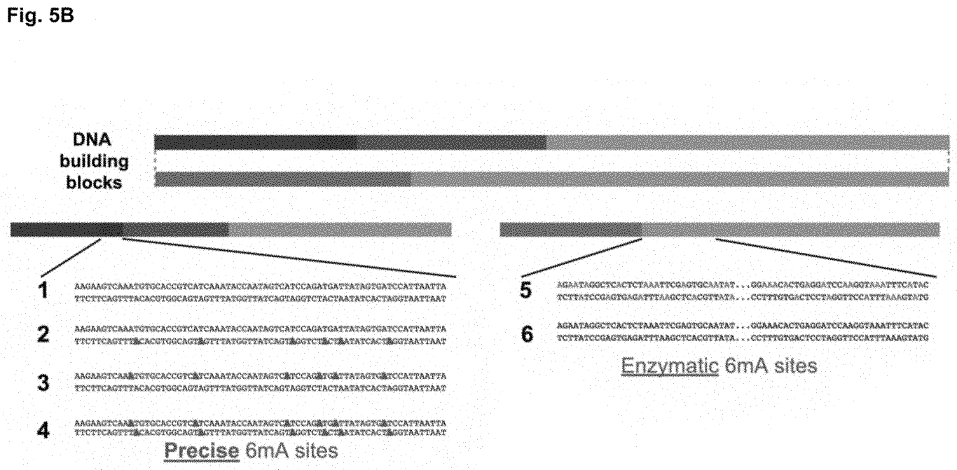

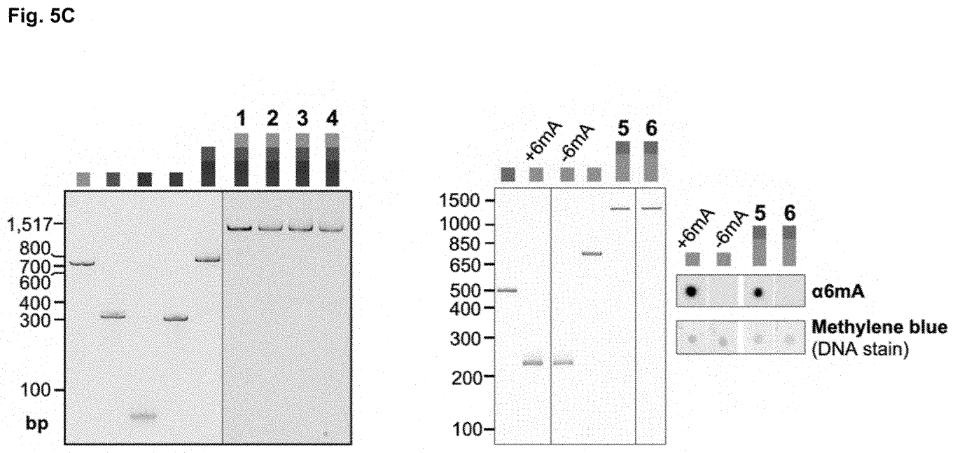

Synthetic Contig1781.0 chromosomes were constructed from “building blocks” of native chromosome sequence (FIGS. 5B and 5C). The dark blue building block in FIG. 5B was prepared by annealing synthetic oligonucleotides, while all other building blocks were generated by PCR-amplification from genomic DNA using Phusion DNA polymerase (New England Biolabs). All oligonucleotides used for annealing and PCR amplification are listed in Table 2. The PCR-amplified building blocks contain terminal restriction sites for BsaI (New England Biolabs), a type IIS restriction enzyme that cuts distal from these sites. BsaI cleaves within the native DNA sequence, generating custom 4nt 5′ overhangs and releasing the non-native BsaI restriction site as small fragments that are subsequently purified away. The BsaI-generated overhangs are complementary only between adjacent building blocks, conferring specificity in ligation and minimizing undesired by-products. After BsaI digestion, PCR building blocks were purified by phenol:chloroform extraction and ethanol precipitation. Building blocks were then sequentially ligated to each other using T4 DNA ligase (New England Biolabs) and purified by phenol:chloroform extraction and ethanol precipitation. Size selection after each ligation step was performed using polyethylene glycol (PEG) precipitation or Ampure XP beads (Beckman Coulter) to enrich for the large ligated product over its smaller constituents. The size of individual building blocks and their corresponding order of ligation were designed to maximize differences in size between ligated products and individual building blocks. This increases the efficiency in size selection of products over reactants. Chromosomes 1 and 6 in FIG. 5B was generated by full length PCR from genomic DNA. To prepare chromosomes 2-4 in FIG. 5B, the red, dark blue, and purple blocks were first ligated in a 3-piece reaction and purified from the individual components. This product was subsequently ligated with the turquoise building block to obtain the full length chromosome. To prepare chromosomes 5 in FIG. 5B, the red, orange, and emerald building blocks were ligated in a 3-piece reaction and subsequently purified. All chromosomes were subjected to Sanger sequencing to verify ligation junctions. 6 mA was installed in synthetic chromosomes using annealed oligonucleotides, or by incubation of DNA building blocks with EcoGII methyltransferase (New England Biolabs).

### Verification of Synthetic Chromosome Sequences

All chromosomes were dA-tailed using Klenow Fragment (3′->5′ exo-) (New England Biolabs), cloned using a TOPO TA cloning kit (Thermo Fisher) or StrataClone PCR Cloning Kit (Agilent Technologies), transformed into One Shot TOP10 chemically competent E. coli, and sequenced using flanking T7, T3, M13F, or M13R primers.

### Preparation of Oxytricha Histones

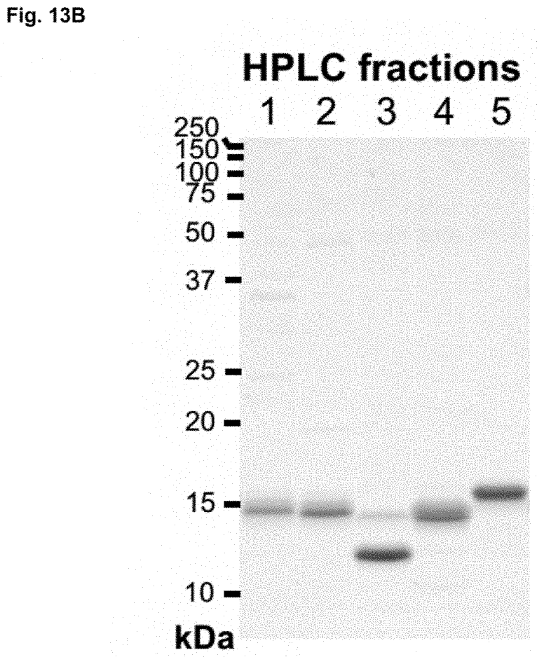

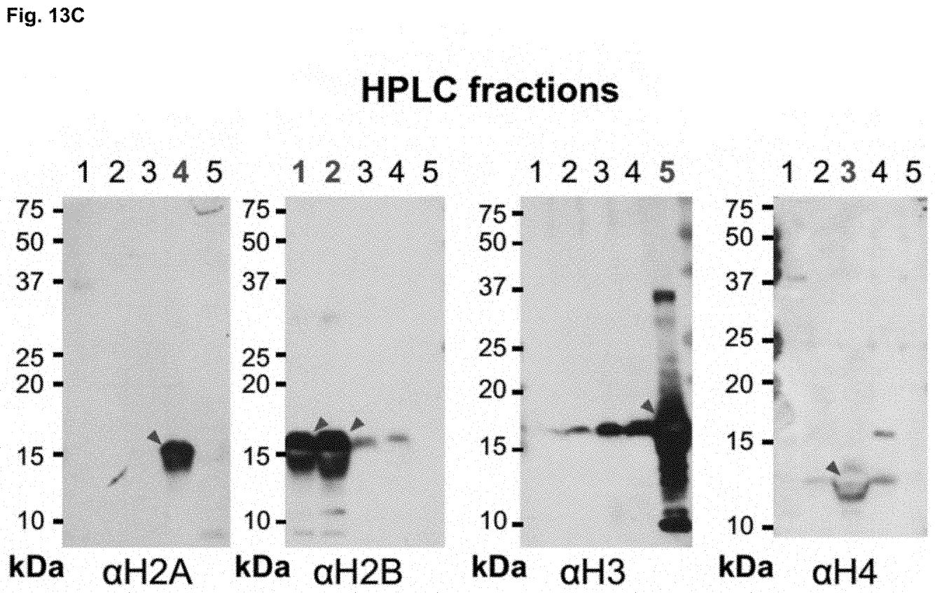

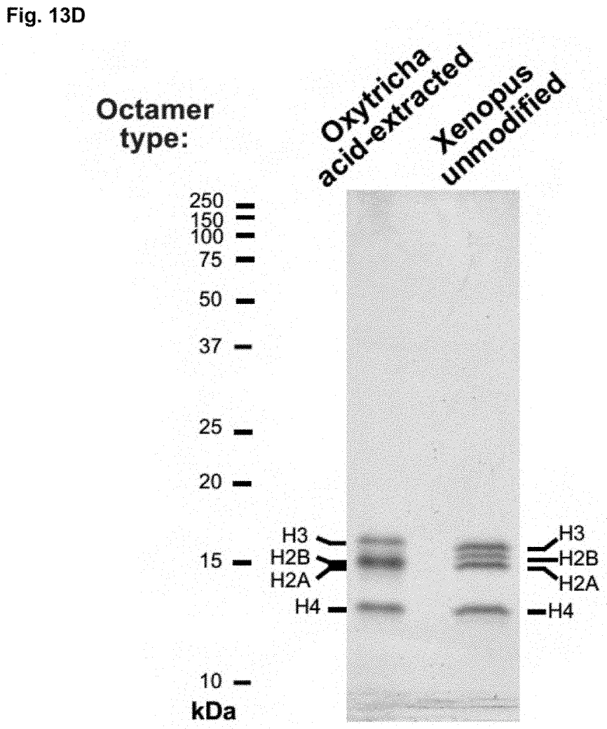

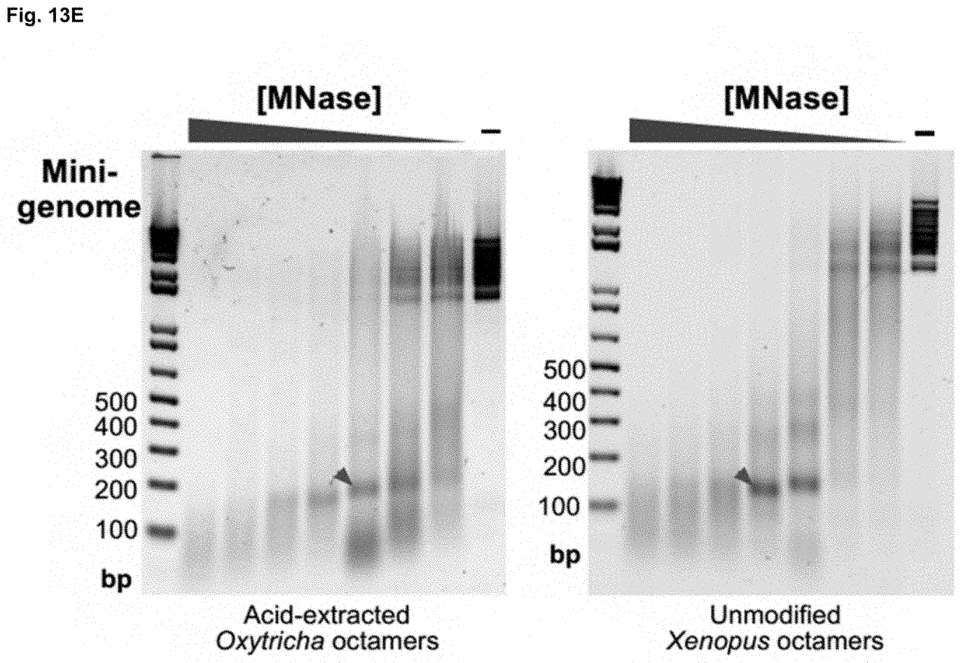

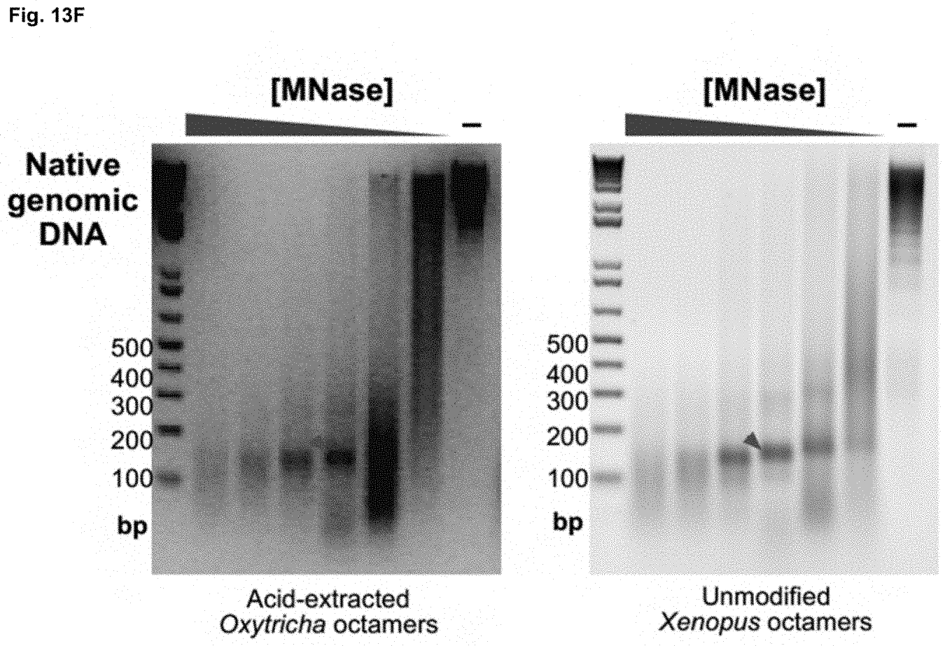

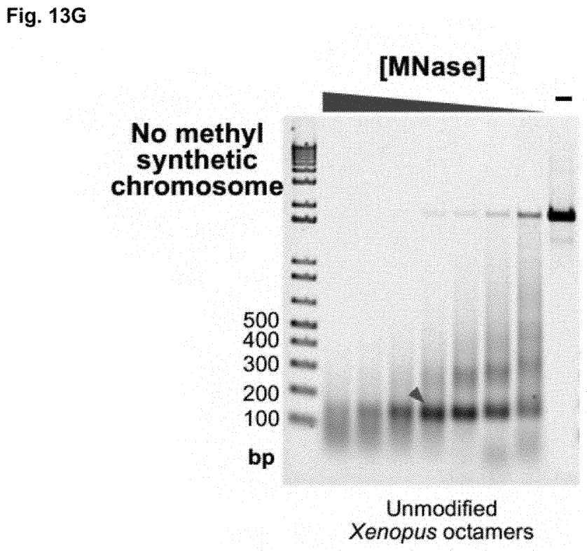

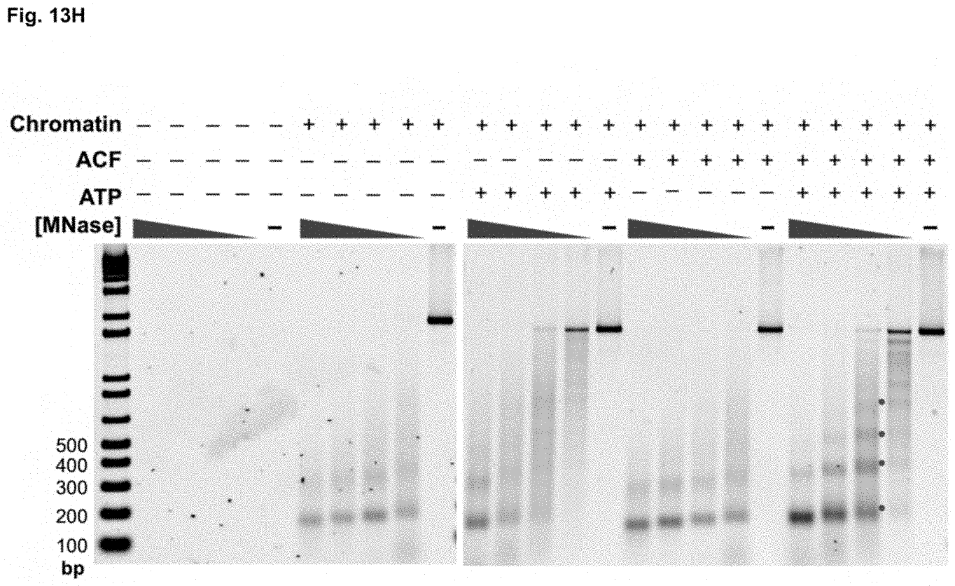

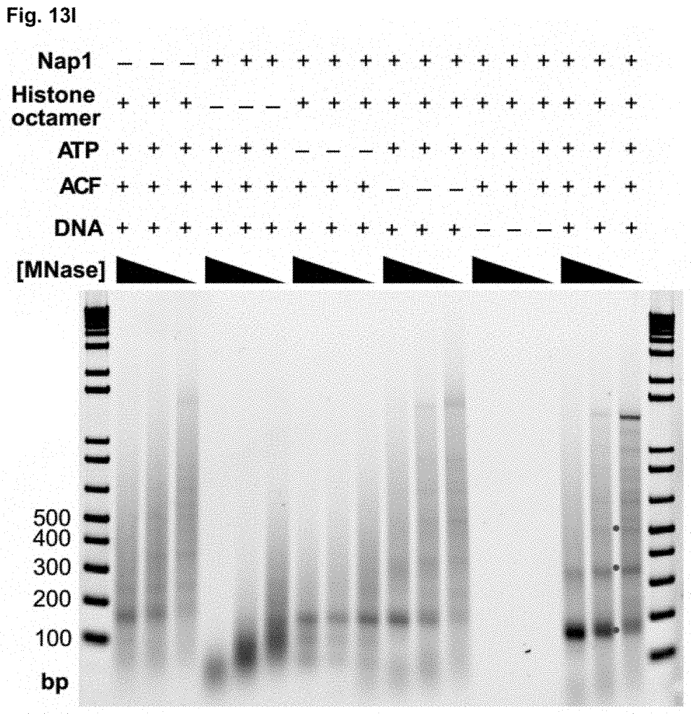

Vegetative Oxytricha trifallax strain JRB310 was cultured as described in the subheading: “Experimental model and subject details” of this study. Cells were starved for 14 hr and subsequently harvested for macronuclear isolation as described in the subheading: “in vivo MNase-seq” of this study. However, formaldehyde fixation was omitted. Purified nuclei were pelleted by centrifugation at 4000×g, resuspended in 0.421 mL 0.4N H₂SO₄ per 10⁶ input cells, and nutated for 3 hr at 4° C. to extract histones. Subsequently, the acid-extracted mixture was centrifuged at 21,000× a for 15 min to remove debris. Proteins were precipitated from the cleared supernatant using trichloroacetic acid (TCA), washed with cold acetone, then dried and resuspended in 2.5% v/v acetic acid. Individual core histone fractions were purified from crude acid-extracts using semi-preparative RP-HPLC (Vydac C18, 12 micron, 10 mM×250 mm) with 40%-65% HPLC solvent B over 50 min (FIG. 13A). The identity of each purified histone fraction was verified by western analysis (FIG. 13C) using antibodies: anti-H2A (Active Motif #39111), anti-H2B (Abcam #ab1790), anti-H3 (Abcam #ab1791), anti-H4 (Active Motif #39269).

### Preparation of Recombinant Xenopus Histones

All RP-HPLC analyses were performed using 0.1% TFA in water (HPLC solvent A), and 90% acetonitrile, 0.1% TFA in water (HPLC solvent B) as the mobile phases. Wild-type Xenopus H4, H3 C110A, H2B and H2A proteins were expressed in BL21(DE3) pLysS E. coli and purified from inclusion bodies through ion exchange chromatography (Debelouchina et al., 2017). Purified histones were characterized by ESI-MS using a MicrOTOF-Q II ESI-Qq-TOF mass spectrometer (Bruker Daltonics). H4: calculated 11,236 Da, observed 11,236.1 Da; H3 C110A: calculated 15,239 Da, observed 15,238.7 Da; H2A: calculated 13,950 Da, observed 13,949.8 Da; H2B: calculated 13,817 Da, observed 13,816.8 Da.

### Preparation of Histone Octamers

Oxytricha and Xenopus histone octamers were respectively refolded from core histones using established protocols (Beh et al., 2015; Debelouchina et al., 2017). Briefly, lyophilized histone proteins (Xenopus modified or wild-type; Oxytricha acid-extracted) were combined in equimolar amounts in 6 M guanidine hydrochloride, 20 mM Tris pH 7.5 and the final concentration was adjusted to 1 mg/mL. The solution was dialyzed against 2M NaCl, 10 mM Tris, 1 mM EDTA, and the octamers were purified from tetramer and dimer species using size-exclusion chromatography on a Superdex 200 10/300 column (GE Healthcare Life Sciences). The purity of each fraction was analyzed by SDS-PAGE. Pure fractions were combined, concentrated and stored in 50% v/v glycerol at −20° C.

### Preparation of Mini-Genome DNA

98 full-length chromosomes were individually amplified from Oxytricha trifallax strain JRB310 genomic DNA using Phusion DNA polymerase (New England Biolabs). Primer pairs are listed in Table 2. Amplified chromosomes were separately purified using a MinElute PCR purification kit (QIAGEN), and then mixed in equimolar ratios to obtain “mini-genome” DNA. The sample was concentrated by ethanol precipitation and adjusted to a final concentration of ˜1.6 mg/mL.

### Preparation of Native Genomic DNA for Chromatin Assembly Starry

Macronuclei were isolated from vegetative Oxytricha trifallax strain JRB310 as described in the subheading “in vivo MNase-seq” of this study. However, cells were not fixed prior to nuclear isolation. Genomic DNA was purified using the Nucleospin Tissue kit (Macherey-Nagel). Approximately 200 μg of genomic DNA was loaded on a 15%-40% linear sucrose gradient and centrifuged in a SW 40 Ti rotor (Beckman Coulter) at 160,070×g for 22.5 hr at 20° C. Sucrose solutions were in 1M NaCl, 20 mM Tris pH 7.5, 5 mM EDTA. Individual fractions from the sucrose gradient were analyzed on 0.9% agarose-TAE gels. Fractions containing high molecular weight DNA that migrated at the mobility limit were discarded as such DNA species were found to interfere with downstream chromatin assembly. All other fractions were pooled, ethanol precipitated, and adjusted to 0.5 mg/mL DNA.

### Chromatin Assembly and Preparation of Mononucleosomal DNA

Chromatin assemblies were prepared by salt gradient dialysis as previously described (Beh et al., 2015; Luger et al., 1999), or using mouse NAP1 histone chaperone and Drosophila ACF chromatin remodeler as previously described (An and Roeder, 2004; Fyodorov and Kadonaga, 2003). Details of each chromatin assembly procedure are listed below. To reduce sample requirements while maintaining adequate DNA concentrations for chromatin assembly, synthetic chromosomes were first mixed with a hundred-fold excess of “buffer” DNA (PCR-amplified Oxytricha Contig17535.0). We verified that nucleosome occupancy in the methylated region (qPCR primer pairs 6 and 7) of the synthetic chromosome is unaffected by the presence of buffer DNA (FIG. 14E). Native and mini-genome DNA were not mixed with buffer DNA prior to chromatin assembly.

For chromatin assembly through salt dialysis: histone octamers and (synthetic chromosome+buffer) DNA were mixed in a 0.8:1 mass ratio, while histone octamers and (native or mini-genome) DNA were mixed in a 1.3:1 mass ratio, each in a 50 μL total volume. Samples were first dialyzed into start buffer (10 mM Tris pH 7.5, 1.4M KCl, 0.1 mM EDTA pH 7.5, 1 mM DTT) for 1 hr at 4° C. Then, 350 mL end buffer (10 mM Tris pH 7.5, 10 mM KCl, 0.1 mM EDTA, 1 mM DTT) was added at a rate of 1mUmin with stirring. The assembled chromatin was dialyzed overnight at 4° C. into 200 mL end buffer, followed by a final round of dialysis in fresh 200 mL end buffer for 1 hr at 4° C. The assembled chromatin was then adjusted to 50 mM Tris pH 7.9, 5 mM CaCl₂) and digested with MNase (New England Biolabs) to mainly mononucleosomal DNA as previously described (Beh et al., 2015).

For chromatin assembly using mouse NAP1 and Drosophila ACF: NAP1 was recombinantly expressed and purified as described in (An and Roeder, 2004). ACF was purchased from Active Motif. 0.49 μM NAP1 and 58 nM histone octamer were first mixed in a 302p1 reaction volume containing 62 mM KCl, 1.2% w/v polyvinyl alcohol (Sigma Aldrich), 1.2% w/v polyethylene glycol 8000 (Sigma Aldrich), 25 mM HEPES-KOH pH 7.5, 0.1 mM EDTA-KOH, 10% v/v glycerol, and 0.01% v/v NP-40. The NAP1-histone mix was incubated on ice for 30 min. Meanwhile, “AM” mix was prepared, consisting of 20 mM ATP (Sigma Aldrich), 200 mM creatine phosphate (Sigma Aldrich). 33.3 mM MgCl₂, 33.3 μg/μl creatine kinase (Sigma Aldrich) in a 56u1 reaction volume. After the 30 min incubation. 5.29 μl of 1.7 μM ACF complex (Active Motif) and the “AM” mix were sequentially added to the NAP1-histone mix. Then, 10.63 μl of native or mini-genome DNA (2.66 μg) was added, resulting in a 374 μl reaction volume. The final mixture was incubated at 27° C. for 2.5 hr to allow for chromatin assembly. Subsequently, CaCl₂ was added to a final concentration of 5 mM, and the chromatin was digested with MNase (New England Biolabs) to mainly mononucleosomal DNA as previously described (Beh et al., 2015).

Mononucleosome-sized DNA from MNase-digested chromatin was gel-purified and used for tiling qPCR on a Viia 7 Real-Time PCR System with Power SYBR Green PCR master mix (Thermo Fisher), or in vitro MNase-seq on an Illumina HiSeq 2500, according to the manufacturer's instructions. qPCR primer sequences are listed in Table 2.

Tiling qPCR Analysis of Nucleosome Occupancy

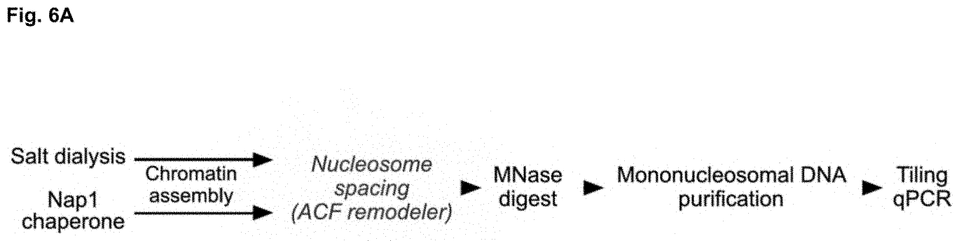

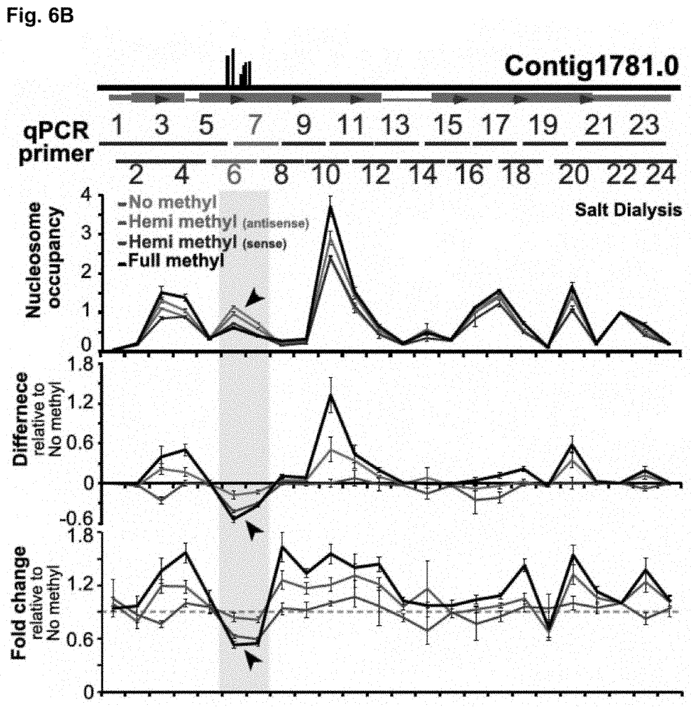

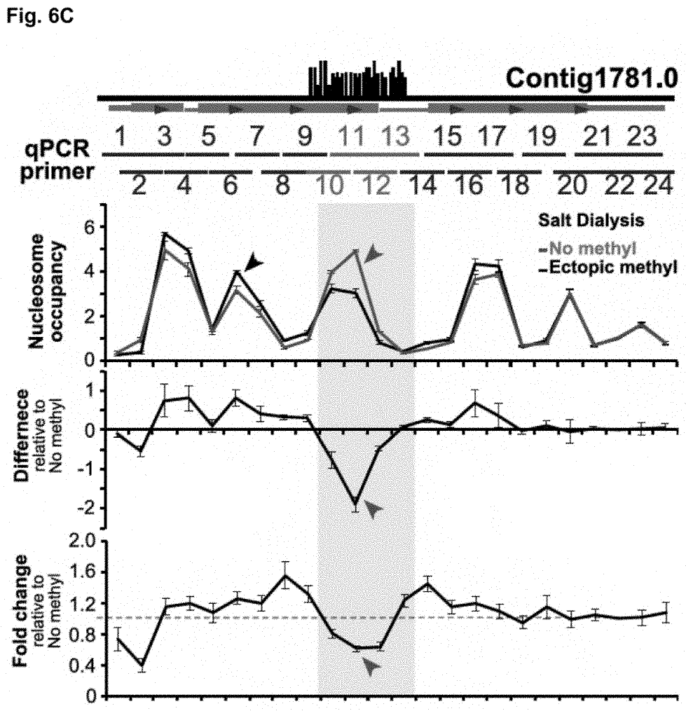

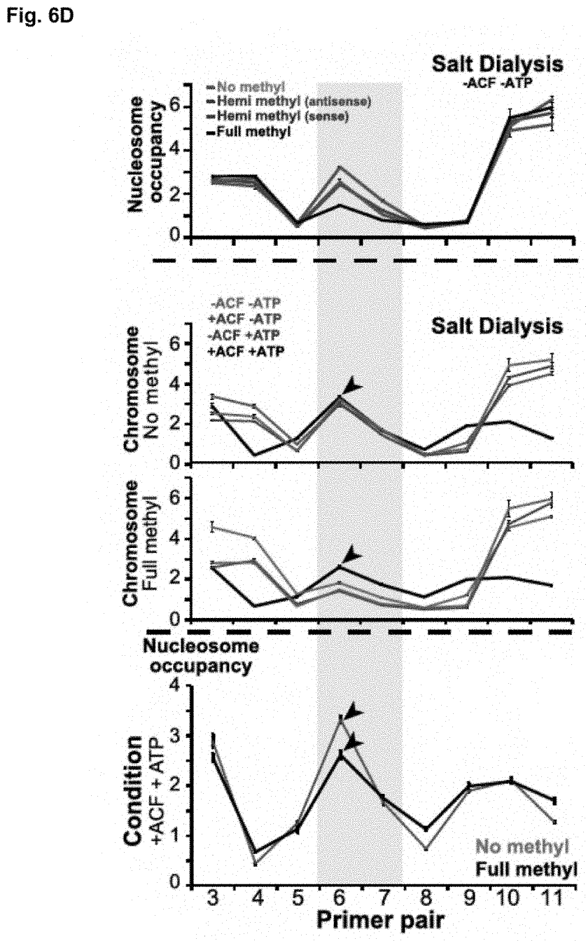

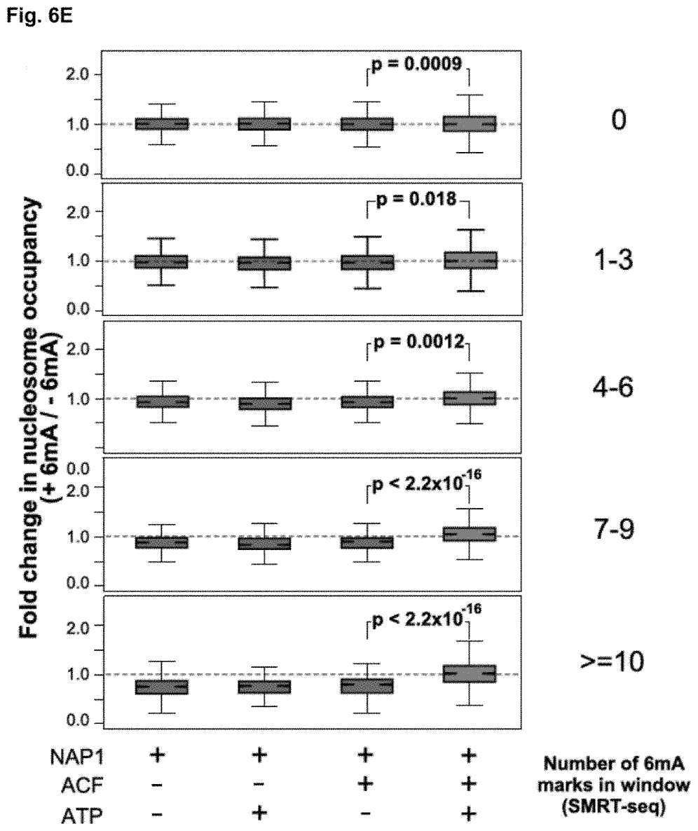

qPCR data were analyzed using the ΔΔCt method (Livak and Schmittgen, 2001). At each locus along the synthetic chromosome, ΔCt=(Ct at locus of interest)−(Ct at qPCR primer pair 22, far from the methylated region). See FIG. 6B for location of qPCR primer pair 22. Separate ΔCt values were calculated from mononucleosomal DNA and the corresponding naked, undigested synthetic chromosome. The ΔΔCt value was calculated from this pair of ΔCt values. This controls for potential variation in PCR amplification efficiency, especially over methylated regions. The fold change in mononucleosomal DNA relative to naked chromosomal DNA at a particular locus is calculated as 2^{-ΔΔCt}, and denotes ‘nucleosome occupancy’ for all presented qPCR data.

### ACF Spacing Assay

ATP-dependent nucleosome spacing was performed in accordance with a previous study (Lieleg et al., 2015). Chromatin was assembled by salt gradient dialysis as described above, and then adjusted to 20 mM HEPES-KOH pH 7.5, 80 mM KCl, 0.5 mM EGTA, 12% v/v glycerol, 10 mM (NH₄)₂SO₄, 2.5 mM DTT. Samples were then incubated for 2.5 hr at 27° C. with 3 mM ATP, 30 mM creatine phosphate, 4 mM MgCl₂, 5 ng/0 creatine kinase, and 11 ng/μL ACF complex (Active Motif). Remodeled chromatin was then adjusted to 5 mM CaCl₂) and subjected to MNase digestion, mononucleosomal DNA purification, and qPCR analysis as described above.

### Phylogenetic Analysis

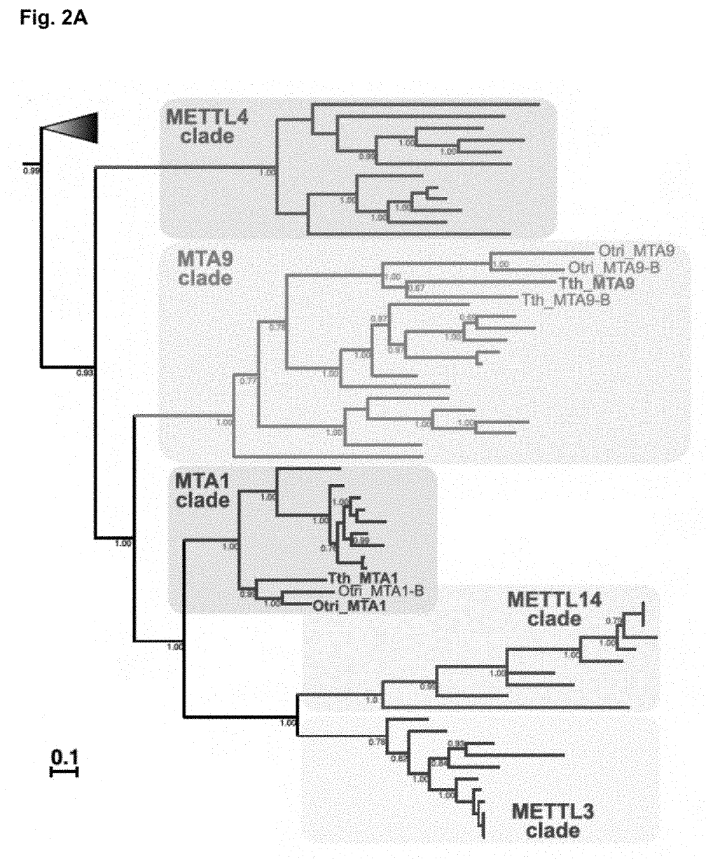

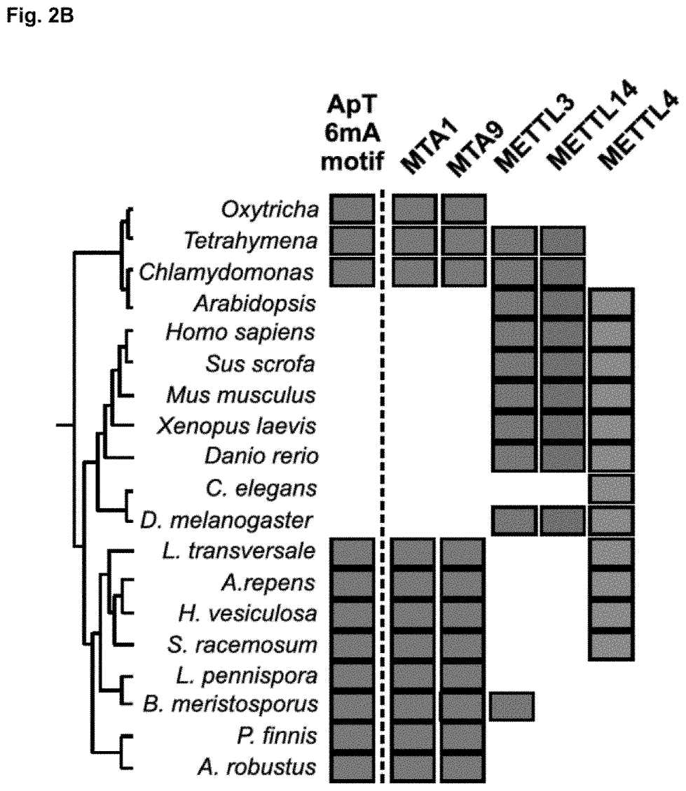

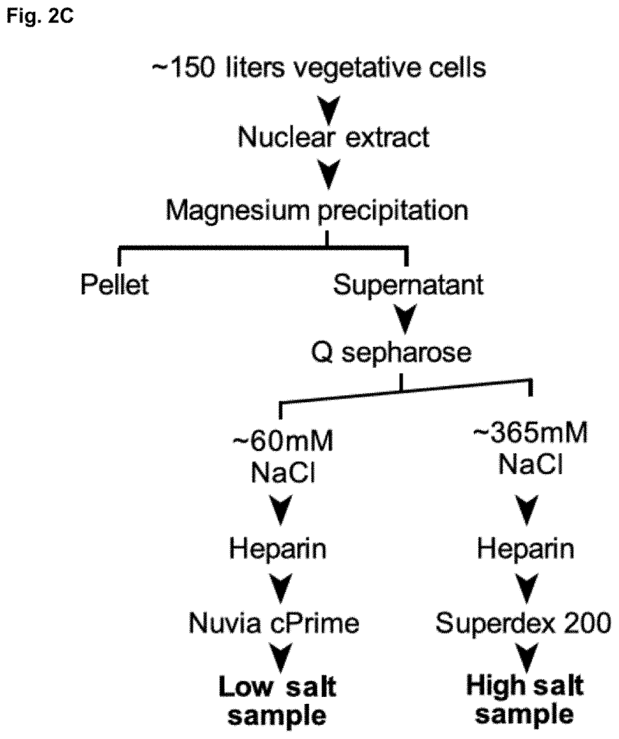

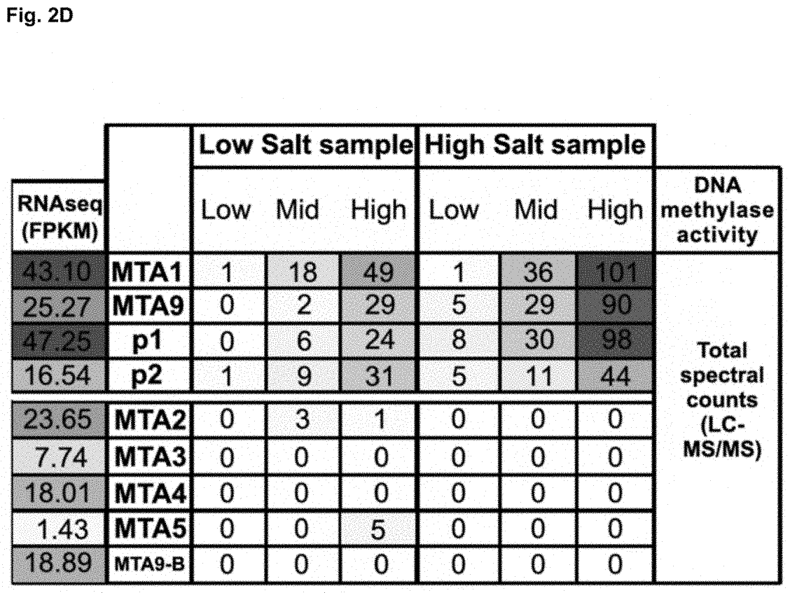

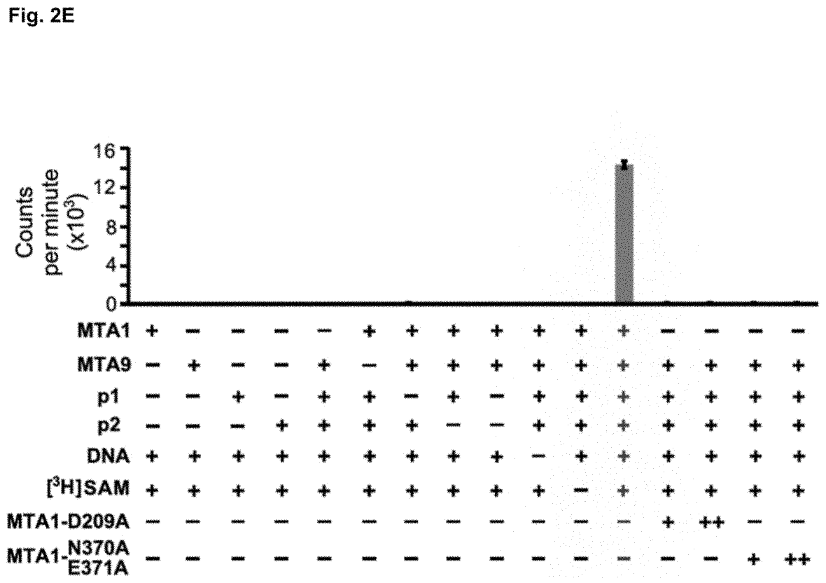

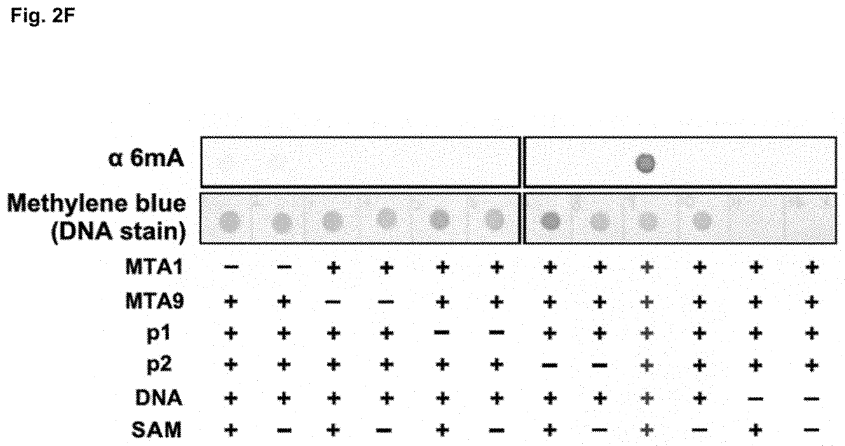

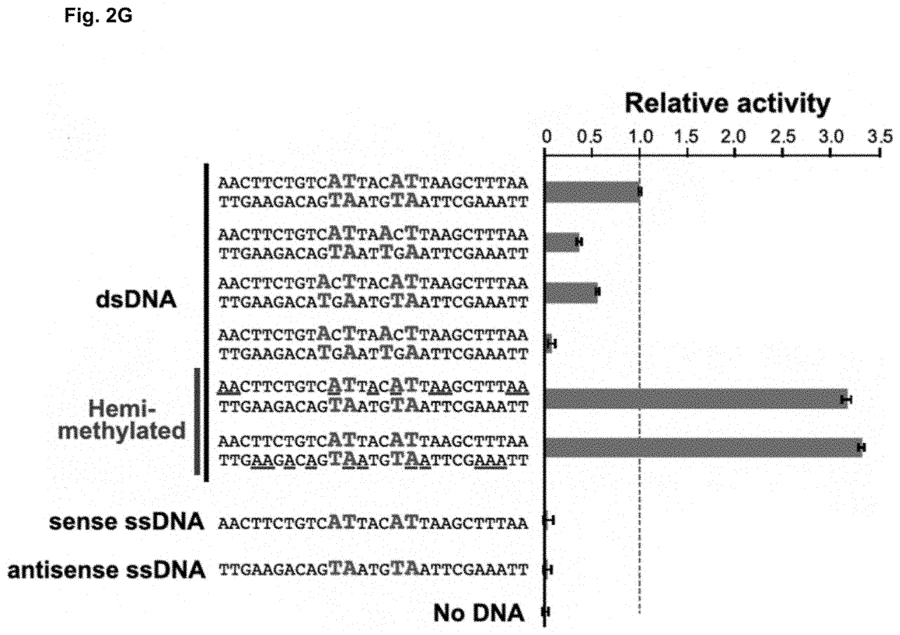

The MTA1 amino acid sequence (UniProt ID: J9IF92 9SPIT) was queried against the NCBI nr database using PSI-BLAST (Altschul et al., 1997; Schaffer et al., 2001) (maximum e-value=1e⁻⁴; enable short queries and filtering of low complexity regions). Retrieved hits were collapsed using CD-HIT (Huang et al., 2010) with minimum sequence identity=0.97 to remove redundant sequences. The resulting sequences were added to existing MT-A70 alignments from (Greer et al., 2015) using MAFFT (-add) (Katoh et al., 2017; Kuraku et al., 2013). Gaps and duplicate sequences were removed from the merged alignment. Only sequences corresponding to the taxa in FIG. 2B were retained. The alignment was then used for phylogenetic tree construction using MrBayes in the CIPRES Science Gateway (Miller et al., 2010) with 5×10⁶ generations. Protein sequences used for MrBayes analysis are given in Table 1.

The above procedure was also used for constructing phylogenetic trees from p1 (UniProt ID: Q22VV9 TETTS) and p2 (UniProt ID: I7M8B9 TETTS). However, protein sequences were aligned using MAFFT without adding to an existing alignment.

Preparation of Nuclear Extracts with DNA Methyltransferase Activity

Vegetative Tetrahymena cells were grown in SSP medium to log-phase (˜3.5×10⁶ cells/mL) and collected by centrifugation at 2,300×g for 5 min in an SLA-3000 rotor. The supernatant was discarded, and cells were resuspended in medium B (10 mM Tris pH 6.75, 2 mM MgCl₂, 0.1M sucrose, 0.05% w/v spermidine trihydrochloride, 4% w/v gum Arabic, 0.63% w/v 1-octanol, and 1 mM PMSF). Gum arabic (Sigma Aldrich) is prepared as a 20% w/v stock and centrifuged at 7,000×g for 30 min to remove undissolved clumps. For each volume of cell culture, one-third volume of medium B was added to the Tetrahymena cell pellet. Cells were resuspended and homogenized in a chilled Waring Blender (Waring PBB212) at high speed for 40 s. The resulting lysate was subsequently centrifuged at 2,750×g for 5 min in an SLA-3000 rotor to pellet macronuclei. The nuclear pellet was washed twice with medium B and then five times in MM medium (10 mM Tris-HCl pH 7.8, 0.25M sucrose, 15 mM MgCl₂, 0.1% w/v spermidine trihydrochloride, 1 mM DTT, 1 mM PMSF). Macronuclei were pelleted between wash steps by centrifuging at 2,500×g for 5 min in an SLA-3000 rotor. Finally, the total number of washed macronuclei was counted with a hemocytometer using a Zeiss ID03 microscope. Nuclear proteins were extracted by vigorously resuspending the pellet in M M salt buffer (10 mM Tris-HCl pH 7.8, 0.25M sucrose, 15 mM MgCl2, 350 mM NaCl, 0.1% w/v spermidine trihydrochloride, 1 mM DTT, 1 mM PMSF). 1 mL M M salt buffer was added per 2.33×108 macronuclei. The viscous mixture was nutated for 45 min at 4° C., and then cleared at 175,000×g for 30 min at 4° C. in a SW 41 Ti rotor. Following this, the supernatant was dialyzed in a Slide-A-Lyzer 3.5K MWCO cassette (Thermo Fisher) overnight at 4° C. against two changes of MM minus medium (10 mM Tris-HCl pH 7.8, 15 mM MgCl₂, 1 mM DTT, 0.5 mM PMSF). The dialysate was then centrifuged at 7,197×g for 1 hr at 4″C to remove precipitates, and dialyzed overnight in a Slide-A-Lyzer 3.5K MWCO cassette (Thermo Fisher) at 4° C. against two changes of MN3 buffer (30 mM Tris-HCl pH 7.8, 1 mM EDTA, 15 mM NaCl, 20% v/v glycerol, 1 mM DTT, 0.5 mM PMSF). The final dialysate was cleared by centrifugation at 7,197 g for 1.5 hr at 4° C., flash frozen, and stored at −80° C. This nuclear extract was used for all subsequent biochemical fractionation and 6 mA methylation assays.

Partial Purification of MTA1c from Nuclear Extracts

Tetrahymena nuclear extracts were passed through a HiTrap O HP column (GE Healthcare) and eluted using a linear aradient of 15 mM to 650 mM NaCl in 30 mM Tris-HCl pH 7.8, 1 mM EDTA, 20% v/v glycerol, 1 mM DTT, 0.5 mM PMSF, over 30 column volumes. Each fraction was assayed for DNA methyltransferase activity using radiolabeled SAM as described in the next section. The DNA methyltransferase activity eluted in two peaks, at ˜60 mM and ˜365 mM NaCl, termed the “low salt sample” and “high salt sample.” Fractions corresponding to each peak were pooled and passed through a HiTrap Heparin HP column (GE Healthcare). Bound proteins were eluted using a linear gradient of 60 mM to 1M NaCl (for the low salt sample) or 350 mM to 1M NaCl (for the high salt sample) over 30 column volumes. Fractions with DNA methyltransferase activity were respectively pooled and dialyzed into 10 mM sodium phosphate pH 6.8, 100 mM NaCl, 10% v/v glycerol, 0.3 mM CaCl₂), 0.5 mM DTT (for the low salt sample); or 30 mM Tris-HCl pH 7.8, 1 mM EDTA, 200 mM NaCl, 10% v/v glycerol, 1 mM DTT, 0.2 mM PMSF (for the high salt sample). The dialyzed low salt sample was passed through a Nuvia cPrime column (Bio-Rad) and eluted using a linear gradient of 100 mM to 1M NaCl in 50 mM sodium phosphate pH 6.8, 10% v/v glycerol, 0.5 mM DTT. Separately, the dialyzed high salt sample was fractionated using a Superdex 200 10/300 GL column (GE Healthcare) in 30 mM Tris-HCl pH 7.8, 1 mM EDTA, 200 mM NaCl, 10% v/v glycerol, 1 mM DTT. Fractions from the Nuvia cPrime and Superdex 200 columns were dialyzed into 30 mM Tris-HCl pH 7.8, 1 mM EDTA, 15 mM NaCl, 20% v/v glycerol, 1 mM DTT, 0.5 mM PMSF and assayed for DNA methyltransferase activity. Those with qualitatively low, medium, and high activity were subjected to mass spectrometry to identify candidate methyltransferase proteins (FIG. 2D; Table 6). This experiment identified four proteins that co-purify with DNA methyltransferase activity—MTA1, MTA9, p1, and p2—and are collectively termed as “MTA1c” in the present disclosure. All four proteins are necessary for 6 mA methylation in vitro.

### Recombinant Expression of MTA1, MTA9, p1, and p2 Proteins

Full length MTA1, MTA9, p1, and p2 open reading frames were codon-optimized for bacterial expression and cloned into a pET-His6-SUMO vector using ligation independent cloning. Protein sequences are listed in Table 3. The vector was a gift from Scott Gradia (Addgene plasmid #29659; http://addgene.org/29659; RRID: Addgene 29659). Mutations in the MTA1 open reading frame was introduced using the OS® Site-Directed Mutagenesis Kit (New England Biolabs). For recombinant expression, pET-His6-SUMO-MTA1 (wild-type and mutant) was transformed into SHuffle T7 competent E. co/i (New England Biolabs); pET-His6-SUMO-MTA9 was transformed into Lemo (DE3) competent E. coli (New England Biolabs); pET-His6-SUMO-p1 and pET-His6-SUMO-p2 were transformed into BL21(DE3) competent E. coli (New England Biolabs). IPTG induction was performed at 16′C overnight. Induced cells were resuspended in 25 ml of lysis buffer B (50 mM Tris pH 7.8, 300 mM NaCl, 5% v/v glycerol, 10 mM imidazole, 5 mM BME, 1 mM PMSF, 0.5× ProBlock Gold Bacterial protease inhibitor cocktail [GoldBio]). The cells were sonicated at 35% amplitude for a total of 4 minutes, with a 10 s off, 10 s cycle using a Model 505 Sonic Dismembrator (Fisherbrand). Lysates were cleared by centrifugation at 30,000 g for 30 min at 4° C., mixed with pre-washed Ni-NTA agarose (Invitrogen), and nutated for 45 min at 4° C. The resin was subsequently washed with lysis buffer and eluted in 50 mM Tris pH 7.8, 300 mM NaCl, 5% v/v glycerol, 400 mM glycerol, 5 mM BME, lx ProBlock Gold bacterial protease inhibitor cocktail [GoldBio]). Eluates were dialyzed into lysis buffer B and then digested with TEV protease (gift from S.H. Sternberg) at 4° C. overnight. The resulting mixture was passed through a fresh batch of Ni-NTA agarose (Invitrogen) to remove cleaved affinity tags. The flow-through containing each recombinant protein was flash frozen and used for all downstream methyltransferase assays.

### Methyltransferase Assays

### Generation of DNA and RNA Substrates

A 954 bp dsDNA PCR product was used in all assays involving Tetrahymena nuclear extract. This substrate was amplified by PCR from Tetrahymena thermophila strain SB210 macronuclear SB210 genomic DNA using PCR primers metGATC F2 and metGATC_R2 (Table 2). The resulting product was purified using Ampure XP beads (Beckman Coulter). This 954 bp region of the genome contains a high level of 6 mA in vivo. Thus, the underlying DNA sequence may be intrinsically amenable to methylation by Tetrahymena MTA1. Note that the amplified 954 bp product is devoid of DNA methylation as unmodified dNTPs were used for PCR. Separately, a 350 bp dsDNA PCR product was used in all assays involving recombinant MTA1, MTA9, p1 and p2. This sequence lacks 5′-NATC-3′ motifs, and was used to reduce background DNA methylation from contaminating Dam methyltransferase in recombinant protein preparations. The 350 bp dsDNA PCR product was amplified from Tetrahymena thermophila strain SB210 macronuclear SB210 genomic DNA using the PCR primers noGATC2 F and noGATC2_R (Table 2), and purified using Ampure XP beads (Beckman Coulter).

For short DNA substrates (<50 bp), oligonucleotides were purchased from Integrated DNA Technologies and either directly used as ssDNA, or annealed with its complementary sequence to obtain dsDNA. To prepare hemimethylated 27 bp dsDNA in FIG. 2G, either strand was methylated using EcoGII methyltransferase (New England BioLabs) before annealing with the complementary sequence.

To generate ˜350nt ssRNA and −350 bp dsRNA, the aforementioned 350 bp dsDNA was first PCR-amplified using primers containing T7 overhangs (primer pairs T7noGATC2_F2/noGATC2_R and T7noGATC2_F2/T7noGATC2_R2 respectively; see Table 2 for primer sequences). Each PCR product was used as a template for in vitro transcription using the HiScribe T7 High Yield RNA Synthesis Kit (New England Biolabs). The synthesized RNA was rigorously treated with DNase (ThermoFisher) purified using acid phenol:chloroform extraction, followed by two rounds of chloroform extraction. Each sample was subsequently ethanol precipitated and resuspended in water for use in methyltransferase assays.

### Radioactive Methyltransferase Assay

For experiments involving nuclear extract, 2.18 μg of 954 bp dsDNA substrate was mixed with 4-8 μl nuclear extract and 0.64 μM 3H-labeled S-adenosyl-L-methionine ([³H]SAM) in 33 mM Tris-HCl pH 7.5. 6 mM EDTA. 4.3 mM BME. in a 15p1 reaction volume. For experiments involving recombinant MTA1c protein components (ie. MTA1, MTA9, p1, and/or p2), ˜3 μM oligonucleotide ssDNA/annealed dsDNA is used. Alternatively, 1.3 μg of 350 bp dsDNA substrate (or an equimolar amount ˜350nt ssRNA, or ˜350 bp dsDNA) was used in place of DNA oligonucleotide substrates. ssRNA was heated at 90° C. for 2 min and snap cooled to minimize secondary structures before mixing with other components of the methyltransferase assay. All samples were incubated overnight at 37° C., and subsequently spotted onto 1 cm×1 cm squares of Hybond-XL membrane (GE Healthcare). Membranes were then washed thrice with 0.2M ammonium bicarbonate, once with distilled water, twice with 100% ethanol, and finally air-dried for 1 hr. Each membrane was immersed in 5 mL Ultima Gold (PerkinElmer) and used for scintillation counting on a TriCarb 2910 TR (Perkin Elmer).

### Non-Radioactive Methyltransferase Assay

For assays involving nuclear extract: 5.5 pg of 954 bp DNA substrate was mixed with 20 nuclear extract and 0.2 mM S-adenosyl-L methionine (NEB) in 33 mM Tris-HCl pH 7.5, 6 mM EDTA, 4.3 mM BME in a 15p1 reaction volume. For assays involving recombinant MTA1c protein components (ie. MTA1, MTA9, p1, and/or p2), 2.6 μg of 350 bp DNA substrate was mixed with 540 nM MTA1, 90 nM MTA9, 1.5 μM p1, 1.0 μM p2 proteins. The band of expected size in each recombinant protein preparation was compared against a series of BSA standards to calculate protein concentration. All methylation reactions were incubated at 37° C. overnight, then purified using a MinElute purification kit (QIAGEN), denatured at 95° C. for 10 min, and snap cooled in an ice water bath. Samples were spotted on a Hybond N+ membrane (GE Healthcare), air-dried for 5 min and UV-cross-linked with 120,000 μJ/cm² exposure using an Ultra-Lum UVC-515 Ultraviolet Multilinker. The cross-linked membrane was blocked in 5% milk in TBST (containing 0.1% v/v Tween) and incubated with 1:1,000 anti-N6-methyladenosine antibody (Synaptic Systems) at 4° C. overnight. The membrane was then washed three times with TBST, incubated with 1:3,000 Goat anti-rabbit HRP antibody (Bio-Rad) at room temperature for 1 hr, washed another three times with 1×TBST, and developed using Amersham ECL Western Blotting Detection Kit (GE Healthcare). This dot blot assay was used to measure 6 mA levels in FIGS. 2F, 3B, 5C, and 10C.

### Quantitative Mass Spectrometry Analysis of dA and 6 mA

10.5 μg Oxytricha or Tetrahymena macronuclear genomic DNA was first digested to nucleosides by mixing with 14p1 DNA degradase plus enzyme (Zymo Research) in a 262.5 μl reaction volume. Samples were incubated at 37° C. overnight, then 70° C. for 20 min to deactivate the enzyme.

The internal nucleoside standards ¹⁵N₅-dA and D₃-6 mA were used to quantify endogenous dA and 6 mA levels in ciliate DNA. ¹⁵N₅-dA was purchased from Cambridge Isotope Laboratories, while D₃-6 mA was synthesized as described in the following section. Nucleoside samples were spiked with 1 ng/μl ¹⁵N₅-dA and 200 pg/μl D₃-6 mA in an autosampler vial. Samples were loaded onto a 1 mm×100 mm C18 column (Ace C18-AR, Mac-Mod) using a Shimadzu HPLC system and PAL auto-sampler (20 μl/injection) at a flow rate of 70 μl/min. The column was connected inline to an electrospray source couple to an LTQ-Orbitrap XL mass spectrometer (Thermo Fisher). Caffeine (2 pmol/μl in 50% Acetonitrile with 0.1% FA) was injected as a lock mass through a tee at the column outlet using a syringe pump at 0.5p1/min (Harvard PHD 2000). Chromatographic separation was achieved with a linear gradient from 10% to 99% B (A: 0.1% Formic Acid, B: 0.1% Formic Acid in Acetonitrile) in 5 min, followed by 5 min wash at 100% B and equilibration for 10 min with 1% B (total 20 min program). Electrospray ionization was achieved using a spray voltage of 4.50 kV aided by sheath gas (Nitrogen) flow rate of 18 (arbitrary units) and auxiliary gas (Nitrogen) flow rate of 2 (arbitrary units). Full scan MS data were acquired in the Orbitrap at a resolution of 60,000 in profile mode from the m/z range of 190-290. A parent mass list was utilized to acquire MS/MS spectra at a resolution of 7500 in the Orbitrap. LC-MS data were manually interpreted in Xcalibur's Qual browser (Thermo, Version 2.1) to visualize nucleoside mass spectra and to generate extracted ion chromatograms by using the theoretical [M+H] within a range of ±2 ppm. Peak areas were extracted in Skyline (Ver. 3.5.0.9319).

### Synthesis of D₃-6 mA Nucleoside

2′-Deoxyadenosine and CD3I were purchased from Sigma Aldrich. Flash chromatography was performed on a Biotage Isolera using silica columns (Biotage SNAP Ultra, HP-Sphere 25 pm). Semi-preparative RP-HPLC was performed on a Hewlett-Packard 1200 series instrument equipped with a Waters XBridge BEH C18 column (5 μm, 10×250 mm) at a flow rate of 4 mL/min, eluting using A (0.1% formic acid in H₂O) and B (0.1% formic acid in 9:1 MeCN/H₂O). ¹H NMR spectra were recorded on a Bruker UltraShield Plus 500 MHz instrument. Data for ¹H NMR are reported as follows: chemical shift (8 ppm), multiplicity (s=singlet, br=broad signal, d=doublet, dd=doublet of doublets) and coupling constant (Hz) where possible. ¹³C NMR spectra were recorded on a Bruker UltraShield Plus 500 MHz.

D₃-6 mA (2′Deoxy-6-[D3]-methyladenosine) were synthesized and purified according to (Schiffers et al., 2017). After an initial purification by flash column chromatography, the methylated compounds were further purified by semipreparative RP-HPLC (linear gradient of 0% to 20% B over 30 min) affording the desired compounds in 14% and 10% yields respectively after lyophilization.

### 2Deoxy-6-[D3]methyladenosine

¹H NMR (500 MHz, D₂O) δ 7.98 (s, 1H), 7.77 (s, 1H), 6.17 (m, 1H), 4.54 (m, 1H), 4.10 (m, 1H), 3.79 (dd, J=12.7, 3.2 Hz, 1H), 3.71 (dd, J=12.7, 4.3 Hz, 1H), 2.60 (m, 1H), 2.44 (ddd, J=14.0, 6.3, 3.3 Hz, 1H).

¹³C NMR (126 MHz, D₂O) δ 154.0, 151.5, 146.1, 138.9, 118.4, 87.3, 84.3, 71.1, 61.6, 39.2, 26.4 ppm. (Peak at 26.4 ppm appears as a broad signal. C-D coupling is not resolved).

HR-MS (ESI+): m/z calculated for [C₁₁H₁₃D₃N₅O₃]⁺ ([M+Hr): 269.1436. found 269.1421.

### Mass Spectrometry Analysis of Proteins in Tetrahymena Nuclear Extracts

Samples where topped up to 200p1 with 50 mM ammonium bicarbonate pH 8. TCEP was added to 5 mM final concentration and left to incubate at 60° C. for 10 min. 15 mM chloroacetamide was then added and left to incubate in the dark at room temperature for 30 min. 1 μg of Trypsin Gold (Promega) was added to each sample and incubated end-over-end at 37° C. for 16 hr. An additional 0.25 μg of Trypsin Gold was added and incubated end-over-end at 37° C. for 3 hr. Samples were acidified by adding TFA to 0.2% final concentration, and desalted using SDB stage-tips (Rappsilber et al., 2007). Samples were dried completely in a speedvac and resuspended in 20p1 of 0.1% formic acid pH 3.5 μl was injected per run using an Easy-nLC 1200 UPLC system. Samples were loaded directly onto a 45 cm long 75 pm inner diameter nano capillary column packed with 1.9 μm C18-AQ (Dr. Maisch, Germany) mated to metal emitter in-line with an Orbitrap Fusion Lumos (Thermo Scientific, USA). The mass spectrometer was operated in data dependent mode with the 120,000 resolution MS1 scan (AGC 4e5, Max IT 50 ms, 400-1500 m/z) in the Orbitrap followed by up to 20 MS/MS scans with CID fragmentation in the ion trap. Dynamic exclusion list was invoked to exclude previously sequenced peptides for 60 s if sequenced within the last 30 s, and maximum cycle time of 3 s was used. Peptides were isolated for fragmentation using the quadrupole (1.6 Da window). Ns was utilized. Ion-trap was operated in Rapid mode with AGC 2e3, maximum IT of 300 msec and minimum of 5000 ions.

Raw files were searched using Byonic (Bern et al., 2012) and Sequest HT algorithms (Eng et al., 1994) within the Proteome Discoverer 2.1 suite (Thermo Scientific, USA). 1 Oppm MS1 and 0.4 Da MS2 mass tolerances were specified. Caramidomethylation of cysteine was used as fixed modification, while oxidation of methionine, pyro-Glu from Gln and deamidation of asparagine were specified as dynamic modifications. Trypsin digestion with maximum of 2 missed cleavages were allowed. Files were searched against the Tetrahymena themophila macronuclear reference proteome (June 2014 build), supplemented with common contaminants (27,099 total entries).

Scaffold (version Scaffold 4.8.7, Proteome Software Inc., Portland, Oreg.) was used to validate MS/MS based peptide and protein identifications. Peptide identifications were accepted if they could be established at greater than 93.0% probability. Peptide Probabilities from Sequest and Byonic were assigned by the Scaffold Local FDR algorithm. Protein identifications were accepted if they could be established at greater than 99.0% probability to achieve an FDR less than 1.0% and contained at least 3 identified peptides. Protein probabilities were assigned by the Protein Prophet algorithm (Nesvizhskii et al., 2003). Proteins that contained similar peptides and could not be differentiated based on MS/MS analysis alone were grouped to satisfy the principles of parsimony.

### Generation of Mta1 Mutant Lines

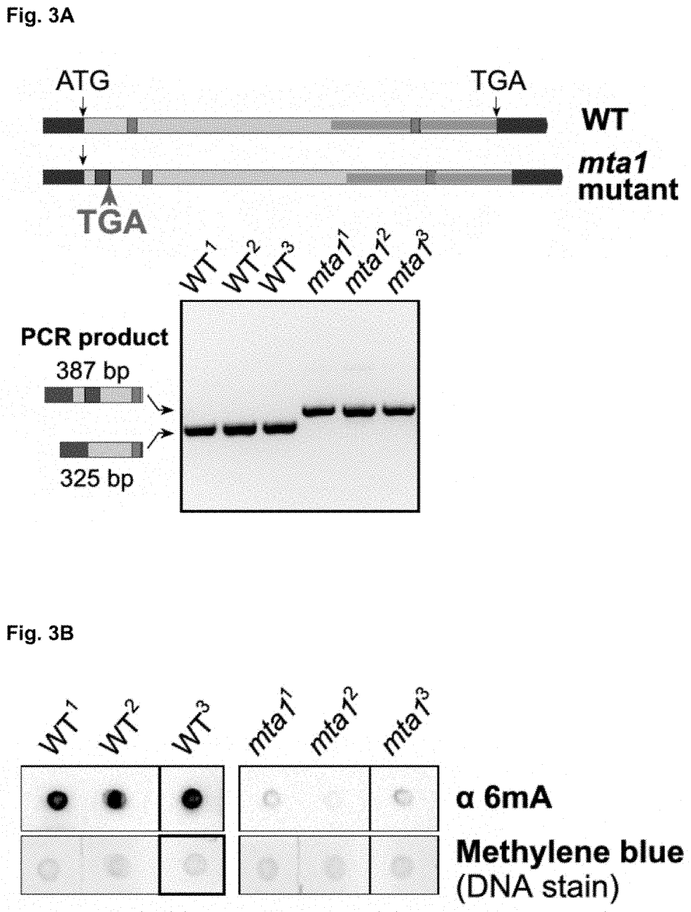

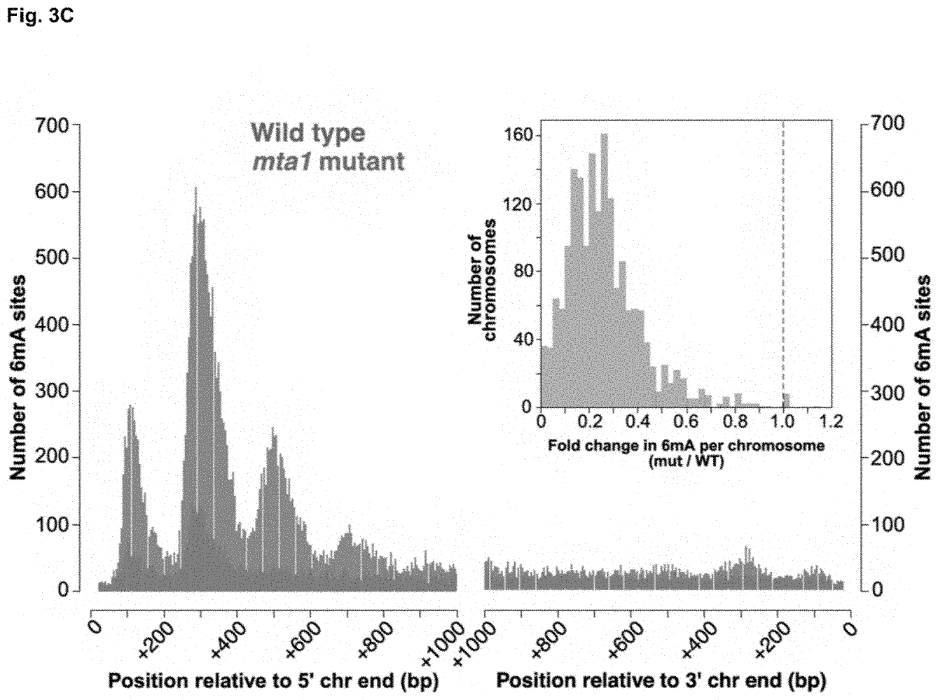

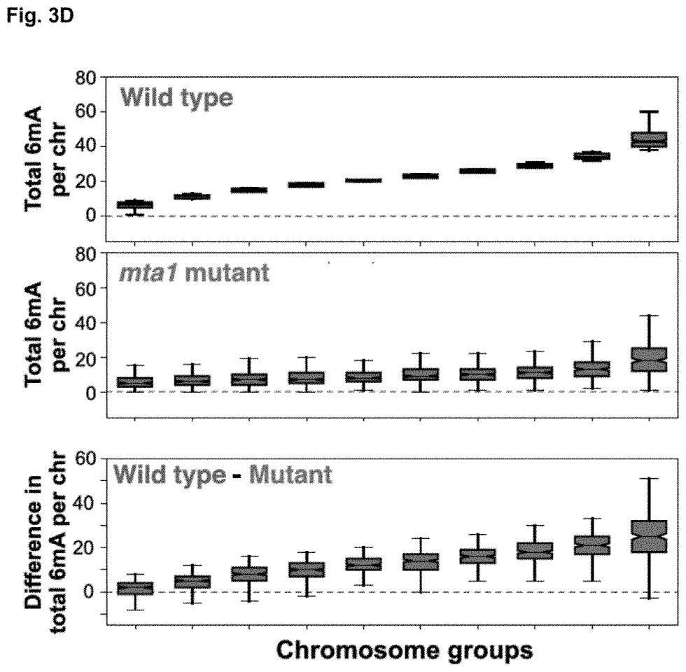

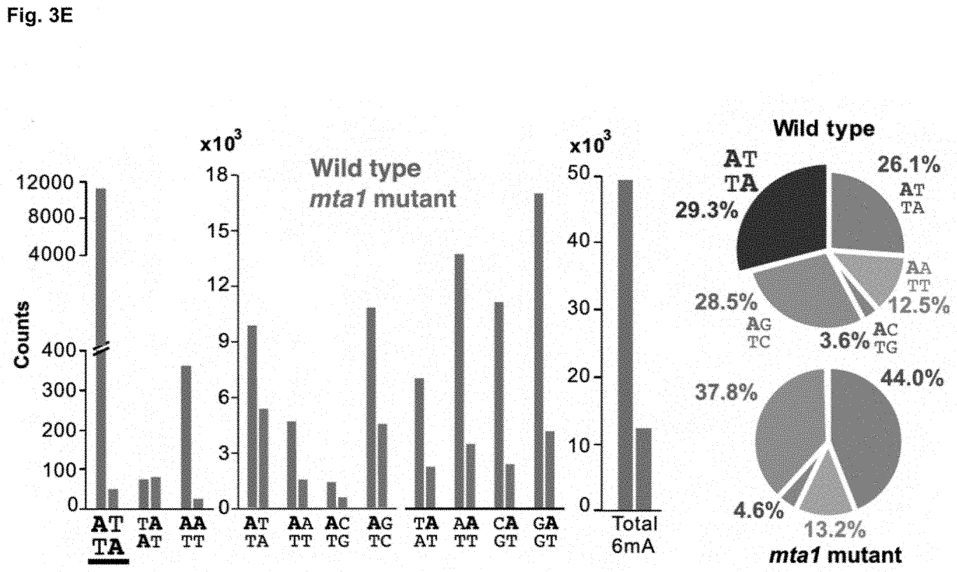

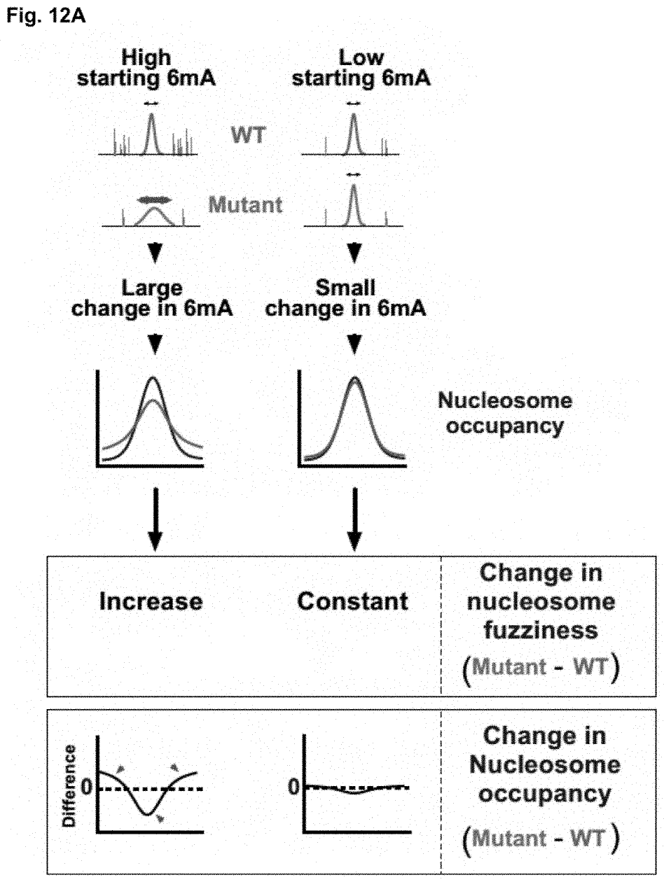

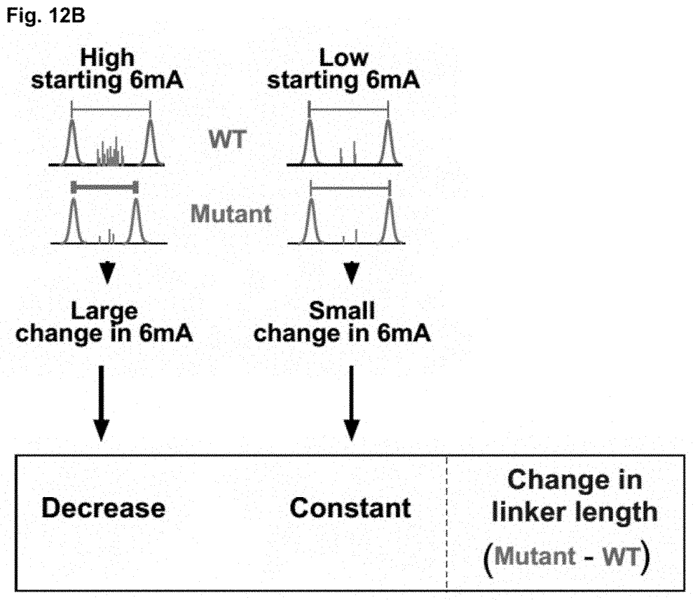

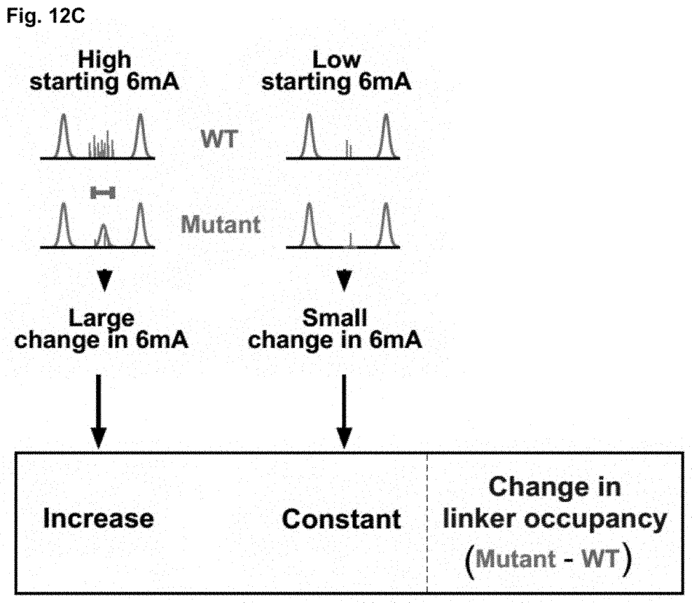

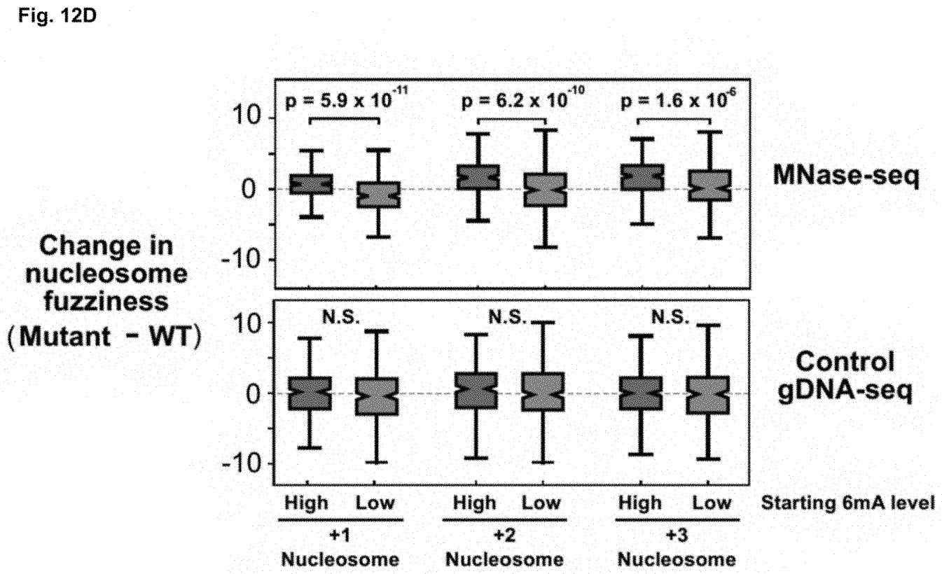

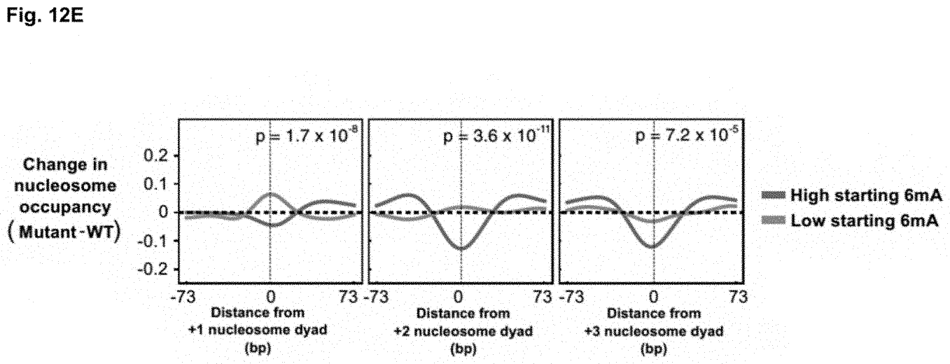

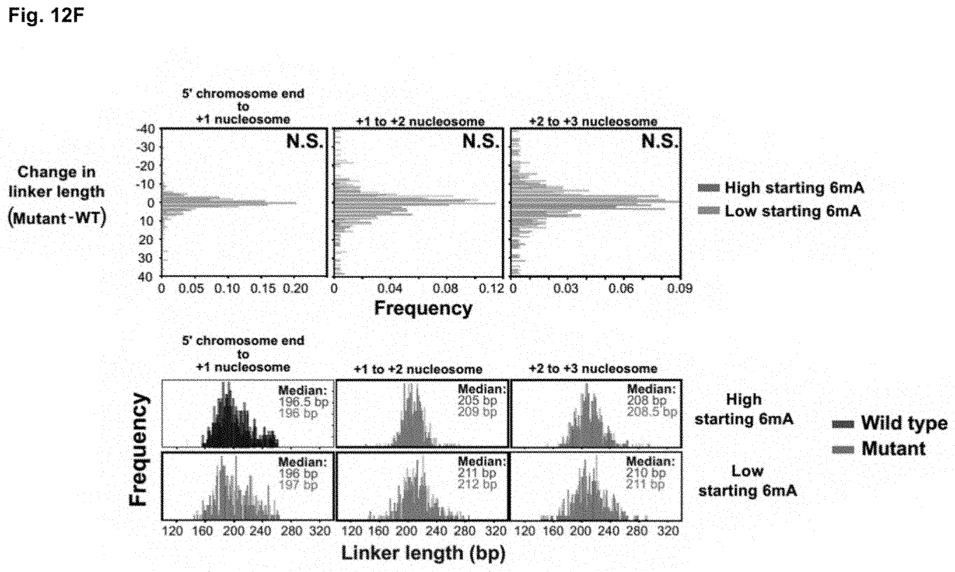

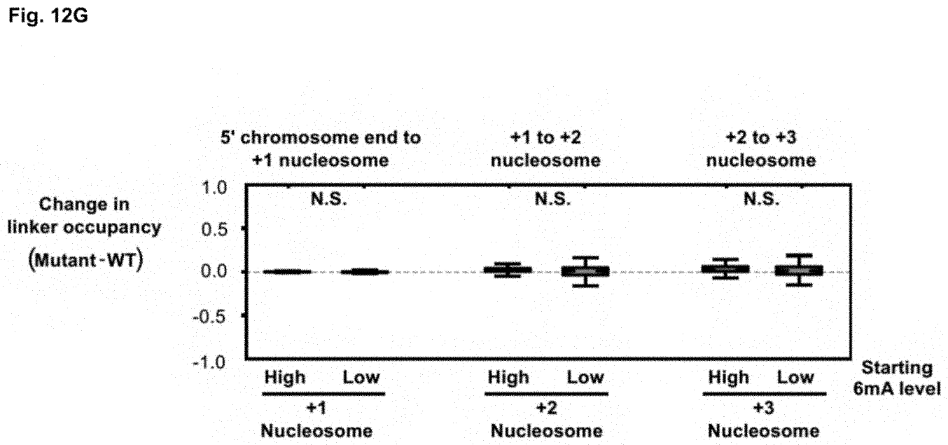

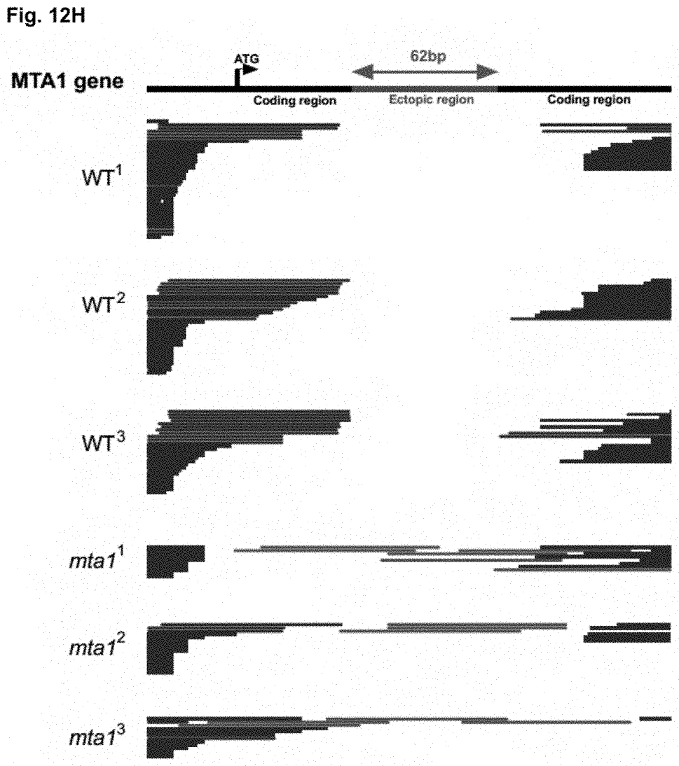

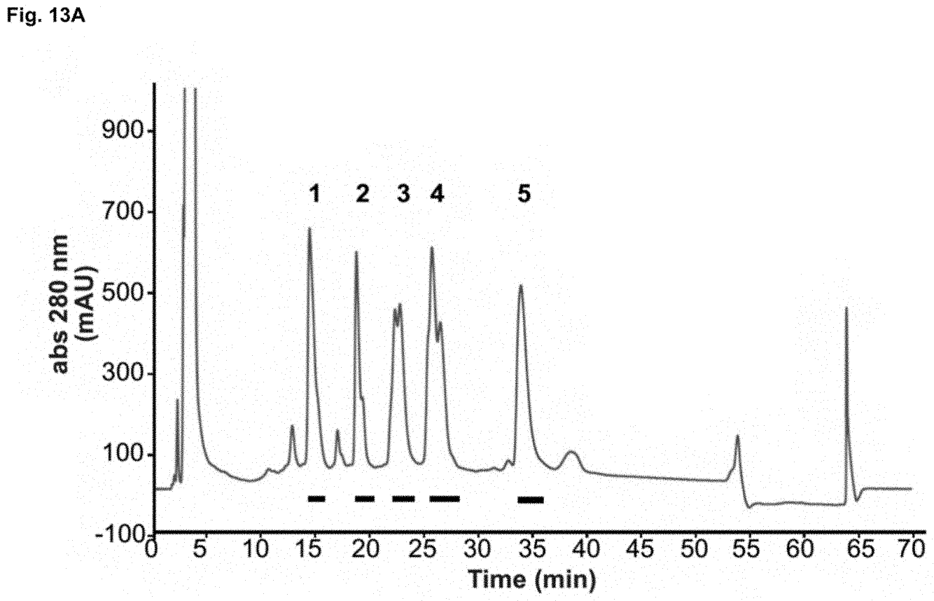

A frameshift mutation in the MTA1 gene was created by inserting a small non-coding DNA segment immediately downstream of the MTA1 start codon (FIGS. 3A and 12H). This non-coding DNA segment belongs to a class of genetic elements that are normally eliminated during the sexual cycle (Chen et al., 2014). When ssRNA homologous to such DNA segments is injected into Oxytricha cells undergoing sexual development, the DNA is erroneously retained (Khurana et al., 2018). This results in disruption of the MTA1 open reading frame. The ectopic DNA segment is propagated through subsequent cell divisions after completion of the sexual cycle. RNaseq analysis confirmed the presence of the ectopic insertion in mta1 mutant transcripts but not wild-type controls (FIG. 12H).

ssRNA was generated by in vitro transcription using a Hi-Scribe T7 High Yield RNA Synthesis Kit (New England Biolabs). The DNA template for in vitro transcription consists of the ectopic DNA segment flanked by 100-200 bp cognate MTA1 sequence. Following DNase treatment, ssRNA was acid-phenol:chloroform extracted and ethanol precipitated. After precipitation, ssRNA was resuspended in nuclease-free water (Ambion) to a final concentration of 1 to 3 mg/mL for injection.

ssRNA Microinjections

Oxytricha cells were mated by mixing 3 mL of each mating type, JRB310 and JRB510, along with 6 mL of fresh Pringsheim media. At 10 to 12 hr post mixing, pairs were isolated and placed in Volvic water with 0.2% bovine serum albumin (Jackson ImmunoResearch Laboratories) (Fang et al., 2012). ssRNA constructs were injected into the macronuclei of paired cells under a light microscope as previously described with DNA constructs (Nowacki et al., 2008). After injection, cells were pooled in Volvic water. At 60 to 72 hr post mixing, the pooled cells were singled out to grow clonal injected cell lines. As clonal population size grew, lines were transferred to 10 cm Petri dishes and grown in Pringsheim media. Only water from the “Volvic” brand has been empirically tested in our laboratory to support Oxytricha growth. Similar products from other vendors have not been tested.

### Survival Analysis of Oxytricha Mta1 Mutants

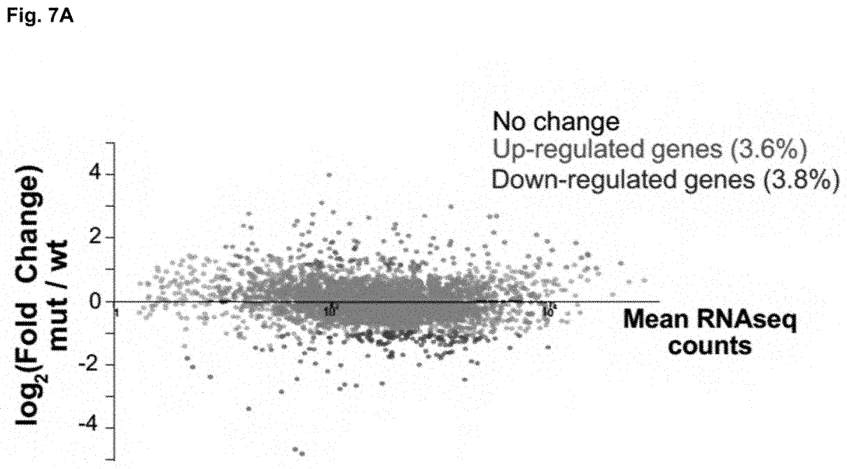

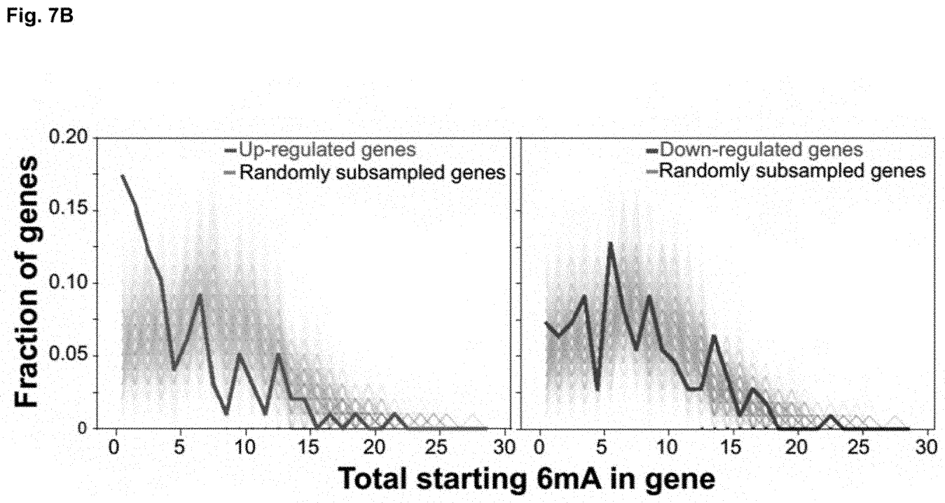

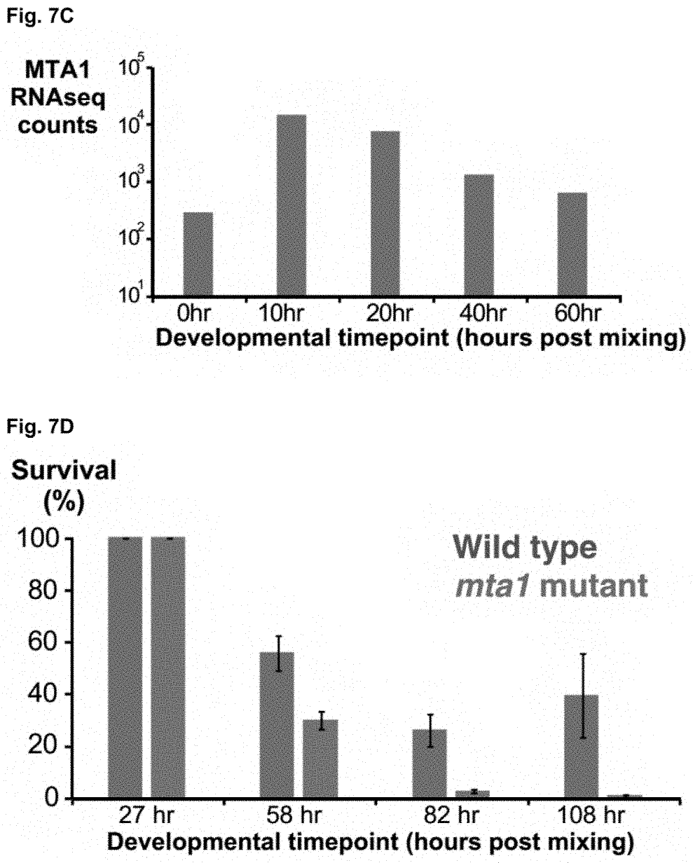

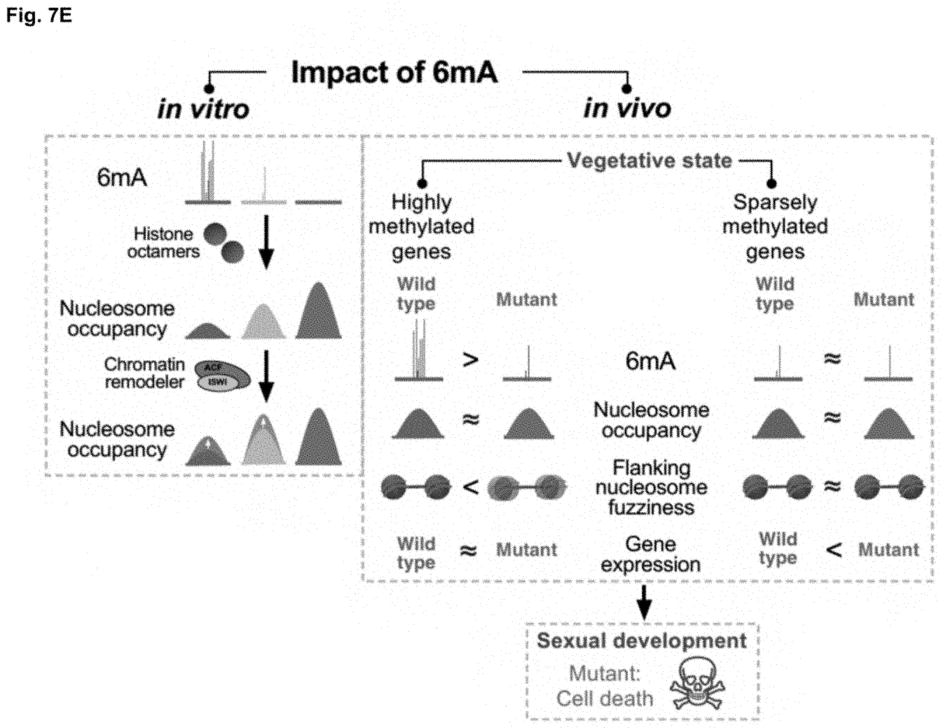

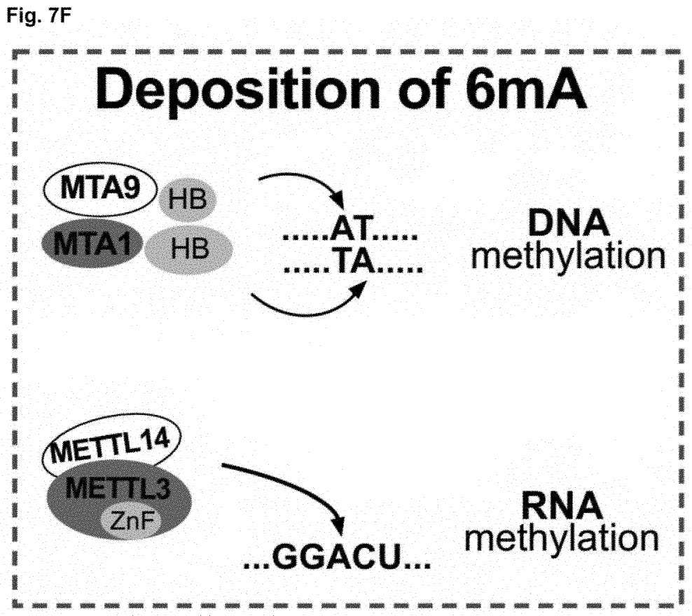

This experiment was performed in FIG. 7D. Wild-type or mutant Oxytricha cells were mixed at 0 hr to induce mating. Since not all cells enter the sexual cycle, mated cells are separated from unmated vegetative cells at 15 hr and transferred into a separate dish. The cells are allowed to rest for 12 hr to account for cell death during transfer. The number of surviving mated cells is counted from 27 hr onward. The total cell number at each time point is normalized to 27 hr data to obtain the percentage survival. An increase in survival at 108 hr is observed in wild-type samples because the cells have completed mating and reverted to the vegetative state, where they can proliferate and increase in number.

### Quantification and Statistical Analysis

All statistical tests were performed in Python (v2.7.10) or R (v3.2.5), and described in the respective Figure and Table legends.

### Data and Software Availability

Oxytricha SMRT-seq data are deposited in SRA under the accession numbers SRA: SRX2335608 and SRX2335607, and GEO: GSE94421. Tetrahymena SMRT-seq and all Oxytricha Illumina data are deposited in NCBI GEO under accession number GEO: GSE94421.

TABLE 1
Protein sequences for phylogenetic tree construction.

Protein sequences for phylogenetic analysis of MT-A70 proteins (including MTA1 and MTA9)
>NP_495127.1 DNA N6-methyl methyltransferase [Caenorhabditis elegans]
(SEQ ID No: 1)
MDTEFAILDEEKYYDSVFKELNLKTRSELYEISSKFMPDSQFEAIKRRGISNRKRKIKETSENSNRMEQMALKIKNVG
TELKIFKKKSILDNNLKSRKAAETALNVSIPSASASSEQIIEFQKSESLSNLMSNGMINNWVRCSGDKPGIIENSDGTK
FYIPPKSTFHVGDVKDIEQYSRAHDLLFDLIIADPPWFSKSVKRKRTYQMDEEVLDCLDIPVILTHDALIAFWITNRIGI
EEEMIERFDKWGMEVVATWKLLKITTQGDPVYDFDNQKHKVPFESLMLAKKKDSMRKFELPENFVFASVPMSVHS
HKPPLLDLLRHFGIEFTEPLELFARSLLPSTHSVGYEPFLLQSEHVFTRNISL
>NP_564080.1 Methyltransferase MT-A70 family protein [Arabidopsis thaliana]
(SEQ ID No: 2)
MAKTDKLAQFLDSGIYESDEFNWFFLDTVRITNRSYTRFKVSPSAYYSRFFNSKQLNQHSSESNPKKRKRKQKNSS
FHLPSVGEQASNLRHQEARLFLSKAHESFLKEIELLSLTKGLSDDNDDDDSSLLNKCCDDEVSFIELGGVWQAPFYE
ITLSFNLHCDNEGESCNEQRVFQVFNNLVVNEIGEEVEAEFSNRRYIMPRNSCFYMSDLHHIRNLVPAKSEEGYNLI
VIDPPWENASAHQKSKYPTLPNQYFLSLPIKQLAHAEGALVALWVTNREKLLSFVEKELFPAWGIKYVATMYWLKV
KPDGTLICDLDLVHHKPYEYLLLGYHFTELAGSEKRSDFKLLDKNQIIMSIPGDFSRKPPIGDILLKHTPGSQPARCLE
>ORY94237.1 MT-A70-domain-containing protein [Syncephalastrum racemosum]
(SEQ ID No: 3)
MIVASSDTCDIVDCEAAFGIDGTVRLRPGDFSLGTPYFTSRLGQKRPRPDDDTLDNTPSDTIHAIVQQLPVMAPDY
WHDRPMEAVVMNAHVHFPSLVSLAEASLRFDPDNDEDEDNRQILRPDMALESLQVFYRHFEHPKDSPILIRVQDAY
YWIPPRTAFMMGSLENIHLPTLGKFDCIVMDPPWPNKSVRRSAHYETQEDIYDLFAIPLPQLAQPNCLVAVWVTNK
PKFIRFVQKLFAAWDVEPLTTWYWLKVTTHGEPVCPIDSPHRKPYEHLILGRKRPVKININDPPALPRVLVSVPSKH
HSRKPPLNDILMRYLPSDARRLELFARCLTPGWTSWGNECLKFQHVDYFYDTNEAMEEGKQK
>ORX58127.1 MT-A70-domain-containing protein [Hesseltinella vesiculosa]
(SEQ ID No: 4)
MANAARRFAQQDELPLDVSQDLQDLPLLDLFNRKVINDSDQCSSLHVASFGQYLVPRHTKFVMSDLDNIDLLRSEN
DVFDLIVMDPPWPNKSVHRSTDYETQDIYDLFHLPIKSLIKNQGLVAVWVTNKPKYRRFILDKLFKAWQMTCVGEW
LWLKVTSSGEPVFPLDSPHRKPYEQLILGRYQPDDTSPTLPNPPQQHVLISVPSIRHSRKPPLGEVLADFLPKQPAC
LELFARCLTPGWTSWGNECLKFQHESYFISNDTPHSPSAS
>ORZ15132.1 MT-A70-like protein, partial [Absidia repens]
(SEQ ID No: 5)
YDLVVMDPPWPNKSVHRSSHYETQDIYDLYQIPLTSLVHKNSLVAVWITNKPKYRRFVMDKLFKSWHVDCVAEWT
WLKVTNDGEPVFPLNSTHRKPYEQLIIGRYNGGSGGGNDNNDSIQEESEVKPIPYQHSIVSVPSKRHSRKPPLQDL
LQPYLPAKPRCLELFARCLTPGWSSWGNECLKFQNEYYYTRIENPLHIDRSDV
>XP_021679935.1 MT-A70-domain-containing protein [Lobosporangium transversale]
(SEQ ID No: 6)
MLHESTVSVLDRLILISHISLQTYLLAKDREGFDIIVMDPPWQNASVDRMSHYRTMDLYELFKIPIPDLLKANGSNVG
GIVAVWITNKAKVKRVVVEKLFPAWGLDLVAHWFWLKVTTKGEPVLSLSNSHRRAYEGVLIGRQRQGSKLSNKTM
HETSASNPVNRLLVSIPAQHSRKPSLNALIEEEFFTSKLESRADRDRNAYVDSEALVKKPLYRLELFARNLEEGVLS
WGNEPLRYQYCGRGASNSQVVQDGYLIPCPIQSELVSQ
>XP_689178.3 methyltransferase-like protein 4 isoform X1 [Danio rerio]
(SEQ ID No: 7)
MSVVCCNSWGWLLDSSSHIDKDFQRCVCYNEANGLEENTHFTCCFKRQYFNILMPHMQQSTAMSGFPLDSGKH
DSAEHEKIELQTRKKRKRKHHDLNTGEIEANIYHDKVRSVVLEGSRALLEAGRQCGYFTEALTESQTISTPSESTSA
HECQLAAFCDLAKQLPLSEESPVHTLSRDGQNPALDLFSSITENPFDCACEITFMRERYLLPPRCRFLLSDVTRMDP
LVNSGDKFDLIVLDPPWENKSVKRSNRYSSLPSSQLKKLPVPALAAPGGLVVTWVTNRAKHRRFVREELYPHWAV
EVLAEWLWVKVTRSGEFVFPLDSQHKKPYEVLVLGRCRSTSDHTDRCSAVNELPDQRLLVSVPSTLHSHKPSLAA
VLKPYIRREPRCLELFARSLQSDWSCWGNEVLKFQHCSYFSRHTDQEPTSDTLQRTHSHLQSTGLLETPETAR
>NP_073751.3 methyltransferase-like protein 4 isoform 1 [Homo sapiens]
(SEQ ID No: 8)
MSVVHQLSAGWLLDHLSFINKINYQLHQHHEPCCRKKEFTTSVHFESLQMDSVSSSGVCAAFIASDSSTKPENDDG
GNYEMFTRKFVFRPELFDVTKPYITPAVHKECQQSNEKEDLMNGVKKEISISIIGKKRKRCVVFNQGELDAMEYHTKI
RELILDGSLQLIQEGLKSGFLYPLFEKQDKGSKPITLPLDACSLSELCEMAKHLPSLNEMEHQTLQLVEEDTSVTEQD
LFLRVVENNSSFTKVITLMGQKYLLPPKSSFLLSDISCMQPLLNYRKTFDVIVIDPPWQNKSVKRSNRYSYLSPLQIQ
QIPIPKLAAPNCLLVTWVTNRQKHLRFIKEELYPSWSVEVVAEWHWVKITNSGEFVFPLDSPHKKPYEGLILGRVQE
KTALPLRNADVNVLPIPDHKLIVSVPCTLHSHKPPLAEVLKDYIKPDGEYLELFARNLQPGWTSWGNEVLKFQHVDY
FIAVESGS
>XP_020951799.1 methyltransferase-like protein 4 isoform X1 [Sus scrofa]
(SEQ ID No: 9)
MSVVHQLSSGWLLDHLSFINKISYELHQHHEPCCSKNEPTSVHLDSLHKDSVFSFGASPAFIASSSKPENDDGGNR
EMSMQKYVFRSELFDVTKPYITSAIHKECQQSNEKEDLANDVKKEASISIKRKKRKRCVVFNQGELDAMEYHTKIRG
LILDGSSQLIQEGLKSGFLHPLSEKCDKCSKPVTLPLDTCSLSELCEMAKHVPSLNEMELQTLQLMEDDISVTEQDLF
SRIVENNSSFTKMITLMGQKYLLPPKSSFLLSDISCIYPLLNCRKTYDVIVIDPPWQNKSVKRSNRYSYLSPLQIKQIPI
PKLAAPNCLVVTWVTNRQKHLRFVKEELYPSWSVEIVAEWHWVKITNSGEFVFPIDSPHKKPYEVLVLGRVRERAA
LLLSRNAEVKELSIPDRKLIVSVPCILHSHKPPLAEVLKDYIKPEGEYLELFARNLQPGWTSWGNEVLKFQHMDYFVA
LESRS
>XP_011245012.1 PREDICTED: methyltransferase-like protein 4 isoform X2 [Mus musculus]
(SEQ ID No: 10)
MSVVHHLPPGWLLDHLSFINKVNYQLCQHQESFCSKNNPTSSVYMDSLQLDPGSPFGAPAMCFAPDFTTVSGND
DEGSCEVITEKYVFRSELFNVTKPYIVPAVHKERQQSNKNENLVTDYKQEVSVSVGKKRKRCIAFNQGELDAMEYH
TKIRELILDGSSKLIQEGLRSGFLYPLVEKQDGSSGCITLPLDACNLSELCEMAKHLPSLNEMELQTLQLMGDDVSVI
ELDLSSQIIENNSSFSKMITLMGQKYLLPPQSSFLLSDISCMQPLLNCGKTFDAIVIDPPWENKSVKRSNRYSSLSPQ
QIKRMPIPKLAAADCLIVTWVTNRQKHLCFVKEELYPSWSVEVVAEWYWVKITNSGEFVFPLDSPHKKPYECLVLG
RVKEKTPLALRNPDVRIPPVPDQKLIVSVPCVLHSHKPPLTGYLNSSFATLIPRVSNNMEYCRVVRTAFIA
>XP_018079135.1 PREDICTED: methyltransferase-like protein 4 [Xenopus laevis]
(SEQ ID No: 11)
MSVVCETSAGWLVDELSLLRKWYQHSTSCQDAAHKKQLYDIKEDLFLILRPHIPVQSTPAPLPILCPETNPGTINQR
KKRKRSCAFNQGELDAMEYHKKIIDFIMEGTQPLLQEGFKRLFLRPVLVNDDDHSQTEPRLCNNPCQLAELCNMAK
CMPLLNPGEHAVQVLERGIYLPQETNVLSCITENKSECPEVIQFMGEKYIIPPKSTFLMSDVSCMEPLLHYKRYNIIVM
DPPWENKSVKRSKRYSSLSPNEIQQLPVPVLAAPDCLVITWVTNKQKHLRFVKEDLYPHWSVKTLGEWHWVKITR
SGEFVFPLDSTHKKPYEVLIIGRFKGAGNSTARKSEICLPPIPERKLIVSVPCKLHSHKPPLSEILKEYVKPDLECLELF
ARNLQPGWTSWGNEVLKFQHIDYFTPVDVED
>NP_650573.1 uncharacterized protein Dmel_CG14906 [Drosophila melanogaster]
(SEQ ID No: 12)
MLKLQKKTEDSKFAVFLDHKTLINEAYDEFKLKSELFQFHAKKTDKGIEEDKTRKRKRKAGVEDASSLEDLHLVNEY
LELLSKPVEPEDSSPMKRHWEDGYNVPQLHGANESGRMQRFLRVDGSRGVYLIPNQSRFFNHNVDNLPALLHQLL
PAYDLIVLDPPWRNKYIRRLKRAKPELGYSMLSNEQLSHIPLSKLTHPRSLVAIWCTNSTLHQLALEQQLLPSWNLR
LLHKLRWYKLSTDHELIAPPQSDLTQKQPYEMLYVACRSDASENYGKDIQQTELIFSVPSIVHSHKPPLLSWLREHLL
LDKDQLEPNCLELFARYLHPHFTSIGLEVLKLMDERLYEVRKVEHCNQEEVN
>tr|A8J2E1|A8J2E1_CHLRE Predicted protein OS = Chlamydomonas reinhardtii OX = 3055
GN = CHLREDRAFT_174824 PE = 3 SV = 1
(SEQ ID No: 13)
MATLPGAAAAAPGANAEVGVPEPSLEPQDALQQRIALAEGLLALNEADAMQAWQQLPREALLEQVAKYRGAVRD
MASALRSSTLPGGVPPHCVPIHANVTTFDWPSLYSHAQFDVIMMDPPWQLATANPTRGVALGYSQLNDDHISRLP
VPQLQRQGGYLFVWVINAKYKWTLDLFDRWGYRLVDEVVWVKMTVNRRLAKSHGYYLQHAKEVCLVAKRGNPP
VPPGCEGGVGSDIIFSERRGQSQKPEEIYHLIEQLVPNGRYLEIFARKNNLRNYWVSIGNEVTGTGLPDEDMQALRD
LHHIPGAVYGKNAPHLVSKLFLYAPNSSREEG
>XP_021880122.1 MT-A70-domain-containing protein [Lobosporangium transversale]
(SEQ ID No: 14)
MLDQINIDIEQLEASLDIDEGKAHSNNASGTGCLIGTGTSSGNASNGAGVADEDLEEEVDDLEEFEAPEWCVPIKAN
VMTYDWDSLAAECQFDVILMDPPWQLATHAPTRGVAIAYQQLPDICIEELPVPKLSSNGFIFIWVINNKYAKAFDLM
RRWGYSYVDDITWVKQTVNRRMAKGHGYYLQHAKETCLVGKKGEDPPGCRHSIGSDVIFSERRGQSQKPEELYE
LIEELVPNGRYLEIFGRKNNLRDYWVTVGNEL
>ORX69627.1 MT-A70-domain-containing protein [Linderina pennispora]
(SEQ ID No: 15)
MDVDSSSPAVVLQALRQREQKIRSRILVLEQEISDLEKRCGVEGSGDAANKVTEADLEEFKAPEWSVPIRANVMNF
DWEKLAQACQFDVILMDPPWQLASQAPTRGVAIAYQQLPDVCIESLPIDLLQTSGFIFIWVINNKYTKAFQLMKQWG
YKYVDDIAWVKQTVNRRMAKGHGYYLQHAKETCLVGKKGPDPPNLRRSVASDVIFSERRGQSQKPEELYEIIEQLV
PGGRYLEIFGRKNNLRDYWVTVGNEL
>ORX98979.1 allantoinase [Basidiobolus meristosporus CBS 931.73]
(SEQ ID No: 16)
MSAIIFTGNRVLFDSTSKVEPATIHVDPWTGRIVKITNKRSTKADFPGIEDKDFVDAGDDLIMPGVIDAHVHLNEPGR
TDWEGFDTATRAAAAGGLTTVIDMPLNSIPPTTTLENLNTKKEAAKPQAWVDVGFYGGVIPGNADQLRPMIAAGVC
GFKCFLIESGVDEFPCVNEEEVRKAFAEFDGTDNVFMFHAEMECDDHSHETAAPQSTDPSAYQTFLQSRPHALEV
KAIEMIIRVCKDFPNVRAHIVHLSSAEALPMIRKAKAEGVKLTVETCYHYLTLNAEDIINGATHFKCCPPIREGSNRELL
WEALLDGTIDYVVSDHSPCTPELKRFDSGDFTAAWGGISSLQFGLSLLWTEAKRRGCTLQDLTRWLSQNTARHAG
ILNRKGRLQIGSDADIVIWSPEETFVVDKKMIHFKNKVTPYENMTLHGAVKKTFVRGRNVYDKSTAQLFSAKPLGNL
LARFQVYSNPITAMPSYAQPPSSDNGDFEEESEDYIESDEVDEDLRELLAKETSLRLRIDSLKEEILKLEREQRGETD
GSKNEGEGGEEEIDLEEFEAPEWCVPIKANVMTFEWKRLAEAAQFDVILMDPPWQLATHAPTRGVAIGYQQLPDV
CIEELPIPLLQKNGFIFIWVINNKYVKAFELMAKWGYRYVDDITWVKQTVNRRMAKGHGYYLQHAKETCLIGKKGED
PPNCRHSVCSDVIFSERRGQSQKPEELYEMIEQLVPNGKYLEIFGRKNNLRDYWVTIGNEL
>ORZ00623.1 MT-A70-domain-containing protein [Syncephalastrum racemosum]
(SEQ ID No: 17)
MSSREESPSSVSGFDLDTIDESTVTDTTLKNLLRREIELQLQIDALQTEILQIEESTAAGKNNKNDEELDPQDLEEFEA
PEWCVPIKANVMTFDWEALASEVQFDVIVADPPWQLATHAPTRGVAIGYQQLPDVCIEEIPIQKLQKNGFIFIWVINN
KYAKAFELMERWGYHYVDDITWVKQTVNRRMAKGHGYYLQHAKETCLVGKKGEDPPNCRHSVGSDVIFSERRG
QSQKPEELYELIEELVPNGKYLEIFGRKNNLRDYWVTVGNEL
>ORZ06213.1 MT-A70-domain-containing protein [Absidia repens]
(SEQ ID No: 18)
MTSDTSAMTADVLNRKRKRSPAMNGDDLSNNSDEADNNTTTGTTTSVDSNENDYQEQDREPILRLPRLNDAKLLE
EVVDDVDYEDQPERYDFDFKKLWLQERGLMERIDGLLKDIARLTDFKGHYRDMVIPSDDEDDLDDEDSKAQYDAP
EWCVPIKANVMTFDWESLGKEVQFDVIMADPPWQLATHAPTRGVAISYQQLPDVCIEDLPLEKLQTNGFLFIWVIN
NKYAKAFEMMEKWGYKYVDDITWVKQTVNRRMAKGHGYYLQHAKETCLVGVKGTLPPYCRRSVGSDVIYSERRG
QSQKPEQIYELIEEMMPGGKYLEIFGRKNNLRDYWITVGNEL
>ORX43344.1 MT-A70-domain-containing protein [Hesseltinella vesiculosa]
(SEQ ID No: 19)
MASESNISRESSPASISSTNSESGIENVQSLTDEDLKQLILKEMNLKEHIEQLQRKISKLTANDLSTNQDSSDADDDLL
NGDETMDDDSSSGSDSEVSGNEDIASVKSSPHAADKSESESESESDEGSSEDGNDEEDEFEAPKWCVPIKANVM
TFDWEKLASETQFDVIVADPPWQLATHAPTRGVAIAYQQLPDVCIEDLPIEKLQTNGFIFIWVINNKYAKAFELMEKM
GYTYVDDITWVKQTVNRRMAKGHGYYLQHAKETCLVGKKGVDPPSCRHSVGSDIIFSERRGQSQKPEELYELIEEL
VPNGKYLEIFGRKNNLRDYWVTVGNEL
>ORX52920.1 MT-A70 protein [Piromyces finnis]
(SEQ ID No: 20)
MMIVANEIDYEEFTAPEWCIPIKANVIDFEWDKLASECQFDAILMDPPWQLATHAPTRGVAIAYQQLPDQFIEELPIE
KLQKNGFIFIWVINNKYVKAFELMKKWGYTFVDDITWVKQTVNRRMAKGHGYYLQHAKETCLVGKKGEDPVGCKH
SISSDVIYSVRRGQSQKPEELYEMIEELIPNGKYLEIFGRKNNLRDYWVTIGNEL
>ORX86973.1 MT-A70-domain-containing protein [Anaeromyces robustus]
(SEQ ID No: 21)
MDEKEVENSVLDSSNIEKSNATTSNMDVDETSNNETSTAIIKSEDGANSYDDFLKLDFTPEEEKDEVLKKLIERETEL
KLKIEKEIEGIKNLELKGFSALTQKDEDVQDIDYEEFTAPEWCIPIKANVIDFEWDKLASECQFDAILMDPPWQLATHA
PTRGVAIAYQQLPDQFIEELPIEKLQKNGFIFIWVINNKYVKAFELMKKWGYTFVDDITWVKQTVNRRMAKGHGYYL
QHAKETCLVGKKGDDPVGCRHKISSDVIYSVRRGQSQKPEELYEMIEELIPNGKYLEIFGRKNNLRDYWVTIGNEL
>XP_001032074.3 MT-a70 family protein [Tetrahymena thermophila SB210]
(SEQ ID No: 22)
MKKEQQFLIFKKSLIIAQKRKEINIKQLKQQFKNFLFVQIFSIIKLKLQDIIIKFKMSKAVNKKGLRPRKSDSILDHIKNKLD
QEFLEDNENGEQSDEDYDQKSLNKAKKPYKKRQTQNGSELVISQQKTKAKASANNKKSAKNSQKLDEEEKIVEEE
DLSPQKNGAVSEDDQQQEASTQEDDYLDRLPKSKKGLQGLLQDIEKRILHYKQLFFKEQNEIANGKRSMVPDNSIPI
CSDVTKLNFQALIDAQMRHAGKMFDVIMMDPPWQLSSSQPSRGVAIAYDSLSDEKIQNMPIQSLQQDGFIFVWAIN
AKYRVTIKMIENWGYKLVDEITWVKKTVNGKIAKGHGFYLQHAKESCLIGVKGDVDNGRFKKNIASDVIFSERRGQS
QKPEEIYQYINQLCPNGNYLEIFARRNNLHDNWVSIGNEL
>EJY88228.1 MT-A70 family protein [Oxytricha trifallax]
(SEQ ID No: 23)
MNQSSQDITTQKSSNGFNPQTQPETLIQVIRKESTFIFKYRKNPYYVPPPISSQTSPNLEVETSNDLNQMSDYEGQI
PNNYEINRNSTQFTNNDDQSDNDFYDNNSITTMQIDTSTAKILNNGPLEYNPDLPNKEQKLKDSQVMQNQPPTATS
TNSQQRTLQELINIMPSIEDISQQCKQQQQLKIQAKANSTQSASTANAANGGKGRKRGRTVRFDQPLLGKVRQRN
GDASDDEEPDEIEMLIRRLHTDILNDARNDPVEQAKKIRQARESQSDQTNSTTQLSVYERMILGSASQQSTDHQPG
EFSNMFRTLEDEQIEINQNFLFDEYDSEDDSIADDKVEIASDDEQMLLQEHKKRGKKYLQDEIVKEEDFDEDDDSDE
DIHMDDLENESLSFDRNNRKSHKPVCKRTREENILDADLGDEKDDEDTIFIDNLPSDEFSIRRQLQDVKSYIKQFEML
FFEEEDSDKEEQLKQITNVQKHEEALQNFKDRSHLKNFWCIPLSSDVREIDWDVLIARQQEHTNGQLFDVITCDPP
WQLSSANPTRGVAIAYETLNDGEILKIPWGRLQKDGFLFIWVINAKYRFALDMMGAHGYRVVDEIQWVKQTCNGKI
AKGHGYYLQHAKEVCLVGCKGDPAILAKKCRSNIESDVIFSERRGQSQKPEEIYELVEALVPNGYYMEIFGRRNNLH
NGWVTVGNEL
>EJY79437.1 MT-A70 family protein [Oxytricha trifallax]
(SEQ ID No: 24)
MHLPMQIITQNMFRQGNQHSCLNRTEILRTPRLTRSTKTELQEQTHFSKLPRRNYLKLQIDMREIQSLVDKKVKESA
AAQQQLSQSGIEDSAIKRSLRPRKVENYKNMLEGDEITLKTIQDEQIEVKRKKREASSQNRLEDEDEDEDMLEVGQ
QIERASDDEDDDDFPISTRRSARKRTRRQDVDEDEEAIEVNQVESSDAEVEIPANDIDTESYTEGTNKRKQKLKAKK
QVLDKKKNKTEGDIDKEDAVEEEETVFIDNLPNDEFEIRRMLKEVKKHIKSLEKQFFEEEDSEKEEELKQINNNSKHE
EALQAFKETSHLKQFWCIPLSVNVTTLDFDLLAKSQMKQGGRLFDVITIDPPWQLSSANPTRGVAIAYDTLNDKEILN
MPFEKVQTDGFLFIWVINAKYRFALEMMEKFGYKLVDEIAWVKQTVNGKIAKGHGYYLQHAKETCLVGVKGNVKGK
ARYNIESDVIFSQRRGQSQKPEEIYEIAEALVPNGYYLEIFGRRNNLHNGWVTIGNEL
>NP_066012.1 N6-adenosine-methyltransferase non-catalytic subunit [Homo sapiens]
(SEQ ID No: 25)
MDSRLQEIRERQKLRRQLLAQQLGAESADSIGAVLNSKDEQREIAETRETCRASYDTSAPNAKRKYLDEGETDEDK
MEEYKDELEMQQDEENLPYEEEIYKDSSTFLKGTQSLNPHNDYCQHFVDTGHRPQNFIRDVGLADRFEEYPKLREL
IRLKDELIAKSNTPPMYLQADIEAFDIRELTPKFDVILLEPPLEEYYRETGITANEKCWTWDDIMKLEIDEIAAPRSFIFL
WCGSGEGLDLGRVCLRKWGYRRCEDICWIKTNKNNPGKTKTLDPKAVFQRTKEHCLMGIKGTVKRSTDGDFIHA
NVDIDLIITEEPEIGNIEKPVEIFHIIEHFCLGRRRLHLFGRDSTIRPGWLTVGPTLTNSNYNAETYASYFSAPNSYLTG
CTEEIERLRPKSPPPKSKSDRGGGAPRGGGRGGTSAGRGRERNRSNFRGERGGFRGGRGGAHRGGFPPR
>NP_964000.2 N6-adenosine-methyltransferase non-catalytic [Mus musculus]
(SEQ ID No: 26)
MDSRLQEIRERQKLRRQLLAQQLGAESADSIGAVLNSKDEQREIAETRETCRASYDTSAPNSKRKCLDEGETDEDK
VEEYKDELEMQQEEENLPYEEEIYKDSSTFLKGTQSLNPHNDYCQHFVDTGHRPQNFIRDVGLADRFEEYPKLRELI
RLKDELIAKSNTPPMYLQADIEAFDIRELTPKFDVILLEPPLEEYYRETGITANEKCWTWDDIMKLEIDEIAAPRSFIFL
WCGSGEGLDLGRVCLRKWGYRRCEDICWIKTNKNNPGKTKTLDPKAVFQRTKEHCLMGIKGTVKRSTDGDFIHA
NVDIDLIITEEPEIGNIEKPVEIFHIIEHFCLGRRRLHLFGRDSTIRPGWLTVGPTLTNSNYNAETYASYFSAPNSYLTG
CTEEIERLRPKSPPPKSKSDRGGGAPRGGGRGGTSAGRGRERNRSNFRGERGGFRGGRGGTHRGGFTPR
>XP_003129279.3 N6-adenosine-methyltransferase subunit METTL14 [Sus scrofa]
(SEQ ID No: 27)
MDSRLQEIRERQKLRRQLLAQQLGAESADSIGAVLNSKDEQREIAETRETCRASYDTSTPNAKRKYQDEGETDEDK
IEEYKDELEMQQEEENLPYEEEIYKDSSTFLKGTQSLNPHNDYCQHFVDTGHRPQNFIRDVGLADRFEEYPKLRELI
RLKDELIAKSNTPPMYLQADIEAFDIRELTPKFDVILLEPPLEEYYRETGITANEKCWTWDDIMKLEIDEIAAPRSFIFL
WCGSGEGLDLGRVCLRKWGYRRCEDICWIKTNKNNPGKTKTLDPKAVFQRTKEHCLMGIKGTVKRSTDGDFIHA
NVDIDLIITEEPEIGNIEKPVEIFHIIEHFCLGRRRLHLFGRDSTIRPGWLTVGPTLTNSNYNAETYASYFSAPNSYLTG
CTEEIERLRPKSPPPKSKSDRGGGAPRGGGRGGTSAGRGRERNRSNFRGERGGFRGGRGGAHRGGFPPR
>XP_018099063.1 PREDICTED: N6-adenosine-methyltransferase subunit METTL14
isoform X2 [Xenopus laevis]
(SEQ ID No: 28)
MNSRLQEIRARQTLRRKLLAQQLGAESADSIGAVLNSKDEQREIAETRETSRASYDTSAAVSKRKLPEEGKADEEV
VQECKDSVEPQKEEENLPYREEIYKDSSTFLKGTQSLNPHNDYCQHFVDTGHRPQNFIRDVGLADRFEEYPKLREL
IRLKDELIAKSNTPPMYLQADLENFDLRELKSEFDVILLEPPLEEYFRETGIAANEKWWTWEDIMKLDIEGIAGSRAFV
FLWCGSGEGLDFGRMCLRKWGFRRSEDICWIKTNKDNPGKTKTLDPKAIFQRTKEHCLMGIKGTVHRSTDGDFIH
ANVDIDLIITEEPEIGNIEKPVEIFHIIEHFCLGRRRLHLFGRDSTIRPDQSWEERLANSGGLREKEFLVGLLLGLLLPTA
TLIQRLMLLTLTLQIHLLLDAQRRSKDSVPKLHLLSQIVALGHREEEDEVEHLQVAERGAGKGTEAVLGETEGISEDV
EDHIGVSLLPVDFKCF
>NP_996954.1 N6-adenosine-methyltransferase non-catalytic subunit [Danio rerio]
(SEQ ID No: 29)
MNSRLQEIRERQKLRRQLLAQQLGAESPDSIGAVLNSKDEQKEIEETRETCRASFDISVPGAKRKCLNEGEDPEED
VEEQKEDVEPQHQEESGPYEEVYKDSSTFLKGTQSLNPHNDYCQHFVDTGHRPQNFIRDGGLADRFEEYPKQRE
LIRLKDELISATNTPPMYLQADPDTFDLRELKCKFDVILIEPPLEEYYRESGIIANERFWNWDDIMKLNIEEISSIRSFVF
LWCGSGEGLDLGRMCLRKWGFRRCEDICWIKTNKNNPGKTKTLDPKAVFQRTKEHCLMGIKGTVRRSTDGDFIH
ANVDIDLIITEEPEMGNIEKPVEIFHIIEHFCLGRRRLHLFGRDSTIRPGWLTVGPTLTNSNFNIEVYSTHFSEPNSYLS
GCTEEIERLRPKSPPPKSMAERGGGAPRGGRGGPAAGRGDRGRERNRPNFRGDRGGFRGRGGPHRGFPPR
>NP_609205.1 methyltransferase like 14 [Drosophila melanogaster]
(SEQ ID No: 30)
MSDVLKSSQERSRKRRLLLAQTLGLSSVDDLKKALGNAEDINSSRQLNSGGQREEEDGGASSSKKTPNEIIYRDSS
TFLKGTQSSNPHNDYCQHFVDTGQRPQNFIRDVGLADRFEEYPKLRELIKLKDKLIQDTASAPMYLKADLKSLDVKT
LGAKFDVILIEPPLEEYARAAPSVATVGGAPRVFWNWDDILNLDVGEIAAHRSFVFLWCGSSEGLDMGRNCLKKW
GFRRCEDICWIRTNINKPGHSKQLEPKAVFQRTKEHCLMGIKGTVRRSTDGDFIHANVDIDLIISEEEEFGSFEKPIEI
FHIIEHFCLGRRRLHLFGRDSSIRPGWLTVGPELTNSNFNSELYQTYFAEAPATGCTSRIELLRPKSPPPNSKVLRG
RGRGFPRGRGRPR
>NP_567348.2 Methyltransferase MT-A70 family protein [Arabidopsis thaliana]
(SEQ ID No: 31)
MKKKQEESSLEKLSTWYQDGEQDGGDRSEKRRMSLKASDFESSSRSGGSKSKEDNKSVVDVEHQDRDSKRERD
GRERTHGSSSDSSKRKRWDEAGGLVNDGDHKSSKLSDSRHDSGGERVSVSNEHGESRRDLKSDRSLKTSSRDE
KSKSRGVKDDDRGSPLKKTSGKDGSEVVREVGRSNRSKTPDADYEKEKYSRKDERSRGRDDGWSDRDRDQEGL
KDNWKRRHSSSGDKDQKDGDLLYDRGREREFPRQGRERSEGERSHGRLGGRKDGNRGEAVKALSSGGVSNEN
YDVIEIQTKPHDYVRGESGPNFARMTESGQQPPKKPSNNEEEWAHNQEGRQRSETFGFGSYGEDSRDEAGEASS
DYSGAKARNQRGSTPGRTNFVQTPNRGYQTPQGTRGNRPLRGGKGRPAGGRENQQGAIPMPIMGSPFANLGMP
PPSPIHSLTPGMSPIPGTSVTPVFMPPFAPTLIWPGARGVDGNMLPVPPVLSPLPPGPSGPRFPSIGTPPNPNMFFT
PPGSDRGGPPNFPGSNISGQMGRGMPSDKTSGGWVPPRGGGPPGKAPSRGEQNDYSQNFVDTGMRPQNFIRE
LELTNVEDYPKLRELIQKKDEIVSNSASAPMYLKGDLHEVELSPELFGTKFDVILVDPPWEEYVHRAPGVSDSMEYW
TFEDIINLKIEAIADTPSFLFLWVGDGVGLEQGRQCLKKWGFRRCEDICWVKTNKSNAAPTLRHDSRTVFQRSKEH
CLMGIKGTVRRSTDGHIIHANIDTDVIIAEEPPYGSTQKPEDMYRIIEHFALGRRRLELFGEDHNIRAGWLTVGKGLSS
SNFEPQAYVRNFADKEGKVWLGGGGRNPPPDAPHLVVTTPDIESLRPKSPMKNQQQQSYPSSLASANSSNRRTT
GNSPQANPNVVVLHQEASGSNFSVPTTPHWVPPTAPAAAGPPPMDSFRVPEGGNNTRPPDDKSFDMYGFN
>PNW88915.1 hypothetical protein CHLRE_01g050600v5 [Chlamydomonas reinhardtii]
(SEQ ID No: 32)
MQDGQGPPGDGRGRGRGRSRGGRIMFAREGGRGPRPMHSDMGPPPPPMGMFPHDPSAMMGGPMPGMPPM
DFTPEMLLTMMGAGLGGPMGLAGPMGMMMPDFGAAAAGAPGGMMVPPGAMMPPPPQPPSGGPGGMGGGGM
GGMGGMMGHQQGMGGAGGPMGLPGGGMGMGMGGGGGGGGGGGYGGRGGHGEAGGGGGGGGRAGGAG
GGGGAGGAAEHLSNDYSQNFVDTGLRPQNFLRDTHLTDRYEEYPKLKELIVRKDRQVSAHATPPLFLRTDLRSTRL
SPELFGTKFDVILVDPPWEEYVRRAPGMVADPEVWSWQDIQALDIEAVADNPCFLFLWCGAEEGLEAGRVCMQK
WGFRRVEDICWIKTNKEGGKGPGGGRRPYLTAANQHPESMLVHTKEHCLMGIKGSVRRATDGHIIHTNVDTDVIV
SEEPELGSTRKPEEMYHIIERFCNGRRRLELFGEDHNIRNGWVTVGRSLTSSNFSAKAYADHFRNRDGSVWVQNT
YGPKPPPGSVILVPTTDEIEDLRPKSPTGPHGGSSFHHSR
>XP_001022374.1 MT-a70 family protein [Tetrahymena thermophila SB210]
(SEQ ID No: 33)
MQPQQNQNQQQQQQQQSQQQQQQNQQLPQLQQSMSSQQQQNQQQEKQIIIKRGTTSKRNDYCQNFVNTHER
PQNFIMNIRPEERFIEYPKLQDLIKFKDDLIKKRNHPPVYLKADLKYYDLSKLGKFDVIMMDPPWKEYEERVQGLPIYS
QYPEKFNSWDLNEIAALPIDEISDKPSFLFLWVGSDHLDQGRELFRKWGYKRCEDIVWVKTNKDKTKEYIELPHSNL
LVRVKEHCLVGLRGDVKRASDSHFIHANIDTDVIVAEEPPLGSTQKPAEIYDIIERFCLGRKRLELFGEVHNVRQGWL
TIGKLLDESNFNQDEYNSWFDGDKTYPQIQTYRGGRYVGTTPDIEQLRPKSPTKNNQMNSNQNMSGSQVSEFDL
GIQQKQQKLNQQF
>NP_009876.1 Kar4p [Saccharomyces cerevisiae S288C]
(SEQ ID No: 34)
MAFQDPTYDQNKSRHINNSHLQGPNQETIEMKSKHVSFKPSRDFHTNDYSNNYIHGKSLPQQHVTNIENRVDGYP
KLQKLFQAKAKQINQFATTPFGCKIGIDSIVPTLNHWIQNENLTFDVVMIGCLTENQFIYPILTQLPLDRLISKPGFLFI
WANSQKINELTKLLNNEIWAKKFRRSEELVFVPIDKKSPFYPGLDQDDETLMEKMQWHCWMCITGTVRRSTDGHLI
HCNVDTDLSIETKDTTNGAVPSHLYRIAENFSTATRRLHIIPARTGYETPVKVRPGWVIVSPDVMLDNFSPKRYKEEI
ANLGSNIPLKNEIELLRPRSPVQKAQ
>XP_001691478.1 predicted protein [Chlamydomonas reinhardtii]
(SEQ ID No: 35)
MRLGGGPGGSELDDLLGKRSVKEKVKVEKGSELLDILSKPTARESARVEQFRTAGGSAIREHCPHLTKDECRRVN
GVPLACHRLHFLRVVQPHTDVALGNCSYLDTCRNMRTCKYVHYRPDPEPDVPGMGSEMARLRASVPKKPVGDG
QTSRGALDPQWINCDVRSFDMTVLGKFGVIMADPPWEIHQDLPYGTMKDDEMVNLNVGCLQDNGVLFLWVTGRA
MELARECMAKWGYKRVDELIWVKTNQLQRLIRTGRTGHWLNHSKEHCLVGIKGSPQLNRYVDTDVVVAEVRETS
RKPDEMYSLLERLSPGTRKLEIFARVHNCKPGWVGLGNQLKNVNLIEPEVRQRFAARYGFEPDASKDCFVN
>NP_192814.1 mRNAadenosine methylase [Arabidopsis thaliana]
(SEQ ID No: 36)
METESDDATITVVKDMRVRLENRIRTQHDAHLDLLSSLQSIVPDIVPSLDLSLKLISSFTNRPFVATPPLPEPKVEKKH
HPIVKLGTQLQQLHGHDSKSMLVDSNQRDAEADGSSGSPMALVRAMVAECLLQRVPFSPTDSSTVLRKLENDQNA
RPAEKAALRDLGGECGPILAVETALKSMAEENGSVELEEFEVSGKPRIMVLAIDRTRLLKELPESFQGNNESNRVVE
TPNSIENATVSGGGFGVSGSGNFPRPEMWGGDPNMGFRPMMNAPRGMQMMGMHHPMGIMGRPPPFPLPLPLP
VPSNQKLRSEEEDLKDVEALLSKKSFKEKQQSRTGEELLDLIHRPTAKEAATAAKFKSKGGSQVKYYCRYLTKEDC
RLQSGSHIACNKRHFRRLIASHTDVSLGDCSFLDTCRHMKTCKYVHYELDMADAMMAGPDKALKPLRADYCSEAE
LGEAQWINCDIRSFRMDILGTFGVVMADPPWDIHMELPYGTMADDEMRTLNVPSLQTDGLIFLWVTGRAMELGRE
CLELWGYKRVEEIIWVKTNQLQRIIRTGRTGHWLNHSKEHCLVGIKGNPEVNRNIDTDVIVAEVRETSRKPDEMYA
MLERIMPRARKLELFARMHNAHAGWLSLGNQLNGVRLINEGLRARFKASYPEIDVQPPSPPRASAMETDNEPMAID
SITA
>EAS00013.2 N6-adenosine-methyltransferase 70 kDa subunit [Tetrahymena thermophila SB210]
(SEQ ID No: 37)
MGSSVKDQEISNKKHKARNSSSGANNNSNSSNYQSSKRDIHQDRSYSKDDSQSRQYNSNNGGGGSSSKNSNRN
SSQQGYNQNSSSNQGQNSEYGGSGSGKNSQANSQRNSSQQGLQQLNQQQQSQQQQQQMLQNQMNSMGMM
NQFQNSFGLMGMQPSQPLQLLNPSMIIPSGKKQKYDFLEFPPSSQHEFRAILLDYFLSDLFDYPMHSAELFENFIEA
FSDIKDSSSFIKKLELIPLLQELNDKKAIKLETCAVGTKLFDFIVDINKDKIKQLSREFSKDRPKFMPILDKKPQPSSSKT
NSSSTTAPPKQAISKREIEDLLKKETGLQKEVITQSKEKSNLLNKISAAEESALAIFRKQGSRRIDYCDCGTRDKCIQIR
NSTVPCNKAHFRKIIRPHTDENLGNCSYLDTCRHMDYCKFVHYELDVDINNMNNDNLLLDGIEKKLNPQWINCDLR
QIDFNILGKFNCIMADPPWDIHMTLPYGTLKDREMKAMRVDLLQEEGVIFLWVTGRAMELGRECLTNWGYRRVEEI
IWVKTNQLQRIIRTGRTGHWLNHSKEHCLVGIKGNPKINRKIDCDVIVSEVRETSRKPDEIYNLIERMCPGGKKIELFG
RPHNTMPGWLTLGNQLPGIYLEDEEIIERYMDAYPDQDISRETMERNRIRMKNENDIDHIYNSHIQNIPPFKTKQLTK
DLQLQQQSSSMQTTQQQSSSQMMPQMQQQQSSQSINSNTDLQMHGNGLYEQE
>ORX92345.1 MT-A70-domain-containing protein [Basidiobolus meristosporus CBS 931.73]
(SEQ ID No: 38)
MKLERALFKMADMWGYNTIGIKREYDNDKSAISVIYFDPRNLRNVQHIEKTLEDICDVDSIDPDIFLDKTTSAQVPSTY
IPNEEARFSEDAEIEKLLSKPSFLEMEAFSSLIGVTELIERKTFREQEAEEMFKAQGNGGFREFCEYLIKEDCKKMNT
SGQPCAMTASILLTNMKLHFRRIMRPQTDLELGDCSYLNTCHRMDTCKYVHYELDDFEHPSSANITKTTIPTSLIFRP
PKKVLPAQWINCDVRKFDFSILGKFSVIMADPPWDIHMTLPYGTMTDDEMKAMAIHKLQDEGLIFLWVTARAMELG
RECLATWGYDRVDEVVWIKTNQLQRLIRTGRTGHWLNHSKEHCLVGIKGDPSRFNIGLACDVLVAEVRETSRKPD
QIYGMIDRLSPGTRKIEIFGRQHNTRPGWFTLGNQLKDVRIVEPEVLEAYNQRYPECPAQLSAIPES
>AJR96662.1 Ime4p [Saccharomyces cerevisiae YJM1248]
(SEQ ID No: 39)
MINDKLVHFLIQNYDDILRAPLSGQLKDVYSLYISGGYDDEMQKLRNDKDEVLQFEQFWNDLQDIIFATPQSIQFDQN
LLVADRPEKIVYLDVFSLKILYNKFHAFYYTLKSSSSSCEEKVSSLTTKPEADSEKDQLLGRLLGVLNWDVNVSNQGL
PREQLSNRLQNLLREKPSSFQLAKERAKYTTEVIEYIPICSDYSHASLLSTAVYIVNNKIVSLQWSKISACQENHPGLI
ECIQSKIHFIPNIKPQTDISLGDCSYLDTCHKLNMCRYIHYLQYIPSCLQERADRETAIENKRIRSNVSIPFYTLGNCSA
HCIKKALPAQWIRCDVRKFDFRVLGKFSVVIADPAWNIHMNLPYGTCNDIELLGLPLHELQDEGIIFLWVTGRAIELG
KESLNNWGYNVINEVSWIKTNQLGRTIVTGRTGHWLNHSKEHLLVGLKGNPKWINKHIDVDLIVSMTRETSRKPDE
LYGIAERLAGTHARKLEIFGRDHNTRPGWFTIGNQLTGNCIYEMDVERKYQEFMKSKTGTSHTGTKKIDKKQPSKL
QQQHQQQYWNNMDMGSGKYYAEAKQNPMNQKHTPFESKQQQKQQFQTLNNLYFAQ
>NP_651204.1 methyltransferase like 3 [Drosophila melanogaster]
(SEQ ID No: 40)
MADAWDIKSLKTKRNTLREKLEKRKKERIEILSDIQEDLTNPKKELVEADLEVQKEVLQALSSCSLALPIVSTQVVEKI
AGSSLEMVNFILGKLANQGAIVIRNVTIGTEAGCEIISVQPKELKEILEDTNDTCQQKEEEAKRKLEVDDVDQPQEKTI
KLESTVARKESTSLDAPDDIMMLLSMPSTREKQSKQVGEEILELLTKPTAKERSVAEKFKSHGGAQVMEFCSHGTK
VECLKAQQATAEMAAKKKQERRDEKELRPDVDAGENVTGKVPKTESAAEDGEIIAEVINNCEAESQESTDGSDTCS
SETTDKCTKLHFKKIIQAHTDESLGDCSFLNTCFHMATCKYVHYEVDTLPHINTNKPTDVKTKLSLKRSVDSSCTLYP
PQWIQCDLRFLDMTVLGKFAVVMADPPWDIHMELPYGTMSDDEMRALGVPALQDDGLIFLWVTGRAMELGRDCL
KLWGYERVDELIWVKTNQLQRIIRTGRTGHWLNHGKEHCLVGMKGNPTNLNRGLDCDVIVAEVRATSHKPDEIYGI
IERLSPGTRKIELFGRPHNIQPNWITLGNQLDGIRLVDPELITQFQKRYPDGNCMSPASANAASINGIQK
>NP_001084701.1 methyltransferase like 3 L homeolog [Xenopus laevis]
(SEQ ID No: 41)
MSDTWSSIQAHKKQLDNLRERLQRRRKDATSQLALDLQSSEGGIAPTFRSDSPVPSASSQPLKGPSGSAEVTPDP
ELEKKLLHHLSDLSLVLPADSVSIQLAITTPDFPVTRQGVESLLQKFAAQELIEVKGWGQEDDDRPTVVTFADYSKLS
AMMGAVAERKGTTIPTGAKKRRLQEADPSASSLSSSLSASASREKKTSEPQKKARKHASHLDLEIESLLSQQSTKE
QQSKKVSQEILELLSTSTAKEQSIVEKFRSRGRAQVQEFCDFGTKEECMKAAGADTPCRKLHFRRIINMHTDESLG
DCSFLNTCFHMDTCKYVHYEIDAWVEPGGTAMGTEAIASLDTPLAKAVGDSSVGRLFPAQWIRCDIRYLDVSILGKF
SVVMADPPWDIHMELPYGTLTDDEMRKLQIPVLQDDGFLFLWVTGRAMELGRECLKLWGYERVDEIIWVKTNQLQ
RIIRTGRTGHWLNHGKEHCLVGVKGSPQGFNRGLDCDVIVAEVRSTSHKPDEIYGMIERLSPGTRKIELFGRPHNIQ
PNWITLGNQLDGIHLLDPDVVAQFKQKYPDGVIGMPKNM
sp|F1R777.1|MTA70_DANRE RecName: Full = N6-adenosine-methyltransferase subunit METTL3:
AltName: Full = N6-adenosine-methyltransferase 70 kDa subunit; Short = MT-A70
(SEQ ID No: 416)
MSDTWSHIQAHKKQLDSLRERLQRRRKDPTQLGTEVGSVESGSARSDSPGPAIQSPPQVEVEHPPDPELEKRLLG
YLSELSLSLPTDSLTITNQLNTSESPVSHSCIQSLLLKFSAQELIEVRQPSITSSSSSTLVTSVDHTKLWAMIGSAGQS
QRTAVKRKADDITHQKRALGSSPSIQAPPSPPRKSSVSLATASISQLTASSGGGGGGADKKGRSNKVQASHLDMEI
ESLLSQQSTKEQQSKKVSQEILELLNTSSAKEQSIVEKFRSRGRAQVQEFCDYGTKEECVQSGDTPQPCTKLHFRR
IINKHTDESLGDCSFLNTCFHMDTCKYVHYEIDSPPEAEGDALGPQAGAAELGLHSTVGDSNVGKLFPSQWICCDIR
YLDVSILGKFAVVMADPPWDIHMELPYGTLTDDEMRKLNIPILQDDGFLFLWVTGRAMELGRECLSLWGYDRVDEII
WVKTNQLQRIIRTGRTGHWLNHGKEHCLVGVKGNPQGFNRGLDCDVIVAEVRSTSHKPDEIYGMIERLSPGTRKIE
LFGRPHNVQPNWITLGNQLDGIHLLDPEVVARFKKRYPDGVISKPKNM
>NP_062826.2 N6-adenosine-methyltransferase catalytic subunit [Homo sapiens]
(SEQ ID No: 42)
MSDTWSSIQAHKKQLDSLRERLQRRRKQDSGHLDLRNPEAALSPTFRSDSPVPTAPTSGGPKPSTASAVPELATD
PELEKKLLHHLSDLALTLPTDAVSICLAISTPDAPATQDGVESLLQKFAAQELIEVKRGLLQDDAHPTLVTYADHSKLS
AMMGAVAEKKGPGEVAGTVTGQKRRAEQDSTTVAAFASSLVSGLNSSASEPAKEPAKKSRKHAASDVDLEIESLL
NQQSTKEQQSKKVSQEILELLNTTTAKEQSIVEKFRSRGRAQVQEFCDYGTKEECMKASDADRPCRKLHFRRIINK
HTDESLGDCSFLNTCFHMDTCKYVHYEIDACMDSEAPGSKDHTPSQELALTQSVGGDSSADRLFPPQWICCDRY
LDVSILGKFAVVMADPPWDIHMELPYGTLTDDEMRRLNIPVLQDDGFLFLWVTGRAMELGRECLNLWGYERVDEII
WVKTNQLQRIIRTGRTGHWLNHGKEHCLVGVKGNPQGFNQGLDCDVIVAEVRSTSHKPDEIYGMIERLSPGTRKIE
LFGRPHNVQPNWITLGNQLDGIHLLDPDVVARFKQRYPDGIISKPKNL
>sp|Q8C3P7.2|MTA70_MOUSE RecName: Full = N6-adenosine-methyltransferase subunit METTL3;
AltName: Full = Methyltransferase-like protein 3; AltName: Full = N6-adenosine-
methyltransferase 70 kDa subunit; Short = MT-A70
(SEQ ID No: 43)
MSDTWSSIQAHKKQLDSLRERLQRRRKQDSGHLDLRNPEAALSPTFRSDSPVPTAPTSSGPKPSTTSVAPELATD
PELEKKLLHHLSDLALTLPTDAVSIRLAISTPDAPATQDGVESLLQKFAAQELIEVKRGLLQDDAHPTLVTYADHSKLS
AMMGAVADKKGLGEVAGTIAGQKRRAEQDLTTVTTFASSLASGLASSASEPAKEPAKKSRKHAASDVDLEIESLLN
QQSTKEQQSKKVSQEILELLNTTTAKEQSIVEKFRSRGRAQVQEFCDYGTKEECMKASDADRPCRKLHFRRIINKH
TDESLGDCSFLNTCFHMDTCKYVHYEIDACVDSESPGSKEHMPSQELALTQSVGGDSSADRLFPPQWICCDIRYL
DVSILGKFAVVMADPPWDIHMELPYGTLTDDEMRRLNIPVLQDDGFLFLWVTGRAMELGRECLNLWGYERVDEIIW
VKTNQLQRIIRTGRTGHWLNHGKEHCLVGVKGNPQGFNQGLDCDVIVAEVRSTSHKPDEIYGMIERLSPGTRKIEL
FGRPHNVQPNWITLGNQLDGIHLLDPDVVARFKQRYPDGIISKPKNL
>XP_003128628.1 N6-adenosine-methyltransferase 70 kDa subunit [Sus scrofa]
(SEQ ID No: 44)
MSDTTWSSIQAHKKQLDSLRERLRRRRKQDSGHLDLRNPEAALSPTFRSDSPVPTVPTSGGPKPSTASAVPELATD
PELEKKLLHHLSDLALTLPTDAVSIRLAISTPDAPATQDGVESLLQKFAAQELIEVKRSLLQDDAHPTLVTYADHSKLS
AMMGAVAEKKGPGEVAGTITGQKRRAEQDSTTVAAFASSLTSSLASSASEVAKEPTKKSRKHAASDVDLEIESLLN
QQSTKEQQSKKVSQEILELLNTTTAKEQSIVEKFRSRGRAQVQEFCDYGTKEECMKASDADRPCRKLHFRRIINKH
TDESLGDCSFLNTCFHMDTCKYVHYEIDACMDSEAPGSKDHTPSQELALTQSVGGDSNADRLFPPQWICCDIRYL
DVSILGKFAVVMADPPWDIHMELPYGTLTDDEMRRLNIPVLQDDGFLFLWVTGRAMELGRECLNLWGYERVDEIIW
VKTNQLQRIIRTGRTGHWLNHGKEHCLVGVKGNPQGNQGLDCDVIVAEVRSTSHKPDEIYGMIERLSPGTRKIEL
FGRPHNVQPNWITLGNQLDGIHLLDPDVVARFKQRYPDGIISKPKNL
>WP_009339935.1 MULTISPECIES: S-adenosylmethionine-binding protein [Afipia]
(SEQ ID No: 45)
MTLPAKDLLSFAGQRRFSTILADPPWQFTNKTGKVAPEHKRLSRYGTMKLDEIMMLPVADIAAPTSHLYLWCPNAL
LPEGLAVMKAWGFNYKSNIVWHKVRKDGGSDGRGVGFYFRNVTEVILFGVRGKNARTLAPGRRQVNLLATRKRE
HSRKPDEQYEIIESCSPGPFLELFARGTRKNWATWGNQADDDYKPTWKTYAHHSRAGLVAAE
>WP_013485562.1 S-adenosylmethionine-binding protein [Ethanoligenens harbinense]
(SEQ ID No: 46)
MSTAKETANNLLQFCGEKKYATVYADPPWRFQNRTGKVAPENKKLNRYPTMDLEDIKALPVGKIAAEKSHLYLWVP
NALLPDGLEVMKAWGFEYKGNIIWEKVRKDGEPDGRGVGFYFRNVTEILLFGIRGGNNRTLAPARSQVNLIRTQKR
EHSRKPDEIITIIESCSPGPYLELFARGDRENWDMWGNQATAEYEPTWNTYKNHTTKETTSGVSGSQSET
>WP_016343787.1 adenine-specific DNA methyltransferase [Mycobacteroides abscessus]
(SEQ ID No: 47)
MAAPLREVNEPPPLPVTDGGFSTILADPPWRFTNRTGKVAPEHRRLDRYSTLSLDEICALGVSDVTADNAHLYLWV
PNALLPDGLRVMEEWGFRYVSNIVWSKVRRDGLPDGRGVGFYFRNTTELLLFGVRGSMRTLQPARSQVNQIVTR
KREHSRKPDEQYELIEACSPGPYLEMFGRYRRPNWAVWGDEANEDVEPRGQTHKGYGGGEITRLPALEPHSRIP
QWLAKPIAAAIKSAYDDGMSIDAIAAETGYSISRVRHLLDQAGAKKRGRGRPAKA
>WP_023133224.1 MULTISPECIES: MT-A70 protein [Rothia]
(SEQ ID No: 48)
MLDPMNTNEEFAPLPTVEGGFQTVLADPPWRFTNRTGKVAPEHHRLGRYGTMSLDEIKALRVGDVTADNAHLYL
WVPNALLPEGLEVMQAWGFRYVSNIIWAKRRKDGGPDGRGVGFYFRNVTEPILFGVKGSMRTLAPGRSTVNMIET
RKREHSRKPDEQYDLIEACSPGPYLELFARYARPGWSVWGNEASNEIEPRGKAQKGYGGGEIDRLPILEPNERMS
EWLSGRVGELLAEEYTKGASVQELANQSGYSIARVRTLLTHSGVPLRGRGRPKKGQVAS
>ETW92643.1 S-adenosylmethionine-binding protein [Candidatus Entotheonella factor]
(SEQ ID No: 49)
MSNSPHSAADDLLACGFPPHSFSTVLADPPWRFTNRTGKMAPEHRRLSRYPTLTLEEIADLPLAQLVQPDSHLYLW
VPNALLAEGLDVMRRWGFTYKTNLVWYKIRRDGGPDRRGVGFYFRNVTELVLFGVRGRMRTLAPGRRQENLLAS
QKQEHSRKPDTFYDLIERCSPGPYLELFARHPRPGWHQFGNEPLVSSS
>AHJ63281.1 Adenine-specific methyltransferase [Granulibacter bethesdensis]
(SEQ ID No: 50)
MTKQPDPIAEFRNQLNGGNFATVLADPPWRFQNRTGKMAPEHRRLSRYGTMELPEIMALPVSEVTAKTAHLYLWV
PNALLPEGLAVMQAWGFNYKSNLVWHKIRKDGGSDGRGVGFYFRNVTELVLFGVKGKNARTEAPGRRQVNLLAT
QKREHSRKPDEFYDIVEACSPGPYLELFARGTRPGWCAWGNQAEEYDITWDTYSHHSQRQSLWVAE
>WP_017364718.1 S-adenosylmethionine-binding protein [Methylococcus capsulatus]
(SEQ ID No: 51)
MTENTLDPAADLLERLGDKRFRTILADPPWQFQNRTGKMAPEHKRLNRYGTMSLEAIAGLPVERLTADTAHLYLWV
PNALLLEGLKVMEAWGFTYKTNLVWHKIRKDGGPDGRGVGFYFRNVTELVLFGVRGKNARTLAAGRRQVNFLAT
RKREHSRKPDEMYGIIEACSPGPYLELFARGARDRWSVWGNEADENYYPRWNTYANHSQAEICPFE
>WP_027700599.1 S-adenosylmethionine-binding protein [Xylella fastidiosa]
(SEQ ID No: 52)
MTKHKANTASDVGRDLLARHGGQRFHTILADPPWQFQNRTGKMAPEHKRLSRYGTMTLDDIMMLPVEQLVTDTA
HLYLWVPNALLPEGIKVLEAWGFSYKSNIVWHKVRKDGGPDGRGVGFYFRNVTELVLFGVRGKNARTLAPGRRQ
VNFLATQKREHSRKPDEFYDIVESCSPGPFLELFARGPRDGWKVWGNQADKYYPTWPTYSNHSQAECELGRVE
MIAQRLLSV
>WP_027488351.1 S-adenosylmethionine-binding protein [Rhizobium undicola]
(SEQ ID No: 53)
MLNRNTDAPSPSDDFTNFISGRKFATIMADPPWQFMNRTGKVAPEHKRLNRYGTMELDAIKALPVATACAPTAHLY
LWVPNALLPEGLEVMKAWGFNYKANIVWHKLRKDGGSDGRGVGFYFRNVTELILFGTRGKNARTLPPGRSQVNYI
GTRKREHSRKPDEQYPLIESCSPGPYLEMFGRGLRKGWTTWGNQADETYEPTWKTYGHNSSTDRLEAAE
>ESK34829.1 hypothetical protein G966_02949 [Escherichia coli UMEA 3323-1]
(SEQ ID No: 54)
MGWFMTKKYTLIYADPPWVYRDKAADGNRGAGFKYPVMSVLDICRLPVWDLADENCLLAMWWVPTQPLEALKVV
EAWGFRLMTMKGFTWIKCGSRQPDKLVMGMGHMTRANSEDCLFAVKGKLPTRINAGIVQSFTAPRLEHSRKPDIV
REKLVQLLGDVSRIELFARQTSHGFDVWGNQCEDPAVQLHPGYALDIGGLTNAFSNAPLSPTDIQGRERAA
>AIF94871.1 Adenine DNA methyltransferase, phage-associated [Escherichia coli
O157:H7 str. SS17]
(SEQ ID No: 55)
MTKKYTLIYADPPWTFRDKATDGQRGASFKYPVMSLLDICRLPVWELAADNCLLAMWWVPTQPLEALKVVEAWG
FRLVTMKGLTWNKCGKRQTDKLVMGMGSTTRANSEDCLFAVKGNLPERINAGIIQSFTAPRLDHSRKPDMAREKL
VQLLGDVPRIELFARHTSHGFDVWGNQCGTPSIEMVPGIVKFLEKTNERKNDVDKGITS
>WP_032715146.1 adenine methylase [Klebsiella aerogenes]
(SEQ ID No: 56)
MTGKYTLIYADPPWSYRDKAADGDRGAGFKYPVMNVMDICRLPVWELSADDCLLAMWWVPTQPVEALKVVEAW
GFRLMTMKGFTWHKINKHKGNSAIGMGHMTRANSEDCLFAVRGKLPERMDASICQHVTAPRLENSRKPDVIREKL
VQLLGDVPRIELFARQSSHGFDVWGNQCIAPAVELLPGCAVPVVKTEAA
>AIA43360.1 DNA methyltransferase [Klebsiella pneumoniae subsp. pneumoniae KPNIH27]
(SEQ ID No: 57)
MNYDLIYCDPPWEYGNRISNGAACNHYSTMSIDDLKFLPVRKLAADNAVLAMWYTGTHNREAVELAESWGFRVRT
MKGFTWVKLNQNAADRFNKALSTGELVDFNDLLEMLDRETRMNGGNHTRSNTEDVLIATRGTGLPRASASVKQV
VHTCLGEHSAKPWEVRNRLEQLYGDVKRIELFAREEWKGWDRWGNQCNNSIEIITGLIKEVNHAA
>WP_009320301.1 DNA methyltransferase [Clostridioides difficile]
(SEQ ID No: 58)
MPAVLFLLELHRRRKGGYKIENNQKYNIIYADPPWRYQQKRLSGAAEHHYPTMSVKDICGLKVEEIAAKDCVLFLWA
TFPQLPEALRVIKAWGFQYKTVAFVWLKQNKSGKGWFFGLGFWTRGNAEICLLAIKGKPHRNSNRVHQFLISPIRG
HSQKPEEAREKIVELMGDLPRVELFAREKTEGWDAWGNEVESDIEISSDTEKEWR
>WP_012115592.1 MT-A70 family protein [Xanthobacter autotrophicus]
(SEQ ID No: 59)
MNGLWQFGDLKMFGYDLIVADPPWDFELYSEAGEGKSAKAHYGTMKLDEIAALRVGDLARGDCLLLLWCCEWMP
PAARQRVLDAWGFTYKTTIIWRKVTRAGKVRMGPGYRARTMHEPVIVATVGNPKHTPFSSVFDGVAREHSRKPEA
FYRMVEAAAPKAARADLFSRQRRDGWDAFGNEVEKFDQPPAEAAE
>KFL31466.1 DNA methyltransferase [Devosia riboflavina]
(SEQ ID No: 60)
MTAWPFGAMPMFSFDVVMADPPWSFDNWSEGGNAKNAKAQYDCMPTPDIKRLPVGHLAAGDCWLWLWATYP
MLPDAIEVMDAWGFRYVTAGPWVKRGTSGKLAMGTGYVLRSCSEIFLIGKNGEPKTHARDVRNVLEAPRREHSRK
PDEAYAMAEKLFGPGRRADLFSRETRPGWTSWGNESTKFDEVAA
>WP_016734162.1 DNA methyltransferase [Rhizobium phaseoli]
(SEQ ID No: 61)
MRLFPDLWPFGDLQPHSFDFIMADPPWKMQEWSDNGDKSKSTQSKYRLMPLDEIKAMPVLDLAAPNCLLWLWAT
NPMLPQALDVLHAWGFTFATAGSWMKTTRNGKQAFGTGYIFRTSNEPILIGKRGEPKTTRSVRSSFPGLAREHSR
KPEEGYREAERLMPRARRLELFSRTNRVGWTTWGDEVGKFGDVA
>KFB10357.1 Adenine-specific methyltransferase [Nitratireductor basaltis]
(SEQ ID No: 62)
MHLFDWPFGDLNPHSFDLIMADPPWAFELRSDKGEGKSAQSHYKCQTLDEIKALPVLDLAAPDCLLWLWATNPML
PQAFEVMAAWGFTFKTAGAWGKTTVNGKLAFGTGYIFRSAHEPILIGTRGEPRTTKSVRSLIMGQVREHSRKPEEA
YAAAEKLIPNARRLELFSRTDRAGWEVWGDEAGKFGEAA
Protein sequences for phylogenetic analysis of p1 proteins
>XP_001009903.1 [Tetrahymena thermophila SB210]
(SEQ ID No: 63)
MSLKKGKFQHNQSKSLWNYTLSPGWREEEVKILKSALQLFGIGKWKKIMESGCLPGKSIGQIY
MQTQRLLGQQSLGDFMGLQIDLEAVFNQNMKKQDVLRKNNCIINTGDNPTKEERKRRIEQNR
KIYGLSAKQIAEIKLPKVKKHAPQYMTLEDIENEKFTNLEILTHLYNLKAEIVRRLAEQGETIAQPS
IIKSLNNLNHNLEQNQNSNSSTETKVTLEQSGKKKYKVLAIEETELQNGPIATNSQKKSINGKRK
NNRKINSDSEGNEEDISLEDIDSQESEINSEEIVEDDEEDEQIEEPSKIKKRKKNPEQESEEDDI
EEDQEEDELVVNEEEIFEDDDDDEDNQDSSEDDDDDED
>EJY79729.1 [Oxytricha trifallax]
(SEQ ID No: 64)
MSSSISAAIIAGNQNKKIAESKSLWNYALSPGWTQQEVEILKIALMKFGVGRWKTIEQSQCLPT
KTMSQMYLQTQRLVGQQSLAEFMGLHLDLEQIFIKNAERQGAGVFRKNGCIINTGDNMTKVQI
AKLRKKNSKIFGLTQPFVQSLHLPKAKVKEWLKVLTLDQILSAKSNFSTAEKIHYLKILENALER
KLKKILRLQELVSIYRPCNIGIVVQKRLGSSIGDEYFEYVDCVKIEEKSVGNLDFALPNRNTDSTS
LNEDFSFLDSTQKPQKLKAGSGRENKRKKMRDGLKDERAQRQSLMEALDEQEFDETKFQDS
>EJY78001.1 [Oxytricha trifallax]
(SEQ ID No: 65)
MSVHHKMADSKSLHNYTLSPGWTREEVDILKIALMKFGIGKWKKIQKSGCLPSKTISQMNLQT
QRLLGQQSLAEFMGLHVYLDRVFRDNSLKTGPEIQRKNNFIINTGNNLTQPEKEKRLRLNKQK
YGLDLAFIKTLRLPKPESATGGKREAILSMDQIFAQKSHFTVVEKLKHLEALKNALCSKLGKIER
RRRNKELSKIYRPLGQLIVVQKNADDQYEFVDIIDENE
>ORX69504.1 [Linderina pennispora]
(SEQ ID No: 66)
MSSATPYAPRSMPTGQRNVVRSNDSASLWNCTLSPGWTQEEVQVLRKALMKFGVGNWMKII
ESECLPGKTIAQMNLQTQRMLGQQSTAEFNGLHLDAFVIGELNSKKQGPGIKRKNNCIVNTGG
KLTRDEVVKRQQKHREQYEVKAEVWRAIVLPKPDNPLILLEKKREELKKVRLELEEIMKQIEET
>ORX78557.1 [Basidiobolus meristosporus CBS 931.73]
(SEQ ID No: 67)
MTDVYKPRSMPVGARNVLRSNDSASLWNCTLSPGWTEPEVHILRKAVMKFGIGNWAKIIESQ
CLFGKTIAQMNLQLQRMLGQQSTAEFAGLHLDPFVIGEINSKKQGPGIKRKNNCIVNTGGKLTR
EEIKRRLLEHKRTYEISEEEWRSIELPKPEDPGAVLIAKKDELKMLEDELLRVVQKIQKAREERR
SKSVDSSSVDGSVDDEARETKRRRK
>EJY73777.1 [Oxytricha trifallax]
(SEQ ID No: 68)
MSHATSHGNSTEKDKKNSGNMVAESKSLWNYALSPQWTPQEVDVLKIALMKFGIGKWTIIDK
SGILPTKTIQQCYLQTQRILGQQSLAEFMGLHVDIDKIALDNRRKNGIRKMGFLVNQGGKLTPE
EKAHYQEINRQKYGLSPEEVETIKLPPPCSVEIYDINKIINPKSKLTTIEKINHCIKLQDALLEKLEN
IKNKKIPTGAGFSSSRVYENMRGYDPQLLLNSHVTGQLDHSMQDLTIDERYSDLDEEEDPLAM
ASIIDSQATPQPQKIKSSVPNKASTTPSAKEMNQIKDIIDSVIAENSAQQSKNLAQEKPKLKFSLV
KATESNLLQSAAQNSDDVVMEEDSKLQHIETFSTVTQTATDQSNSQSKSQNNIASDSLKDSLE
QNDLSKSLTDSLEMQQYSAEKKLNQAPMSKNSDKPKKKRLNKRKLPSDDEFETL
>XP_021883515.1 [Lobosporangium transversale]
(SEQ ID No: 69)
MSSGSTPRSMTAGARNILRSNDSASLWNYTVAPGWSMKEAEILRKALMKFGIGNWSKIIESN
CLVGKTNAQMNLQTQRMLGQQSTAEFAGLHIDPRVIGQKNSLIQGDHIRRKNGCIVNTGAKLS
REEIRRRVAENKEQYELPEEEWSSIELPLPDDPHLLLEAKKSEKVRLELELKNVQRQIAMLRKV
GRKFETGSESPKTELDDDERDEFIEDQPLGKRARIEA
>EJY81929.1 [Oxytricha trifallax]
(SEQ ID No: 70)
MSSSISAAIMAGNQNKKIAESKSLWNYALSPGWTQQEVEILKIALMKFGVGRWSAINKSGVLP
TKQIQQCYLQTQRLIGQQSLAEFMGLHLDIDRIAADNKQKRGIRKQGFLVNQGCKLTPEEKDEL
RKINQEKYGLSAEHVEAIKLPAPCHLVEIFQIDKIMHPRSTLSTMDKIKHLIKLEDALKSKLEMIRE
GKRQQKFEQLQQKLKTTEASGRGSVTRVQRQMSDLHLGSSHQNRNSDLDEENDESVMIIDE
SQQENLTPKGKAQAMLTHQKYNEVTQTMIKQGDDSRQQQHLPLDSTSASVSNPSSTSKSST
MKSNSMKQSETAIASMKPSSIGKKTKVDSSFVTKQSNQQSTAPIQKQAHQQNLDRNRSELGS
TFAQQASVDTQNSNNQGTSTASGNFISQSDDEEALMPKLKRRRVEDSE
>EJY76686.1 [Oxytricha trifallax]
(SEQ ID No: 71)
MRVYLKFCNRKQIHYTHTMSSSISAAIMAGNQNKKIAESKSLWNYALSPGWTQQEVEILKIALM
KFGVGRWSAINKSGVLPTKQIQQCYLQTQRLIGQQSLAEFMGLHLDIDRIAADNKQKRGIRKQ
GFLVNQGCKLTPEEKDELRKINQEKYGLTAEHVEAIKLPAPCHLVEIFQIDKIMHPRSTLSTMDK
IKHLIKLEDALKSKLEMIREGKRQQKFEQLQQKLKTTEASGRGSVTRVQRQMSDLHLGSAHQN
RNSDLDEENDQSVMIIDESQQQNLTPKGKAQTMLTNQTQTMKKQADDSRDEQHLPLISTSAS
VSNPSSTSKSSALKLNSMKQSDTAIASMKPSSSGKKTKVDSSFVSKQSNQQSTSYSETNVDT
QNSNNQGTSTASGNFISQSDDEEALMPKLKRRRVEDSE
>EJY80746.1 [Oxytricha trifallax]
(SEQ ID No: 72)
MRVYLKFCNRKQIHYTHTMSSSISAAIMAGNQNKKIAESKSLWNYALSPGWTQQEVEILKIALM
KFGVGRWSAINKSGVLPTKQIQQCYLQTQRLIGQQSLAEFMGLHLDIDRIAADNKQKRGIRKQ
GFLVNQGCKLTPEEKDELRKINQEKYGLTAEHVEAIKLPAPCHLVEIFQIDKIMHPRSTLSTMDK
IKHLIKLEDALKSKLEMIREGKRQQKFEQLQQKLKTTEASGRGSVTRVQRQMSDLHLGSAHQN
RNSDLDEENDQSVMIIDESQQQNLTPKGKAQTMLTNQTQTMKKQADDSREEQHLPLNSTSAS
VSNPSSTSKSSALKLNSMKQSDTAIASMKPSSSGKKTKVDSSFVSKQSNQQSTGPIQKQAHQ
QNLDRNRSELGSTFAQQTNVDTQNSNNQGTSTASGNFISQSDDEEALMPKLKRRRVKDSE
>ORX56566.1 [Piromyces finnis]
(SEQ ID No: 73)
MSIPKPRSMPVGFRNILRPNDSTSLWNCTLSPGWTQEESDILRDALIFYGIGNWKDIIEHGCLP
DKTNAQMNLQLQRMLGQQSTAEFQNLHIDPYEIGKINSQKQGPNIRRKNGFIINTGGKLSREDI
KRKIQENKENYELPEEVWSKIVLPNREVVTINEKRQKLNKLEEELDSVLKQIVNRRRELRGMTP
LKETEMKSIVNRSNQNDTKTEEKEIKEEESTTVNEEKIENTETSSISIISTNENEQSENISSSSPIV
KSEQKKKRVVSRRKNKRRVNSDDEDFLPPGKSRSKRTRRTPKKSSN
>ORX79686.1 [Anaeromyces robustus]
(SEQ ID No: 74)
MSIPKPRSMPTGFRNILRPNDSTSLWNCTLSPGWTQEESDILRDALIYYGIGNWKDIIEHGCLP
DKTNAQMNLQLQRMLGQQSTAEFQNLHIDPYVIGKINSQKQGPNIRRKNGFIINTGGKLSREDI
RRKIQENKENYELPKEEWSKIVLPNREVVIKNKVQEAINEKREKLNKLEDELDSVLKAIVNRRR
ELRGMIPLKDSEMKSLVNRSAKNEGENKTETTNNEESNNTNNSDDIKDENNETSTSSHIFTNN
DNELSENNSSSSSSNSISNKKKRFLRREVRRGKRRYNYDDDDFMPSGNRSRKSRKI
>ORZ01404.1 [Syncephalastrum racemosum]
(SEQ ID No: 75)
MSNNKENNVNKPRSMTAGARNVLRSNDSTSLWNCTLSPGWTQDESEVLRKALMKFGVGNW
AKIIESGCLPGKTNAQMNLQLQRLLGQQSTAEFAGLHIDPKVIGEKNSKIQGPHIKRKNNCIVNT
GDKLSRDKLRARVMSNKEEYELPEEVWKNIELPKVKDPLMLLEGKKEEMRKLKTELEKVQAKI
QQLRQAQPARVQELQSQIEVARSPSPSAPDSPALSV
>XP_001698763.1 [Chlamydomonas reinhardtii]
(SEQ ID No: 76)
MAFAAALAEKRGPRVGDAASLWNFTPAPGWSREEVQILRLCLMKHGVGQWMQILSTGLLPG
KLIQQLNGQTQRLLGQQSLAAYTGLKVDVDRIRVDNETRTDATRKAGLIINDGPNLTKEMKEK
MRQDAVAKYGLTPEQVAEVDEQLAEIAAAFNPASTSAAAGAGSGAAAAGQAAAAGSGAGGS
GNLMAQPTEQLSAEQLGQLLLRLRNRLACLVDRARGRAGLPPRTAPRWATEAAAAACLAAM
AAAEASAPQAPAAAAGGQEGAAGPVMVSVPFSREVLAEATACRVRSGTAAGARGNAPGAQ
GGVRKRTSKGGKAKGGDREWSPEGEENTAPQPRGGGKRKSGAVAGGEEADGVASGRAKR
ASRPKRGSSKHDPYVDDNDYGDEGIDPFDVGDDLDDMNPHGRYGNGGGRRADPSEAISALT
AMGFTQSKARGALRECNFNVELAVEWLFANCL
>PNW76495.1 [Chlamydomonas reinhardtii]
(SEQ ID No: 77)
MAFAAALAEKRGPRVGDAASLWNFTPAPGWSREEVQILRLCLMKHGVGQWMQILSTGLLPG
KLIQQLNGQTQRLLGQQSLAAYTGLKVDVDRIRVDNETRTDATRKAGLIINDGPNLTKEMKEK
MRQDAVAKYGLTPEQVAEVDEQLAEIAAAFNPASTSAAAGAGSGAAAAGQAAAAGSGAGGS
GQAATAADAGGAAGRGTGSAGGAAAAAPPRNALAISTGVLAATLLDASLGNLMAQPTEQLSA
EQLGQLLLRLRNRLACLVDRARGRAGLPPRTAPRWATEAAAAACLAAMAAAEASAPQAPAAA
AGGQEGAAGPVMVSVPFSREVLAEATACRVRSGTAAGARGNAPGAQGGVRKRTSKGGKAK
GGDREWSPEGEENTAPQPRGGGKRKSGAVAGGEEADGVASGRAKRASRPKRGSSKHDPY
VDDNDYGDEGIDPFDVGDDLDDMNPHGRYGNGGGRRADPSEAISALTAMGFTQSKARGALR
ECNFNVELAVEWLFANCL
>ORZ17038.1 [Absidia repens]
(SEQ ID No: 78)
MSSPSSPSPIKPRSMLTGSRNVVRSNDSASLWNCTLSPGWNEEQSETLRHAVMKYGIGNWA
KIIDSGYLPGKTNAQMNLQLQRLLGQQSTAEFAGLHIDPKVIGEQNSRIQGPEIRRKNNTIVNTG
DKLSREALRERILRNKEKYELPESVWQAIELEHVTDEDALLEEKKKTLREMKSQLKVVQRQIKN
LEFMHPLHAAKLKFELEKLAPSSSTSSSSSSPSPSSSSSPSSSSSKPSVSGTEEEMREAVDEE
RGSDEEIDELVEETDEEETSVSPKVGTRTKKVRTN
>ORX56339.1 [Hesseltinella vesiculosa]
(SEQ ID No: 79)
MIANSTATPKPRSMKAGARNVLRSNDSASLWNCTLSPGWTEQESEILRQLAIKFGIGNWAKIIE
SDCLPGKTNAQMNLQLQRLLGQQSTAEFAGLHIDPKVIGEKNSKIQGPHIKRKNTTIVNTGGKL
SREELRERQAKNKEMYEMPKSAWDSIDLDELRDMNSLKLKKKEDKDALKKQKLTQLKTKLTK
SQNNLKKVQAELKQIAMVDPERVAELKKELSRASSPLSNEVSVIEESPAKKQRTS
>ORX54764.1 [Piromyces finnis]
(SEQ ID No: 80)
MVVEKDLAQENKIKEELNKKHEWVKEMRKKFCVRKEFENTKNLILEDGTLNQEYFRLSKGTVL
KTNEVRKWTSIERNLLIKGIEKYGIGHFREISESLLPKWSGNDLRIKTIHLIGRQNLKLYKDWKG
GEEDIKREYNRNKEIGLKCNAWKNNCLIDDGNGKVKEMIEATEPKH
>ORX84766.1 [Anaeromyces robustus]
(SEQ ID No: 81)
MVVEKETNKENIKNIKEELDKKHAWVKEMRKKFCVRKEFENTKILILEDGTLNQDYFRLSKGTV
LKTNEVRKWTSIERGLLIKGIEKYGIGHFREISENLLPKWSGNDLRIKTIHLIGRQNLKLYKDWK
GNEEDIKREYNRNKEIGLKCNAWKNNCLVDDGHGKVKAMIEATENN
>ORY98423.1 [Syncephalastrum racemosum]
(SEQ ID No: 82)
MMTATDEDVDMKDVDIKLESNQETEQKILTPEEQKEKEKQDWIRQLRLKFCIRPEYEITKNMIF
PDGTLNQDYFRPPKGAKVEEARKWTEVEKELLIQGIEKYGIGNFGEVSKALLPAWSTNDLRIK
CIRLIGRQNLQLYRGWKGNADDIAREYNRNKELGLKYGTWKQGVLVYDDDGLVEKEILAQDA
AAKGEDVDMN
>XP_021886199.1 [Lobosporangium transversale]
(SEQ ID No: 83)
MEINQEQLPSSSSILHPTSTSSSSSPSPSPSPASPKPERVFDARQRRINEIRLKFCIRDEFPITK
NMIHPDGTLNQDYFRPPRGSKPVEVARKWTDKERELLIKGIEKYGIGHFREISEEFLPLWSGN
DLRIKTMRLVGRQNLQLYKDWKGNEQDLAREFELNKAIGLKYGAWKAGTLVADDDGLVAKAI
EEQWPGSNSGTGKTTAVIGISSEENSEVSTPLNDEDVDME
>ORY01319.1 [Basidiobolus meristosporus CBS 931.73]
(SEQ ID No: 84)
MEVDQNDSSVAKETAEQPETPEISKELLERQEWIKNMRLQFCVRPEFEVTKNIIHEDGMLNQE
YFLPPKGAKLEAEPERKWTETERNLLIQGIQQYGIGHFREISEALLPQWSGNDLRVKSMRLMG
RQNLQLYKDWKGSIEDIEREYERNKAIGLKYNTWKNSTLVYDDAGLVLKAIEASEPKP
>ORZ26026.1 [Absidia repens]
(SEQ ID No: 85)
MAIDSLQDTEDDRTNDQNDESRESSPTPLSPEEQAQKERHEDWINQIRLKFCIRPEFEVTKNIIH
PDGRLNQEYFHPPKGYKPEDARKWTETEKQLLIKGIEEHGIGNFGLISKESLPKWSTNDLRVK
CIRLIGRQNLQLYRGWKGNADDITREYERNKEIGLKYGTWKQGVLVYDDDGMVEKELLATAAT
PADSMSMEEDEDMATD
>ORX67568.1 [Linderina pennispora]
(SEQ ID No: 86)
MDTASPDDGAIAQPMLGVEDADFWRQKQEWVKQMRLQFSRRPEFPETHNMIDDEGMLNQE
YFQPPKDAVAPKERKWGDDEKRRLLEGIEKHGIGHFREISEESLPEWSGNDLRMKAIRLMGR
QNLQLYKGWKGDAAAIGLKHGTWKGGALVYDDDGVVLKAIQESNRANPP
>XP_001699352.1 [Chlamydomonas reinhardtii]
(SEQ ID No: 87)
MAACSAACDSHVVPQPSPGSWGMPEDRDNYIVQMRRRYSPAGMLNADGSINQDFFKPRRV
VLVADRAKWGDAEREGLYKGLEVHGVGKWREINRDYLKGQWDDQQVRIRAARLLGSQSLVR
YMGWKGSKAKVDAEYAKNKAIGEATGCWKAGQLVEDDHGSVRKYFEAQQAGGEQ
Protein sequences for phylogenetic analysis of p2 proteins
>XP_001017830.3 [Tetrahymena thermophila SB210]
(SEQ ID No: 88)
MNQMGVIAIKRKQSYQLNVKINYINTAHQIKKPCQYIQKCILFRLLYKFCKQLIPLNFNLFLIFYFY
HLLFHLIFNYLLKFAKKINKLIRNQRKNREKKEAFKHKKIQININHYNYLKQNIQQVGIIFQNKKSK
LTLKLVQKKSLSEYYRKIKMKKNGKSQNQPLDFTQYAKNMRKDLSNQDICLEDGALNHSYFLT
KKGQYWTPLNQKALQRGIELFGVGNWKEINYDEFSGKANIVELELRTCMILGINDITEYYGKKIS
EEEQEEIKKSNIAKGKKENKLKDNIYQKLQQMQ
>XP_001699352.1 [Chlamydomonas reinhardtii]
(SEQ ID No: 89)
MAACSAACDSHVVPQPSPGSWGMPEDRDNYIVQMRRRYSPAGMLNADGSINQDFFKPRRV
VLVADRAKWGDAEREGLYKGLEVHGVGKWREINRDYLKGQWDDQQVRIRAARLLGSQSLVR
YMGWKGSKAKVDAEYAKNKAIGEATGCWKAGQLVEDDHGSVRKYFEAQQAGGEQ
>EJY77156.1 [Oxytricha trifallax]
(SEQ ID No: 90)
MSTAKQQQAQQHLLPKHSNMRVGSVSNELDYAKRNYIIKMRQSFIEVNKNIYFEDGSLNFKYF
NVKKGHYWSKEINEELIKGVIKYGATNYKDIKNKMEIFKKEWSETEIRLRICRLLKCYNLKVYEG
HKFNSREEILEQATLNKEEAIKQKKICGGILYNPPHEQDDGIMSSYFNLKNKNNTPVKASAQ
>ORZ26026.1 [Absidia repens]
(SEQ ID No: 91)
MAIDSLQDTEDDRTNDQNDESRESSPTPLSPEEQAQKERHDWINQIRLKFCIRPEFEVTKNIIH
PDGRLNQEYFHPPKGYKPEDARKWTETEKQLLIKGIEEHGIGNFGLISKESLPKWSTNDLRVK
CIRLIGRQNLQLYRGWKGNADDITREYERNKEIGLKYGTWKQGVLVYDDDGMVEKELLATAAT
PADSMSMEEDEDMATD
>ORY96423.1 [Syncephalastrum racemosum]
(SEQ ID No: 92)
MMTATDEDVDMKDVDIKLESNQETEQKILTPEEQKEKEKQDWIRQLRLKFCIRPEYEITKNMIF
PDGTLNQDYFRPPKGAKVEEARKWTEVEKELLIQGIEKYGIGNFGEVSKALLPAWSTNDLRIK
CIRLIGRQNLQLYRGWKGNADDIAREYNRNKELGLKYGTWKQGVLVYDDDGLVEKEILAQDA
AAKGEDVDMN
>XP_021886199.1 [Lobosporangium transversale]
(SEQ ID No: 93)
MEINQEQLPSSSSILHPTSTSSSSSPSPSPSPASPKPERVFDARQRRINEIRLKFCIRDEFPITK
NMIHPDGTLNQDYFRPPRGSKPVEVARKWTDKERELLIKGIEKYGIGHFREISEEFLPLWSGN
DLRIKTMRLVGRQNLQLYKDWKGNEQDLAREFELNKAIGLKYGAWKAGTLVADDDGLVAKAI
EEQWPGSNSGTGKTTAVIGISSEENSEVSTPLNDEDVDME
>ORY01319.1 [Basidiobolus meristosporus CBS 931.73]
(SEQ ID No: 94)
MEVDQNDSSVAKETAEQPETPEISKELLERQEWIKNMRLQFCVRPEFEVTKNIIHEDGMLNQE
YFLPPKGAKLEAEPERKWTETERNLLIQGIQQYGIGHFREISEALLPQWSGNDLRVKSMRLMG
RQNLQLYKDWKGSIEDIEREYERNKAIGLKYNTWKNSTLVYDDAQLVLKAIEASEPKP
>ORX67568.1 [Linderina pennispora]
(SEQ ID No: 95)
MDTASPDDGAIAQPMLGVEDADFWRQKQEWVKQMRLQFSRRPEFPETHNMIDDEGMLNQE
YFQPPKDAVAPKERKWGDDEKRRLLEGIEKHGIGHFREISEESLPEWSGNDLRMKAIRLMGR
QNLQLYKGWKGDAAAIGLKHGTWKGGALVYDDDGVVLKAIQESNRANPP
>ORX84766.1 [Anaeromyces robustus]
(SEQ ID No: 96)
MVVEKETNKENIKNIKEELDKKHAWVKEMRKKFCVRKEFENTKILILEDGTLNQDYFRLSKGTV
LKTNEVRKWTSIERGLLIKGIEKYGIGHFREISENLLPKWSGNDLRIKTIHLIGRQNLKLYKDWK
GNEEDIKREYNRNKEIGLKCNAWKNNCLVDDGHGKVKAMIEATENN
>ORX54764.1 [Piromyces finnis]
(SEQ ID No: 97)
MVVEKDLAQENKIKEELNKKHEWVKEMRKKFCVRKEFENTKNLILEDGTLNQEYFRLSKGTVL
KTNEVRKWTSIERNLLIKGIEKYGIGHFREISESLLPKWSGNDLRIKTIHLIGRQNLKLYKDWKG
GEEDIKREYNRNKEIGLKCNAWKNNCLIDDGNGKVKEMIEATEPKH
>ORX56334.1 [Hesseltinella vesiculosa]
(SEQ ID No: 98)
MLAGDAELVEKPHNALNAEDTEMEDVDHSSHPDTTVDLSPEQLRLQEKQAWINQMRLKFCV
REEFEITKNMIHPDGILNQDYFKPPKKSKKKKSKSKSKGTDETKDDTEAKGEDNKEDEDME
>PNW76495.1 [Chlamydomonas reinhardtii]
(SEQ ID No: 99)
MAFAAALAEKRGPRVGDAASLWNFTPAPGWSREEVQILRLCLMKHGVGQWMQILSTGLLPG
KLIQQLNGQTQRLLGQQSLAAYTGLKVDVDRIRVDNETRTDATRKAGLIINDGPNLTKEMKEK
MRQDAVAKYGLTPEQVAEVDEQLAEIAAAFNPASTSAAAGAGSGAAAAGQAAAAGSGAGGS
GQAATAADAGGAAGRGTGSAGGAAAAAPPRNALAISTGVLAATLLDASLGNLMAQPTEQLSA
EQLGQLLLRLRNRLACLVDRARGRAGLPPRTAPRWATEAAAAACLAAMAAAEASAPQAPAAA
AGGQEGAAGPVMVSVPFSREVLAEATACRVRSGTAAGARGNAPGAQGGVRKRTSKGGKAK
GGDREWSPEGEENTAPQPRGGGKRKSGAVAGGEEADGVASGRAKRASRPKRGSSKHDPY
VDDNDYGDEGIDPFDVGDDLDDMNPHGRYGNGGGRRADPSEAISALTAMGFTQSKARGALR
ECNFNVELAVEWLFANCL
>XP_001698763.1 [Chlamydomonas reinhardtii]
(SEQ ID No: 100)
MAFAAALAEKRGPRVGDAASLWNFTPAPGWSREEVQILRLCLMKHGVGQWMQILSTGLLPG
KLIQQLNGQTQRLLGQQSLAAYTGLKVDVDRIRVDNETRTDATRKAGLIINDGPNLTKEMKEK
MRQDAVAKYGLTPEQVAEVDEQLAEIAAAFNPASTSAAAGAGSGAAAAGQAAAAGSGAGGS
GNLMAQPTEQLSAEQLGQLLLRLRNRLACLVDRARGRAGLPPRTAPRWATEAAAAACLAAM
AAAEASAPQAPAAAAGGQEGAAGPVMVSVPFSREVLAEATACRVRSGTAAGARGNAPGAQ
GGVRKRTSKGGKAKGGDREWSPEGEENTAPQPRGGGKRKSGAVAGGEEADGVASGRAKR
ASRPKRGSSKHDPYVDDNDYGDEGIDPFDVGDDLDDMNPHGRYGNGGGRRADPSEAISALT
AMGFTQSKARGALRECNFNVELAVEWLFANCL
>XP_011237366.1 [Mus musculus]
(SEQ ID No: 101)
MPRRQAEAMDIDAEREKITQEIQELERILYPGSTSVHFEVSESSLSSDSEADSLPDEDLETAGA
PILEEEGSSESSNDEEDPKDKALPEDPETCLQLNMVYQEVIREKLAEVSQLLAQNQEQQEEILF
DLSGTKCPKVKDGRSLPSYMYIGHFLKPYFKDKVTGVGPPANEETREKATQGIKAFEQLLVTK
WKHWEKALLRKSVVSDRLQRLLQPKLLKLEYLHEKQSRVSSELERQALEKQIKEAEKEIQDIN
QLPEEALLGNRLDSHDWEKISNINFEGARSAEEIRKFWQSSEHPSISKQEWSTEEVERLKAIA
ATHGHLEWHLVAEELGTSRSAFQCLQKFQQYNKTLKRKEWTEEEDHMLTQLVQEMRVGNHI
PYRKIVYFMEGRDSMQLIYRWTKSLDPSLKRGFWAPEEDAKLLQAVAKYGAQDWFKIREEVP
GRSDAQCRDRYIRRLHFSLKKGRWNAKEEQQLIQLIEKYGVGHWARIASELPHRSGSQCLSK
WKILARKKQHLQRKRGQRPRHSSQWSSSGSSSSSSEDYGSSSGSDGSSGSENSDVELEAS
LEKSRALTPQQYRVPDIDLWVPTRLITSQSQREGTGCYPQHPAVSCCTQDASQNHHKEGSTT
VSAAEKNQLQVPYETHSTVPRGDRFLHFSDTHSASLKDPACKPVLKVPLEKMPKLIRTRPPTQ
SHTLMKERPKQPLLPSSRSGSDPGNNTAGPHLRQLWHGTYQNKQRRKRQALHRRLLKHRLL
LAVIPWVGDINLACTQAPRRPATVQTKADSIRMQLECARLASTPVFTLLIQLLQIDTAGMEVV
RERKSQPPALLQPGTRNTQPHLLQASSNAKNNTGCLPSMTGEQTAKRASHKGRPRLGSCRT
EATPFQVPVAAPRGLRPKPKTVSELLREKRLRESHAKKATQALGLNSQLLVSSPVILQPPLLPV
PHGSPVVGPATSSVELSVPVAPVMVSSSPSGSWPVGGISATDKQPPNLQTISLNPPHKGTQV
AAPAAFRSLALAPGQVPTGGHLSTLGQTSTTSQKQSLPKVLPILRAAPSLTQLSVQPPVSGQP
LATKSSLPVNWVLTTQKLLSVQVPAVVGLPQSVMTPETIGLQAKQLPSPAKTPAFLEQPPAST
DTEPKGPQGQEIPPTPGPEKAALDLSLLSQESEAAIVTWLKGCQGAFVPPLGSRMPYHPPSL
CSLRALSSLLLQKQDLEQKASSLAASQAAGAQPDPKAGALQASLELVQRQFRDNPAYLLLKTR
FLAIFSLPAFLATLPPNSIPTTLSPDVAVVSESDSEDLGDLELKDRARQLDCMACRVQASPAAP
DPVQSHLVSPGQRAPSPGEVSAPSPLDASDGLDDLNVLRTRRARHSRR
>XP_006497966.1 [Mus musculus]
(SEQ ID No: 102)
MPRRQAEAMDIDAEREKITQEIQELERILYPGSTSVHFEVSESSLSSDSEADSLPDEDLETAGA
PILEEEGSSESSNDEEDPKDKALPEDPETCLQLNMVYQEVIREKLAEVSQLLAQNQEQQEEILF
DLSGTKCPKVKDGRSLPSYMYIGHFLKPYFKDKVTGVGPPANEETREKATQGIKAFEQLLVTK
WKHWEKALLRKSVVSDRLQRLLQPKLLKLEYLHEKQSRVSSELERQALEKQIKEAEKEIQDIN
QLPEEALLGNRLDSHDWEKISNINFEGARSAEEIRKFWQSSEHPSISKQEWSTEEVERLKAIA
ATHGHLEWHLVAEELGTSRSAFQCLQKFQQYNKTLKRKEWTEEEDHMLTQLVQEMRVGNHI
PYRKIVYFMEGRDSMQLIYRWTKSLDPSLKRGFWAPEEDAKLLQAVAKYGAQDWFKIREEVP
GRSDAQCRDRYIRRLHFSLKKGRWNAKEEQQLIQLIEKYGVGHWARIASELPHRSGSQCLSK
WKILARKKQHLQRKRGQRPRHSSQWSSSGSSSSSSEDYGSSSGSDGSSGSENSDVELEAS
LEKSRALTPQQYRVPDIDLWVPTRLITSQSQREGTGCYPQHPAVSCCTQDASQNHHKEGSTT
VSAAEKNQLQVPYETHSTVPRGDRFLHFSDTHSASLKDPACKSHTLMKERPKQPLLPSSRSG
SDPGNNTAGPHLRQLWHGTYQNKQRRKRQALHRRLLKHRLLLAVIPWVGDINLACTQAPRRP
ATVQTKADSIRMQLECARLASTPVFTLLIQLLQIDTAGCMEVVRERKSQPPALLQPGTRNTQP
HLLQASSNAKNNTGCLPSMTGEQTAKRASHKGRPRLGSCRTEATPFQVPVAAPRGLRPKPK
TVSELLREKRLRESHAKKATQALGLNSQLLVSSPVILQPPLLPVPHGSPVVGPATSSVELSVPV
APVMVSSSPSGSWPVGGISATDKQPPNLQTISLNPPHKGTQVAAPAAFRSLALAPGQVPTGG
HLSTLGQTSTTSQKQSLPKVLPILRAAPSLTQLSVQPPVSGQPLATKSSLPVNWVLTTQKLLSV
QVPAVVGLPQSVMTPETIGLQAKQLPSPAKTPAFLEQPPASTDTEPKGPQGQEIPPTPGPEKA
ALDLSLLSQESEAAIVTWLKGCQGAFVPPLGSRMPYHPPSLCSLRALSSLLLQKQDLEQKASS
LAASQAAGAQPDPKAGALQASLELVQRQFRDNPAYLLLKTRFLAIFSLPAFLATLPPNSIPTTLS
PDVAVVSESDSEDLGDLELKDRARQLDCMACRVQASPAAPDPVQSHLVSPGQRAPSPGEVS
APSPLDASDGLDDLNVLRTRRARHSRR
>EJY86254.1 [Oxytricha trifallax]
(SEQ ID No: 103)
MSVHHKMADSKSLHNYTLSPGWTREEVDILKIALMKFGIGKWKKIQKSGCLPSKTISQMNLQT
QRLLGQQSLAEFMGLHVYLDRVFRDNSLKTGPEIQRKNNFIINTGNNLTQPEKEKRLRLNKQK
YGLDLAFIKTLRLPKPESATGGKREAILSMDQIFAQKSHFTVVEKLKHLEALKNALCSKLGKIER
RRRNKELSKIYRPLCQLIVVQKNADDQYEFVDIIDENE
>ORX69504.1 [Linderina pennispora]
(SEQ ID No: 104)
MSSATPYAPRSMPTGQRNVVRSNDSASLWNCTLSPGWTQEEVQVLRKALMKFGVGNWMKII
ESECLPGKTIAQMNLQTQRMLGQQSTAEFNGLHLDAFVIGELNSKKQGPGIKRKNNCIVNTGG
KLTRDEVVKRQQKHREQYEVKAEVWRAIVLPKPDNPLILLEKKREELKKVRLELEEIMKQIEET
EKLVDVPEHAPGTKRARE
>NP_003077.2 [Homo sapiens]
(SEQ ID No: 105)
MDVDAEREKITQEIKELERILDPGSSGSHVEISESSLESDSEADSLPSEDLDPADPPISEEERW
GEASNDEDDPKDKTLPEDPETCLQLNMVYQEVIQEKLAEANLLLAQNREQQEELMRDLAGSK
GTKVKDGKSLPPSTYMGHFMKPYFKDKVTGVGPPANEDTREKAAQGIKAFEELLVTKWKNW
EKALLRKSVVSDRLQRLLQPKLLKLEYLHQKQSKVSSELERQALEKQGREAEKEIQDINQLPEE
ALLGNRLDSHDWEKISNINFEGSRSAEEIRKFWQNSEHPSINKQEWSREEEERLQAIAAAHGH
LEWQKIAEELGTSRSAFQCLQKFQQHNKALKRKEWTEEEDRMLTQLVQEMRVGSHIPYRRIV
YYMEGRDSMQLIYRWTKSLDPGLKKGYWAPEEDAKLLQAVAKYGEQDWFKIREEVPGRSDA
QCRDRYLRRLHFSLKKGRWNLKEEEQLIELIEKYGVGHWAKIASELPHRSGSQCLSKWKIMM
GKKQGLRRRRRRARHSVRWSSTSSSGSSSGSSGGSSSSSSSSSEEDEPEQAQAGEGDRAL
LSPQYMVPDMDLWVPARQSTSQPWRGGAGAWLGGPAASLSPPKGSSASQGGSKEASTTA
AAPGEETSPVQVPARAHGPVPRSAQASHSADTRPAGAEKQALEGGRRLLTVPVETVLRVLRA
NTAARSCTQKEQLRQPPLPTSSPGVSSGDSVARSHVQWLRHRATQSGQRRWRHALHRRLL
NRRLLLAVTPWVGDVVVPCTQASQRPAVVQTQADGLREQLQQARLASTPVFTLFTQLFHIDT
AGCLEVVRERKALPPRLPQAGARDPPVHLLQASSSAQSTPGHLFPNVPAQEASKSASHKGSR
RLASSRVERTLPQASLLASTGPRPKPKTVSELLQEKRLQEARAREATRGPVVLPSQLLVSSSVI
LQPPLPHTPHGRPAPGPTVLNVPLSGPGAPAAAKPGTSGSWQEAGTSAKDKRLSTMQALPL
APVFSEAEGTAPAASQAPALGPGQISVSCPESGLGQSQAPAASRKQGLPEAPPFLPAAPSPT
PLPVQPLSLTHIGGPHVATSVPLPVTWVLTAQGLLPVPVPAVVSLPRPAGTPGPAGLLATLLPP
LTETRAAQGPRAPALSSSWQPPANMNREPEPSCRTDTPAPPTHALSQSPAEADGSVAFVPG
EAQVAREIPEPRTSSHADPPEAEPPWSGRLPAFGGVIPATEPRGTPGSPSGTQEPRGPLGLE
KLPLRQPGPEKGALDLEKPPLPQPGPEKGALDLGLLSQEGEAATQQWLGGQRGVRVPLLGS
RLPYQPPALCSLRALSGLLLHKKALEHKATSLVVGGEAERPAGALQASLGLVRGQLQDNPAYL
LLRARFLAAFTLPALLATLAPQGVRTTLSVPSRVGSESEDEDLLSELELADRDGQPGCTTATC
PIQGAPDSGKCSASSCLDTSNDPDDLDVLRTRHARHTRKRRRLV
>XP_016870547.1 [Homo sapiens]
(SEQ ID No: 106)
MDVDAEREKITQEIKELERILDPGSSGSHVEISESSLESDSEADSLPSEDLDPADPPISEEERW
GEASNDEDDPKDKTLPEDPETCLQLNMVYQEVIQEKLAEANLLLAQNREQQEELMRDLAGSK
GTKVKDGKSLPPSTYMGHFMKPYFKDKVTGVGPPANEDTREKAAQGIKAFEELLVTKWKNW
EKALLRKSVVSDRLQRLLQPKLLKLEYLHQKQSKVSSELERQALEKQGREAEKEIQDINQLPEE
ALLGNRLDSHDWEKISNINFEGSRSAEEIRKFWQNSEHPSINKQEWSREEEERLQAIAAAHGH
LEWQKIAEELGTSRSAFQCLQKFQQHNKALKRKEWTEEEDRMLTQLVQEMRVGSHIPYRRIV
YYMEGRDSMQLIYRWTKSLDPGLKKGYWAPEEDAKLLQAVAKYGEQDWFKIREEVPGRSDA
QCRDRYLRRLHFSLKKGRWNLKEEEQLIELIEKYGVGHWAKIASELPHRSGSQCLSKWKIMM
GKKQGLRRRRRRARHSVRWSSTSSSGSSSGSSGGSSSSSSSSSEEDEPEQAQAGEGDRAL
LSPQYMVPDMDLWVPARQSTSQPWRGGAGAWLGGPAASLSPPKGSSASQGGSKEASTTA
AAPGEETSPVQVPARAHGPVPRSAQASHSADTRPAGAEKQALEGGRRLLTVPVETVLRVLRA
NTAARSCTQWLRHRATQSGQRRWRHALHRRLLNRRLLLAVTPWVGDVVVPCTQASQRPAV
VQTQADGLREQLQQARLASTPVFTLFTQLFHIDTAGCLEVVRERKALPPRLPQAGARDPPVHL
LQASSSAQSTPGHLFPNVPAQEASKSASHKGSRRLASSRVERTLPQASLLASTGPRPKPKTV
SELLQEKRLQEARAREATRGPVVLPSQLLVSSSVILQPPLPHTPHGRPAPGPTVLNVPLSGPG
APAAAKPGTSGSWQEAGTSAKDKRLSTMQALPLAPVFSEAEGTAPAASQAPALGPGQISVSC
PESGLGQSQAPAASRKQGLPEAPPFLPAAPSPTPLPVQPLSLTHIGGPHVATSVPLPVTWVLT
AQGLLPVPVPAVVSLPRPAGTPGPAGLLATLLPPLTETRAAQGPRAPALSSSWQPPANMNRE
PEPSCRTDTPAPPTHALSQSPAEADGSVAFVPGEAQVAREIPEPRTSSHADPPEAEPPWSGR
LPAFGGVIPATEPRGTPGSPSGTQEPRGPLGLEKLPLRQPGPEKGALDLEKPPLPQPGPEKG
ALDLGLLSQEGEAATQQWLGGQRGVRVPLLGSRLPYQPPALCSLRALSGLLLHKKALEHKAT
SLVVGGEAERPAGALQASLGLVRGQLQDNPAYLLLRARFLAAFTLPALLATLAPQGVRTTLSV
PSRVGSESEDEDLLSELELADRDGQPGCTTATCPIQGAPDSGKCSASSCLDTSNDPDDLDVL
RTRHARHTRKRRRLV
>XP_020936800.1 [Sus scrofa]
(SEQ ID No: 107)
MDVDAEREKISKEIKELERILDPGSSGINDDVSESSLDSDSEAESLPDDDADATGPLLSEDERW
GDASNDEDDAKERALPEDPETCLQLNMVYQEVVREKLAEVSLLLAQNREQQEEVSWALAGS
GGRRVKDGRSPPARLYVGHFMKPYFKDKVTGAGPPANEDTREKAAQGVKAFEELLVTKWKS
WEKALLRKAVVSDRLQRLLQPKLLKLEYLQQKQSRATSDAERQALEKQVREAEKEVQDISQL
PEEALLGHRLDSHDWEKIANVNFEGGRSAEETRKFWQNHEHPSINKQEWSAQEVDRLKAIAA
KHGHLRWQEIAEELGTRRSAFQCLQKYQQHNAALKRREWTQEEDRMLTQLVQAMGVGSHIP
YRRIAYYMEGRDSTQLIYRWTKSLDPALKKGLWAPEEDAKLLQAVAKYGEQDWFKIREEVPG
RSDAQCRDRYLRRLRLSLKKGRWSAQEEERLLELIGKHGVGHWAKIASELPHRTDSQCLSK
WKIMARKQQSRGRRRRRPLRRVCWSSSSEDSEDSGDSGGSSSSSSSSEDVEPEGAPEARA
DGPAPPSAQHPVPDMDLWVPTRQSARVPWGVGPGAWPGHRSASPRPPEGSDVAPGEEAG
RAQAPSETPSASLRGGGCPRSADARPSGSEGLADEGPRRPLTVPLETVLRVLRTNTAALCRA
LKEKLRRPRLLGSPLGPSPSDGSVARPRVQPRWRRRHALQRRLLERQLLMAVSPWVGDVTL
PCAPWRPAVLHRRADGIGKQLQGARLASTPVFTLLIQLFRIDTAGCMEVVRERRAQPPALPSG
GRVPSSARNSPGHLFQNGSARGAAKKSASHSGGGGPQSAPAPSGPRPKPKTVSELLREKRL
REARARKAAQGPAVLPPQGLLSSPAILQPLPPQQLPVSGAVLSGPGGPAVASPGAPGPWAS
AKEGPPSLHALALAPASMAAGVTPAAPRAPALGPSQVPASCHLSSLGQSQAPATSRKQGLPE
APPFLPAAPSPIQLPVQPRSLTPALAAHTGASHVVASTPLPVTWVLTAQGLLPVPAVVGLPRP
AGPPDPEGLSGTPPPSLTETRAGRGPKQPPAHVSVGPDPPAKTPPTAQSPAEGDGDVAHGP
GGPSCPGEAQVAGEASVPRTLSPAKPLADHPEAEPCGSSQLPLPGGLSPGGAPTRHQGLER
PPPPWPGPEKGAPDLRLLSQESEAAVRGWLTGQRGVCVPPLASRLPYQPPTLCSLRALSGLL
LHKKALEHRAASLVPSGAAGAQQAPLGQVRERLQSSPAYLLLKARFLAAFALPALLATLPPHG
VPTTLSAAAGVDSESDDDSLDELELADNGGPLGGWPSGRQAGPAAPTPTQGAPGEGSAAP
GLDSDDLDILRTRHAWHARKRRRLV
>XP_021883515.1 [Lobosporangium transversale]
(SEQ ID No: 108)
MSSGSTPRSMTAGARNILRSNDSASLWNYTVAPGWSMKEAEILRKALMKFGIGNWSKIIESN
CLVGKTNAQMNLQTQRMLGQQSTAEFAGLHIDPRVIGQKNSLIQGDHIRRKNGCIVNTGAKLS
REEIRRRVAENKEQYELPEEEWSSIELPLPDDPHLLLEAKKSEKVRLELELKNVQRQIAMLRKV
GRKFETGSESPKTELDDDERDEFIEDQPLGKRARIEA
>EJY76686.1 [Oxytricha trifallax]
(SEQ ID No: 109)
MRVYLKFCNRKQIHYTHTMSSSISAAIMAGNQNKKIAESKSLWNYALSPGWTQQEVEILKIALM
KFGVGRWSAINKSGVLPTKQIQQCYLQTQRLIGQQSLAEFMGLHLDIDRIAADNKQKRGIRKQ
GFLVNQGCKLTPEEKDELRKINQEKYGLTAEHVEAIKLPAPCHLVEIFQIDKIMHPRSTLSTMDK
IKHLIKLEDALKSKLEMIREGKRQQKFEQLQQKLKTTEASGRGSVTRVQRQMSDLHLGSAHQN
RNSDLDEENDQSVMIIDESQQQNLTPKGKAQTMLTNQTQTMKKQADDSRDEQHLPLISTSAS
VSNPSSTSKSSALKLNSMKQSDTAIASMKPSSSGKKTKVDSSFVSKQSNQQSTSYSETNVDT
QNSNNQGTSTASGNFISQSDDEEALMPKLKRRRVEDSE
>EJY73777.1 [Oxytricha trifallax]
(SEQ ID No: 110)
MSHATSHGNSTEKDKKNSGNMVAESKSLWNYALSPGWTPQEVDVLKIALMKFGIGKWTIIDK
SGILPTKTIQQCYLQTQRILGQQSLAEFMGLHVDIDKIALDNRRKNGIRKMGFLVNQGGKLTPE
EKAHYQEINRQKYGLSPEEVETIKLPPPCSVEIYDINKIINPKSKLTTIEKINHCIKLQDALLEKLEN
IKNKKIPTGAGFSSSRVYENMRGYDPQLLLNSHVTGQLDHSMQDLTIDERYSDLDEEEDPLAM
ASIIDSQATPQPQKIKSSVPNKASTTPSAKEMNQIKDIIDSVIAENSAQQSKNLAQEKPKLKFSLV
KATESNLLQSAAQNSDDVVMEEDSKLQHIETFSTVTQTATDQSNSQSKSQNNIASDSLKDSLE
QNDLSKSLTDSLEMQQYSAEKKLNQAPMSKNSDKPKKKRLNKRKLPSDDEFETL
>EJY79729.1 [Oxytricha trifallax]
(SEQ ID No: 111)
MSSSISAAIIAGNQNKKIAESKSLWNYALSPGWTQQEVEILKIALMKFGVGRWKTIEQSQCLPT
KTMSQMYLQTQRLVGQQSLAEFMGLHLDLEQIFIKNAERQGAGVFRKNGCIINTGDNMTKVQI
AKLRKKNSKIFGLTQPFVQSLHLPKAKVKEWLKVLTLDQILSAKSNFSTAEKIHYLKILENALER
KLKKILRLQELVSIYRPCNIGIVVQKRLGSSIGDEYFEYVDCVKIEEKSVGNLDFALPNRNTDSTS
LNEDFSFLDSTQKPQKLKAGSGRENKRKKMRDGLKDERAQRQSLMEALDEQEFDETKFQDS
DGEMPDLNM
>EJY81929.1 [Oxytricha trifallax]
(SEQ ID No: 112)
MSSSISAAIMAGNQNKKIAESKSLWNYALSPGWTQQEVEILKIALMKFGVGRWSAINKSGVLP
TKQIQQCYLQTQRLIGQQSLAEFMGLHLDIDRIAADNKQKRGIRKQGFLVNQGCKLTPEEKDEL
RKINQEKYGLSAEHVEAIKLPAPCHLVEIFQIDKIMHPRSTLSTMDKIKHLIKLEDALKSKLEMIRE
GKRQQKFEQLQQKLKTTEASGRGSVTRVQRQMSDLHLGSSHQNRNSDLDEENDESVMIIDE
SQQENLTPKGKAQAMLTHQKYNEVTQTMIKQGDDSRQQQHLPLDSTSASVSNPSSTSKSST
MKSNSMKQSETAIASMKPSSIGKKTKVDSSFVTKQSNQQSTAPIQKQAHQQNLDRNRSELGS
TFAQQASVDTQNSNNQGTSTASGNFISQSDDEEALMPKLKRRRVEDSE
>EJY80746.1 [Oxytricha trifallax]
(SEQ ID No: 113)
MRVYLKFCNRKQIHYTHTMSSSISAAIMAGNQNKKIAESKSLWNYALSPGWTQQEVEILKIALM
KFGVGRWSAINKSGVLPTKQIQQCYLQTQRLIGQQSLAEFMGLHLDIDRIAADNKQKRGIRKQ
GFLVNQGCKLTPEEKDELRKINQEKYGLTAEHVEAIKLPAPCHLVEIFQIDKIMHPRSTLSTMDK
IKHLIKLEDALKSKLEMIREGKRQQKFEQLQQKLKTTEASGRGSVTRVQRQMSDLHLGSAHQN
RNSDLDEENDQSVMIIDESQQQNLTPKGKAQTMLTNQTQTMKKQADDSREEQHLPLNSTSAS
VSNPSSTSKSSALKLNSMKQSDTAIASMKPSSSGKKTKVDSSFVSKQSNQQSTGPIQKQAHQ
QNLDRNRSELGSTFAQQTNVDTQNSNNQGTSTASGNFISQSDDEEALMPKLKRRRVKDSE
>ORX78557.1 [Basidiobolus meristosporus CBS 931.73]
(SEQ ID No: 114)
MTDVYKPRSMPVGARNVLRSNDSASLWNCTLSPGWTEPEVHILRKAVMKFGIGNWAKIIESQ
CLFGKTIAQMNLQLQRMLGQQSTAEFAGLHLDPFVIGEINSKKQGPGIKRKNNCIVNTGGKLTR
EEIKRRLLEHKRTYEISEEEWRSIELPKPEDPGAVLIAKKDELKMLEDELLRVVQKIQKAREERR
SKSVDSSSVDGSVDDEARETKRRRK
>ORX79686.1 [Anaeromyces robustus]
(SEQ ID No: 115)
MSIPKPRSMPTGFRNILRPNDSTSLWNCTLSPGWTQEESDILRDALIYYGIGNWKDIIEHGCLP
DKTNAQMNLQLQRMLGQQSTAEFQNLHIDPYVIGKINSQKQGPNIRRKNGFIINTGGKLSREDI
RRKIQENKENYELPKEEWSKIVLPNREVVIKNKVQEAINEKREKLNKLEDELDSVLKAIVNRRR
ELRGMIPLKDSEMKSLVNRSAKNEGENKTETTNNEESNNTNNSDDIKDENNETSTSSHIFTNN
DNELSENNSSSSSSNSISNKKKRFLRREVRRGKRRYNYDDDDFMPSGNRSRKSRKI
>ORX56566.1 [Piromyces finnis]
(SEQ ID No: 116)
MSIPKPRSMPVGFRNILRPNDSTSLWNCTLSPGWTQEESDILRDALIFYGIGNWKDIIEHGCLP
DKTNAQMNLQLQRMLGQQSTAEFQNLHIDPYEIGKINSQKQGPNIRRKNGFIINTGGKLSREDI
KRKIQENKENYELPEEVWSKIVLPNREVVTINEKRQKLNKLEEELDSVLKQIVNRRRELRGMTP
LKETEMKSIVNRSNQNDTKTEEKEIKEEESTTVNEEKIENTETSSISIISTNENEQSENISSSSPIV
KSEQKKKRVVSRRKNKRRVNSDDEDFLPPGKSRSKRTRRTPKKSSN
>XP_001009903.1 [Tetrahymena thermophila SB210]
(SEQ ID No: 117)
MSLKKGKFQHNQSKSLWNYTLSPGWREEEVKILKSALQLFGIGKWKKIMESGCLPGKSIGQIY
MQTQRLLGQQSLGDFMGLQIDLEAVFNQNMKKQDVLRKNNCIINTGDNPTKEERKRRIEQNR
KIYGLSAKQIAEIKLPKVKKHAPQYMTLEDIENEKFTNLEILTHLYNLKAEIVRRLAEQGETIAQPS
IIKSLNNLNHNLEQNQNSNSSTETKVTLEQSGKKKYKVLAIEETELQNGPIATNSQKKSINGKRK
NNRKINSDSEGNEEDISLEDIDSQESEINSEEIVEDDEEDEQIEEPSKIKKRKKNPEQESEEDDI
EEDQEEDELVVNEEEIFEDDDDDEDNQDSSEDDDDDED
>XP_020936799.1 [Sus scrofa]
(SEQ ID No: 118)
MDVDAEREKISKEIKELERILDPGSSGINDDVSESSLDSDSEAESLPDDDADATGPLLSEDERW
GDASNDEDDAKERALPEDPETCLQLNMVYQEVVREKLAEVSLLLAQNREQQEEVSWALAGS
GGRRVKDGRSPPARLYVGHFMKPYFKDKVTGAGPPANEDTREKAAQGVKAFEELLVTKWKS
WEKALLRKAVVSDRLQRLLQPKLLKLEYLQQKQSRATSDAERQALEKQVREAEKEVQDISQL
PEEALLGHRLDSHDWEKIANVNFEGGRSAEETRKFWQNHEHPSINKQEWSAQEVDRLKAIAA
KHGHLRWQEIAEELGTRRSAFQCLQKYQQHNAALKRREWTQEEDRMLTQLVQAMGVGSHIP
YRRIAYYMEGRDSTQLIYRWTKSLDPALKKGLWAPEEDAKLLQAVAKYGEQDWFKIREEVPG
VTFEARAFPASRQRTSLPCAPLWPPALWVSRLGNRRGGRQPRGFSRTPRSVCRRYLRRLRL
SLKKGRWSAQEEERLLELIGKHGVGHWAKIASELPHRTDSQCLSKWKIMARKQQSRGRRRR
RPLRRVCWSSSSEDSEDSGDSGGSSSSSSSSEDVEPEGAPEARADGPAPPSAQHPVPDMD
LWVPTRQSARVPWGVGPGAWPGHRSASPRPPEGSDVAPGEEAGRAQAPSETPSASLRGG
GCPRSADARPSGSEGLADEGPRRPLTVPLETVLRVLRTNTAALCRALKEKLRRPRLLGSPLGP
SPSDGSVARPRVQPRWRRRHALQRRLLERQLLMAVSPWVGDVTLPCAPWRPAVLHRRADG
IGKQLQGARLASTPVFTLLIQLFRIDTAGCMEVVRERRAQPPALPSGGRVPSSARNSPGHLFQ
NGSARGAAKKSASHSGGGGPQSAPAPSGPRPKPKTVSELLREKRLREARARKAAQGPAVLP
PQGLLSSPAILQPLPPQQLPVSGAVLSGPGGPAVASPGAPGPWASAKEGPPSLHALALAPAS
MAAGVTPAAPRAPALGPSQVPASCHLSSLGQSQAPATSRKQGLPEAPPFLPAAPSPIQLPVQ
PRSLTPALAAHTGASHVVASTPLPVTWVLTAQGLLPVPAVVGLPRPAGPPDPEGLSGTPPPSL
TETRAGRGPKQPPAHVSVGPDPPAKTPPTAQSPAEGDGDVAHGPGGPSCPGEAQVAGEAS
VPRTLSPAKPLADHPEAEPCGSSQLPLPGGLSPGGAPTRHQGLERPPPPWPGPEKGAPDLR
LLSQESEAAVRGWLTGQRGVCVPPLASRLPYQPPTLCSLRALSGLLLHKKALEHRAASLVPS
GAAGAQQAPLGQVRERLQSSPAYLLLKARFLAAFALPALLATLPPHGVPTTLSAAAGVDSESD
DDSLDELELADNGGPLGGWPSGRQAGPAAPTPTQGAPGEGSAAPGLDSDDLDILRTRHAWH
ARKRRRLV
>XP_009300052.1 [Danio rerio]
(SEQ ID No: 119)
MKCLSVNMTHLSRDSWLYTHDVQVTYNSFIKVSPCPKMASDDLRAQRDKIQREILALESTLGA
DSSIADQLSSDNSSDYESDDSGPTVKRVERDDLETERLRIQREIEELENALGADAALENVLQD
SDHDTDSSEDSADDLELPQNVETCLQMNLVYQEVLKEKLAELEQLLIENQQQQKEIEVQLSGP
GNSIFSVPGVPPQKQFLGYFLKPYFKDKLTGLGPPANEETKERMKHGSIPVDNLKIKRWEGW
QKTLLTNAVARDTMKRMLQPKLSKMEYLSNKLCRAEGEEKEQLKAQIELIEKQIAEIRTLKDDQ
LLGDLQDDHDWDKISNIDFEGLRQADDLKRFWQNFLHPSINKSVWKQDEIYKLQAVAEEFKM
CHWDKIAEALGTNRTAFMCFQTYQRYISKTFRRTHWTEEEDDLLRELVEKMRIGNFIPYIQMS
HFMVGRDGSQLAYRWTSVLDPSLKKGPWSKEEDQLLRNAVAKYGTREWGRIRTEVPGRTD
SACRDRYLDCLRETVKKGTWSYAEMELLKEKVAKYGVGKWAKIASEIPNRVDAQCLHKWKL
MTRSKKPLKRPLSSITTSYPRNKRQKLLKTVKEEMFFNSSSDDESQINYMNSDESDDLAEDEN
LEIPQKEYVQTEMKEWIPRNAMVWTITPGSFRTLWVRLPTNEEELRESTKESGLGSDSSENS
ACPNDEPIMERNTILDRFGDVERTYVGMNTVVLHRRTDDEKAMFKVCMSDVKQFIQMKATEF
AVKKKKKIKNKKRTLRDVFSLNTDLQKAVIPWIGNVIISTPANEAIFCEGDIVGIKAASIRLQKTSV
FTFFIKAFHVDVNGCRTVIEIHKKLDIKMPLAINGNPKPTPISTSPKTVAVLLQQSKAASEHKKPA
EPSQQPSLPPSQKPSLPPAQQPTQPPSLPPSVPPSQQPTLPPPSQPSQPPPQPPSLPPSQPP
AQQPPQQPSLPPPQPPSLPPPQPPSLPTSQQQSLPPSQQHSLPPFQNPSLPPSQQPSLPPS
KQPPQPLPVRQITTPTLIYPNNLVITNPNMEGEVQHLVFKGLLLPQQPSKAVSHIPLPVMQPKT
PAQPIVVSKSPSVQDSNSVKSSKRICKPTKKAQALMEQSKVKSRKKEPQKQNQGNKNVVFPT
VTLQTSPVIKILSPARLVQVTGLSPNFSSNQTINMPDKSLTIKSPQPCSSGNLHQSAPVVVHSS
TNPTFVHSSVSNVSRDNLNVSSTINISPRVSRDALNPTSFLNSTTFPLPQNLSVQQSVQIVPQIP
INVVHKATCTKAAKTSSDSSSDESVVKQHQLSPSTGRSIPPAVFNIQPNPSTPPTLSSGPVIFN
PNNKVVAPKLCGLNVSSSQLPTVSTQKTKYRPIRPLGPLPVVAPPSRKVTSMSRIRAQSEGEP
LISLRDLPAAGVNFDSHLIFPEKSSEVDDWMDGKGGIPLPHLDTSLPYLPPSAATIKTMTDLLRA
KQPLLLAAKKVLPAQYQDECNEEVEVEAIRKVVAERFASNPAYLLCKARFLSCFTLPALLATINP
CEERQLLSEDDEEDDHLATINPSEEHQSSTEDDEEDLQTNERSQPPTARTELNMNENEASAK
QFSGIGPKRQRNQRIKRLIK

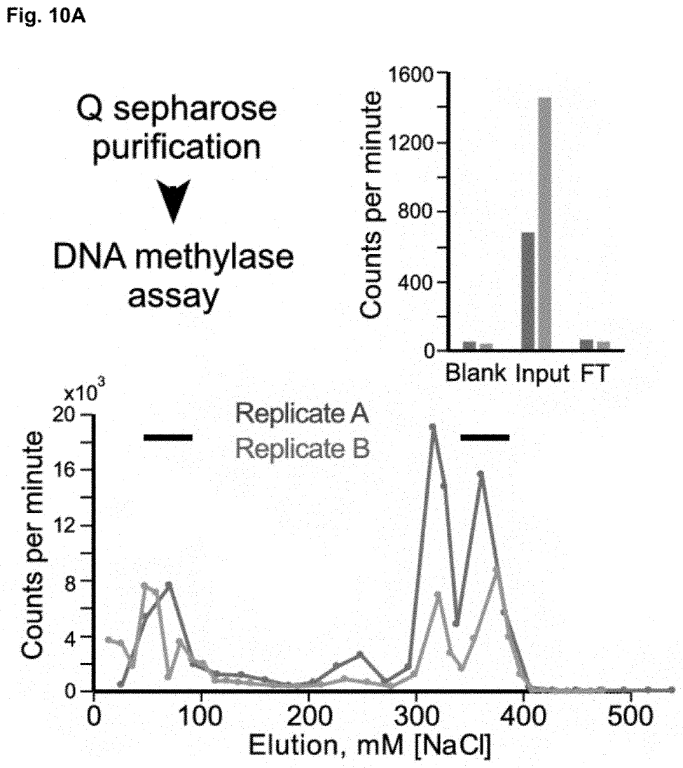

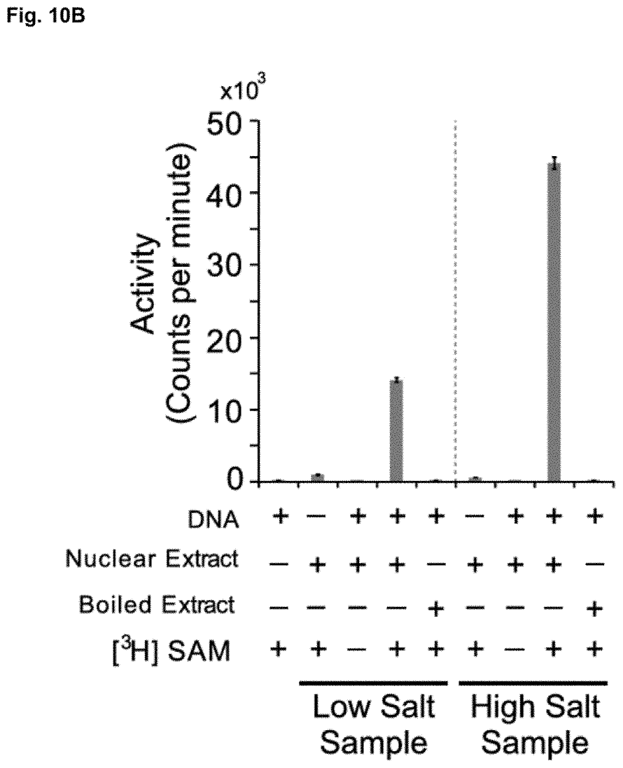

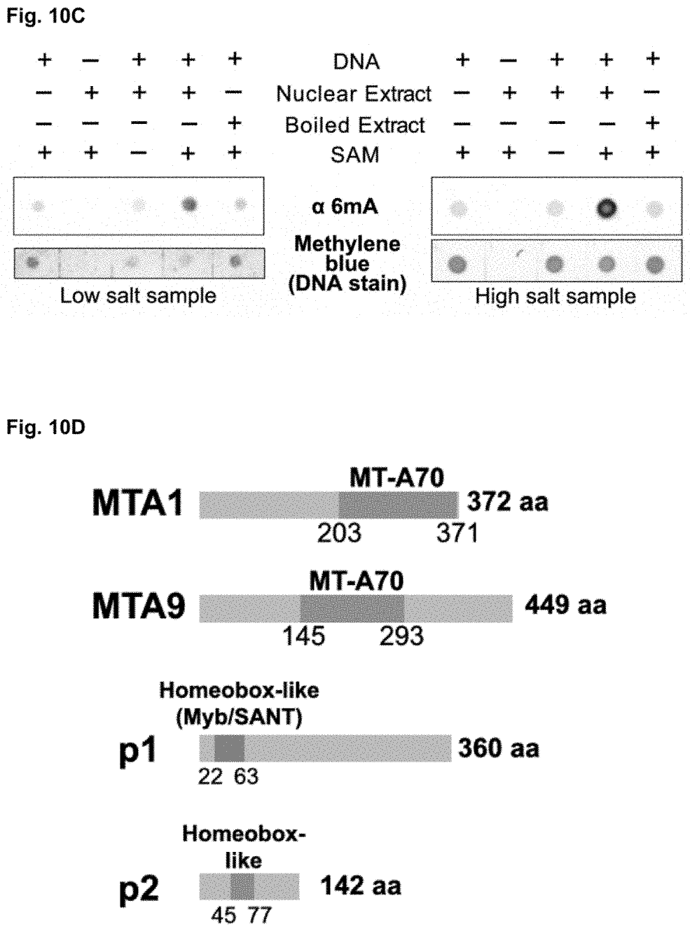

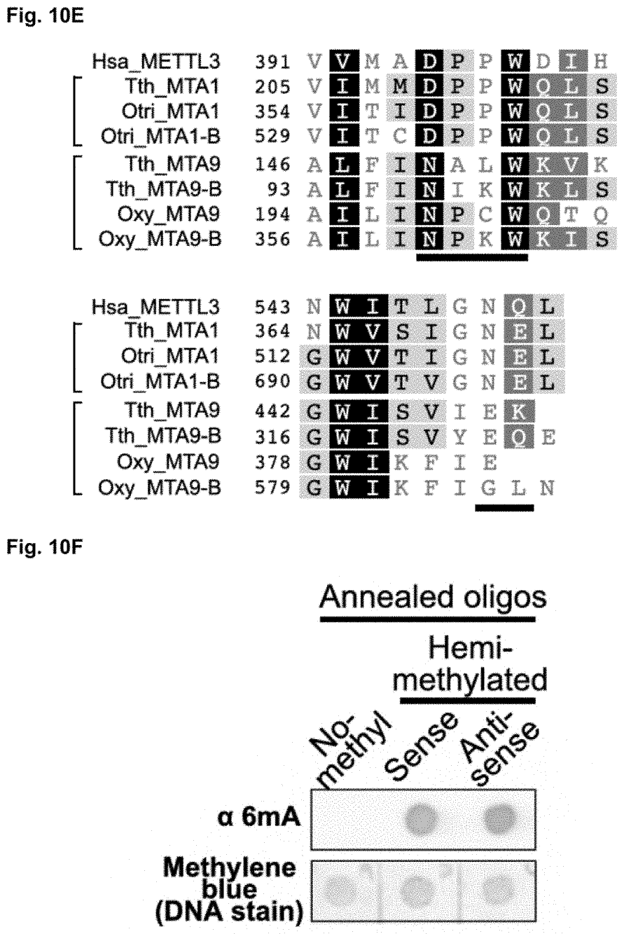

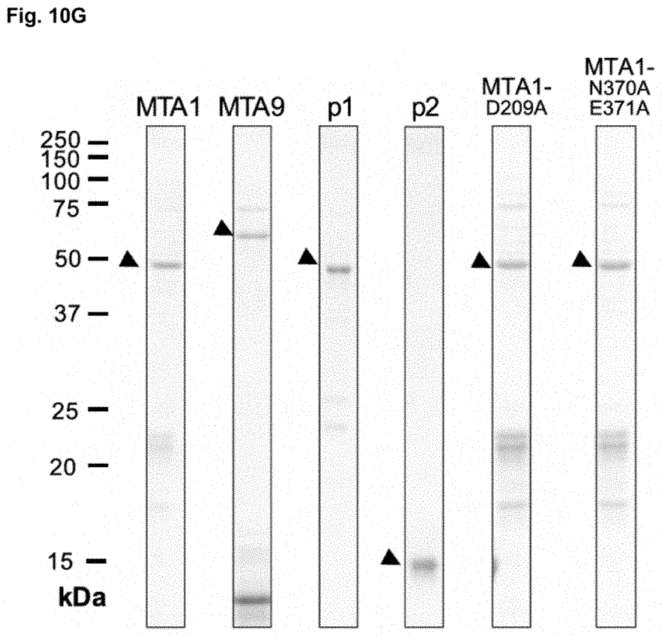

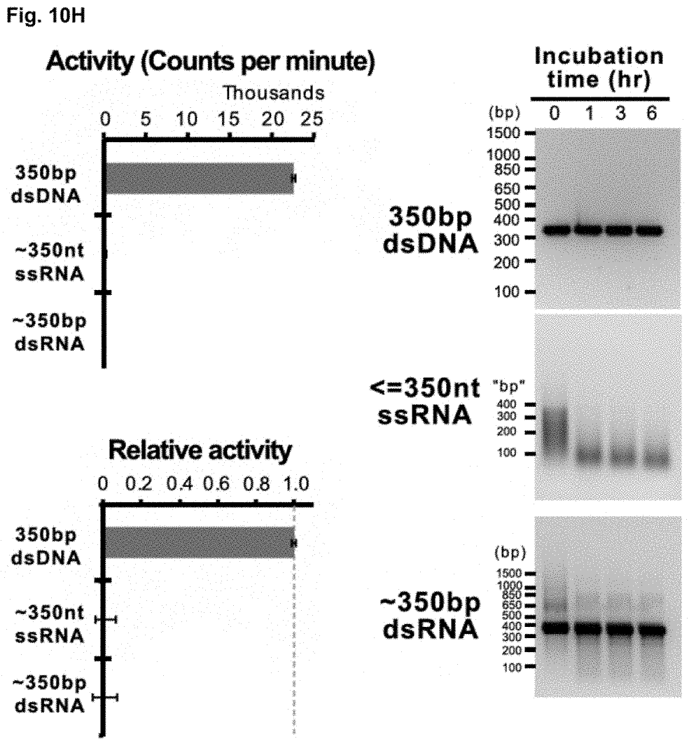

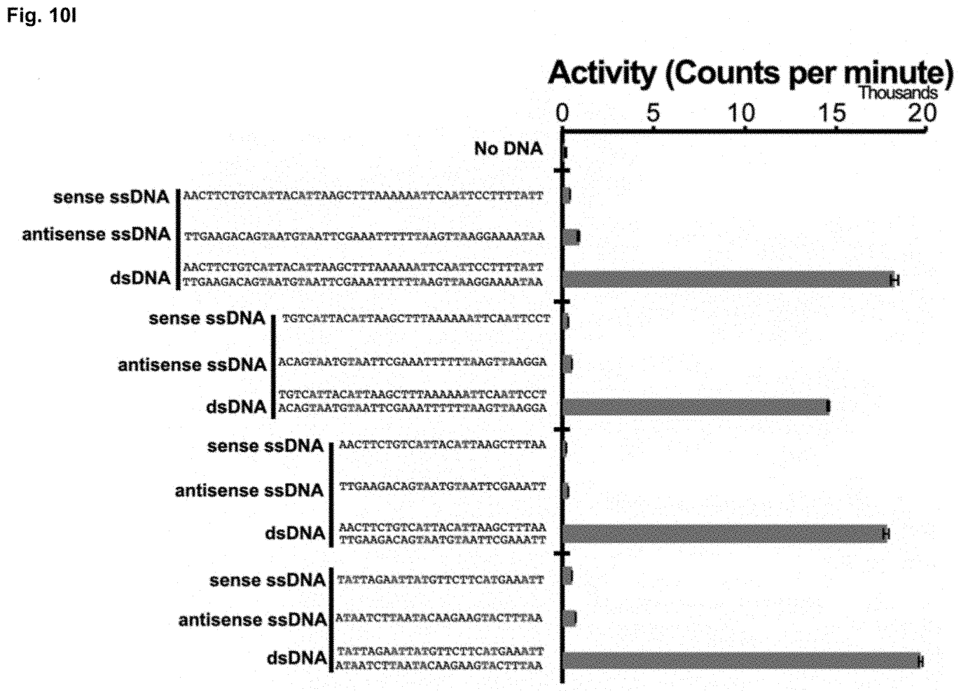

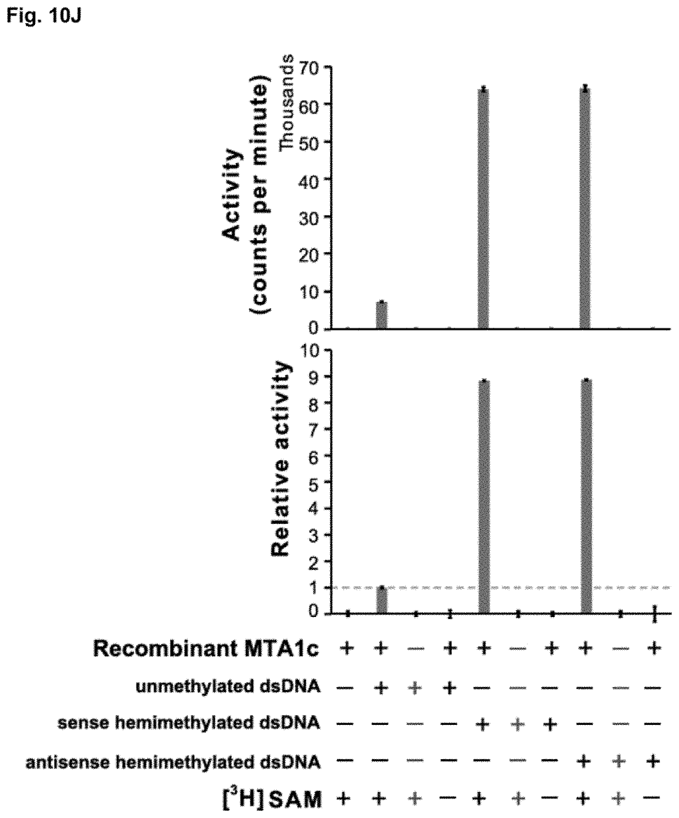

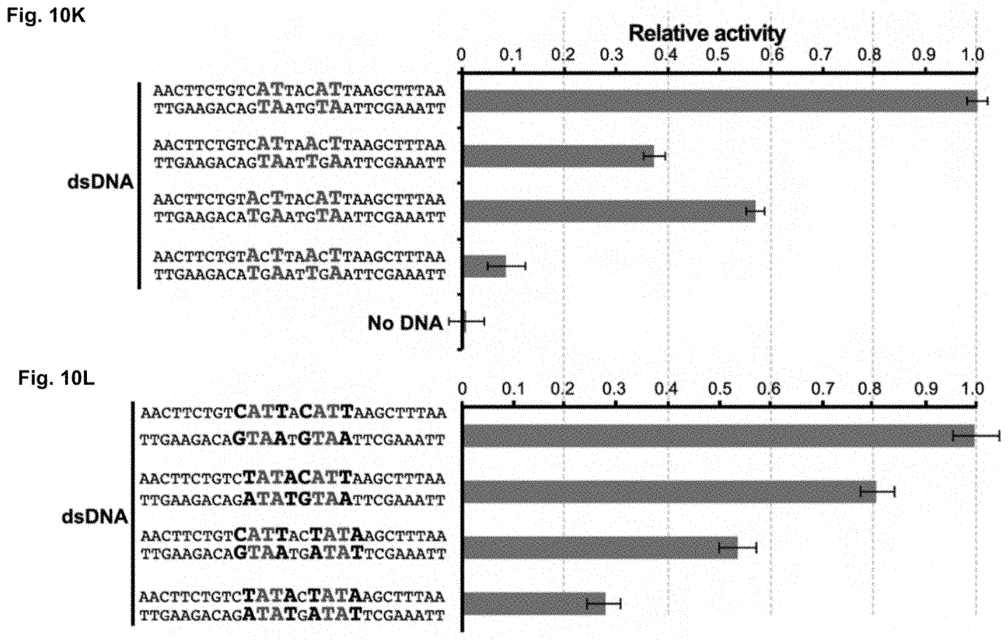

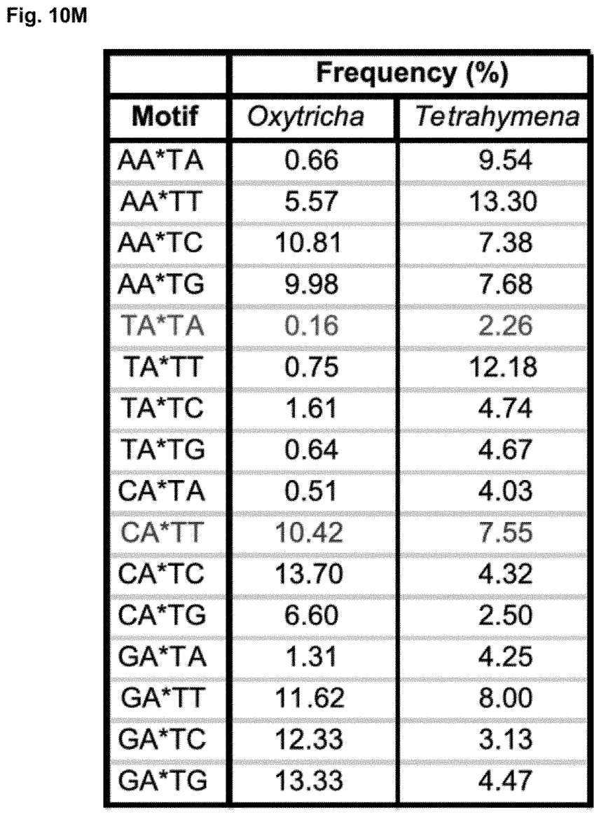

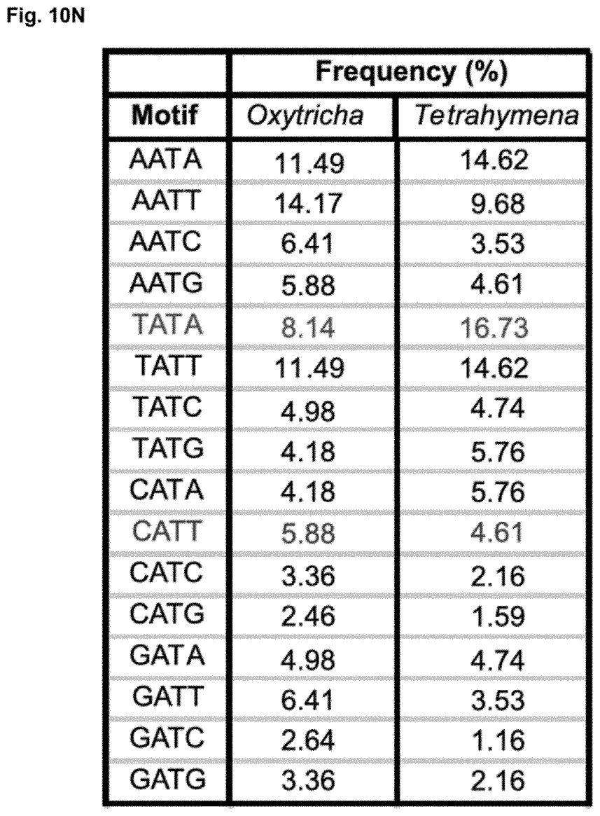

TABLE 2
Primer sequences.

| Name | Sequence (5′ to 3′) | Description |
|---|---|---|
| 1781.0_qF24 | ACTAGTCTTAAATATGAGAAAGATGATTTGAA | Contig1781.0 tiling qPCR |
| | TAAGAT (SEQ ID No: 120) | primers |
| 1781.0_qR24 | ATCCTAGCAATATTATCTACTTATAATTCTATT | Contig1781.0 tiling qPCR |
| | GACTATTAG (SEQ ID No: 121) | primers |
| 1781.0_qF23 | CTAATTAACTAATAGTCAATAGAATTATAAGT | Contig1781.0 tiling qPCR |
| | AGATAATATTGCT (SEQ ID No: 122) | primers |
| 1781.0_qR23 | CATTAAATCATTAACAGAGTAATGTCGTCATA | Contig1781.0 tiling qPCR |
| | TATTTGTC (SEQ ID No: 123) | primers |
| 1781.0_qF22 | TTTAGTGAGCATAGACAAATATATGACGACAT | Contig1781.0 tiling qPCR |
| | TACTC (SEQ ID No: 124) | primers |
| 1781.0_qR22 | GCGGAGATGTCTTTTTGACCTTTTGATAG | Contig1781.0 tiling qPCR |
| | (SEQ ID No: 125) | primers |
| 1781.0_qF21 | ATGTTAACATGCTTATTATTACTATCAAAAGG | Contig1781.0 tiling qPCR |
| | TCAA (SEQ ID No: 126) | primers |
| 1781.0_qR21 | GGCTGCTACTGATATTTATGTTCTTTATGTTT | Contig1781.0 tiling qPCR |
| | A (SEQ ID No: 127) | primers |
| 1781.0_qF20 | CAAAGAACACGAAGCTCATAAACATAAAGAA | Contig1781.0 tiling qPCR |
| | CAT (SEQ ID No: 128) | primers |
| 1781.0_qR20 | TGGAGCAAATGCTGCTAATAACGAG (SEQ ID | Contig1781.0 tiling qPCR |
| | No: 129) | primers |
| 1781.0_qF19 | ACCTCCAGCAGCTCCGTTTCTATTATTTG | Contig1781.0 tiling qPCR |
| | (SEQ ID No: 130) | primers |
| 1781.0_qR19 | GGCCTGGGTATTTTCCCTGCTTTA (SEQ ID | Contig1781.0 tiling qPCR |
| | No: 131) | primers |
| 1781.0_qF18 | CTTCCCAGGTAAAATTTAAGGTAAATAAAGCA | Contig1781.0 tiling qPCR |
| | GG (SEQ ID No: 132) | primers |
| 1781.0_qR18 | TCAAGGTGGAGGACTCTTCGGTAAC (SEQ ID | Contig1781.0 tiling qPCR |
| | No: 133) | primers |
| 1781.0_qF17 | ATTACGAACCCACTACCTGAATTATTGTTACC | Contig1781.0 tiling qPCR |
| | G (SEQ ID No: 134) | primers |
| 1781.0_qR17 | AAACGTCCTGCAGGACAACGC (SEQ ID No: | Contig1781.0 tiling qPCR |
| | 135) | primers |
| 1781.0_qF16 | TTGATTGAAGTTTTAATTTGGTACTGGGC | Contig1781.0 tiling qPCR |
| | (SEQ ID No: 136) | primers |
| 1781.0_qR16 | TTGGATGCTGATCTGTTTTGTTTAGAAAG | Contig1781.0 tiling qPCR |
| | (SEQ ID No: 137) | primers |
| 1781.0_qF15 | TTGGGATTTCTTAACTGGATTTCTTTCTAAAC | Contig1781.0 tiling qPCR |
| | (SEQ ID No: 138) | primers |
| 1781.0_qR15 | CTGCTTAAATTAAGTACTTCTATGTTTGAAAT | Contig1781.0 tiling qPCR |
| | TAATGTTC (SEQ ID No: 139) | primers |
| 1781.0_qF14 | CAATTAAAACACGTTGAACATTAATTTCAAAC | Contig1781.0 tiling qPCR |
| | ATAG (SEQ ID No: 140) | primers |
| 1781.0_qR14 | TGAGGATCCAAGGTAAATTTCATACAATC | Contig1781.0 tiling qPCR |
| | (SEQ ID No: 141) | primers |
| 1781.0_qF13 | GACTGCATGTATATGCTAATGATTGTATGAAA | Contig1781.0 tiling qPCR |
| | TTTAC (SEQ ID No: 142) | primers |
| 1781.0_qR13 | AGTGGCATTTCCAAGGAAACATTAATAC | Contig1781.0 tiling qPCR |
| | (SEQ ID No: 143) | primers |
| 1781.0_qF12 | CAGTGTTTCCCTTTGTGTAAATGGG (SEQ ID | Contig1781.0 tiling qPCR |
| | No: 144) | primers |
| 1781.0_qR12 | TCAGTGGATAAACTAGCCTAAGGAAACAC | Contig1781.0 tiling qPCR |
| | (SEQ ID No: 145) | primers |
| 1781.0_qF11 | TTTTACAGACTGGACACAGTAGTGTTTCC | Contig1781.0 tiling qPCR |
| | (SEQ ID No: 146) | primers |
| 1781.0_qR11 | CCAGTGGTATCAACATGCGGTCATC (SEQ ID | Contig1781.0 tiling qPCR |
| | No: 147) | primers |
| 1781.0_qF10 | GATATATACACTCCCAGCAGTAAAGATGACC | Contig1781.0 tiling qPCR |
| | (SEQ ID No: 148) | primers |
| 1781.0_qR10 | GAATAGGCTCACTCTAAATTCGAGTGC (SEQ | Contig1781.0 tiling qPCR |
| | ID No: 149) | primers |
| 1781.0_qF9 | ATTCGCTAGGTCTAAGCAAATATTGCAC | Contig1781.0 tiling qPCR |
| | (SEQ ID No: 150) | primers |
| 1781.0_qR9 | TAAATAGCCAAAACAACCAATAAAATTAACAA | Contig1781.0 tiling qPCR |
| | TAACCTC (SEQ ID No: 151) | primers |
| 1781.0_qF8 | CTTTTTGAGGGCGAGGTTATTGTTAATTTTAT | Contig1781.0 tiling qPCR |
| | TG (SEQ ID No: 152) | primers |
| 1781.0_qR8 | GATCCATTAATTACAGAAATAAATAATAGGCA | Contig1781.0 tiling qPCR |
| | GCATA (SEQ ID No: 153) | primers |
| 1781.0_qF7 | ATATTGCCTGAATTATTATGCTGCCTATTATT | Contig1781.0 tiling qPCR |
| | TATT (SEQ ID No: 154) | primers |
| 1781.0_qR7 | AAATGTGCACCGTCATCAAATACC (SEQ ID | Contig1781.0 tiling qPCR |
| | No: 155) | primers |
| 1781.0_qF6 | GGATCACTATAATCATCTGGATGACTATTGG | Contig1781.0 tiling qPCR |
| | (SEQ ID No: 156) | primers |
| 1781.0_qR6 | AAGTGTAATGTAGTTTCAATGGTAGTGATGT | Contig1781.0 tiling qPCR |
| | G (SEQ ID No: 157) | primers |
| 1781.0_qF5 | TGACTTCTTCCAGTGGATTCACATC (SEQ ID | Contig1781.0 tiling qPCR |
| | No: 158) | primers |
| 1781.0_qR5 | GCCAATTAATTCATTTGTTCGTAGAGATATGT | Contig1781.0 tiling qPCR |
| | AA (SEQ ID No: 159) | primers |
| 1781.0_qF4 | CACTTTATAATAAATAAGAATTATTACATATCT | Contig1781.0 tiling qPCR |
| | CTACGAACAA (SEQ ID No: 160) | primers |
| 1781.0_qR4 | CTCACCAGTAATTTGCAGACACC (SEQ ID | Contig1781.0 tiling qPCR |
| | No: 161) | primers |
| 1781.0_qF3 | GGCTGACTGGGGTTGAGTTAATC (SEQ ID | Contig1781.0 tiling qPCR |
| | No: 162) | primers |
| 1781.0_qR3 | AATATAAACAAAATGGAATATACAAAACTTGA | Contig1781.0 tiling qPCR |
| | ATAAGAAATAG (SEQ ID No: 163) | primers |
| 1781.0_qF2 | GAGACTGAGGATCTATTTCTTATTCAAGTTTT | Contig1781.0 tiling qPCR |
| | G (SEQ ID No: 164) | primers |
| 1781.0_qR2 | ATTAATACATTATTAACTTAAATATAAATATTT | Contig1781.0 tiling qPCR |
| | AAAGAATTATGAACAATAAT (SEQ ID | primers |
| | No: 165) | |
| 1781.0_qF1 | CATTTTGTTTATATTATTGTTCATAATTCTTTA | Contig1781.0 tiling qPCR |
| | AATATTTATATTTAAGTTAAT(SEQ ID | primers |
| | No: 166) | |
| 1781.0_qR1 | ACAAGATAACATTGCTAATTTTCAATAAATTA | Contig1781.0 tiling qPCR |
| | AATTAATACATT (SEQ ID No: 167) | primers |
| 1781.0_F | CCCCAAAACCCCAAAACCCCACTAGTCTTAA | Primer pair for amplifying |
| | ATATGAGAAAGATGATTTGAATAAG (SEQ ID | chromosome, to be added to |
| | No: 168) | mini-genome |
| 1781.0_R | CCCCAAAACCCCAAAACCCCACAAGATAACA | |
| | TTGCTAATTTTCAATAAATTAAAT (SEQ ID | |
| | No: 169) | |
| 15118.0_F | CCCCAAAACCCCAAAACCCCGATTTATGAAA | Primer pair for amplifying |
| | GTGCTGTATTATTAAGGAATG (SEQ ID No: | chromosome, to be added to |
| | 170) | mini-genome |
| 15118.0_R | CCCCAAAACCCCAAAACCCCATTATTCCTAC | |
| | TTTTAGCTATATTAGAAATTCG (SEQ ID No: | |
| | 171) | |
| 1339.1_F | CCCCAAAACCCCAAAACCCCATGATGATACA | Primer pair for amplifying |
| | TAGATTCATTAAAATAAAAAAAAG (SEQ ID | chromosome, to be added to |
| | No: 172) | mini-genome |
| 1339.1_R | CCCCAAAACCCCAAAACCCCTTAGATGAATT | |
| | AAATAAAGAATTCAAATAAATAC (SEQ ID | |
| | No: 173) | |
| 20718.0_F | CCCCAAAACCCCAAAACCCCATGAATCTGAA | Primer pair for amplifying |
| | ATCGGGCAGTTGAATACG (SEQ ID | chromosome, to be added to |
| | No: 174) | mini-genome |
| 20718.0_R | CCCCAAAACCCCAAAACCCCATTTATCATAAT | |
| | TATAGAGAAGATAGTGATGC (SEQ ID No: | |
| | 175) | |
| 20822.0_F | CCCCAAAACCCCAAAACCCCATGAGAGTTTG | Primer pair for amplifying |
| | TGAAAAATTAAGTTTG (SEQ ID No: | chromosome, to be added to |
| | 176) | mini-genome |
| 20822.0_R | CCCCAAAACCCCAAAACCCCTATATTAAATAT | |
| | CAAGAAAAAGTAAAAAGACAG (SEQ ID No: | |
| | 177) | |
| 21162.0_F | CCCCAAAACCCCAAAACCCCAAGTCTCATTT | Primer pair for amplifying |
| | TGGTTAGTGATGTTTGGATTG (SEQ ID No: | chromosome, to be added to |
| | 178) | mini-genome |
| 21162.0_R | CCCCAAAACCCCAAAACCCCGTATGATCGAT | |
| | GAATACAAAATCAAGTTGGAAG (SEQ ID No: | |
| | 179) | |
| 11991.0_F | CCCCAAAACCCCAAAACCCCACTTAAAAGGA | Primer pair for amplifying |
| | TTGCATGATTGTAAGGGAAATGTG (SEQ ID | chromosome, to be added to |
| | No: 180) | mini-genome |
| 11991.0_R | CCCCAAAACCCCAAAACCCCAATAATCGCAC | |
| | TTACATTATATCTGGAGAAATG (SEQ ID No: | |
| | 181) | |
| 5079.0_F | CCCCAAAACCCCAAAACCCCTTCTACTAAATT | Primer pair for amplifying |
| | TCATTGATTTTTTTCAATTTC (SEQ ID | chromosome, to be added to |
| | No: 182) | mini-genome |
| 5079.0_R | CCCCAAAACCCCAAAACCCCATTTGATAGAA | |
| | TAGAAGAGAAATTATGGAATG (SEQ ID No: | |
| | 183) | |
| 13665.0_F | CCCCAAAACCCCAAAACCCCAAGTATAAATA | Primer pair for amplifying |
| | AGGGAGTTGATATATAATATACTT (SEQ ID | chromosome, to be added to |
| | No: 184) | mini-genome |
| 13665.0_R | CCCCAAAACCCCAAAACCCCATGAGAATTCC | |
| | TATTCAAAAATGAAAAAGTAGATTG (SEQ ID | |
| | No: 185) | |
| 22365.0_F | CCCCAAAACCCCAAAACCCCATAAGGTAGTA | Primer pair for amplifying |
| | TATTTTTATTAAGGATTGGAAATTA (SEQ ID | chromosome, to be added to |
| | No: 186) | mini-genome |
| 22365.0_R | CCCCAAAACCCCAAAACCCCATAAGACTAAA | |
| | TTTATTGAAATTATCTTGTTAATAG (SEQ ID | |
| | No: 187) | |
| 21620.0_F | CCCCAAAACCCCAAAACCCCTTGAGCCAATA | Primer pair for amplifying |
| | CTGAAAAGGATGATAGTGAATAGTG (SEQ ID | chromosome, to be added to |
| | No: 188) | mini-genome |
| 21620.0_R | CCCCAAAACCCCAAAACCCCTCATTTTTTAAA | |
| | TTGGATAGTAAGAAAAATTATAATAAAG (SEQ | |
| | ID No: 189) | |
| 15049.0_F | CCCCAAAACCCCAAAACCCCAAGGAATAAAA | Primer pair for amplifying |
| | TTCAATTCCAAAATGTAAGGTGAG (SEQ ID | chromosome, to be added to |
| | No: 190) | mini-genome |
| 15049.0_R | CCCCAAAACCCCAAAACCCCGTTAAAAGAAC | |
| | CAAGTGATATATTATAAGCCA (SEQ ID No: | |
| | 191) | |
| 16562.0_F | CCCCAAAACCCCAAAACCCCTTTATCAATTAT | Primer pair for amplifying |
| | AAATAAAAAGTTTTAAGTCTATTTTTAA (SEQ | chromosome, to be added to |
| | ID No: 192) | mini-genome |
| 16562.0_R | CCCCAAAACCCCAAAACCCCATAAGACAAAT | |
| | GCAACTTTATAAAGTAAATAAATTATC (SEQ | |
| | ID No: 193) | |
| 22360.0_F | CCCCAAAACCCCAAAACCCCAATGCAACATT | Primer pair for amplifying |
| | TACTTTTAACATTAGAGATTATC (SEQ ID | chromosome, to be added to |
| | No: 194) | mini-genome |
| 22360.0_R | CCCCAAAACCCCAAAACCCCATAAGAGCAAA | |
| | AGTTAATATAAAAATTCAAGGTG (SEQ ID | |
| | No: 195) | |
| 15836.0_F | CCCCAAAACCCCAAAACCCCGATTTGCACAG | Primer pair for amplifying |
| | TTAATTTGAATTTGGTATTTG (SEQ ID No: | chromosome, to be added to |
| | 196) | mini-genome |
| 15836.0_R | CCCCAAAACCCCAAAACCCCTCATTTTTAGTA | |
| | TTTTAAATATCATTTAGTTTTAAGTAA (SEQ | |
| | ID No: 197) | |
| 2324.0_F | CCCCAAAACCCCAAAACCCCTTGATTGATTC | Primer pair for amplifying |
| | CTGAATACAAATGAAATAATATAAAG (SEQ ID | chromosome, to be added to |
| | No: 198) | mini-genome |
| 2324.0_R | CCCCAAAACCCCAAAACCCCAAGACCAAAAT | |
| | AAAGAGGAATAATGAGAAGTAC (SEQ ID No: | |
| | 199) | |
| 22404.0_F | CCCCAAAACCCCAAAACCCCATGTAGAATTA | Primer pair for amplifying |
| | ATATGAGAACATCATTTTTTAAGC (SEQ ID | chromosome, to be added to |
| | No: 200) | mini-genome |
| 22404.0_R | CCCCAAAACCCCAAAACCCCATAATGTAAGA | |
| | AATCTGATACAATAGAGAGATAAAC (SEQ ID | |
| | No: 201) | |
| 15403.0_F | CCCCAAAACCCCAAAACCCCGAATGGAAAAT | Primer pair for amplifying |
| | TTGTATGAAGTTCAGAGAGAAAG (SEQ ID | chromosome, to be added to |
| | No: 202) | mini-genome |
| 15403.0_R | CCCCAAAACCCCAAAACCCCATAAGATTATC | |
| | AGTTATAAAAATTGATAGGGGATG (SEQ ID | |
| | No: 203) | |
| 17795.0_F | CCCCAAAACCCCAAAACCCCATCATACGATA | Primer pair for amplifying |
| | TCTTAAGTGTTGATCTGAATTAAAT (SEQ ID | chromosome, to be added to |
| | No: 204) | mini-genome |
| 17795.0_R | CCCCAAAACCCCAAAACCCCGTTAGGTTTAA | |
| | GAGTAGAAATAAAAGGAGATAAG (SEQ ID | |
| | No: 205) | |
| 11141.0_F | CCCCAAAACCCCAAAACCCCTCTCACTATCT | Primer pair for amplifying |
| | TTTGTAAAAAGTTGGTAGAT (SEQ ID | chromosome, to be added to |
| | No: 206) | mini-genome |
| 11141.0_R | CCCCAAAACCCCAAAACCCCGTTGGTTTAGA | |
| | ATAAAGAATTGTATTAACCAAATTTAT (SEQ | |
| | ID No: 207) | |
| 22342.0_F | CCCCAAAACCCCAAAACCCCGTGAATTAAAA | Primer pair for amplifying |
| | TATAAACGAATAAGATATAAAGATTG (SEQ ID | chromosome, to be added to |
| | No: 208) | mini-genome |
| 22342.0_R | CCCCAAAACCCCAAAACCCCTTAATTACTGA | |
| | ATTGTTTATTATAAGATTATAAG (SEQ ID | |
| | No: 209) | |
| 2240.0_F | CCCCAAAACCCCAAAACCCCGTAATGAATAA | Primer pair for amplifying |
| | ATTGTAAAGGTAAATTGCAA (SEQ ID | chromosome, to be added to |
| | No: 210) | mini-genome |
| 2240.0_R | CCCCAAAACCCCAAAACCCCAATGGCAAACA | |
| | TTTAAAATAAATATTAATATAAATTAC (SEQ | |
| | ID No: 211) | |
| 3531.0_F | CCCCAAAACCCCAAAACCCCTAAAAGGAAAA | Primer pair for amplifying |
| | CAAATAGAAGAAACTGAA (SEQ ID No: | chromosome, to be added to |
| | 212) | mini-genome |
| 3531.0_R | CCCCAAAACCCCAAAACCCCATTTGGATATT | |
| | ATGATTAGCAGTTTAGTG (SEQ ID No: | |
| | 213) | |
| 4701.0_F | CCCCAAAACCCCAAAACCCCTTTAAATAAAAA | Primer pair for amplifying |
| | TCGCATGAATTAAATGCAAG (SEQ ID | chromosome, to be added to |
| | No: 214) | mini-genome |
| 4701.0_R | CCCCAAAACCCCAAAACCCCTAGGTAAATGC | |
| | AAATTGGAGAATTTCCAATAG (SEQ ID No: | |
| | 215) | |
| 20883.0_F | CCCCAAAACCCCAAAACCCCATATTAAGAAT | Primer pair for amplifying |
| | TGTGTAATTTTTGAGTAAATTG (SEQ ID No: | chromosome, to be added to |
| | 216) | mini-genome |
| 20883.0_R | CCCCAAAACCCCAAAACCCCATTTAGTAGAA | |
| | TCTTCAATAAATAAGCGTTATTG (SEQ ID | |
| | No: 217) | |
| 15191.0_F | CCCCAAAACCCCAAAACCCCTAGCATTAAAT | Primer pair for amplifying |
| | TTGTAAAAAGAATGAAATTTAATAT (SEQ ID | chromosome, to be added to |
| | No: 218) | mini-genome |
| 15191.0_R | CCCCAAAACCCCAAAACCCCAATATACATGA | |
| | TTTTAGATAAACAACAAATAAT (SEQ ID No: | |
| | 219) | |
| 19342.0_F | CCCCAAAACCCCAAAACCCCATCAAGAATGG | Primer pair for amplifying |
| | ATTAGAATTTTTAATGCTTTGC (SEQ ID No: | chromosome, to be added to |
| | 220) | mini-genome |
| 19342.0_R | CCCCAAAACCCCAAAACCCCGAGGAACTAG | |
| | GGATTACTCATTTTACTTCAG (SEQ ID No: | |
| | 221) | |
| 15245.0_F | CCCCAAAACCCCAAAACCCCATGCATGTAAT | Primer pair for amplifying |
| | TTTCTGTCAAAATTGAGTAAATAG (SEQ ID | chromosome, to be added to |
| | No: 222) | mini-genome |
| 15245.0_R | CCCCAAAACCCCAAAACCCCGTAAGCTAAAT | |
| | AAGTAGACTAAATAGGTAG (SEQ ID | |
| | No: 223) | |
| 6109.0_F | CCCCAAAACCCCAAAACCCCAACCGCAAATA | Primer pair for amplifying |
| | GAATATATAAAGGATAATTTA (SEQ ID No: | chromosome, to be added to |
| | 224) | mini-genome |
| 6109.0_R | CCCCAAAACCCCAAAACCCCGAAGTACTAAA | |
| | AATAAAAAGTAAAGTATTAAAATAAAATC | |
| | (SEQ ID No: 225) | |
| 22610.0_F | CCCCAAAACCCCAAAACCCCGTAGACAGATT | Primer pair for amplifying |
| | TTCCAGTTTATAGCTGTGTTTG (SEQ ID No: | chromosome, to be added to |
| | 226) | mini-genome |
| 22610.0_R | CCCCAAAACCCCAAAACCCCTTTATGAATTTT | |
| | CTTAAATCTGTAAATAAATAAAATAAT (SEQ | |
| | ID No: 227) | |
| 11875.0_F | CCCCAAAACCCCAAAACCCCGTATGTTAATT | Primer pair for amplifying |
| | TTATGCTTTAAATGATAGTTTA (SEQ ID No: | chromosome, to be added to |
| | 228) | mini-genome |
| 11875.0_R | CCCCAAAACCCCAAAACCCCTGGATTCCATT | |
| | TTGAAGAATAATTTATTAAC (SEQ ID | |
| | No: 229) | |
| 15329.0_F | CCCCAAAACCCCAAAACCCCTTGTTTCGATT | Primer pair for amplifying |
| | ATATTCAAAATAGGAAATTTAG (SEQ ID No: | chromosome, to be added to |
| | 230) | mini-genome |
| 15329.0_R | CCCCAAAACCCCAAAACCCCATGAATTTCAA | |
| | TAACTTTTTATGAAAATGAATTTA (SEQ ID | |
| | No: 231) | |
| 20179.0_F | CCCCAAAACCCCAAAACCCCTAGGAAGAAAA | Primer pair for amplifying |
| | TCTTGTGTGCAATTTGAGATTAAC (SEQ ID | chromosome, to be added to |
| | No: 232) | mini-genome |
| 20179.0_R | CCCCAAAACCCCAAAACCCCTTGATAAAAAC | |
| | ATAGATTAAATACTAGTGTATAAA (SEQ ID | |
| | No: 233) | |
| 9936.0_F | CCCCAAAACCCCAAAACCCCATATGGAATAT | Primer pair for amplifying |
| | TTAATTTGATTTAAATGAAACGAAATA (SEQ | chromosome, to be added to |
| | ID No: 234) | mini-genome |
| 9936.0_R | CCCCAAAACCCCAAAACCCCTTGTAACAGTA | |
| | AATAGAATATTTTAATTACCAAAAC (SEQ ID | |
| | No: 235) | |
| 16267.0_F | CCCCAAAACCCCAAAACCCCTCATTTTAGAA | Primer pair for amplifying |
| | TTATCTGTACTTAATTATTTTG (SEQ ID No: | chromosome, to be added to |
| | 236) | mini-genome |
| 16267.0_R | CCCCAAAACCCCAAAACCCCATGAGCATGTT | |
| | ATTTTACTTCATTAGTCAATTTG (SEQ ID | |
| | No: 237) | |
| 4488.0_F | CCCCAAAACCCCAAAACCCCATGAAATGAAT | Primer pair for amplifying |
| | TCTAAGATTGAATTGCATG (SEQ ID | chromosome, to be added to |
| | No: 238) | mini-genome |
| 4488.0_R | CCCCAAAACCCCAAAACCCCAGAAGAGATCA | |
| | ATAAATTGAGAAGGAATTG (SEQ ID | |
| | No: 239) | |
| 8551.0_F | CCCCAAAACCCCAAAACCCCGTGTTACAATT | Primer pair for amplifying |
| | TGCGTTTGAAATAGTTGGTTGATA (SEQ ID | chromosome, to be added to |
| | No: 240) | mini-genome |
| 8551.0_R | CCCCAAAACCCCAAAACCCCATATGGTAAAA | |
| | ATTGAAGAAAGAAATTCAAGAGAA (SEQ ID | |
| | No: 241) | |
| 11746.0_F | CCCCAAAACCCCAAAACCCCGTATTGATGAT | Primer pair for amplifying |
| | AAAATTGTATACAAGTTGATAG (SEQ ID No: | chromosome, to be added to |
| | 242) | mini-genome |
| 11746.0_R | CCCCAAAACCCCAAAACCCCTAGATGCTTAA | |
| | TTATTAAGAAGATTCTGGAATG (SEQ ID No: | |
| | 243) | |
| 22291.0_F | CCCCAAAACCCCAAAACCCCATAAACCAATG | Primer pair for amplifying |
| | TAATTAATTTATTGGGTGTGTTG (SEQ ID | chromosome, to be added to |
| | No: 244) | mini-genome |
| 22291.0_R | CCCCAAAACCCCAAAACCCCTTAGATTAAATT | |
| | TAGAGAGTTATAGAAATGTAGTAAAT (SEQ ID | |
| | No: 245) | |
| 17535.0_F | CCCCAAAACCCCAAAACCCCATCTCAATTTAT | Primer pair for amplifying |
| | AAAATCAGAATAAGAGATTGTC (SEQ ID No: | chromosome, to be added to |
| | 246) | mini-genome |
| 17535.0_R | CCCCAAAACCCCAAAACCCCAGAATAAAACA | |
| | ACTGAAGTAAATATGAGTTAC (SEQ ID No: | |
| | 247) | |
| 15372.0_F | CCCCAAAACCCCAAAACCCCTTTCAAATATAA | Primer pair for amplifying |
| | AATAAACAGAAGAATGGCAAACG (SEQ ID | chromosome, to be added to |
| | No: 248) | mini-genome |
| 15372.0_R | CCCCAAAACCCCAAAACCCCAAATTCAATATT | |
| | AAATGAAATAATTTTCAAAAGTG (SEQ ID | |
| | No: 249) | |
| 13537.0_F | CCCCAAAACCCCAAAACCCCATGAGATCAAA | Primer pair for amplifying |
| | TTTTTTTATTAAAATTCTTC (SEQ ID | chromosome, to be added to |
| | No: 250) | mini-genome |
| 13537.0_R | CCCCAAAACCCCAAAACCCCTTGGATTCATA | |
| | TTTTTGTTTAAGGCTTAGATA (SEQ ID No: | |
| | 251) | |
| 22613.0_F | CCCCAAAACCCCAAAACCCCATTAGAAAAGA | Primer pair for amplifying |
| | GGATTTCAATAAAAGCAAATAT (SEQ ID No: | chromosome, to be added to |
| | 252) | mini-genome |
| 22613.0_R | CCCCAAAACCCCAAAACCCCATCGATTTATT | |
| | ATTGTTGAATTTAAAAGTATTGAA (SEQ ID | |
| | No: 253) | |
| 12585.0_F | CCCCAAAACCCCAAAACCCCGAGAGGTTTGA | Primer pair for amplifying |
| | TAAGTAGAATTAGTAAAATCTATAAAG (SEQ | chromosome, to be added to |
| | ID No: 254) | mini-genome |
| 12585.0_R | CCCCAAAACCCCAAAACCCCATTAGTACTAT | |
| | TTTCATAGATCTATGTATAAATTGAA (SEQ ID | |
| | No: 255) | |
| 5317.0_F | CCCCAAAACCCCAAAACCCCAATGGAAAGAT | Primer pair for amplifying |
| | AAACAGATTTTAATTTGGAAATAAAAT (SEQ | chromosome, to be added to |
| | ID No: 256) | mini-genome |
| 5317.0_R | CCCCAAAACCCCAAAACCCCTTTAAGCAGTA | |
| | TTTCTAAAATGTTGATGAAATAAAAAT (SEQ | |
| | ID No: 257) | |
| 17894.0_F | CCCCAAAACCCCAAAACCCCATAAGATAAAA | Primer pair for amplifying |
| | TTTAACGAAAAAAAGTTAAGTC (SEQ ID No: | chromosome, to be added to |
| | 258) | mini-genome |
| 17894.0_R | CCCCAAAACCCCAAAACCCCATAAGATGAAA | |
| | TATAGAGATAATTGAGCCTA (SEQ ID | |
| | No: 259) | |
| 3513.0_F | CCCCAAAACCCCAAAACCCCAATTACATATTA | Primer pair for amplifying |
| | ATGTACTTATGATAGAATG (SEQ ID | chromosome, to be added to |
| | No: 260) | mini-genome |
| 3513.0_R | CCCCAAAACCCCAAAACCCCTAATGATCAAA | |
| | TAACCTGAGTTAAAGAAG (SEQ ID | |
| | No: 261) | |
| 16420.0_F | CCCCAAAACCCCAAAACCCCAAATTATGAAA | Primer pair for amplifying |
| | ATAGACACTAATTGGATGTTC (SEQ ID No: | chromosome, to be added to |
| | 262) | mini-genome |
| 16420.0_R | CCCCAAAACCCCAAAACCCCTGATTCGTCAT | |
| | ATGAAATTGAAAAGGAGTAAAT (SEQ ID No: | |
| | 263) | |
| 1084.1_F | CCCCAAAACCCCAAAACCCCAGCGCCATGAA | Primer pair for amplifying |
| | TCTGATGCATTTATTTTAAG (SEQ ID | chromosome, to be added to |
| | No: 264) | mini-genome |
| 1084.1_R | CCCCAAAACCCCAAAACCCCGTAGATCATTT | |
| | ATGTAAAAGATTTTGAGAGATG (SEQ ID No: | |
| | 265) | |
| 22651.0_F | CCCCAAAACCCCAAAACCCCATACAATTATTA | Primer pair for amplifying |
| | TAAATGAAAAAGCGCACTAATC (SEQ ID No: | chromosome, to be added to |
| | 266) | mini-genome |
| 22651.0_R | CCCCAAAACCCCAAAACCCCATAGTTACTAT | |
| | GAAAGGACTGGTACATAGAAATAATAG (SEQ | |
| | ID No: 267) | |
| 8670.0_F | CCCCAAAACCCCAAAACCCCTTAAGTCAATA | Primer pair for amplifying |
| | TCTAAATCAAATATTAGTAGTATAAT (SEQ ID | chromosome, to be added to |
| | No: 268) | mini-genome |
| 8670.0_R | CCCCAAAACCCCAAAACCCCGTCATATGGTT | |
| | TTATAAAATAAAATTGAGATTTTTTTG (SEQ | |
| | ID No: 269) | |
| 19107.0_F | CCCCAAAACCCCAAAACCCCATAAGGATAAA | Primer pair for amplifying |
| | TTCTATCATATAAGTGGAAGTGC (SEQ ID | chromosome, to be added to |
| | No: 270) | mini-genome |
| 19107.0_R | CCCCAAAACCCCAAAACCCCATTCTTGAATA | |
| | TTGATTATGCATATTGTGTAAAATAG (SEQ ID | |
| | No: 271) | |
| 21021.0_F | CCCCAAAACCCCAAAACCCCAAGCGTTGAAT | Primer pair for amplifying |
| | TTTTTATAATATATGATAAAC (SEQ ID | chromosome, to be added to |
| | No: 272) | mini-genome |
| 21021.0_R | CCCCAAAACCCCAAAACCCCTTAATGCCAAT | |
| | AAACAGATGAAAGTAGAGTTATAG (SEQ ID | |
| | No: 273) | |
| 15004.0_F | CCCCAAAACCCCAAAACCCCATAGAGAGTGT | Primer pair for amplifying |
| | TTTATTGAAGGACAGAGAATATTG (SEQ ID | chromosome, to be added to |
| | No: 274) | mini-genome |
| 15004.0_R | CCCCAAAACCCCAAAACCCCGAGCGTAAGAA | |
| | ATATTCTTAGATAAATGGAAACTG (SEQ ID | |
| | No: 275) | |
| 18789.0_F | CCCCAAAACCCCAAAACCCCATGGCAATATC | Primer pair for amplifying |
| | TTTGCGTGTTTCTGGC (SEQ ID | chromosome, to be added to |
| | No: 276) | mini-genome |
| 18789.0_R | CCCCAAAACCCCAAAACCCCATAAGAATAAA | |
| | TTAAAGAAGATTTGAGAAAGATATGC (SEQ ID | |
| | No: 277) | |
| 1335.1_F | CCCCAAAACCCCAAAACCCCAAATGCTAAAA | Primer pair for amplifying |
| | ATAATGAAAAATCTGAGGG (SEQ ID | chromosome, to be added to |
| | No: 278) | mini-genome |
| 1335.1_R | CCCCAAAACCCCAAAACCCCTAATGACAGGT | |
| | TTAGTAATAATTTAGCTG (SEQ ID | |
| | No: 279) | |
| 17286.0_F | CCCCAAAACCCCAAAACCCCACGACTTAACA | Primer pair for amplifying |
| | TTGCTGTTAAATATTCAGAAAT (SEQ ID No: | chromosome, to be added to |
| | 280) | mini-genome |
| 17286.0_R | CCCCAAAACCCCAAAACCCCTAAAATTGGAA | |
| | AGGGGCAAATTTGCTTATGA (SEQ ID No: | |
| | 281) | |
| 7278.0_F | CCCCAAAACCCCAAAACCCCATGAGTAATAT | Primer pair for amplifying |
| | ATACAAATTTTAAATGTATTTTGATTTA (SEQ | chromosome, to be added to |
| | ID No: 282) | mini-genome |
| 7278.0_R | CCCCAAAACCCCAAAACCCCATTGAGTGAGT | |
| | ATTTTTATATTTATTGCGAGTTA (SEQ ID | |
| | No: 283) | |
| 7752.0_F | CCCCAAAACCCCAAAACCCCACAATAGGCAT | Primer pair for amplifying |
| | ATTTAATAATTAATTGTTAAAG (SEQ ID No: | chromosome, to be added to |
| | 284) | mini-genome |
| 7752.0_R | CCCCAAAACCCCAAAACCCCACTCATTATAT | |
| | AAGGCTGAAAAAATCAGAGG (SEQ ID No: | |
| | 285) | |
| 244.1_F | CCCCAAAACCCCAAAACCCCTAAATGTAAGA | Primer pair for amplifying |
| | GTAAACTATCATATGAAAG (SEQ ID | chromosome, to be added to |
| | No: 286) | mini-genome |
| 244.1_R | CCCCAAAACCCCAAAACCCCATAATGCGAAA | |
| | TATTCATCAGAGTAAATAATG (SEQ ID No: | |
| | 287) | |
| 20383.0_F | CCCCAAAACCCCAAAACCCCATACGTCATGA | Primer pair for amplifying |
| | TTATAAGATTATTATAGAATGCTTAC (SEQ ID | chromosome, to be added to |
| | No: 288) | mini-genome |
| 20383.0_R | CCCCAAAACCCCAAAACCCCTCTTGTAAAAT | |
| | AATAAGTTTAAGAAATTGAATTTAG (SEQ ID | |
| | No: 289) | |
| 331.1_F | CCCCAAAACCCCAAAACCCCATAATATCAAA | Primer pair for amplifying |
| | TTAATGAATATTTATCAATTTTATTAAT (SEQ | chromosome, to be added to |
| | ID No: 290) | mini-genome |
| 331.1_R | CCCCAAAACCCCAAAACCCCCCCTAATGTCC | |
| | ATAATTTATGTATCAAATAAGG (SEQ ID No: | |
| | 291) | |
| 22208.0_F | CCCCAAAACCCCAAAACCCCATGATGGTGGA | Primer pair for amplifying |
| | GGAGTGAAGATAAATTAGAATG (SEQ ID No: | chromosome, to be added to |
| | 292) | mini-genome |
| 22208.0_R | CCCCAAAACCCCAAAACCCCAAAGTGCAATA | |
| | AAAAGAGTGAAAATAAATTTTTG (SEQ ID | |
| | No: 293) | |
| 21398.0_F | CCCCAAAACCCCAAAACCCCATATACCAATG | Primer pair for amplifying |
| | TTAAAAATGAATATTGATATAGAATAG (SEQ | chromosome, to be added to |
| | ID No: 294) | mini-genome |
| 21398.0_R | CCCCAAAACCCCAAAACCCCATAATACAAAG | |
| | TAAAATTGTTTTTTATAGTTCATAA (SEQ ID | |
| | No: 295) | |
| 11890.0_F | CCCCAAAACCCCAAAACCCCACATAGTGAAT | Primer pair for amplifying |
| | GAATTAATGAATAAGTTTGAG (SEQ ID No: | chromosome, to be added to |
| | 296) | mini-genome |
| 11890.0_R | CCCCAAAACCCCAAAACCCCGTGATAATAAA | |
| | TTCCTGAGTATATAGTTTAAGAAG (SEQ ID | |
| | No: 297) | |
| 13521.0_F | CCCCAAAACCCCAAAACCCCGTGATTGCATT | Primer pair for amplifying |
| | TTTTTGCGAAATATTTGC (SEQ ID No: | chromosome, to be added to |
| | 298) | mini-genome |
| 13521.0_R | CCCCAAAACCCCAAAACCCCTGAGTTCTCAT | |
| | GTAATAAAAGAATCCATG (SEQ ID No: | |
| | 299) | |
| 3511.0_F | CCCCAAAACCCCAAAACCCCATGATGCTACA | Primer pair for amplifying |
| | AAAACGCTATATAATCTATAAC (SEQ ID No: | chromosome, to be added to |
| | 300) | mini-genome |
| 3511.0_R | CCCCAAAACCCCAAAACCCCTTGAACTTTCA | |
| | ATAGATGTTTGATTAAATTC (SEQ ID | |
| | No: 301) | |
| 22209.0_F | CCCCAAAACCCCAAAACCCCAAAGATATGTG | Primer pair for amplifying |
| | GCTGGATTTTAAAATATGGTTG (SEQ ID No: | chromosome, to be added to |
| | 302) | mini-genome |
| 22209.0_R | CCCCAAAACCCCAAAACCCCAAGACTAATGA | |
| | ATTTGAGAATTATAAAATAATGAATC (SEQ ID | |
| | No: 303) | |
| 18924.0_F | CCCCAAAACCCCAAAACCCCATCAACTTTAA | Primer pair for amplifying |
| | TTCATTGTAGGAATTAAAGATGTAATAC (SEQ | chromosome, to be added to |
| | ID No: 304) | mini-genome |
| 18924.0_R | CCCCAAAACCCCAAAACCCCGTGAGAACAAA | |
| | TAATAATAAAAATAAAGGAATTAA (SEQ ID | |
| | No: 305) | |
| 14977.0_F | CCCCAAAACCCCAAAACCCCAATTCTTTATCT | Primer pair for amplifying |
| | GAATTAGATAAGAATTCATAAGC (SEQ ID | chromosome, to be added to |
| | No: 306) | mini-genome |
| 14977.0_R | CCCCAAAACCCCAAAACCCCGTGAGTATGCA | |
| | ATAGATTGTTAATTAAATTTG (SEQ ID No: | |
| | 307) | |
| 18694.0_F | CCCCAAAACCCCAAAACCCCAAGTTGCTAAA | Primer pair for amplifying |
| | AATAGTTGATAGCAACAAGTTAT (SEQ ID | chromosome, to be added to |
| | No: 308) | mini-genome |
| 18694.0_R | CCCCAAAACCCCAAAACCCCTGGATGTGTTT | |
| | TTTTCCAAATTAATGAACAAAAATTAAA (SEQ | |
| | ID No: 309) | |
| 13237.0_F | CCCCAAAACCCCAAAACCCCAACATTCTAAA | Primer pair for amplifying |
| | TTTCTTCTTTATAAGATTATTG (SEQ ID No: | chromosome, to be added to |
| | 310) | mini-genome |
| 13237.0_R | CCCCAAAACCCCAAAACCCCATCTAAACTAA | |
| | TCTGAAACCAAAGATAGTATG (SEQ ID No: | |
| | 311) | |
| 21338.0_F | CCCCAAAACCCCAAAACCCCGTTATCCATAT | Primer pair for amplifying |
| | ATACGTAAGCATTTTGCGATTG (SEQ ID No: | chromosome, to be added to |
| | 312) | mini-genome |
| 21338.0_R | CCCCAAAACCCCAAAACCCCGAAACCTATGC | |
| | ATTATTTTTAAAGAAATATTAAATTAA (SEQ | |
| | ID No: 313) | |
| 215.1_F | CCCCAAAACCCCAAAACCCCTCGTACATTAA | Primer pair for amplifying |
| | TAGTTGAAATTGCTTTTATTAAATTG (SEQ ID | chromosome, to be added to |
| | No: 314) | mini-genome |
| 215.1_R | CCCCAAAACCCCAAAACCCCGTAGTCTAAAA | |
| | TAAATTTTATTTTGGGTTTTAA (SEQ ID No: | |
| | 315) | |
| 13236.0_F | CCCCAAAACCCCAAAACCCCGTTAAATGATA | Primer pair for amplifying |
| | ATCATAGCAAAATTGCGGTAT (SEQ ID No: | chromosome, to be added to |
| | 316) | mini-genome |
| 13236.0_R | CCCCAAAACCCCAAAACCCCAAGGATAAATA | |
| | TTGAAAGTAAATGTTCTAATTAATTTGC (SEQ | |
| | ID No: 317) | |
| 16827.0_F | CCCCAAAACCCCAAAACCCCAGAAATGAAAA | Primer pair for amplifying |
| | GAATGATTTTTGAGGGGATTC (SEQ ID No: | chromosome, to be added to |
| | 318) | mini-genome |
| 16827.0_R | CCCCAAAACCCCAAAACCCCTAAAGGCAAAA | |
| | GTCGATTTAAATGCTCAGTTTC (SEQ ID No: | |
| | 319) | |
| 15136.0_F | CCCCAAAACCCCAAAACCCCTTAAGGCTAAA | Primer pair for amplifying |
| | ATACTTGTTTTACTAGAGAAC (SEQ ID No: | chromosome, to be added to |
| | 320) | mini-genome |
| 15136.0_R | CCCCAAAACCCCAAAACCCCATAAATCAAAT | |
| | TAAATTGCATAACATGAAC (SEQ ID | |
| | No: 321) | |
| 115.1_F | CCCCAAAACCCCAAAACCCCAGAGGATGTAA | Primer pair for amplifying |
| | ATTACAATAAATCGTAAAAAC (SEQ ID No: | chromosome, to be added to |
| | 322) | mini-genome |
| 115.1_R | CCCCAAAACCCCAAAACCCCTTCTAAAAAAT | |
| | ATAAAGATAAATTGACGTC (SEQ ID | |
| | No: 323) | |
| 21295.0_F | CCCCAAAACCCCAAAACCCCATCCAGTTGAA | Primer pair for amplifying |
| | ATCTAAAACAATTTTGTATATTTAAAG (SEQ | chromosome, to be added to |
| | ID No: 324) | mini-genome |
| 21295.0_R | CCCCAAAACCCCAAAACCCCTTAAGAGATTG | |
| | CATTATAAATAAGATAGGATTC (SEQ ID No: | |
| | 325) | |
| 16269.0_F | CCCCAAAACCCCAAAACCCCATTGATTGATA | Primer pair for amplifying |
| | AACTTGGAAGTTAAGAAAGATTTG (SEQ ID | chromosome, to be added to |
| | No: 326) | mini-genome |
| 16269.0_R | CCCCAAAACCCCAAAACCCCATGAATAACAG | |
| | ATGGAATGCTTCAAGATATG (SEQ ID No: | |
| | 327) | |
| 644.1_F | CCCCAAAACCCCAAAACCCCAAATGTTAGTA | Primer pair for amplifying |
| | TTTGAATTAAAGAGAGGTAAAAC (SEQ ID | chromosome, to be added to |
| | No: 328) | mini-genome |
| 644.1_R | CCCCAAAACCCCAAAACCCCTTATGAAAATG | |
| | AAATGGTTTTGATTGGCTAATAA (SEQ ID | |
| | No: 329) | |
| 5586.0_F | CCCCAAAACCCCAAAACCCCATGAGTAAAAT | Primer pair for amplifying |
| | TTAGCTTAAGTAATGTAAGAATC (SEQ ID | chromosome, to be added to |
| | No: 330) | mini-genome |
| 5586.0_R | CCCCAAAACCCCAAAACCCCATATATCAAAA | |
| | TATCAACATTTTTTTGTGTGATTGTTAC (SEQ | |
| | ID No: 331) | |
| 13085.0_F | CCCCAAAACCCCAAAACCCCTTGATGAAATT | Primer pair for amplifying |
| | TGAAAATGAATAGAGAGTAC (SEQ ID No: | chromosome, to be added to |
| | 332) | mini-genome |
| 13085.0_R | CCCCAAAACCCCAAAACCCCGTAATGCTACA | |
| | TTTGCAAAAAAGTACAAACAG (SEQ ID No: | |
| | 333) | |
| 13838.0_F | CCCCAAAACCCCAAAACCCCGTAAGGCCAGA | Primer pair for amplifying |
| | ATCAATGAATAAAAAGGTC (SEQ ID | chromosome, to be added to |
| | No: 334) | mini-genome |
| 13838.0_R | CCCCAAAACCCCAAAACCCCGAAAAGGGAG | |
| | ATTTACAAAAATTTGTAGATGTTATATTG | |
| | (SEQ ID No: 335) | |
| 1415.1_F | CCCCAAAACCCCAAAACCCCATTGATCATTA | Primer pair for amplifying |
| | ATAAAGAAGAATTGCTAATAT (SEQ ID No: | chromosome, to be added to |
| | 336) | mini-genome |
| 1415.1_R | CCCCAAAACCCCAAAACCCCAATGCGATGAA | |
| | ATGTTTTTTATTATGAAAAG (SEQ ID | |
| | No: 337) | |
| 19468.0_F | CCCCAAAACCCCAAAACCCCAAGGAAGTTCA | Primer pair for amplifying |
| | ATGCTATTTAGCAAATTAGG (SEQ ID | chromosome, to be added to |
| | No: 338) | mini-genome |
| 19468.0_R | CCCCAAAACCCCAAAACCCCTTGATTCAAAA | |
| | TATGCACAAGATTAAAAATTCAC (SEQ ID | |
| | No: 339) | |
| 20407.0_F | CCCCAAAACCCCAAAACCCCATAAGAAAGAT | Primer pair for amplifying |
| | AAGTTGCAATTAAATAATAAGG (SEQ ID No: | chromosome, to be added to |
| | 340) | mini-genome |
| 20407.0_R | CCCCAAAACCCCAAAACCCCATGAAGACAAG | |
| | TCTGATGAAAATAGAATGG (SEQ ID No: | |
| | 341) | |
| 19922.0_F | CCCCAAAACCCCAAAACCCCATAGTCTTAAA | Primer pair for amplifying |
| | ATTTTATACTATCATGAAATAATATTAAG (SEQ | chromosome, to be added to |
| | ID No: 342) | mini-genome |
| 19922.0_R | CCCCAAAACCCCAAAACCCCGTAAGTCTAAA | |
| | GTTTAACAGTTTTTAGTAAATATC (SEQ ID | |
| | No: 343) | |
| 20459.0_F | CCCCAAAACCCCAAAACCCCTTATGCTAGTT | Primer pair for amplifying |
| | GAGTGATTGAAAATATATTTGTGC (SEQ ID | chromosome, to be added to |
| | No: 344) | mini-genome |
| 20459.0_R | CCCCAAAACCCCAAAACCCCTTGACGTAGAA | |
| | TAATGGGCTTATAGAAG (SEQ ID No: | |
| | 345) | |
| 20493.0_F | CCCCAAAACCCCAAAACCCCTTAATCAACTC | Primer pair for amplifying |
| | ACTTTACCCACTAATCAAACAC (SEQ ID No: | chromosome, to be added to |
| | 346) | mini-genome |
| 20493.0_R | CCCCAAAACCCCAAAACCCCATATTTAAGAT | |
| | ATACAGAAATATAGAGAATACAAC (SEQ ID | |
| | No: 347) | |
| 9925.0_F | CCCCAAAACCCCAAAACCCCATTGGATCAAT | Primer pair for amplifying |
| | TTTGAAGAGAATTCATGGAAAAT (SEQ ID | chromosome, to be added to |
| | No: 348) | mini-genome |
| 9925.0_R | CCCCAAAACCCCAAAACCCCATCAGAAAAAA | |
| | TATTTGAAAATTCGATAAAGC (SEQ ID No: | |
| | 349) | |
| 22456.0_F | CCCCAAAACCCCAAAACCCCATTTCACTTTAT | Primer pair for amplifying |
| | TTATATATAGATTTGAAATTAAAGTT (SEQ ID | chromosome, to be added to |
| | No: 350) | mini-genome |
| 22456.0_R | CCCCAAAACCCCAAAACCCCAGTTGACATGT | |
| | TATTTCCAAATTTTCATGGATA (SEQ ID No: | |
| | 351) | |
| 17712.0_F | CCCCAAAACCCCAAAACCCCATGATAACAGG | Primer pair for amplifying |
| | AATATTTTATAAAATAGTTAAG (SEQ ID No: | chromosome, to be added to |
| | 352) | mini-genome |
| 17712.0_R | CCCCAAAACCCCAAAACCCCTCACTCTATGC | |
| | AATAAATTTGTTGATATATT (SEQ ID No: | |
| | 353) | |
| 11116.0_F | CCCCAAAACCCCAAAACCCCTTAAAAAAAGA | Primer pair for amplifying |
| | ATAGTTGGAATAAAAATGAATTT (SEQ ID | chromosome, to be added to |
| | No: 354) | mini-genome |
| 11116.0_R | CCCCAAAACCCCAAAACCCCAATAGATAAAG | |
| | ATGCCTTTTTTAATAAGTATTTAAC (SEQ ID | |
| | No: 355) | |
| 19275.0_F | CCCCAAAACCCCAAAACCCCGAGAGGATAAA | Primer pair for amplifying |
| | TTTATATGAAAATAAAAATAAAGC (SEQ ID | chromosome, to be added to |
| | No: 356) | mini-genome |
| 19275.0_R | CCCCAAAACCCCAAAACCCCATAAATAAGAA | |
| | ATTTTAAGAATAACGGGCAAATTAG (SEQ ID | |
| | No: 357) | |
| 21217.0_F | CCCCAAAACCCCAAAACCCCTTGAATTTTAAA | Primer pair for amplifying |
| | TAAACTTCTTTGTATGATTTAAATG (SEQ ID | chromosome, to be added to |
| | No: 358) | mini-genome |
| 21217.0_R | CCCCAAAACCCCAAAACCCCATAGATTACTT | |
| | TTCAAAGAATTTCTTGACATTC (SEQ ID No: | |
| | 359) | |
| 10537.0_F | CCCCAAAACCCCAAAACCCCAAAGCAAAGAA | Primer pair for amplifying |
| | ATCTGATGTTTTATTAGAAAAAGTG (SEQ ID | chromosome, to be added to |
| | No: 360) | mini-genome |
| 10537.0_R | CCCCAAAACCCCAAAACCCCATGAGATGATA | |
| | ATATTGCCTTTTTGCATATAAT (SEQ ID No: | |
| | 361) | |
| 22670.0_F | CCCCAAAACCCCAAAACCCCATCCTTATACA | Primer pair for amplifying |
| | AATTCAGAAAACTTAGCAAAT (SEQ ID No: | chromosome, to be added to |
| | 362) | mini-genome |
| 22670.0_R | CCCCAAAACCCCAAAACCCCGTGGAGAATTT | |
| | TCTAAAGAATTTTCGGAAATTTG (SEQ ID | |
| | No: 363) | |
| 1781.0_F | CCCCAAAACCCCAAAACCCCACTAGTCTTAA | PCR primers for amplifying |
| | ATATGAGAAAGATGATTTGAATAAG (SEQ ID | synthetic chromosome 1 and 6 |
| | No: 364) | in FIG. 5B |
| 1781.0_R | CCCCAAAACCCCAAAACCCCACAAGATAACA | |
| | TTGCTAATTTTCAATAAATTAAAT (SEQ ID | |
| | No: 365) | |
| 1781.0_Purple_F | GTCAGTGGTCTCAGTATGAAATTTACCTTGG | PCR primers for amplifying |
| | ATCCTCAGTGTTTCCCTTTGTG (SEQ ID No: | purple DNA building block in |
| | 366) | synthetic chromosomes 2-4 in |
| 1781.0_Purple_R | AACGCTCGGTCTCGCAGAAATAAATAATAGG | FIG. 5B |
| | CAGCATAATAATTCAGG (SEQ ID No: | |
| | 367) | |
| 1781.0_red_F | GTCAGTGGTCTCTCCAGTGGATTCACATCAC | PCR primers for amplifying |
| | TACCATTG (SEQ ID No: 368) | red DNA building block in |
| 1781.0_red_R | CCCCAAAACCCCAAAACCCCACAAGATAACA | synthetic chromosomes 2-4 |
| | TTGCTAATTTTCAATAAATTAAAT (SEQ ID | in FIG. 5B |
| | No: 369) | |
| 1781.0_turquoise_F | CCCCAAAACCCCAAAACCCCACTAGTCTTAA | PCR primers for amplifying |
| | ATATGAGAAAGATGATTTGAATAAG (SEQ ID | turquoise DNA building block |
| | No: 370) | in synthetic chromosomes 2-4 |
| 1781.0_turquoise_R | ACGCTCGGTCTCGATACAATCATTAGCATAT | in FIG. 5B |
| | ACATGCAGTCTGCTTAAATTAAG (SEQ ID | |
| | No: 371) | |
| DarkBlue_6mA_top | TCTGTAATTAATGGATCACTATAATCATCTGG | Oligos for annealing to make |
| | ATGACTATTGGTATTTGATGACGGTGCACAT | blue DNA building block in |
| | TTGACTTCTT (SEQ ID No: 372) | synthetic chromosomes 2-4 in |
| DarkBlue_6mA_bottom | ATTAATTACCTAGTGATATTAGTAGACCTACT | FIG. 5B. Bold red nucleotides |
| | GATAACCATAAACTACTGCCACGTGTAAACT | represent 6mA. |
| | GAAGAAGGTC (SEQ ID No: 373) | |
| 1781.0_red2_F | AGCCTAGGTCTCGTTCTTTTTGAGGGCGAGG | PCR primers for amplifying |
| | TTATTGTTAAT (SEQ ID No: 374) | red DNA building block in |
| 1781.0_red2_R | CCCCAAAACCCCAAAACCCCACAAGATAACA | synthetic chromosome 5 in |
| | T (SEQ ID No: 375) | FIG. 5B |
| 1781.0_orange_F | TAGTCAGGTCTCTAGAATAGGCTCACTCTAA | PCR primers for amplifying |
| | ATTCGAGTGCAAT (SEQ ID No: 376) | orange DNA building block in |
| 1781.0_orange_R | TCTACTGGTCTCAGTATGAAATTTACCTTGGA | synthetic chromosome 5 in FIG. |
| | TCCTCAGTGT (SEQ ID No: 377) | 5B |
| 1781.0_emerald_F | ATCGTAGGTCTCAATACAATCATTAGCATATA | PCR primers for amplifying |
| | CATGCAGT (SEQ ID No: 378) | emerald DNA building block in |
| 1781.0_emerald_R | CCCCAAAACCCCAAAACCCCACTAGTCTTAA | synthetic chromosome 5 in FIG. |
| | AT (SEQ ID No: 379) | 5B |
| 17535.0_F | CCCCAAAACCCCAAAACCCCATCTCAATTTAT | PCR primers for amplifying |
| | AAAATCAGAATAAGAGATTGTC (SEQ ID No: | “buffer” chromosome |
| | 380) | (Contig17535.0) for use in |
| 17535.0_R | CCCCAAAACCCCAAAACCCCAGAATAAAACA | chromatin assembly |
| | ACTGAAGTAAATATGAGTTAC (SEQ ID No: | |
| | 381) | |
| 12701assay_F | AAGAAGAACTAGCCAGCTCTCACTCAGTTC | PCR primers for assaying the |
| | (SEQ ID No: 382) | presence of ectopic DNA |
| 12701assay_R | TGTCTATCTCATCAGGCTCATCAGCATAGG | insertion in mta1 mutants |
| | (SEQ ID No: 383) | |
| 12701_firstround_T7_F | AAGAAGAACTAGCCAGCTCTCACTCAGTTC | PCR primers to generate DNA |
| | (SEQ ID No: 384) | template for ssRNA in vitro |
| 12701_firstround_T7_R | CCTCTCTGCCCACTAAATTATTCTGACAGC | transcription. This ssRNA is |
| | (SEQ ID No: 385) | injected into Oxytricha cells to |
| | | induce ectopic DNA retention |
| | | in MTA1 gene. PCR product is |
| | | amplified from Oxytricha |
| | | gDNA of cell strain JRB310. |
| | | The resulting PCR product is |
| | | subjected to a second round |
| | | of PCR amplification using |
| | | primers |
| | | “12701_secondround_T7_F” |
| | | and |
| | | “12701_secondround_T7_R”. |
| | | The final, second round PCR |
| | | product is then used for |
| | | ssRNA in vitro transcription. |
| 12701_secondround_T7_F | CTACTTGATATAATACGACTCACTATAGGGAA | PCR primers for second round |
| | TTCCTAAGGGGAGTGAAGCCAACAACAG | amplification of DNA template, |
| | (SEQ ID No: 386) | to be used for ssRNA in vitro |
| 12701_secondround_T7_R | TGTCTATCTCATCAGGCTCATCAGCATAGG | transcription. Forward primer |
| | (SEQ ID No: 387) | contains T7 promoter |
| | | sequence, which is required |
| | | for subsequent in vitro |
| | | transcription. |
| metGATC_F2 | GTGCTATGCATTTTAAATTTATTCGCATTGAA | PCR primers for amplification |
| | GA (SEQ ID No: 388) | of DNA substrate for use in |
| metGATC_R2 | ATTCAGAATTTTAGTGTGTGGAGTATGATAGT | 6mA methyltransferase assay |
| | A (SEQ ID No: 389) | involving Tetrahymena nuclear |
| noGATC2_F | GGTCTATATTATTTTAGTATTCTTTCTATAAAT | PCR primers for amplifying |
| | G (SEQ ID No: 390) | 350 bp dsDNA substrate for |
| noGATC2_R | GTTACAAGAATATAAGAAAAGAAAGGGTGAA | methyltransferase assays |
| | TAGG (SEQ ID No: 391) | involving recombinant proteins |
| | | (in FIGS. 2E, 2F, and 10H) |
| T7noGATC2_F2 | TAATACGACTCACTATAGGG | PCR primers for amplifying |
| | GGTCTATATTATTTTAGTATTCTTTC (SEQ ID | DNA ~350 bp dsDNA template |
| | No: 392) | with T7 overhangs at one end, |
| noGATC2_R | GTTACAAGAATATAAGAAAAGAAAGGGTGAA | for subsequent ssRNA |
| | TAGG (SEQ ID No: 393) | production by in vitro |
| | | transcription |
| T7noGATC2_F2 | TAATACGACTCACTATAGGG | PCR primers for amplifying |
| | GGTCTATATTATTTTAGTATTCTTTC (SEQ ID | DNA ~350 bp dsDNA template |
| | No: 394) | with T7 overhangs at the 5′ |
| T7noGATC2_R2 | TAATACGACTCACTATAGGG | and 3′ ends, for subsequent |
| | GTTACAAGAATATAAGAAAAG (SEQ ID No: | dsRNA production by in vitro |
| | 395) | transcription |
| noGATC_F3 | AACTTCTGTCATTACATTAAGCTTTAA (SEQ | DNA oligonucleotides for use |
| | ID No: 396) | in DNA methyltransferase |
| noGATC_R3 | TTAAAGCTTAATGTAATGACAGAAGTT (SEQ | assays in FIGS. 2G, 10I, |
| | ID No: 397) | 10J, 10K, 10L |
| noGATC_F12 | AACTTCTGTCATTAACTTAAGCTTTAA (SEQ | |
| | ID No: 398) | |
| noGATC_R12 | TTAAAGCTTAAGTTAATGACAGAAGTT (SEQ | |
| | ID No: 399) | |
| noGATC_F13 | AACTTCTGTACTTACATTAAGCTTTAA (SEQ | |
| | ID No: 400) | |
| noGATC_R13 | TTAAAGCTTAATGTAAGTACAGAAGTT (SEQ | |
| | ID No: 401) | |
| noGATC_F14 | AACTTCTGTACTTAACTTAAGCTTTAA (SEQ | |
| | ID No: 402) | |
| noGATC_R14 | TTAAAGCTTAAGTTAAGTACAGAAGTT (SEQ | |
| | ID No: 403) | |
| noGATC_F1 | AACTTCTGTCATTACATTAAGCTTTAAAAAAT | |
| | TCAATTCCTTTTATT (SEQ ID No: 404) | |
| noGATC_R1 | AATAAAAGGAATTGAATTTTTTAAAGCTTAAT | |
| | GTAATGACAGAAGTT (SEQ ID No: 405) | |
| noGATC_F2 | TGTCATTACATTAAGCTTTAAAAAATTCAATT | |
| | CCT (SEQ ID No: 406) | |
| noGATC_R2 | AGGAATTGAATTTTTTAAAGCTTAATGTAATG | |
| | ACA (SEQ ID No: 407) | |
| noGATC_F3 | AACTTCTGTCATTACATTAAGCTTTAA (SEQ | |
| | ID No: 408) | |
| noGATC_R3 | TTAAAGCTTAATGTAATGACAGAAGTT (SEQ | |
| | ID No: 409) | |
| noGATC_F8 | TATTAGAATTATGTTCTTCATGAAATT (SEQ | |
| | ID No: 410) | |
| noGATC_R8 | AATTTCATGAAGAACATAATTCTAATA (SEQ | |
| | ID No: 411) | |

TABLE 3
Recombinant protein sequences.

>MTA1 (manually curated from Tetrahymena DB gene
ID: TTHERM_00704040)
(SEQ ID No: 412)
MSKAVNKKGLRPRKSDSILDHIKNKLDQEFLEDNENGEQSDEDYDQKS
LNKAKKPYKKRQTQNGSELVISQQKTKAKASANNKKSAKNSQKLDEEE
KIVEEEDLSPQKNGAVSEDDQQQEASTQEDDYLDRLPKSKKGLQGLLQ
DIEKRILHYKQLFFKEQNEIANGKRSMVPDNSIPICSDVTKLNFQALI
DAQMRHAGKMFDVIMMDPPWQLSSSQPSRGVAIAYDSLSDEKIQNMPI
QSLQQDGFIFVWAINAKYRVTIKMIENWGYKLVDEITWVKKTVNGKIA
KGHGFYLQHAKESCLIGVKGDVDNGRFKKNIASDVIFSERRGQSQKPE
EIYQYINQLCPNGNYLEIFARRNNLHDNWVSIGNEL
>MTA9 (manually curated from Tetrahymena DB gene
ID: TTHERM_00301770)
(SEQ ID No: 413)
MAPKKQEQEPIRLSTRTASKKVDYLQLSNGKLEDFFDDLEEDNKPARN
RSRSKKRGRKPLKKADSRSKTPSRVSNARGRSKSLGPRKTYPRKKNLS
PDNQLSLLLKWRNDKIPLKSASETDNKCKVVNVKNIFKSDLSKYGANL
QALFINALWKVKSRKEKEGLNINDLSNLKIPLSLMKNGILFIWSEKEI
LGQIVEIMEQKGFTYIENFSIMFLGLNKCLQSINHKDEDSQNSTASTN
NTNNEAITSDLTLKDTSKFSDQIQDNHSEDSDQARKQQTPDDITQKKN
KLLKKSSVPSIQKLFEEDPVQTPSVNKPIEKSIEQVTQEKKFVMNNLD
ILKSTDINNLFLRNNYPYFKKTRHTLLMFRRIGDKNQKLELRHQRTSD
VVFEVTDEQDPSKVDTMMKEYVYQMIETLLPKAQFIPGVDKHLKMMEL
FASTDNYRPGWISVIEK
>p1 (manually curated from Tetrahymena DB gene
ID: TTHERM_00161750)
(SEQ ID No: 414)
MSLKKGKFQHNQSKSLWNYTLSPGWREEEVKILKSALQLFGIGKWKKI
MESGCLPGKSIGQIYMQTQRLLGQQSLGDFMGLQIDLEAVFNQNMKKQ
DVLRKNNCIINTGDNPTKEERKRRIEQNRKIYGLSAKQIAEKLPKVKK
HAPQYMTLEDIENEKFTNLEILTHLYNLKAEIVRRLAEQGETIAQPSI
IKSLNNLNHNLEQNQNSNSSTETKVTLEQSGKKKYKVLAIEETELQNG
PIATNSQKKSINGKRKNNRKINSDSEGNEEDISLEDIDSQESEINSEE
IVEDDEEDEQIEEPSKIKKRKKNPEQESEEDDIEEDQEEDELVVNEEE
IFEDDDDDEDNQDSSEDDDDDED
>p2 (manually curated from Tetrahymena DB gene
ID: TTHERM_00439330)
(SEQ ID No: 415)
MKKNSKSQNQPLDFTQYAKNMRKDLSNQDICLEDGALNHSYFLTKKGQ
YWTPLNQKALQRGIELFGVGNWKEINYDEFSGKANIVELELRICMILG
INDITEYYGKKISEEEQEEIKKSNIAKGKKENKLKDNIYQKLQQMQ

Sequences were manually curated by mapping RNaseq reads to reference gene annotations and verifying the accuracy of predicted exon boundaries.

### Example 2

### Epigenomic Profiles of Chromatin and Transcription in Oxytricha

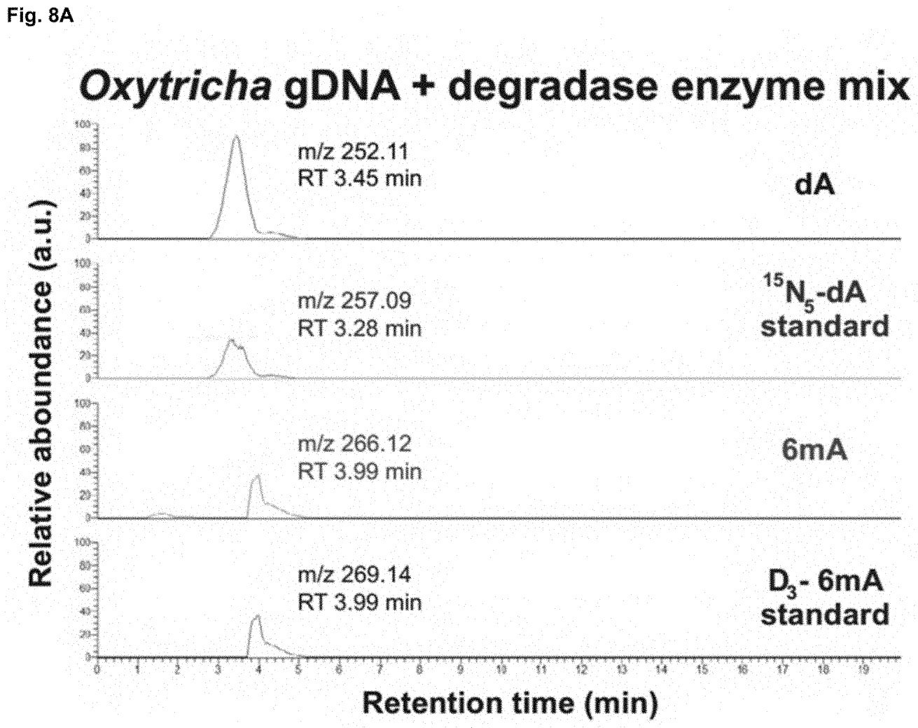

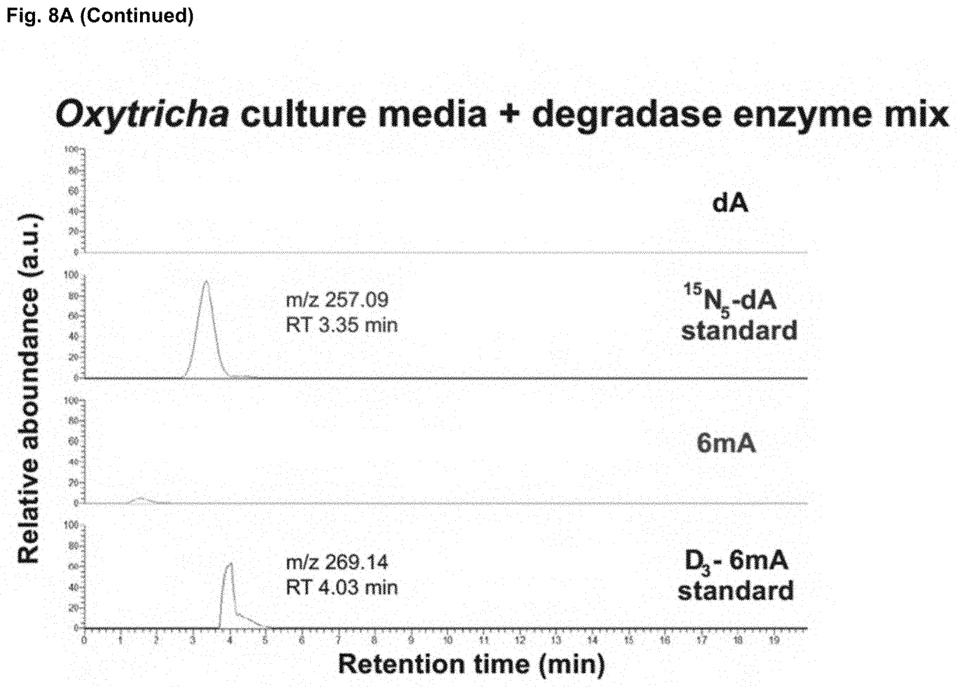

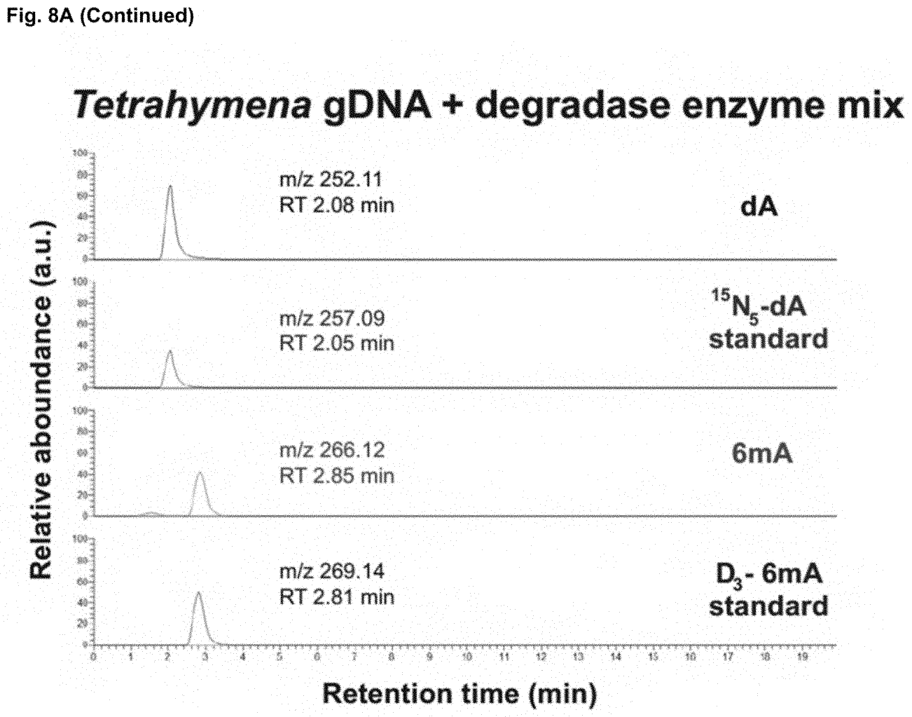

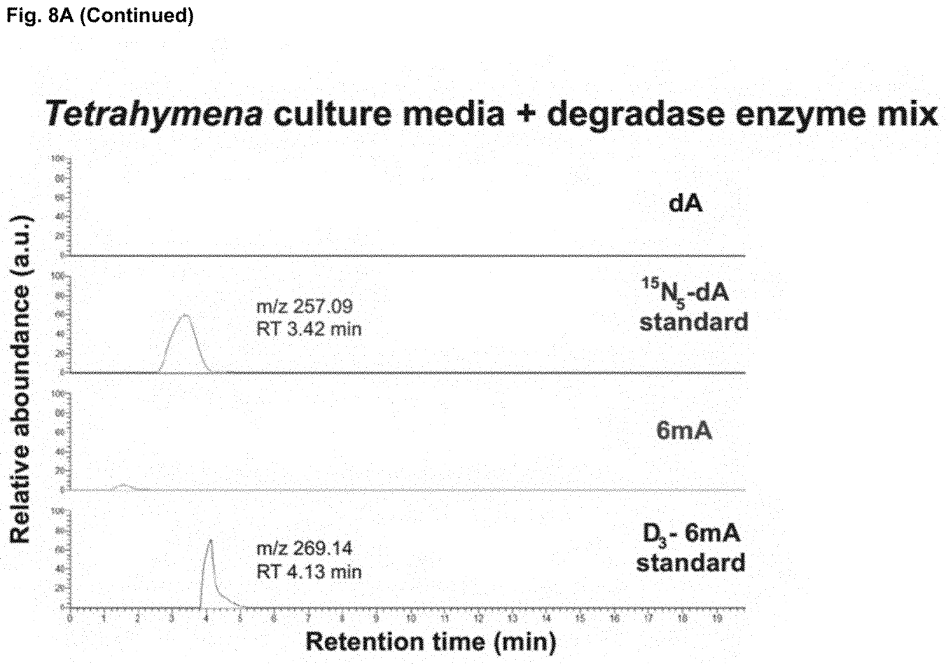

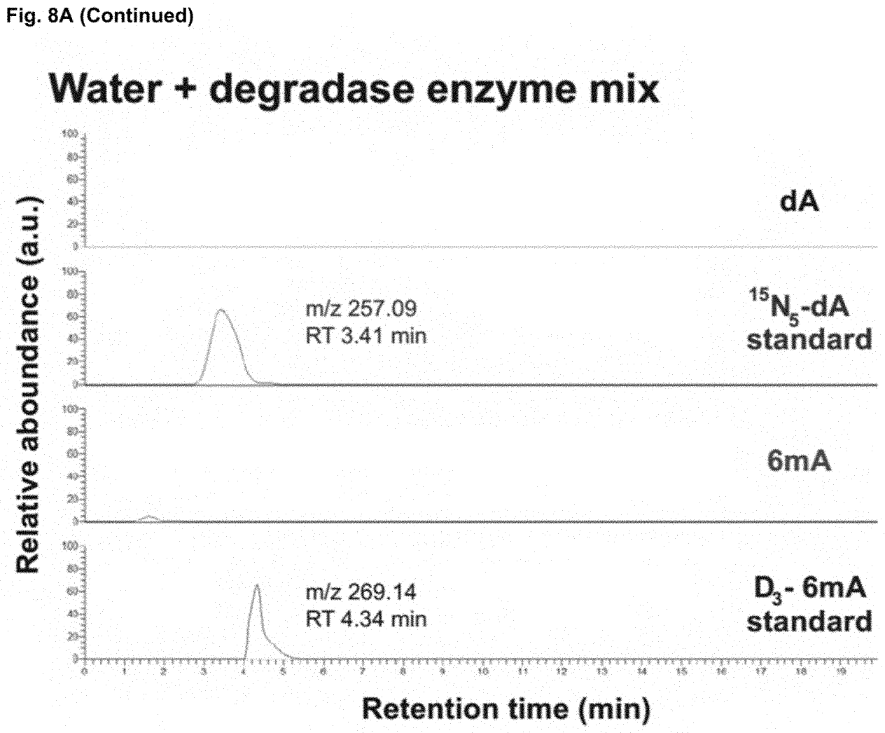

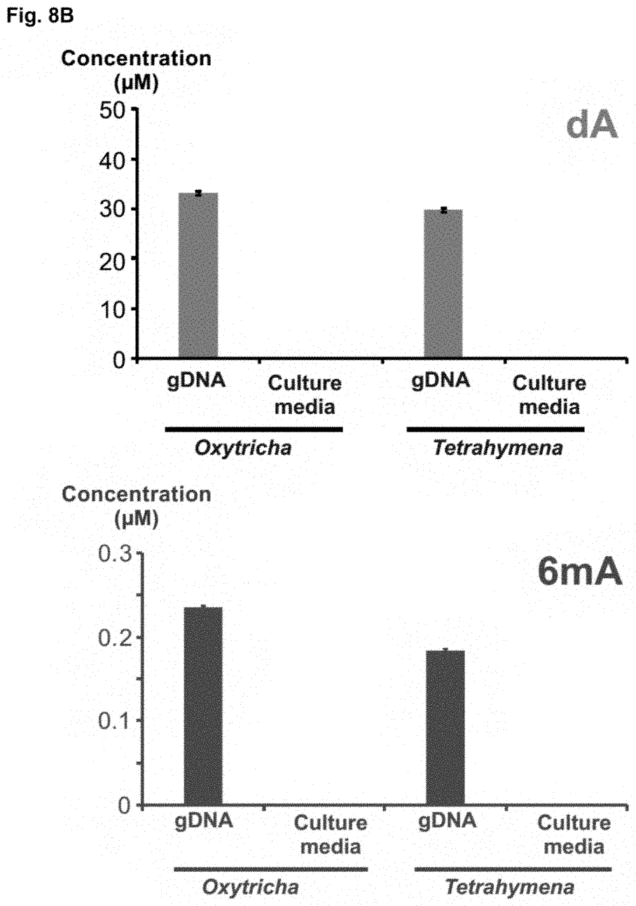

We generated genome-wide in vivo maps of nucleosome positioning, transcription, and 6 mA in the macronuclei of asexually growing (vegetative) Oxytricha trifallax cells using Mnase sequencing (MNase-seq), poly(Ar RNA sequencing (RNA-seq), transcriptional start site sequencing (TSS-seq), and single-molecule real-time sequencing (SMRT-seq) (FIGS. 1A-1E). The smallest Oxytricha chromosome is only 430 bp in length, with a single well-positioned nucleosome. Strikingly, 6 mA is enriched in three consecutive nucleosome-depleted regions directly downstream of transcription start sites (TSSs; FIG. 1A). Each region contains varying levels of 6 mA (FIG. 1B), with the +1/+2 nucleosome linker being most densely methylated (Table 4). In general, highly transcribed chromosomes tend to bear more 6 mA, suggesting a positive role of this DNA modification in gene regulation (FIG. 1C). The majority of methylation marks are located within an ApT motif (FIGS. 1D and 1E). 6 mA occurs on sense and antisense strands with approximately equal frequency, indicating that the underlying methylation machinery does not function strand-specifically. Quantitative LC-MS/MS analysis confirmed the presence of 6 mA in Oxytricha (FIGS. 8A and 8B; see Example 1).

TABLE 4
Descriptive statistics of 6mA distribution in the genome.

| | Number of 6mA sites | | | | | | | | | |
|---|---|---|---|---|---|---|---|---|---|---|
| | Oxytricha | | | | | Tetrahymena | | | | |
| | | | | | Standard | | | | | Standard |
| | Minimum | Maximum | Median | Mean | Deviation | Minimum | Maximum | Median | Mean | Deviation |
| Methyl | 0 | 14 | 2 | 2.03 | 2.27 | 0 | 27 | 10 | 9.66 | 6.10 |
| Cluster 1 | | | | | | | | | | |
| Methyl | 0 | 24 | 6 | 5.99 | 4.24 | 0 | 26 | 9 | 8.78 | 5.78 |
| Cluster 2 | | | | | | | | | | |
| Methyl | 0 | 16 | 2 | 2.49 | 2.91 | 0 | 25 | 5 | 5.75 | 5.53 |
| Cluster 3 | | | | | | | | | | |

Properties of 6 mA distribution in nucleosome linkers. In Oxytricha, methyl cluster 1=between 5′ chromosome end and +1 nucleosome; methyl cluster 2=between +1 and +2 nucleosome; methyl cluster 3=between +2 and +3 nucleosome. In Tetrahymena, methyl cluster 1=between +1 and +2 nucleosome; methyl cluster 2=between +2 and +3 nucleosome; methyl cluster 3=between +3 and +4 nucleosome. Consensus +1/+2/+3/+4 nucleosome positions: 193, 402, 618, 837 bp downstream of Oxytricha 5′ chromosome ends; 112, 304, 497, 698 bp downstream of Tetrahymena TSSs.

### Example 3

### Purification and Reconstitution of the Ciliate 6 mA Methyltransferase, MTA1c

To uncover the functions of 6 mA in vivo, we set out to identify and disrupt putative 6 mA methytransferases (MTases). The Oxytricha genome encodes a large number of candidate methyltransferases (Table 5), rendering it impractical to test gene function, one at a time or in combination. To identify the ciliate 6 mA MTase, we undertook a biochemical approach by fractionating nuclear extracts and identifying candidate proteins that co-purified with DNA methylase activity. The organism of choice for this experiment was Tetrahymena thermophila, a ciliate that divides significantly faster than Oxytricha (˜2 h versus 18 h; Cassidy-Hanley, 2012; Laughlin et al., 1983). This faster growth time rendered it feasible to culture large amounts of Tetrahymena cells for nuclear extract preparation. Tetrahymena and Oxytricha exhibit similar genomic localization and 6 mA abundance (FIGS. 8A-8B and 9A-9F). We thus reasoned that the enzymatic machinery responsible for 6 mA deposition is conserved between Tetrahymena and Oxytricha, and that Tetrahymena could serve as a tractable biochemical system for identifying the ciliate 6 mA MTase.

We prepared nuclear extracts from log-phase Tetrahymena cells, since 6 mA could be readily detected at this developmental stage through quantitative MS and PacBio sequencing (FIGS. 8A-8B and 9A-9F). Nuclear extracts were incubated with radiolabeled S-adenosyl-L-methionine (SAM) and PCR-amplified DNA substrate to assay for DNA methylase activity. Passage of the nuclear extract through an anion exchange column resulted in the elution of two distinct peaks of DNA methylase activity, both of which were heat sensitive (FIGS. 2C, 10A, and 10B). Western blot analysis confirmed that both peaks of activity mediate methylation on 6 mA (FIG. 10C). The resulting fractions were further purified and subjected to MS. Only four proteins-termed MTA1, MTA9, p1, and p2-were detected at higher abundance in fractions with high DNA methylase activity (FIGS. 2C and 2D). p1 and p2 contain homeobox-like domains, suggesting a DNA binding function for an undetermined process (FIG. 10D). On the other hand, MTA1 and MTA9 are both MT-A70 proteins. Such domains are widely known to mediate m6A RNA methylation in eukaryotes (Liu et al., 2014). MTA1 and MTA9 received the large majority of peptide matches, relative to all other MT-A70 genes encoded by the Tetrahymena genome (FIG. 2D; Table 6). Curiously, although poly(A)-selected RNA transcripts were present from all MT-A70 genes (FIG. 2D), almost all peptides in fractions with high DNA methylase activity corresponded to MTA1 and MTA9. The poly(A)⁺ RNA expression profiles of MTA1, MTA9, p1, and p2 are remarkably similar (FIG. 9K), peaking early in the sexual cycle. This coincides with a sharp increase in nuclear 6 mA, as evidenced from immunostaining (Wang et al., 2017). Accumulation of MTA1, MTA9, p1, and p2 therefore correlates with the presence of 6 mA in vivo.

We next investigated the phylogenetic relationship of MTA1 and MTA9 to other eukaryotic MT-A70 domain-containing proteins. Two widely studied mammalian MT-A70 proteins—METTL3 and METTL14 (Ime4 and Kar4 in yeast)-form a heterodimeric complex that is responsible for m6A methylation on mRNA. METTL3 is the catalytically active subunit, while METTL14 functions as an RNA-binding scaffold protein (Sledi arid Jinek, 2016; Wang et al., 2016a, 2016b). MTA1 and MTA9 derive from distinct monophyletic clades, outside of those that contain mammalian METTL3, METTL14, and C. elegans DAMT-1 (METTL4) (FIG. 2A). Thus, MTA1 and MTA9 are divergent MT-A70 family members that are phylogenetically distinct from all previously studied RNA and DNA N6-methyladenine MTases. We then asked whether MTA1 and MTA9 are also present in other eukaryotes with a similar occurrence of 6 mA in ApT motifs as Tetrahymena. We queried the genomes of Oxytricha, green algae, and eight basal yeast species, all of which exhibit this distinct methylation pattern (as evidenced from FIGS. 1A-1E; FIGS. 9A-9E; Fu et al., 2015; Mondo et al., 2017). For all of these taxa, we can identify MT-A70 homologs that are monophyletic with MTA1 and MTA9 (FIG. 2B). On the other hand, MT-A70 homologs from multicellular eukaryotes, including Arabidopsis, C. elegans, Drosophila, and mammals, grouped exclusively with METTL3, METTL14, and METTL4 lineages, but not MTA1 or MTA9. None of these latter genomes exhibit a consensus ApT dinucleotide methylation motif for 6 mA (Greer et al., 2015; Koziol et al., 2016; Liang et al., 2018; Liu et al., 2016; Wu et al., 2016; Xiao et al., 2018; Zhang et al., 2015). We note that the absence of an ApT dinucleotide motif is based on data from a limited number of cell types, developmental stages, and culture conditions tested in these studies. Nonetheless, within the scope of currently available data, the presence of MTA1 and MTA9 correlates with the distinctive genomic localization of 6 mA within ApT motifs.

We then sought to determine whether MTA1 and/or MTA9 are bona fide 6 mA methyltransferases. MTA1, but not MTA9, contains a catalytic DPPW motif (FIG. 10E)—a hallmark of N6-adenosine methyltransferases (Iyer et al., 2016). Surprisingly, recombinant full-length Tetrahymena MTA1 and MTA9 (FIG. 10G) showed no detectable DNA methyltransferase activity, individually or together (FIG. 2E). Examination of the MTA1 and MTA9 sequences revealed that neither protein possesses a predicted nucleic acid binding domain (FIG. 10D). In contrast, METTL3, which catalyzes m6A methylation on RNA, contains two tandem CCCH-type zinc finger motifs, necessary for RNA binding (Huang et al., 2019; Wang et al., 2016a). Additional co-factors may thus be necessary for MTA1/7 to engage DNA substrates. Indeed, the p1 and p2 proteins that co-elute with MTA1/7 in nuclear extracts possess homeobox-like domains predicted to bind DNA. We then tested whether these accessory factors, in addition to MTA1/7, are necessary for 6 mA methylation. Strikingly, mixing recombinant, full-length p1, p2, MTA1, and MTA9 resulted in robust 6 mA methylation in vitro (FIGS. 2E and 2F). This activity was abolished when each protein was omitted, indicated that all four are necessary for 6 mA methylation. Furthermore, MTA1 harboring a D209A mutation in the catalytic DPPW motif showed no activity, even in the presence of MTA9, p1, and p2 (FIG. 2E). We also created double mutations in MTA1 (N370A, E371A), which lie in the conserved region that interacts with the 2′ and 3′-hydroxyl groups of the ribose moiety in the SAM cofactor (FIG. 2E). This mutant protein also exhibited no 6 mA methylase activity. Taken together, we find that four proteins—MTA1, MTA9, p1, and p2—are necessary for 6 mA methylation in vitro, with MTA1 the likely catalytic subunit. Henceforth, we refer to these four proteins as the putative MTA1 complex (MTA1c).

Purification of the MTA1c proteins from an E. coli overexpression system raises the possibility of methyltransferase activity arising from contaminating Dam methylase; however, we exclude this possibility for three reasons. (1) The DNA substrate used in this assay does not contain 5′-NATC-3′ sites, which are recognized and methylated by Dam methylase (Horton et al., 2006). (2) Methyltransferase activity was only observed when all four recombinant proteins were incubated with DNA. If contaminating Dam methylase were present in one or more of these protein preparations, then background activity should be observed when subsets of these proteins are used in the assay. 3) Mutation of MTA1 catalytic residues leads to loss of methylation, which is also inconsistent with contaminating methyltransferase activity.

TABLE 5
Candidate genes in ciliates.
MT-A70 genes in Oxytricha trifallax

| | Gene name in | |
|---|---|---|
| UniProt ID | this study | OxyDB gene name |
| J9IF92_9SPIT | MTA1 | Contig12701.0.0.g16 |
| J9IGS7_9SPIT | TAMT-1 | Contig17486.0.g100 |
| J9J9V7_9SPIT | MTA1-B | Contig16314.0.g25 |
| J9HW68_9SPIT | MTA9 | Contig1237.1.g126 |
| J9IMU5_9SPIT | MTA9-B | Contig17413.0.g36 |

MT-A70 genes in Tetrahymena thermophila

| | Gene name in | Tetrahymena Genome |
|---|---|---|
| UniProt ID | this study | Database gene name |
| Q22GC0_TETTS | MTA1 | TTHERM_00704040 |
| Q23TW8_TETTS | MTA2 | TTHERM_00962190 |
| I7LVP8_TETTS | MTA3/TAMT-1-B | TTHERM_00136470 |
| I7MGX6_TETTS | MTA4 | TTHERM_00558100 |
| Q23RE0_TETTS | MTA5/TAMT-1 | TTHERM_00388490 |
| I7MIF9_TETTS | MTA9 | TTHERM_00301770 |
| Q22XT1_TETTS | MTA9-B | TTHERM_01005150 |

METTL16 homologs in Oxytricha trifallax

| UniProt ID | OxyDB gene name |
|---|---|
| J9F3J7_9SPIT | Contig11945.0.g48 |
| J9J5P9_9SPIT | Contig7462.0.g41 |
| J9III0_9SPIT | Contig4244.0.g39 |

N6AMT1 homologs in Oxytricha trifallax

| UniProt ID | OyDB gene name |
|---|---|
| J9IFV1_9SPIT | Contig7751.0.g12 |

Accessory factor genes in Tetrahymena thermophila

| | Gene name in | Tetrahymena Genome |
|---|---|---|
| UniProt ID | this study | Database gene name |
| Q22VV9_TETTS | p1 | TTHERM_00161750 |
| I7M8B9_TETTS | p2 | TTHERM_00439330 |

ISWI homologs in Oxytricha trifallax and Tetrahymena thermophile

| | | Tetrahymena Genome |
|---|---|---|
| UniProt ID | OxyDB gene name | Database gene name |
| I7M280_TETTS | | TTHERM_00137610 |
| J9FBJ2_9SPIT | Contig11737.0.g12 | |

The Uniprot ID of each gene is listed. The Oxytricha macronuclear genome encodes five genes belonging to the MT-A70 family (Iyer et al., 2016; Swart et al., 2013). Such genes commonly function as RNA m6 A MTases in eukaryotes, having evolved from m.MunI-like MTases in bacterial restriction-modification systems (Iyer et al., 2016). An MT-A70 gene belonging to the METTL4 subclade, DAMT1, is a putative 6 mA methyltransferase in C. elegans (Greer et al., 2015). However, none of the Oxytricha MT-A70 genes in this Table cluster together with METTL4 on a phylogenetic tree (FIGS. 2A and 9G). The Oxytricha genome also contains homologs of a structurally distinct RNA m6 A MTase, METTL16, which was reported to methylate U6 snRNA (Table 5) (Pendleton et al., 2017; Warda et al., 2017). Another candidate, N6AMT1—which does not contain an MT-A70 domain—was recently found to mediate DNA 6 mA methylation in human cells (Xiao et al., 2018). An N6AMT1 homolog is also present in the Oxytricha genome. Accessory factors refer to the p1 and p2 proteins, which are necessary for 6 mA methylation by MTA1 and MTA9 in vitro. The UniProt IDs of putative ISWI homologs in Oxytricha and Tetrahymena are also listed.

TABLE 6
Mass spectrometry analysis of MTA1, MTA9, p1, and p2 proteins.
Data from Low Salt Fraction

| | Gene name in | % of protein covered by peptide |
|---|---|---|
| UniProt ID | this study | data from LC-MS/MS experiment |
| Q22GC0_TETTS | MTA1 | 78.8% |
| I7MIF9_TETTS | MTA9 | 46.3% |
| Q22W9_TETTS | p1 | 41.9% |
| I7M8B9_TETTS | p2 | 81.7% |

Data from High Salt Fraction

| | Gene name in | % of protein covered by peptide |
|---|---|---|
| UniProt ID | this study | data from LC-MS/MS experiment |
| Q22GC0_TETTS | MTA1 | 69.9% |
| I7MIF9_TETTS | MTA9 | 72.2% |
| Q22VV9_TETTS | p1 | 55.3% |
| I7M8B9_TETTS | p2 | 93.4% |

Percentage of each polypeptide that is covered by peptide data is calculated. “Low Salt Sample” and “High Salt Sample” correspond to partially purified nuclear extracts that elute as two distinct peaks of activity from a Q sepharose anion exchange column (FIG. 2C).

### Example 4

MTA1c Preferentially Methylates ApT Dinucleotides in dsDNA

We next investigated the substrate preferences of MTA1c. First, in vitro transcription was performed to generate doublestranded RNA (dsRNA) and single-stranded RNA (ssRNA) from the input dsDNA substrate. We found that MTA1c methylates dsDNA but not dsRNA or ssRNA of the same sequence, indicating that it is selective for DNA over RNA (FIG. 10H). We then generated a series of dsDNA substrates by annealing oligonucleotide pairs of different length and sequence. All of these substrates are bona fide Tetrahymena genomic DNA sequences. In each case, MTA1c can methylate the annealed dsDNA but not ssDNA (FIGS. 2G and 10I).

Since 6 mA methylation mainly lies in ApT dinucleotides in vivo (FIGS. 1D and 9D), we asked whether MTA1c preferentially methylates this motif. To test this, we used a 27 bp dsDNA substrate with two ApT dinucleotides in its native sequence (FIG. 2G). We disrupted one or both ApT motifs (FIG. 2G) by mutually swapping the 5′ A with a neighboring base 5′-CAT-3′→5′-ACT-3′. Disrupting both ApT dinucleotides resulted in >10-fold reduced methylation, while disrupting only one motif led to a 2- to 4-fold loss (FIGS. 2G and 10K).

Given that 6 mA occurs on both strands of genomic DNA in vivo (FIGS. 1E and 9E), we asked whether pre-existing methylation of one strand affects MTA1c activity. DNA oligonucleotides were nonspecifically methylated with 6 mA using EcoGII (Murray et al., 2018), a bacterial 6 mA methyltransferase. After rigorous purification, samples were annealed to an unmethylated, complementary strand to yield hemimethylated dsDNA (FIG. 10F). MTA1c activity was 3- to 3.5-fold higher on hemimethylated substrates, relative to unmethylated dsDNA (FIG. 2G). This effect was similar between dsDNA substrates pre-methylated on the sense or antisense strand, consistent with the lack of an overt strand bias in 6 mA locations in vivo (FIGS. 1E and 9E). Importantly, the increase in MTA1c activity cannot be attributed to contaminating EcoGII in hemimethylated substrates, since no activity was observed in the absence of MTA1c (FIG. 10J). Thus, pre-existing 6 mA methylation stimulates MTA1c, indicative of a positive feedback loop.

We then asked whether MTA1c activity is modulated not only by the dinucleotide motif sequence per se, but also by flanking sequences. This may manifest as the wide variation in frequency of DNA 4-mer containing a methylated ApT dinucleotide 5′-NA*TN-3′ in vivo (FIG. 10M). To test this, we used a dsDNA substrate containing two ApT dinucleotides, both within a 5′-CATT-3′. Swapping of the ApT motif with the adjacent downstream DNA residue produced substrates containing 5′-TATA-3′ (FIG. 10L). Substrates with this change at both locations had 4-fold less MTA1c activity, and an intermediate effect when only one dinucleotide was altered (FIG. 10L). These data indicate that 5′-CATT-3′ is the preferred methylation substrate, consistent with the higher frequency of methylated 5-CA*TT-3′ versus 5-TA*TA-3′ in both Tetrahymena and Oxytricha genomic DNA (FIG. 10M). The difference in frequency of methylated sequences cannot simply be attributed to the higher frequency of the 4nt 5′-CATT-3′ motif versus 5′-TATA-3′ in the genome, because the opposite trend is observed (FIG. 10N). Thus, MTA1c is sensitive to variation in DNA sequences flanking the ApT dinucleotide motif.

### Example 5

### MTA1 is Necessary for 6 mA Methylation In Vivo

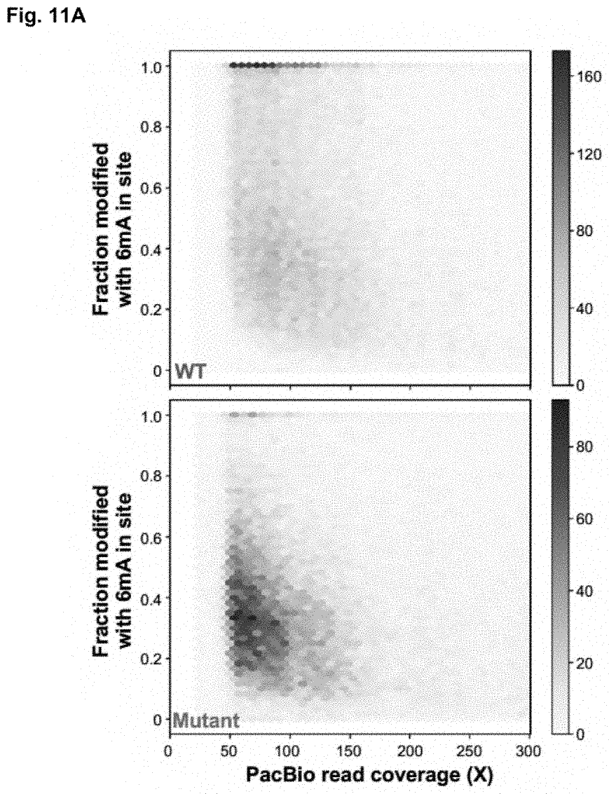

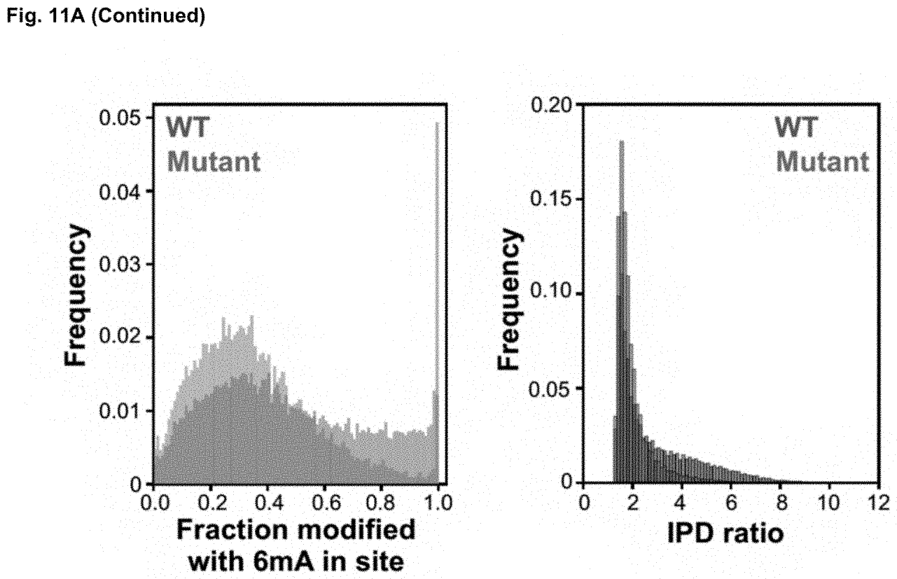

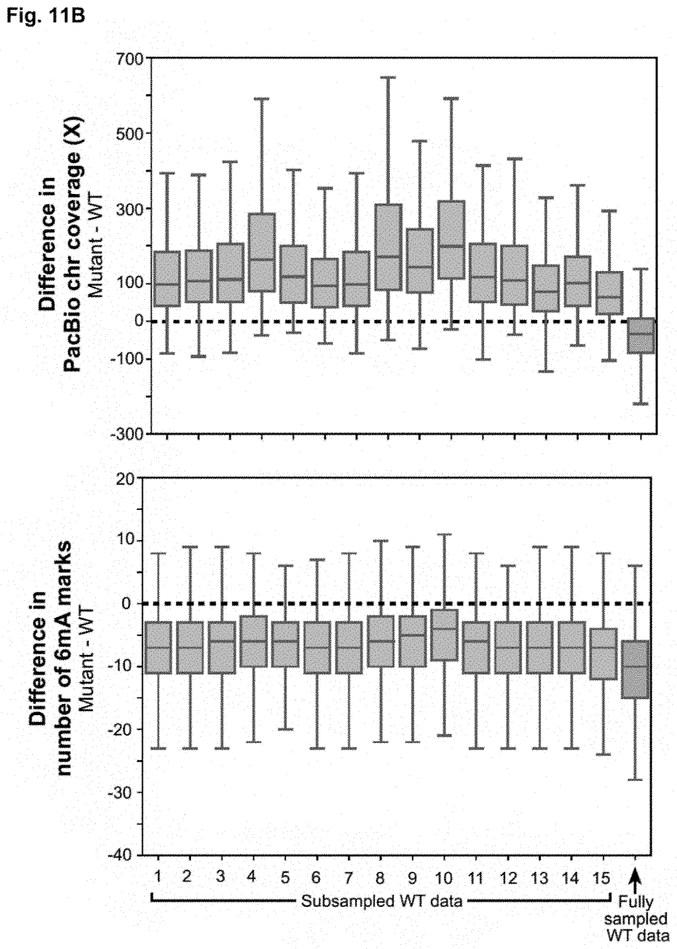

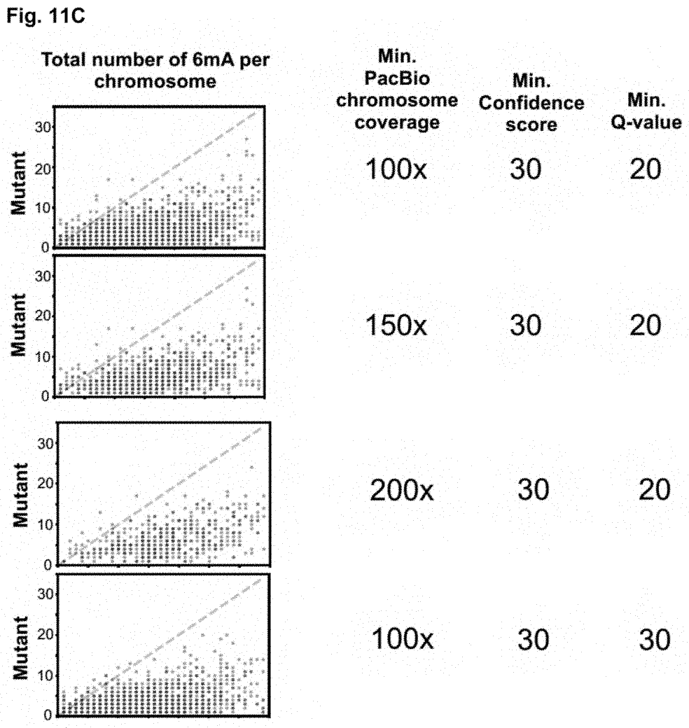

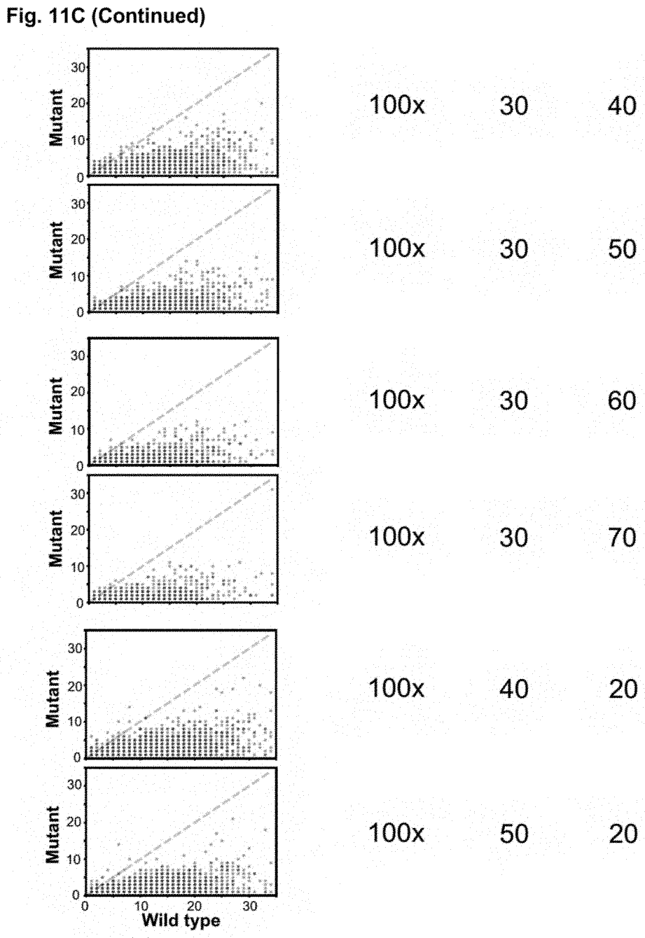

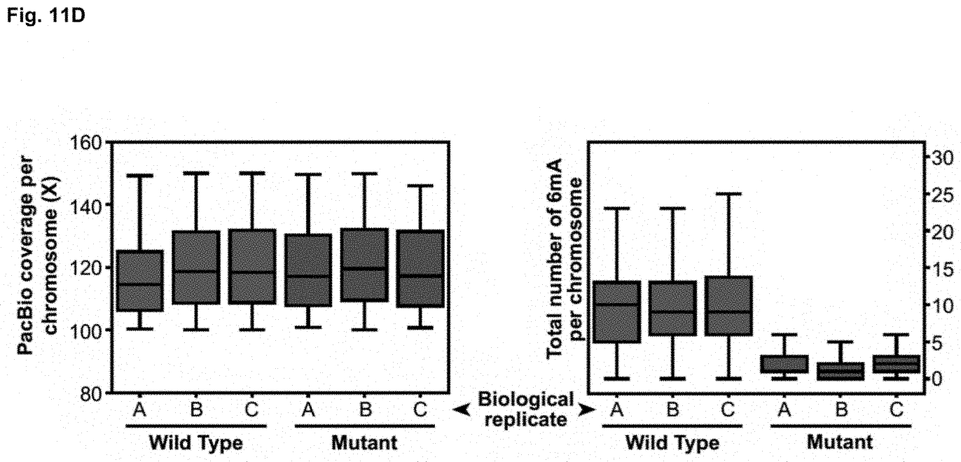

Having established that MTA1c is a 6 mA methyltransferase, we tested the role of MTA1c in mediating 6 mA methylation in vivo in Oxytricha, for which we have ease of generating mutants. The genome-wide localization of 6 mA is conserved between Oxytricha and Tetrahymena (FIGS. 1A-1E and 9A-9F), implying similar underlying enzymatic machinery. Indeed, all four component genes—MTA1, MTA9, p1, and p2—are clearly conserved between both species (FIGS. 9G-9J). The DPPW catalytic motif is also completely conserved in Tetrahymena and Oxytricha MTA1 but not MTA9, suggesting that MTA1 is the likely catalytic subunit of MTA1c in both ciliates (FIG. 10E). To abrogate MTA1c function, we disrupted the Oxytricha MTA1 gene by inserting an ectopic DNA sequence 49 bp downstream of the start codon, resulting in a frameshift mutation and loss of the C-terminal MTase domain (FIG. 3A). Oxytricha has two MTA1 paralogs, named MTA1 and MTA1-B (FIGS. 2A and 9G). We focused on MTA1 because MTA1-B is not expressed in vegetative Oxytricha cells (Swart et al., 2013), which we used to profile 6 mA locations via SMRT-seq. Dot blot analysis confirmed a significant reduction in bulk 6 mA levels in mutant lines (FIG. 3B). We then examined 6 mA positions at high resolution using SMRT-seq to understand how the DNA methylation landscape is altered in mta1 mutants. Notably, these mutants exhibit genome-wide loss of 6 mA, with complete abolishment of the dimethylated ApT motif, and reduction in frequency of all other methylated dinucleotide motifs (FIGS. 3C-3E). These findings are consistent across all biological replicates and are robust to wide variation in SMRT-seq parameters for calling 6 mA modifications (FIGS. 11B-11D). It cannot be attributed to variation in sequencing coverage between wild-type and mutant lines. The loss of methylated ApT dinucleotides in mta1 mutants is consistent with our in vitro data suggesting that MTA1c primarily methylates ApT sites (FIGS. 2G and 10K). The Inter Pulse Duration ratio (degree of polymerase slowing during PacBio sequencing due to presence of a modified base) and estimated fractional methylation also decreased significantly at called 6 mA sites in mta1 mutants (p<2.2×10-16, Wilcoxon rank-sum test) (FIG. 11A). MTA1 is therefore necessary for a significant proportion of in vivo 6 mA methylation events in Oxytricha.

What are the phenotypic consequences of 6 mA loss in vivo? It has been proposed that DNA methylation—including 6 mA and cytosine methylation—is involved in nucleosome organization (Fu et al., 2015; Huff and Zilberman, 2014). We thus asked whether nucleosome organization is altered in mta1 mutants. We quantified nucleosome “fuzziness,” defined as the SD of MNase-seq read locations surrounding the called nucleosome peak (Lai and Pugh, 2017; Mavrich et al., 2008). A poorly positioned nucleosome consists of a shallow and wide peak of MNase-seq reads, manifested by a high fuzziness score. Nucleosomes were first grouped according to the change in flanking 6 mA between wild-type and mta1 mutant cells (FIGS. 12A-12G). The nucleosomes that experience large changes in flanking 6 mA exhibit significantly greater increase in fuzziness, compared to nucleosomes with little change in flanking 6 mA (FIGS. 12A and 12D). Such nucleosomes also exhibit changes in occupancy that are consistent with an increase in fuzziness (FIGS. 12A and 12E). These results are robust to variation in MNase digestion (FIGS. 14C and 14D). On the other hand, nucleosome linkers do not change in length or occupancy, even though 6 mA is lost from these regions (FIGS. 12B, 12C, 12F, and 12G). We conclude that 6 mA exerts subtle effects on nucleosome organization in vivo.

### Example 6

### 6 mA Disfavors Nucleosome Occupancy Across the Genome In Vitro but not in Vivo

Multiple factors, including 6 mA, DNA sequence, and chromatin remodeling complexes, may collectively contribute to nucleosome organization in vivo. The effect of 6 mA could therefore be masked by these elements. We next sought to determine whether 6 mA directly impacts nucleosome organization. To this end, we assembled chromatin in vitro using Oxytricha gDNA, which contains cognate 6 mA. To obtain a matched negative control lacking DNA methylation, 98 complete chromosomes were amplified using PCR (FIG. 4A), purified and subsequently mixed together in stoichiometric ratios to obtain a “mini-genome” (FIG. 4B). These chromosomes collectively reflect overall genome properties, including AT content, chromosome length, and transcriptional activity (Table 7). Native genomic DNA (containing 6 mA) and amplified mini-genome DNA (lacking 6 mA) were each assembled into chromatin in vitro using Xenopus or Oxytricha histone octamers (FIGS. 13A-13F) and analyzed using MNase-seq. We computed nucleosome occupancy from the native genome and mini-genome samples across 199,795 overlapping DNA windows, spanning all base pairs in the 98 chromosomes. This allowed the direct comparison of nucleosome occupancy in each window of identical DNA sequence, with and without 6 mA (FIGS. 4C and 4D). Windows exhibit lower nucleosome occupancy with increasing 6 mA, confirming the quantitative nature of this effect. Furthermore, similar trends were observed for both native Oxytricha and recombinant Xenopus histones, suggesting that the effects of 6 mA on nucleosome organization arise mainly from intrinsic features of the histone octamer rather than from species-specific variants (FIGS. 4C and 4D). These results are also robust to the extent of MNase digestion of reconstituted chromatin (FIG. 14A).

We then directly compared the impact of 6 mA on nucleosome occupancy in vitro and in vivo. Loss of 6 mA in vitro is achieved by mini-genome construction, while loss in vivo is achieved by the mta1 mutation. For each overlapping DNA window, we calculated the difference in nucleosome occupancy: (1) between native genome and mini-genome DNA in vitro, and (2) between wild-type and mta1 mutants in vivo (FIG. 4C). Nucleosome occupancy is indeed lower in the presence of 6 mA methylation in vitro (FIGS. 4C and 4D). In contrast, no change in nucleosome occupancy is observed in vivo (FIGS. 4C and 4E). This result is consistent with our earlier analysis of linker occupancy in mta1 mutants (FIGS. 12C and 12G). We note that highly methylated DNA windows show greater change in 6 mA relative to mta1 mutants (FIG. 3D). Yet, these windows do not change in nucleosome occupancy in vivo. We conclude that 6 mA methylation locally disfavors nucleosome occupancy in vitro, but that this intrinsic effect can be overcome by endogenous chromatin factors in vivo.

TABLE 7
Descriptive statistics of reference genomes.

| | Native genomic DNA | Mini-genome DNA |
|---|---|---|
| Chromosome | 2449 +/− 742 | 2107 +/− 778 |
| length (bp) | Min = 1155 | Min = 1201 |
| | Max = 6494 | Max = 4659 |
| SMRT-seq | 177.4 +/− 117.0 | 205.3 +/− 136.1 |
| coverage (x) | Min = 75.1 | Min = 77.8 |
| | Max = 1392.6 | Max = 918.4 |
| Total number | 46,322 | 2,344 |
| of 6mA marks | | |
| in genome | | |
| 6mA sites per | 12 +/− 8 | 24 +/− 16 |
| chromosome | Min = 0 | Min = 0 |
| | Max = 73 | Max = 73 |
| AT content (%) | 67.8 +/− 3.0 | 66.5 +/− 2.7 |
| | Min = 55.7 | Min = 60.2 |
| | Max = 76.2 | Max = 72.2 |
| RNAseq | 34.4 +/− 75.2 | 53.7 +/− 71.5 |
| (FPKM) | Min = 0.0 | Min = 0.1 |
| | Max = 1444.5 | Max = 424.8 |

Properties of Oxytricha chromosomes in native genomic DNA and mini-genome DNA. “+/−” indicates one standard deviation above or below the mean.

### Example 7

### Modular Synthesis of Epigenetically Defined Chromosomes

The above experiments used kinetic signatures from SMRT-seq data to infer the presence of 6 mA marks in genomic DNA. We next sought to confirm that 6 mA is directly responsible for disfavoring nucleosomes in vitro, and to understand how this effect could be overcome by cellular factors. 6 mA-containing oligonucleotides were annealed and subsequently ligated with DNA building blocks to form full-length chromosomes. Importantly, these chromosomes contain 6 mA at all locations identified by SMRT-seq in vivo. The representative chromosome, Contig1781.0, is 1.3 kb, contains a clearly defined TSS, and encodes a single highly transcribed gene with a predicted RING finger domain. The length and gene structure are characteristic of typical Oxytricha chromosomes (FIG. 5A). We independently validated the location of 6 mA in vivo by sequencing chromosomal DNA immunoprecipitated with an anti-6 mA antibody (FIG. 5A).

Four chromosome variants were synthesized, with cognate 6 mA sites on neither, one, or both DNA strands (chromosomes 1-4 in FIGS. 5B and 5C). Chromatin was assembled by salt dialysis with either Oxytricha or Xenopus nucleosomes and subsequently digested with MNase to obtain mononucleosomal DNA (FIGS. 6A and 13G). Tiling qPCR was used to quantify nucleosome occupancy at ˜50 bp increments along the entire length of the synthetic chromosome (FIG. 6B). The fully methylated locus exhibits a ˜46% reduction in nucleosome occupancy relative to the unmethylated variant, while hemimethylated chromosomes containing half the number of 6 mA marks showed intermediate nucleosome occupancy at the corresponding region (FIG. 6B). The reduction in nucleosome occupancy was confined to the methylated region and not observed across the rest of the chromosome. Similar trends were observed when chromatin was assembled using the NAP1 histone chaperone (FIG. 14F. top panel). indicating that this effect is not an artifact of the salt dialysis method. Furthermore, moving 6 mA to an ectopic location (chromosome 5 in FIGS. 5B and 5C) decreases nucleosome occupancy at that site (FIG. 6C). We conclude that 6 mA directly disfavors nucleosome occupancy in a local, quantitative manner in vitro.

### Example 8

### Chromatin Remodelers Restore Nucleosome Occupancy Over 6 mA Sites

Nucleosome occupancy in vivo is influenced not only by DNA sequences but also by trans-acting factors such as ATP-dependent chromatin remodeling factors (Struhl and Segal, 2013). We used synthetic, methylated chromosomes to test how the well-studied chromatin remodeler ACF responds to 6 mA in native DNA. ACF generates regularly spaced nucleosome arrays in vitro and in vivo (Clapier and Cairns, 2009; Ito et al., 1997). Its catalytic subunit ISWI is conserved across eukaryotes, including Oxytricha and Tetrahymena (Table 5). Synthetic chromosomes were assembled into chromatin by salt dialysis as before and then incubated with ACF in the presence of ATP (FIGS. 13H and 6D). We find that ACF partially—but not completely—restores nucleosome occupancy over the methylated locus in an ATP-dependent manner (FIG. 6D). This effect is observed when ACF was added to chromatin assembled by salt dialysis or the NAP1 histone chaperone (FIGS. 6D and 14F). ACF also restores nucleosome occupancy over methylated loci in native genomic DNA (FIGS. 6E and 13I), indicating that the effect is not restricted to a single chromosome. This result is robust to the extent of MNase digestion (FIG. 14B). Although the heterologous system used here may differ from endogenous chromatin assembly factors in Oxytricha, our experiment illustrates the principle that trans-acting factors can counteract or even overcome the effect of 6 mA on nucleosome organization.

### Example 9

### Disruption of MTA1 Impacts Gene Expression and Sexual Development

Since mta1 mutants exhibit genome-wide loss of 6 mA, we assayed these cells for transcriptional changes by poly(A)⁺ RNAseq. Only a small minority of genes show significant changes in gene expression (10% false discovery rate [FDR]; FIG. 7A). To examine the methylation status of these differentially expressed genes, we grouped them according to “starting” methylation level, as defined by the total number of 6 mA marks near the TSS in wild-type cells. Genes exhibit two distinct transcriptional responses: those with low starting levels of 6 mA exhibit a small change in 6 mA between wild-type and mutant cells (FIG. 3D) and tend to be significantly upregulated in mutant lines (p=2.8×10⁻⁹, Fisher's exact test; FIG. 7B). Surprisingly, genes with high starting 6 mA are not enriched in differentially expressed genes (p>0.1, Fisher's exact test), even though they exhibit greater loss of 6 mA in mutants (FIG. 3D). Steady-state RNA-seq levels are therefore largely robust to drastic changes in 6 mA levels. Since most, but not all, 6 mA is lost from mta1 mutants (FIG. 3C), it is also possible that residual DNA methylation across the genome sufficiently buffers genes from changes in transcription.

Because the aforementioned phenotypic changes were assayed in vegetative Oxytricha cells, we asked whether MTA1 may play roles outside of this developmental state. MTA1 transcript levels are markedly upregulated in the sexual cycle, as assayed by poly(A). RNA-seq (FIG. 7C). Strikingly, mta1 mutants fail to complete the sexual cycle when induced to mate and display complete lethality (FIG. 7D). Our data do not exclude the possibility that m6A RNA methylation, in addition to 6 mA DNA methylation, is also impacted by MTA1 loss during development. Further studies would clarify the role of MTA1 in these pathways.

### Example 10

### Discussion

The present disclosure has identified MTA1c as a conserved, hitherto undescribed 6 mA methyltransferase. It consists of two MT-A70 proteins (MTA1/MTA9) and two homeobox-like proteins (p1/p2). The composition of MTA1c provides immediate insights into how it specifically methylates DNA (FIG. 7F). MTA1 likely mediates transfer of the methyl group from SAM to the acceptor adenine moiety, given that it contains conserved amino acid residues implicated in catalysis and SAM binding (FIG. 10E). Indeed, we show that these residues are necessary for its activity (FIG. 2E). While MTA1 constitutes the catalytic center, it lacks a CCCH-type zinc finger domain that is necessary for RNA binding in the canonical m6A methyltransferase METTL3. Instead, nucleic acid binding is likely assumed by the homeobox-like domains in p1 and p2, which are known to specifically engage dsDNA through helix-turn-helix motifs.

The observation that MTA1c is more active in the presence of pre-methylated DNA templates is reminiscent of the CpG methyltransferase DNMT1. Yet, MTA1c and DNMT1 exhibit distinct protein domain architectures. Further biochemical studies are required to elucidate the molecular basis of this property. A distinct MT-A70 protein, named TAMT-1, was recently reported to act as a 6 mA methyltransferase in Tetrahymena, (Luo et al., 2018), suggesting that multiple enzymes mediate 6 mA deposition. It remains to be determined how MTA1c and TAMT-1 collectively mediate DNA methylation at various developmental stages, and whether cross-talk occurs between these enzymes.

In addition to identifying the ciliate 6 mA methyltransferase, we investigated the function of 6 mA in vitro by building epigenetically defined chromosomes. We show that 6 mA directly disfavors nucleosome occupancy in a local, quantitative manner, independent of DNA sequence (FIG. 7E). Our experiments do not reveal exactly how 6 mA disfavors nucleosome occupancy. Early studies suggest that 6 mA destabilizes dA:dT base pairing, leading to a decrease in the melting temperature of DNA (Engel and von Hippel, 1978). Whether this or some other property of 6 mA contributes to lowered nucleosome stability awaits further investigation.

Intriguingly, nucleosome organization exhibits only subtle changes after genome-wide loss of 6 mA (FIG. 7E). Only a small set of genes (<10%) is transcriptionally dysregulated. It is possible that residual 6 mA in mta1 mutants could mask relevant phenotypes. Nonetheless, our results caution against interpreting 6 mA function solely based on correlation with genomic elements. We also find that 6 mA intrinsically disfavors nucleosomes in vitro, but—crucially—this effect can be overridden by distinct factors in vitro and in vivo. We propose that phased nucleosome arrays are first established in vivo, which then restrict MTA1-mediated methylation to linker regions due to steric hindrance. This in turn decreases the fuzziness of flanking nucleosomes, reinforcing chromatin organization. Therefore, 6 mA tunes nucleosome organization in vivo. Our data do not support the hypothesis that nucleosome phasing is established by predeposited 6 mA.

More broadly, our work showcases the utility of Oxytricha chromosomes for advancing chromatin biology. By extending current technologies (Muller et al., 2016), it should be feasible to introduce both modified nucleosomes and DNA methylation in a site-specific manner on full-length chromosomes. Such “designer” chromosomes will serve as powerful tools for studying DNA-templated processes such as transcription within a fully native DNA environment.

### REFERENCES

The following references, to the extent that they provide exemplary procedural or other details supplementary to those set forth herein, are specifically incorporated herein by reference.
Altschul, S. F., Madden, T. L., Schaffer, A. A., Zhang, J., Zhang, Z., Miller, W., and Lipman, D. J. (1997). Gapped BLAST and PSI-BLAST: a new generation of protein database search programs. Nucleic Acids Res. 25, 3389-3402.
Ammermann, D., Steinbruck, G., Baur, R., and Wohlert, H. (1981). Methylated bases in the DNA of the ciliate Stylonychia mytilus. Eur. J. Cell Biol. 24, 154-156.
An, W., and Roeder, R. G. (2004). Reconstitution and transcriptional analysis of chromatin in vitro. Methods Enzymol. 377, 460-474.
Batut, P., Dobin, A., Plessy, C., Carninci, P., and Gingeras, T. R. (2013). Highfidelity promoter profiling reveals widespread alternative promoter usage and transposon-driven developmental gene expression. Genome Res. 23, 169-180.
Beh, L. Y., Muller, M. M., Muir, T. W., Kaplan, N., and Landweber, L. F. (2015). DNA-guided establishment of nucleosome patterns within coding regions of a eukaryotic genome. Genome Res. 25, 1727-1738.
Beh et al., Identification of a DNA N6-Adenine Methyltransferase Complex and Its Impact on Chromatin Organization, Cell (2019), https://doi.org/10.1016/j.ce11.2019.04.028.
Bern, M., Kil, Y. J., and Becker, C. (2012). Byonic: Advanced Peptide and Protein Identification Software. Curr. Protoc. Bioinformatics. 13, 13.20.
Blankenberg, D., Von Kuster, G., Coraor, N., Ananda, G., Lazarus, R., Mangan, M., Nekrutenko, A., and Taylor, J. (2010). Galaxy: a web-based genome analysis tool for experimentalists. Curr. Protoc. Mol. Biol. 19, 19.10.1-19.10.21.
Bracht, J. R., Fang, W., Goldman, A. D., Dolzhenko, E., Stein, E. M., and Landweber, L. F. (2013). Genomes on the edge: programmed genome instability in ciliates. Cell 152, 406-416.
Bromberg, S., Pratt, K., and Hattman, S. (1982). Sequence specificity of DNA adenine methylase in the protozoan Tetrahymena thermophila. J. Bacteriol. 150, 993-996.
Brownell, J. E., Zhou, J., Ranalli, T., Kobayashi, R., Edmondson, D. G., Roth, S. Y., and Allis, C. D. (1996). Tetrahymena histone acetyltransferase A: a homolog to yeast Gcn5p linking histone acetylation to gene activation. Cell 84, 843-851.
Cassidy-Hanley, D. M. (2012). Tetrahymena in the Laboratory: Strain Resources, Methods for Culture, Maintenance, and Storage. Methods Cell Biol. 109, 237-276.
Chen, X., Bracht, J. R., Goldman, A. D., Dolzhenko, E., Clay, D. M., Swart, E. C., Perlman, D. H., Doak, T. G., Stuart, A., Amemiya, C. T., et al. (2014). The architecture of a scrambled genome reveals massive levels of genomic rearrangement during development. Cell 158, 1187-1198.
Clapier, C. R., and Cairns, B. R. (2009). The biology of chromatin remodeling complexes. Annu. Rev. Biochem. 78, 273-304.
Cummings, D. J., Tait, A., and Goddard, J. M. (1974). Methylated bases in DNA from Paramecium aurelia. Biochim. Biophys. Acta 374, 1-11.
Debelouchina, G. T., Gerecht, K., and Muir, T. W. (2017). Ubiquitin utilizes an acidic surface patch to alter chromatin structure. Nat. Chem. Biol. 13, 105-110.
Eisen, J. A., Coyne, R. S., Wu, M., Wu, D., Thiagarajan, M., Wortman, J. R., Badger, J. H., Ren, Q., Amedeo, P., Jones, K. M., et al. (2006). Macronuclear genome sequence of the ciliate Tetrahymena thermophila, a model eukaryote. PLoS Biol. 4, e286.
Eng, J. K., McCormack, A. L., and Yates, J. R. (1994). An approach to correlate tandem mass spectral data of peptides with amino acid sequences in a protein database. J. Am. Soc. Mass Spectrom. 5, 976-989.
Engel, J. D., and von Hippel, P. H. (1978). Effects of methylation on the stability of nucleic acid conformations. Studies at the polymer level. J. Biol. Chem. 253, 927-934.
Fang, W., Wang, X., Bracht, J. R., Nowacki, M., and Landweber, L. F. (2012). Piwi-interacting RNAs protect DNA against loss during Oxytricha genome rearrangement. Cell 151, 1243-1255.
Finn, R. D., Clements, J., Arndt, W., Miller, B. L., Wheeler, T. J., Schreiber, F., Bateman, A., and Eddy, S. R. (2015). HMMER web server 2015 update. Nucleic Acids Res. 43 (W1), W30-W38.
Fioravanti, A., Fumeaux, C., Mohapatra, S. S., Bompard, C., Brilli, M., Frandi, A., Castric, V., Villeret, V., Viollier, P. H. P., and Biondi, E. G. (2013). DNA binding of the cell cycle transcriptional regulator GcrA depends on N6-adenosine methylation in Caulobacter crescentus and other Alphaproteobacteria. PloS Genet. 9, e1003541.
Fu, Y., Luo, G.-Z., Chen, K., Deng, X., Yu, M., Han, D., Hao, Z., Liu, J., Lu, X., Dore, L. C., et al. (2015). N6-methyldeoxyadenosine marks active transcription start sites in Chlamydomonas. Cell 161, 879-892.
Fyodorov, D. V., and Kadonaga, J. T. (2003). Chromatin assembly in vitro with purified recombinant ACF and NAP-1. Methods Enzymol. 371, 499-515.
Giardine, B., Riemer, C., Hardison, R. C., Burhans, R., Elnitski, L., Shah, P., Zhang, Y., Blankenberg, D., Albert, I., Taylor, J., et al. (2005). Galaxy: a platform for interactive large-scale genome analysis. Genome Res. 15, 1451-1455.
Goecks, J., Nekrutenko, A., and Taylor, J.; Galaxy Team (2010). Galaxy: a comprehensive approach for supporting accessible, reproducible, and transparent computational research in the life sciences. Genome Biol. 11, R86.
Gorovsky, M A., Hattman, S., and Pleger, G. L. (1973). (6 N)methyl adenine in the nuclear DNA of a eucaryote, Tetrahymena pyriformis. J. Cell Biol. 56, 697-701.
Gottschling, D. E., and Cech, T. R. (1984). Chromatin structure of the molecular ends of Oxytricha macronuclear DNA: phased nucleosomes and a telomeric complex. Cell 38, 501-510.
Greer, E. L., Blanco, M. A., Gu, L., Sendinc, E., Liu, J., Aristizabal-Corrales, D., Hsu, C.-H., Aravind, L., He, C., and Shi, Y. (2015). DNA Methylation on N6-Adenine in C. elegans. Cell 161, 868-878.
Haberle, V., Forrest, A. R. R., Hayashizaki, Y., Carninci, P., and Lenhard, B. (2015). CAGEr: precise TSS data retrieval and high-resolution promoterome mining for integrative analyses. Nucleic Acids Res. 43, e51.
Hattman, S., Kenny, C., Berger, L, and Pratt, K. (1978). Comparative study of DNA methylation in three unicellular eucaryotes. J. Bacteriol. 135, 1156-1157.
Horton, J. R., Liebert, K., Bekes, M., Jeltsch, A., and Cheng, X. (2006). Structure and substrate recognition of the Escherichia coli DNA adenine methyltransferase. J. Mol. Biol. 358, 559-570.
Huang, Y., Niu, B., Gao, Y., Fu, L., and U, W. (2010). CD-HIT Suite: a web server for clustering and comparing biological sequences. Bioinformatics 26, 680-682.
Huang, J., Dong, X., Gong, Z., Qin, L-Y., Yang, S., Zhu, Y.-L, Wang, X., Zhang, D., Zou, T., Yin, P., et al. (2019). Solution structure of the RNA recognition domain of METTL3-METTL14 N6-methyladenosine methyltransferase. Protein Cell 10, 272-284.
Huff, J. T., and Zilberman, D. (2014). Dnmt1-independent CG methylation contributes to nucleosome positioning in diverse eukaryotes. Cell 156, 1286-1297.
Ito, T., Bulger, M., Pazin, M. J., Kobayashi, R., and Kadonaga, J. T. (1997). ACF, an ISWI-containing and ATP-utilizing chromatin assembly and remodeling factor. Cell 90, 145-155.
Iyer, L M., Zhang, D., and Aravind, L (2016). Adenine methylation in eukaryotes: Apprehending the complex evolutionary history and functional potential of an epigenetic modification. BioEssays 38, 27-40.
Karrer, K. M., and VanNuland, T. A. (1999). Nucleosome positioning is independent of histone H1 in vivo. J. Biol. Chem. 274, 33020-33024.
Katoh, K., Rozewicki, J., and Yamada, K. D. (2017). MAFFT online service: multiple sequence alignment, interactive sequence choice and visualization. Brief. Bioinform.
Kharchenko, P. V., Tolstorukov, M. Y., and Park, P. J. (2008). Design and analysis of ChIP-seq experiments for DNA-binding proteins. Nat. Biotechnol. 26, 1351-1359.
Khurana, J. S., Wang, X., Chen, X., Perlman, D. H., and Landweber, L F. (2014). Transcription-independent functions of an RNA polymerase II subunit, Rpb2, during genome rearrangement in the ciliate, Oxytricha trifallax. Genetics 197, 839-849.
Khurana, J. S., Clay, D. M., Moreira, S., Wang, X., and Landweber, L. F. (2018). Small RNA-mediated regulation of DNA dosage in the ciliate Oxytricha. RNA 24, 18-29.
Koziol, M. J., Bradshaw, C. R., Allen, G. E., Costa, A. S. H., Frezza, C., and Gurdon, J. B. (2016). Identification of methylated deoxyadenosines in vertebrates reveals diversity in DNA modifications. Nat. Struct. Mol. Biol. 23, 24-30.
Kuraku, S., Zmasek, C. M., Nishimura, O., and Katoh, K. (2013). aLeaves facilitates on-demand exploration of metazoan gene family trees on MAFFT sequence alignment server with enhanced interactivity. Nucleic Acids Res. 41, W22-W28.
Lai, W. K. M., and Pugh, B. F. (2017). Understanding nucleosome dynamics and their links to gene expression and DNA replication. Nat. Rev. Mol. Cell Biol. 18, 548-562.
Langmead, B., and Salzberg, S. L. (2012). Fast gapped-read alignment with Bowtie 2. Nat. Methods 9, 357-359.
Laughlin, T. J., Henry, J. M., Phares, E. F., Long, M. V., and Olins, D. E. (1983). Methods for the Large-Scale Cultivation of an Oxytricha (Ciliophora: Hypotrichide). J. Protozool. 30, 63-64.
Lauth, M. R., Spear, B. B., Neumann, J., and Prescott, D. M. (1976). DNA of ciliated protozoa: DNA sequence diminution during macronuclear development of Oxytricha. Cell 7, 67-74.
Lawn, R. M., Neumann, J. M., Herrick, G., and Prescott, D. M. (1978). The genesize DNA molecules in Oxytricha. Cold Spring Harb. Symp. Quant. Biol. 42, 483-492.
Liang, Z., Shen, L., Cui, X., Bao, S., Geng, Y., Yu, G., Liang, F., Xie, S., Lu, T., Gu, X., and Yu, H. (2018). DNA N6-Adenine Methylation in Arabidopsis thaliana. Dev. Cell 45, 406-416.
Lieleg, C., Ketterer, P., Nuebler, J., Ludwigsen, J., Gerland, U., Dietz, H., Mueller-Planitz, F., and Korber, P. (2015). Nucleosome spacing generated by ISWI and CHD1 remodelers is constant regardless of nucleosome density. Mol. Cell. Biol. 35, 1588-1605.
Liu, Y., Tavema, S. D., Muratore, T. L, Shabanowitz, J., Hunt, D. F., and Allis, C. D. (2007). RNAi-dependent H3K27 methylation is required for heterochromatin formation and DNA elimination in Tetrahymena. Genes Dev. 21, 1530-1545.
Liu, J., Yue, Y., Han, D., Wang, X., Fu, Y., Zhang, L, Jia, G., Yu, M., Lu, Z., Deng, X., et al. (2014). A METTL3-METTL14 complex mediates mammalian nuclear RNA N6-adenosine methylation. Nat. Chem. Biol. 10, 93-95.
Liu, J., Zhu, Y., Luo, G.-Z., Wang, X., Yue, Y., Wang, X., Zong, X., Chen, K., Yin, H., Fu, Y., et al. (2016). Abundant DNA 6 mA methylation during early embryogenesis of zebrafish and pig. Nat. Commun. 7,13052.
Livak, K. J., and Schmittgen, T. D. (2001). Analysis of relative gene expression data using real-time quantitative PCR and the 2(−Delta Delta C(T)) Method. Methods 25, 402-408.
Lugar, K., Rcchatcincr, T. J., and Richmond, T. J. (1900). Proparation of nucleosome core particle from recombinant histones. Methods Enzymol. 304, 3-19.
Luo, G.-Z., Blanco, M. A., Greer, E. L., He, C., and Shi, Y. (2015). DNA N(6)-methyladenine: a new epigenetic mark in eukaryotes? Nat. Rev. Mol. Cell Biol. 16, 705-710.
Luo, G.-Z., Hao, Z., Luo, L, Shen, M., Sparvoli, D., Zheng, Y., Zhang, Z., Weng, X., Chen, K., Cui, Q., et al. (2018). N6-methyldeoxyadenosine directs nucleosome positioning in Tetrahymena DNA. Genome Biol. 19, 200.
Mavrich, T. N., loshikhes, I. P., Venters, B. J., Jiang, C., Tomsho, L P., Qi, J., Schuster, S. C., Albert, I., and Pugh, B. F. (2008). A barrier nucleosome model for statistical positioning of nucleosomes throughout the yeast genome. Genome Res. 18, 1073-1083.
Miao, W., Xiong, J., Bowen, J., Wang, W., Liu, Y., Braguinets, O., Grigull, J., Pearlman, R. E., Orias, E., and Gorovsky, M A. (2009). Microarray analyses of gene expression during the Tetrahymena thermophila life cycle. PLoS ONE 4,e4429.
Miller, M A., Pfeiffer, W., and Schwartz, T. (2010). Creating the CIPRES Science Gateway for Inference of Large Phylogenetic Trees. Proceedings of the Gateway Computing Environments Workshop (GCE), 14 Nov. 2010, New Orleans, La. pp. 1-8.
Mondo, S. J., Dannebaum, R. O., Kuo, R. C., Louie, K. B., Bewick, A. J., LaButti, K., Haridas, S., Kuo, A., Salamov, A., Ahrendt, S. R., et al. (2017). Widespread adenine N6-methylation of active genes in fungi. Nat. Genet. 49, 964-968.
Mortazavi, A., Williams, B. A., McCue, K., Schaeffer, L., and Wold, B. (2008). Mapping and quantifying mammalian transcriptomes by RNA-Seq. Nat. Methods 5, 621-628.
M. M., Fierz, B., Bittova, L., Liszczak, G., and Muir, T. W. (2016). A two-state activation mechanism controls the histone methyltransferase Suv39h1. Nat. Chem. Biol. 12, 188-193.
Murray, i.A., Morgan, R. D., Luyten, Y., Fomenkov, A., Correa, i.R., jr., Dai, Allaw, M. B., Zhang, X., Cheng, X., and Roberts, R. J. (2018). The non-specific adenine DNA methyltransferase M.EcoGll. Nucleic Acids Res. 46, 840-848.
Nesvizhskii, A. I., Keller, A., Kolker, E., and Aebersold, R. (2003). A statistical model for identifying proteins by tandem mass spectrometry. Anal. Chem. 75, 4646-4658.
Nowacki, M., Vijayan, V., Zhou, Y., Schotanus, K., Doak, T. G., and Landweber, L. F. (2008). RNA-mediated epigenetic programming of a genome-rearrangement pathway. Nature 451, 153-158.
Pendleton, K. E., Chen, B., Liu, K., Hunter, 0. V., Xie, Y., Tu, B. P., and Conrad, N. K. (2017). The U6 snRNA m6A Methyltransferase METTL16 Regulates SAM Synthetase Intron Retention. Cell 169, 824-835.
Pratt, K., and Hattman, S. (1981). Deoxyribonucleic acid methylation and chromatin organization in Tetrahymena thermophila. Mol. Cell. Biol. 1, 600-608.
Prescott, D. M. (1994). The DNA of ciliated protozoa. Microbiol. Rev. 58, 233-267.
Rae, P. M., and Spear, B. B. (1978). Macronuclear DNA of the hypotrichous ciliate Oxytricha fallax. Proc. Natl. Acad. Sci. USA 75, 4992-4996.
Rappsilber, J., Mann, M., and Ishihama, Y. (2007). Protocol for micro-purification, enrichment, pre-fractionation and storage of peptides for proteomics using StageTips. Nat. Protoc. 2, 1896-1906.
Schaffer, A. A., Aravind, L, Madden, T. L, Shavirin, S., Spouge, J. L., Wolf, Y. I., Koonin, E. V., and Altschul, S. F. (2001). Improving the accuracy of PSI-BLAST protein database searches with composition-based statistics and other refinements. Nucleic Acids Res. 29, 2994-3005.
Schiffers, S., Ebert, C., Rahimoff, R., Kosmatchev, O., Steinbacher, J., Bohne, A.-V., Spada, F., Michalakis, S., Nickelsen, J., Mailer, M., and Carell, T. (2017). Quantitative LC-MS Provides No Evidence for m6 dA or m4 dC in the Genome of Mouse Embryonic Stem Cells and Tissues. Angew. Chem. Int. Ed. Engl. 56, 11268-11271.
Sledz, P., and Jinek, M. (2016). Structural insights into the molecular mechanism of the m(6)A writer complex. eLife 5. Published online Sep. 14, 2016. https://doi.org/10.7554/eLife.18434.
Strahl, B. D., Ohba, R., Cook, R. G., and Allis, C. D. (1999). Methylation of histone H3 at lysine 4 is highly conserved and correlates with transcriptionally active nuclei in Tetrahymena. Proc. Natl. Acad. Sci. USA 96, 14967-14972.
Struhl, K., and Segal, E. (2013). Determinants of nucleosome positioning. Nat. Struct. Mol. Biol. 20, 267-273.
Swart, E. G., Bracht, J. R., Magrini, V., Minx, P., Chen, X., Zhou, Y., Khurana, J. S., Goldman, A D., Nowacki, M., Schotanus, K., et al. (2013). The Oxytricha trifallax macronuclear genome: a complex eukaryotic genome with 16,000 tiny chromosomes. PLoS Biol. 11, e1001473.
Tavema, S. D., Coyne, R. S., and Allis, C. D. (2002). Methylation of histone h3 at lysine 9 targets programmed DNA elimination in tetrahymena. Cell 110, 701-711.
Wada, R. K., and Spear, B. B. (1980). Nucleosomal organization of macronuclear chromatin in Oxytricha fallax. Cell Differ. 9, 261-268.
Wang, P., Doxtader, K. A., and Nam, Y. (2016a). Structural Basis for Cooperative Function of Mettl3 and Mettl14 Methyltransferases. Mol. Cell 63, 306-317.
Wang, X., Feng, J., Xue, Y., Guan, Z., Zhang, D., Liu, Z., Gong, Z., Wang, Q., Huang, J., Tang, C., et al. (2016b). Structural basis of N(6)-adenosine methylation by the METTL3-METTL14 complex. Nature 534, 575-578.
Wang, Y., Chen, X., Sheng, Y., Liu, Y., and Gao, S. (2017). N6-adenine DNA methylation is associated with the linker DNA of H2A2-containing well-positioned nucleosomes in Pol II-transcribed genes in Tetrahymena. Nucleic Acids Res. 45, 11594-11606.
Warda, A. S., Kretschmer, J., Heckert, P., Lenz, C., Urlaub, H., Hobartner, C., Sloan, K. E., and Bohnsack, M. T. (2017). Human METTL16 is a N6-methyladenosine (m6A) methyltransferase that targets pre-mRNAs and various noncoding RNAs. EMBO Rep. 18, 2004-2014.
Wei, Y., Mizzen, C. A., Cook, R. G., Gorovsky, M. A., and Allis, C. D. (1998). Phosphorylation of histone H3 at serine 10 is correlated with chromosome condensation during mitosis and meiosis in Tetrahymena. Proc. Natl. Acad. Sci. USA 95, 7480-7484.
Wu, T. P., Wang, T., Seetin, M. G., Lai, Y., Zhu, S., Lin, K., Liu, Y., Byrum, S. D., Mackintosh, S. G., Zhong, M., et al. (2016). DNA methylation on N(6)-adenine in mammalian embryonic stem cells. Nature 532, 329-333.
Xiao, R., and Moore, D. D. (2011). Dam IP: Using Mutant DNA Adenine Methyltransferase to Study DNA-Protein Interactions In Vivo. Curr. Protoc. Mol. Biol. 21. https://doi.org/10.1002/0471142727.mb2121s94.
Xiao, C.-L., Zhu, S., He, M., Chen, D., Zhang, Q., Chen, Y., Yu, G., Liu, J., Xie, S.-Q., Luo, F., et al. (2018). N6-Methyladenine DNA Modification in the Human Genome. Mol. Cell 71, 306-318.
Xiong, J., Lu, X., Lu, Y., Zeng, H., Yuan, D., Feng, L, Chang, Y., Bowen, J., Gorovsky, M., Fu, C., and Miao, W. (2011). Tetrahymena Gene Expression Database (TGED): a resource of microarray data and co-expression analyses for Tetrahymena. Sci. China Life Sci. 54, 65-67.
Xiong, J., Lu, X., Zhou, Z., Chang, Y., Yuan, D., Tian, M., Zhou, Z., Wang, L, Fu, C., Orias, E., and Miao, W. (2012). Transcriptome analysis of the model protozoan, Tetrahymena thermophila, using Deep RNA sequencing. PLoS ONE 7, e30630.
Yao, B., Cheng, Y., Wang, Z., Li, Y., Chen, L, Huang, L., Zhang, W., Chen, D., Wu, H., Tang, B., and Jin, P. (2017). DNA N6-methyladenine is dynamically regulated in the mouse brain following environmental stress. Nat. Commun. 8, 1122.
Yao, B., Li, Y., Wang, Z., Chen, L., Poidevin, M., Zhang, C., Lin, L, Wang, F., Bao, H., Jiao, B., et al. (2018). Active N 6-Methyladenine Demethylation by DMAD Regulates Gene Expression by Coordinating with Polycomb Protein in Neurons. Mol. Cell 71, 848-857.
Yerlici, V. T., and Landweber, L. F. (2014). Programmed Genome Rearrangements in the Ciliate Oxytricha. Microbiol. Spectr. 2. Published online December 2014. 10.1128/microbiolspec.MDNA3-0025-2014.
Zhang, Z., and Pugh, B. F. (2011). High-resolution genome-wide mapping of the primary structure of chromatin. Cell 144, 175-186.
Zhang, G., Huang, H., Liu, D., Cheng, Y., Liu, X., Zhang, W., Yin, R., Zhang, D., Zhang, P., Liu, J., et al. (2015). N6-methyladenine DNA modification in Drosophila. Cell 161, 893-906.
Zhou, C., Wang, C., Liu, H., Zhou, Q., Liu, Q., Guo, Y., Peng, T., Song, J., Zhang, J.,
Chen, L., et al. (2018). Identification and analysis of adenine N6-methylation sites in the rice genome. Nat. Plants 4, 554-563.

The embodiments described in this disclosure can be combined in various ways. Any aspect or feature that is described for one embodiment can be incorporated into any other embodiment mentioned in this disclosure. While various novel features of the inventive principles have been shown, described and pointed out as applied to particular embodiments thereof, it should be understood that various omissions and substitutions and changes may be made by those skilled in the art without departing from the spirit of this disclosure. Those skilled in the art will appreciate that the inventive principles can be practiced in other than the described embodiments, which are presented for purposes of illustration and not limitation.

What is claimed is:

1. A method of treating or ameliorating the effects of a disease characterized by an abnormal level of m6dA in a subject, comprising administering to the subject an amount of MTA1c or any components thereof effective to modulate m6dA levels in the subject.

2. The method according to claim 1, wherein the modulation comprises restoring m6dA levels to normal or near-normal ranges in the subject.

3. The method according to claim 1, wherein the disease is a cancer.

4. The method according to claim 3, wherein the cancer is gastric cancer or liver cancer.

5. The method according to claim 4, further comprising administering to the subject one or more of anti-gastric cancer and anti-liver cancer drugs.

6. The method according to claim 1, furthering comprising co-administering to the subject an epigenetic agent.

7. The method according to claim 6, wherein the epigenetic agent is selected from the group consisting of methylation inhibiting drugs, Bromodomain inhibitors, histone acetylase (HAT) inhibitors, protein methyltransferase inhibitors, histone methylation inhibitors, histone deacetlyase (HDAC) inhibitors, histone acetylases, histone deacetlyases, and combinations thereof.

8. A pharmaceutical composition comprising MTA1c or any components thereof that is effective to modulate m6dA levels in a subject in need thereof and a pharmaceutically acceptable carrier, diluent, adjuvant or vehicle.

9. A method of modifying a nucleic acid from a cell, the cell derived from a multicellular eukaryote, comprising the steps of:
(a) obtaining the nucleic acid from the cell; and
(b) contacting the nucleic acid with MTA1c or any components thereof under conditions effective to methylate the nucleic acid.

10. The method according to claim 9, wherein the methylated nucleic acid is effective to modulate nucleosome organization and transcription.

11. The method according to claim 9, wherein the modification is a DNA N6-adenine methylation.

12. The method according to claim 11, wherein the DNA N6-adenine methylation is one or more of dimethylated AT (5′-A*T-3′/3′-TA*-5′), dimethylated TA (5′-TA*-3′/3′-A*T-5′), dimethylated AA (5′-A*A*-3′/3′-TT-5′), methylated AT (5′-A*T-3′/3′-TA-5′), methylated AA (5′-A*A-3′/3′-TT-5′), methylated AC (5′-A*C-3′/3′-TG-5′), methylated AG (5′-A*G-3′/3′-TC-5′), methylated TA (5′-TA*-3′/3′-AT-5′), methylated AA (5′-AA*-3′/3′-TT-5′), methylated CA (5′-CA*-3′/3′-GT-5′), and methylated GA (5′-GA*-3′/3′-CT-5′).

13. The method according to claim 9, wherein the MTA1c or any components thereof comprises a mutation effective to abrogate dimethylation of the nucleic acid.

14. The method according to claim 13, wherein the mutation comprises loss of a C-terminal methyltransferase domain.

15. The method according to claim 9, wherein the MTA1c or any components thereof is obtained from ciliates, algae, or basal fungi.

16. The method according to claim 9, wherein the MTA1c or any components thereof is obtained from Oxytricha or Tetrahymena.

17. A cell line obtained from a multicellular eukaryote comprising a nucleic acid encoding MTA1c or any components thereof and/or an MTA1c protein complex or any components thereof.

18. The eukaryotic cell according to claim 17, wherein the nucleic acid encoding MTA1c or any components thereof is operably linked to a recombinant expression vector.

19. A method of identifying protein binding sites on DNA comprising the steps of:
(a) providing DNA;
(b) contacting the DNA with MTA1c or any components thereof under conditions effective to methylate the DNA;
(c) contacting the DNA with one or more proteins;
(d) contacting the DNA with an enzyme effective to hydrolyze the DNA in positions where no protein binding occurs;
(e) removing the DNA bound protein; and
(f) isolating and sequencing the DNA fragments.

20. The method according to claim 19, wherein the one or more proteins comprise histone octamers.
